(19) United States
(12) Patent Application Publication
(10) Pub. No.: US 2025/0043290 A1
(43) Pub. Date: Feb. 6, 2025

(54) RNA SEQUENCES THAT INDUCE FLUORESCENCE OF SMALL MOLECULE FLUOROPHORES, MOLECULAR COMPLEXES, SENSORS, AND METHODS OF USE THEREOF

(71) Applicant: CORNELL UNIVERSITY, Ithaca, NY (US)

(72) Inventors: Samie R. JAFFREY, New York, NY (US); Sourav Kumar DEY, New York, NY (US)

(21) Appl. No.: 18/711,287

(22) PCT Filed: Nov. 23, 2022
(86) PCT No.: PCT/US2022/050921

§ 371 Date: May 17, 2024

Related U.S. Application Data
(60) Provisional application No. 63/282,347, filed on Nov. 23, 2021.

(51) Int. Cl. C12N 15/115; C12Q 1/6816; G01N 33/50; G01N 33/58

(52) U.S. Cl. CPC C12N 15/115; C12Q 1/6816; G01N 33/5038; G01N 33/582; C12N 2310/16; C12N 2310/531

(57) ABSTRACT

The present disclosure relates to “Squash” and “Beetroot” nucleic acid aptamer molecules comprising certain nucleotide sequences and variations thereof. Also disclosed are molecular complexes comprising a fluorophore molecule and a nucleic acid aptamer molecule disclosed herein, isolated host cells comprising the molecular complexes, kits comprising a fluorophore and a nucleic acid aptamer, constructed DNA molecules encoding a nucleic acid aptamer molecule, expression systems, transgenic host cells, methods of detecting target molecules, RNA-based metabolite sensors, RNA-based ratiometric metabolite sensors, systems comprising RNA-based ratiometric metabolite sensors, and methods of generating a randomized aptamer library.

This application claims benefit of U.S. Provisional Application Ser. No. 63/282,347, filed Nov. 23, 2021, which is hereby incorporated by reference in its entirety.

This invention was made with government support under RO1 NS064516 and R35 NS 111631 awarded by National Institute of Neurological Disorders and Stroke. The government has certain rights in the invention.

### FIELD

The present disclosure relates to RNA sequences that induce fluorescence of small molecule fluorophores, molecular complexes, sensors, and methods of use thereof.

### BACKGROUND

Genetically encoded metabolite sensors reveal dynamic changes in metabolite concentrations in single cells in real time. Most sensors comprise fluorescent proteins flanking a metabolite-binding domain (Sanford and Palmer, “Recent Advances in Development of Genetically Encoded Fluorescent Sensors,” in Methods in Enzymology 589:1-49 (2017)). Metabolite binding induces conformational changes that reposition the fluorescent proteins, thus altering the Förster resonance energy transfer (FRET) between these proteins (Lindenburg and Merkx, “Engineering Genetically Encoded FRET Sensors,” Sensors 14:11691-11713 (2014)). Metabolite sensors rely on ratiometric fluorescence, in which fluorescence is measured at two excitation/emission wavelengths, and the ratio is used to establish metabolite levels. Ratiometric probes are thus ‘self-calibrating,’ which allows them to produce signals that are independent of probe concentration. This overcomes cell-to-cell variability in sensor expression levels, and also resolves the problem of different fluorescence levels in thin versus thick parts of a cell (Palmer et al., “Design and Application of Genetically Encoded Biosensors,” Trends Biotechnol. 29:144-152 (2011)). The lack of proteins that undergo suitable metabolite-induced conformational changes limits the overall number of sensors available for researchers (Lechner et al., “Strategies for Designing Non-Natural Enzymes and Binders,” Curr. Opin. Chem. Biol. 47:67-76 (2018)).

In addition to protein-based sensors, sensors can be composed of RNA (Paige et al., “Fluorescence Imaging of Cellular Metabolites with RNA,” Science 335:1194 (2012) and Kellenberger et al., “Hammond MC RNA-Based Fluorescent Biosensors for Live Cell Imaging of Second Messengers Cyclic di-GMP and cyclic AMP-GMP,” J. Am. Chem. Soc. 135, 4906-4909 (2013)). RNA-based sensors utilize a metabolite-binding RNA aptamer connected to a fluorogenic aptamer, such as Broccoli, Spinach, or Corn (Kellenberger et al., “Hammond MC RNA-Based Fluorescent Biosensors for Live Cell Imaging of Second Messengers Cyclic di-GMP and cyclic AMP-GMP,” J. Am. Chem. Soc. 135, 4906-4909 (2013); Sun et al., “Intracellular Imaging with Genetically Encoded RNA-based Molecular Sensors,” Nanomaterials 9:233 (2019); Kim & Jaffrey, “A Fluorogenic RNA-Based Sensor Activated by Metabolite-Induced RNA Dimerization,” Cell Chem. Biol. 26:1725-1731 (2019); and Li et al., “Imaging Intracellular S-Adenosyl Methionine Dynamics in Live Mammalian Cells with a Genetically Encoded Red Fluorescent RNA-Based Sensor,” J. Am. Chem. Soc. 142:14117-14124 (2020)). Metabolite binding allosterically induces folding of the fluorogenic RNA aptamer, allowing it to bind and activate the fluorescence of its otherwise non-fluorescent cognate fluorophore (Paige et al., “Fluorescence Imaging of Cellular Metabolites with RNA,” Science 335:1194 (2012) and Kellenberger et al., “Hammond MC RNA-Based Fluorescent Biosensors for Live Cell Imaging of Second Messengers Cyclic di-GMP and cyclic AMP-GMP,” J. Am. Chem. Soc. 135:4906-4909 (2013)). RNA-based sensors have mostly been used in bacteria where the RNA is stable and can thus accumulate to sufficient concentrations for fluorescence detection (Sun et al., “Intracellular Imaging with Genetically Encoded RNA-Based Molecular Sensors,” Nanomaterials 9:233 (2019) and Ortega et al., “A Synthetic RNA-Based Biosensor for Fructose-1,6-Bisphosphate that Reports Glycolytic Flux,” Cell Chem. Biol. 288(11):1554-1568 (2021)). RNA-based sensors have recently been used in mammalian cells as a result of an expression system that allow RNA-based sensors to be expressed as highly stable circular RNA (Litke and Jaffrey, “Highly Efficient Expression of Circular RNA Aptamers in Cells Using Autocatalytic Transcripts,” Nat. Biotechnol. 37:667-675 (2019)). However, ratiometric sensors have not yet been developed for mammalian cells, which limit the usefulness of RNA-based sensors.

The present disclosure is directed to overcoming these and other deficiencies in the art.

### SUMMARY

One aspect of the present disclosure is directed to a Squash nucleic acid aptamer molecule comprising the nucleotide sequence of:
(i) GGC UAC AAG GUG AGC CCA AUA AUA CGG UUU GGG UUA GGA UAG GAA GUA GAG CCG UAA ACU CUC UAA GCG GUA GUC (SEQ ID NO: 1);
(ii) GGC UAC AAG GUG AGC CCA AUA AUA GGG UUU GGG UUA GGA UAG GAA GUA GAG CCC UAA ACU CUC UAA GCG GUA GUC (SEQ ID NO: 2);
(iii) GGC UAC AAG GUG AGC CCA AUA AUA NNN UUU GGG UUA GGA UAG GAA GUA GAG NNN UAA ACU CUC UAA GCG GUA GUC (SEQ ID NO: 3), where N at positions 25 and 54 are complementary to each other, N at positions 26 and 53 are complementary to each other, and N at positions 27 and 52 are complementary to each other, optionally where the complementary positions base pair with each other; or
(iv) GGC UAC AAG GUG NNN NNN NUA AUA NNN UNN NNN NNA GGA UAG GAN NNN NNN NNN UAA ANN NNN NAA GCG GUA GUC (SEQ ID NO: 4),
   where N at positions 25 and 54 are complementary to each other,
   N at positions 26 and 53 are complementary to each other,
   N at positions 27 and 52 are complementary to each other,
   N at positions 13 and 35 are complementary to each other,
   N at positions 14 and 34 are not complementary to each other,
   N at positions 15 and 33 are complementary to each other,
   N at positions 16 and 32 are complementary to each other,
   N at positions 17 and 31 are complementary to each other,
   N at positions 18 and 30 are complementary to each other,
   N at positions 19 and 29 are complementary to each other
   N at positions 45 and 64 are complementary to each other,
   N at positions 46 and 63 are complementary to each other,
   N at positions 48 and 62 are complementary to each other,
   N at positions 49 and 61 are complementary to each other,
   N at positions 50 and 60 are complementary to each other, and/or
   N at positions 51 and 59 are complementary to each other, optionally where the complementary positions base pair with each other.

Another aspect of the present disclosure is directed to a core Squash nucleic acid aptamer molecule comprising the nucleotide sequence of:
(i) AAG GUG AGC CCA AUA AUA CGG UUU GGG UUA GGA UAG GAA GUA GAG CCG UAA ACU CUC UAA GCG (SEQ ID NO: 5);
(ii) AAG GUG AGC CCA AUA AUA GGG UUU GGG UUA GGA UAG GAA GUA GAG CCC UAA ACU CUC UAA GCG (SEQ ID NO: 6);
(iii) AAG GUG AGC CCA AUA AUA NNN UUU GGG UUA GGA UAG GAA GUA GAG NNN UAA ACU CUC UAA GCG (SEQ ID NO: 7), where N at positions 19 and 48 are complementary to each other, N at positions 20 and 47 are complementary to each other, and N at positions 21 and 46 are complementary to each other, optionally where the complementary positions base pair with each other; or
(iv) AAG GUG NNN NNN NUA AUA NNN UNN NNN NNA GGA UAG GAN NNN NNN NNN UAA ANN NNN NAA GCG (SEQ ID NO: 8), where N at positions 19 and 48 are complementary to each other,
N at positions 20 and 47 are complementary to each other,
N at positions 21 and 46 are complementary to each other,
N at positions 7 and 29 are complementary to each other,
N at positions 8 and 28 are not complementary to each other,
N at positions 9 and 27 are complementary to each other,
N at positions 10 and 26 are complementary to each other,
N at positions 11 and 25 are complementary to each other,
N at positions 12 and 24 are complementary to each other,
N at positions 13 and 23 are complementary to each other,
N at positions 39 and 58 are complementary to each other,
N at positions 40 and 57 are complementary to each other,
N at positions 41 and 56 are complementary to each other,
N at positions 43 and 55 are complementary to each other,
N at positions 44 and 54 are complementary to each other, and/or
N at positions 45 and 53 are complementary to each other, optionally where the complementary sequences base pair with each other.

Another aspect of the present disclosure is directed to an extended Squash nucleic acid aptamer molecule comprising the nucleotide sequence of:

(i)
(SEQ ID NO: 9)
GCC UAG GCU UCA AGG UGG CCC AAU GAU AUG GUU
UGG GUU AGG AUA GGA AUA AGA GCC UUA AAC UCU
UCA AAG CGG AAG UCU AGG C;
(ii)
(SEQ ID NO: 10)
GCC UAG GCU UCA AGG UGA GCC CAA UAA UAU GGU
UUG GGU UAG GAU AGG AAG AAG AGC CUU AAA CUC
UCU AAG CGG AAG UCU AGG C;
(iii)
(SEQ ID NO: 11)
GCC UAG GCU ACA AGG UGA GCC CAA UAA UAU GGU
UUG GGU UAG GAU AGG AAG UAG AGC CUU AAA CUC
UCU AAG CGG UAG UCU AGG C;
or
(iv)
(SEQ ID NO: 12)
GGU AGG CUA CAA GGU GAG CCC AAU AAU ACG GUU
UGG GUU AGG AUA GGA AGU AGA GCC GUA AAC UCU
CUA AGC GGU AGU CUA CC.

Another aspect of the present disclosure is directed to a consensus Squash nucleic acid aptamer molecule comprising the nucleotide sequence of:
(i) GGU AGG CUA CNN NNN NAG CCC AAU AAU ACG GUU UGG GUU NNN NNN NNN AGU AGA GCC GUA AAC UCU CUN NNN NGU AGU CUA CC (SEQ ID NO: 13), where N at each of positions 11-16, 40-48, and 69-73 can be any single nucleotide insertion of any length;
(ii) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 8 forms a stem loop, n at position 18 forms a stem loop, n at positions 1 and 24 form a stem;
(iii) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 1 has the sequence of CUAC, n at position 8 has the sequence of AGC CCA AUA AUA CGG UUU GGG UU (SEQ ID NO: 16), n at position 18 has the sequence of AGU AGA GCC GUA AAC UCU CU (SEQ ID NO: 17), and n at position 24 has the sequence of GUA G;
(iv) CUA CAA GGU GAG CCC AAU AAU ACG GUU UGG GUU AGG AUA GGA AGU AGA GCC GUA AAC UCU CUA AGC GGU AG (SEQ ID NO: 19);
(v) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 1 has the sequence of GGU CCA GAU GCC UUG UAA CCG AAA GGG ACA C (SEQ ID NO: 20), n at position 8 has the sequence of AGC CCA AUA AUA CGG UUU GGG U (SEQ ID NO: 21), n at position 18 has the sequence of AGU AGA GCC GUA AAC UCU CU (SEQ ID NO: 17), and n at position 24 has the sequence of GUG UCG AAA GGA UGG ACC (SEQ ID NO: 25);
(vi) GGU CCA GAU GCC UUG UAA CCG AAA GGG ACA CAA GGU GAG CCC AAU AAU ACG GUU UGG GUU AGG AUA GGA AGU AGA GCC GUA AAC UCU CUA AGC GGU GUC GAA AGG AUG GAC C (SEQ ID NO: 26);
(vii) nNA GNU GAN NNN NNN NNN NNN NNN NNN NNU AGG AUA GGA ANN NNN NNN NNN NNN NNN NUN AGC Gn (SEQ ID NO: 27), where n at positions 1 and 65 can be a nucleotide insertion of 1-200 nucleotide bases; N at positions 2, 5, 9-29, 41-58, and 60 is any nucleotide base; N at positions 9-29 form a step loop; and N at positions 41-58 form a stem loop comprising a bulge in the stem, optionally where n at positions 1 and 65 form a stem;
(viii) nNA GNU GAN UAG GAU AGG AAN UNA GCG n (SEQ ID NO: 28), where n at positions 1 and 28 can be a nucleotide insertion of 1-200 nucleotide bases; N at positions 2, 5, and 23 are any nucleotide base; N at position 9 is a nucleotide insertion of 1-500 nucleotide bases where the insertion forms a stem loop; and N at position 21 is a nucleotide insertion of 1-500 nucleotide bases where the insertion forms a stem loop comprising a bulge in the stem, optionally where n at positions 1 and 28 form a stem;
(ix) nNA GNU GAN NNN NNN NNN NNN NNN NNN NNU AGG AUA GGA ANN NNN NNN NNN NNN NNN UNA GCG n (SEQ ID NO: 29), where n at positions 1 and 64 can be a nucleotide insertion of 1-200 nucleotide bases; N at positions 2, 5, 9-29, 41-57, and 59 is any nucleotide base; N at positions 9-29 forms a step loop; and N at positions 41-57 forms a stem loop, optionally where n at positions 1 and 64 form a stem;
(x) nNA GNU GAN UAG GAU AGG AAN UNA GCG n (SEQ ID NO: 30), where n at positions 1 and 28 can be a nucleotide insertion of 1-200 nucleotide bases; N at positions 2, 5, and 23 are any nucleotide base; N at position 9 is a nucleotide insertion of 1-500 nucleotide bases where the insertion forms a stem loop; and N at position 21 is a nucleotide insertion of 1-500 nucleotide bases where the insertion forms a stem loop, optionally where n at positions 1 and 28 form a stem; or
(xi) nAA GGU GAG CCC AAU AAU ACG GUU UGG GUU AGG AUA GGA AGU AGA GCC GUA AAC UCU CUA AGC Gn (SEQ ID NO: 31), where n at positions 1 and 65 can be a nucleotide insertion of 1-200 nucleotide bases, optionally where n at positions 1 and 65 forms a stem.

Another aspect of the present disclosure relates to a Beetroot nucleic acid aptamer molecule comprising the nucleotide sequence of:
(i) GGU GGG UGG UGU GGA GGA GUA (SEQ ID NO: 32);
(ii) nGG UGG GUG GUG UGG AGG AGU An (SEQ ID NO: 33), where n at positions 1 and 23 is a nucleotide insertion of 1-200 nucleotide bases, optionally where n at position 1 forms a stem with n at position 23;
(iii) NNN NNN GGU GGG UGG UGU GGA GGA GUA NNN NNN (SEQ ID NO: 34), where N at positions 1 and 33 are complementary to each other and form a base pair,
   N at positions 2 and 32 are complementary to each other and form a base pair,
   N at positions 3 and 31 are complementary to each other and form a base pair,
   N at positions 4 and 30 are complementary to each other and form a base pair,
   N at positions 5 and 29 are complementary to each other and form a base pair, and/or
   N at positions 6 and 28 are complementary to each other and form a base pair, optionally where N at positions 1-6 forms a stem with N at positions 28-33; or
(iv) nnn nNN NNN NGG UGG GUG GUG UGG AGG AGU ANN NNN N (SEQ ID NO: 35), where n at positions 1-4 is any nucleotide base,
   N at positions 5 and 37 are complementary to each other and form a base pair,
   N at positions 6 and 36 are complementary to each other and form a base pair,
   N at positions 7 and 35 are complementary to each other and form a base pair,
   N at positions 8 and 34 are complementary to each other and form a base pair,
   N at positions 9 and 33 are complementary to each other and form a base pair, and/or
   N at positions 10 and 32 are complementary to each other and form a base pair, optionally where N at positions 5-10 forms a stem with N at positions 32-37.

Another aspect of the present disclosure is directed to a molecular complex comprising a fluorophore molecule comprising a methyne bridge between a substituted aromatic ring system and a substituted imidazol(thi)one, oxazol(thi)one, pyrrolin(thi)one, or furan(thi)one; and the nucleic acid aptamer molecule according to the present disclosure bound specifically to the fluorophore molecule. The fluorophore molecule has substantially enhanced fluorescence, in comparison to the fluorophore molecule prior to specific binding, upon exposure to radiation of suitable wavelength.

Another aspect of the present disclosure relates to an isolated host cell comprising the molecular complex as described herein.

Another aspect of the present disclosure is related to a kit comprising a fluorophore comprising a methyne bridge between a substituted aromatic ring system and a substituted imidazol(thi)one, oxazol(thi)one, pyrrolin(thi)one, or furan(thi)one ring; and a nucleic acid aptamer molecule according to the present disclosure.

Another aspect of the present disclosure relates to a constructed DNA molecule encoding the nucleic acid aptamer molecule according to the present disclosure.

Another aspect of the present disclosure relates to an expression system comprising an expression vector into which is inserted a DNA molecule according to the present disclosure.

Another aspect of the present disclosure relates to a transgenic host cell comprising the expression system according to the present disclosure.

Another aspect of the present disclosure relates to a method of detecting a target molecule. This method involves forming a molecular complex comprising a nucleic acid aptamer molecule and fluorophore molecule according to the present disclosure; exciting the fluorophore molecule with radiation of appropriate wavelength; and detecting fluorescence by the fluorophore molecule, whereby fluorescence by the fluorophore identifies presence of the target molecule.

Another aspect of the present disclosure relates to an RNA-based metabolite sensor comprising (i) a metabolite-binding aptamer portion and (ii) a regulated aptamer portion comprising the nucleic acid aptamer molecule according to the present disclosure and a transducer domain, where the regulated aptamer portion is linked to the metabolite-binding aptamer portion by the transducer domain.

Another aspect of the present disclosure relates to an RNA-based ratiometric metabolite sensor comprising: (i) a regulated fluorescence activating aptamer comprising the RNA-based metabolite sensor according to the present disclosure and (ii) a constitutive fluorescence activating aptamer.

Another aspect of the present disclosure relates to a system comprising the RNA-based ratiometric metabolite sensor according to the present disclosure; a first fluorophore molecule; and a second fluorophore molecule.

Another aspect of the present disclosure relates to a method of generating a randomized aptamer library. This method involves providing a DNA sequence encoding a riboswitch aptamer and modifying the DNA sequence encoding the riboswitch aptamer by introducing deletions, point mutations, and/or insertions or random nucleotides to generate a library comprising a plurality of modified sequences.

Another aspect of the present disclosure relates to a compound having a structure methyl (Z)-4-(3,5-difluoro-4-hydroxybenzylidene)-1-methyl-5-oxo-4,5-dihydro-1H-imidazole-2-carboxylate (DFAME).

In the present disclosure, RNA-based ratiometric sensors for metabolite imaging in live mammalian cells are described. Such ratiometric sensors are composed of two fluorogenic aptamers, one of which is constitutively fluorescent and provides signal normalization, while the other produces fluorescence in proportion to S-adenosylmethionine (SAM) levels. Squash, a fluorogenic aptamer that exhibits orange fluorescence, which is spectrally separated from the green fluorescence of Broccoli, which is used as the normalizer, is disclosed. Squash was developed using a novel approach to obtain highly folded fluorogenic aptamers, since poor RNA folding is a major factor that limits overall fluorescence. In this approach, a naturally occurring well-folded adenine RNA aptamer was evolved using systematic evolution of ligands by exponential enrichment (SELEX) to bind and activate the fluorescence of green fluorescent protein (GFP)-like fluorophores. Squash was fused to a SAM-binding aptamer to generate Squash-SAM sensors that produce orange fluorescence in proportion to SAM levels. Using the ratiometric SAM sensor, the distinct metabolic pathways that control SAM levels were tested and cell-state specific heterogeneity in intracellular SAM metabolism was uncovered.

### BRIEF DESCRIPTION OF THE DRAWINGS

FIGS. 1A-1E demonstrate the evolution of the add A-riboswitch aptamer into fluorogenic aptamer Squash. FIG. 1A is a schematic illustrating the strategy for evolution of the add A-riboswitch aptamer. Indicated are helices P1-P3, junctional strands (J1/2, J2/3, J3/1), kissing loop (gray lines), and adenine interactions with the aptamer (dotted line) (5′-CUU CAU AUA AUC CUA AUG AUA UGG UUU GGG AGU UUC UAC CAA GAG CCU UAA ACU CUU GAU UAU GAA G-3′ (SEQ ID NO:36)). The chemical structure of adenine and fluorophores are shown. Three regions (Region 1: 5′-UAU AAU-3′ (SEQ ID NO:37)), Region 2: 5′-AGU UUC UAC C-3′ (SEQ ID NO:38); Region 3: 5′-GAU UAU-3′ SEQ ID NO:39), which include J1/2, J2/3, and J3/1 were randomized in both size and sequence resulting in a sprouts/clips library for SELEX. Each nucleotide in the colored regions was replaced using a combination of random stochastic deletion (designated “N”) and random stochastic insertion (designated “n”). FIG. 1B is schematic showing a three dimensional structure of the add A-riboswitch aptamer highlighting the adenine-binding pocket (PDB 1Y26). The mutagenized regions are shown in the same color as FIG. 1A. Adenine is indicated by the arrow. FIG. 1C is a schematic illustrating the evolution of the add A-riboswitch aptamer into Squash, a DFHBI-1T-binding aptamer. The sprouts/clips library was selected for binding to DFHBI-conjugated agarose resulting in aptamer 9-1. Aptamer 9-1 was subjected to two consecutive rounds of directed evolution generating DE2-6. A pair of mutations was introduced in DE2-6 to strengthen the kissing loop interaction resulting in Squash. FIG. 1D is a graph showing fluorescence activation of DFHBI-1T by different aptamer intermediates that led to Squash. The values inside the bars indicate fold-activation of DFHBI-1T fluorescence by the corresponding aptamer. Data represent mean values±s.d. for n=3 independent experiments. FIG. 1E is an alignment demonstrating that the Squash aptamer shows ligand-binding pocket expansion compared to the parental add A-riboswitch aptamer (add A: 5′-UAU AAU CCU AAU GAU AUG GUU UGG GAG UUU CUA CCA AGA GCC UUA AAC UCU UGA UUA U-3′ (SEQ ID NO:40). Aptamers at different stages of Squash evolution are aligned (9-1: 5′-AGG UGG CCC AAU GAU AUG GUU UGG GUU AGG AUA GGA AUA AGA GCC UUA AAC UCU UCA AAG CG-3′ (SEQ ID NO:41); DE1-2: 5′-AGG UGA GCC CAA UAA UAU GGU UUG GGU UAG GAU AGG AAG AAG AGC CUU AAA CUC UCU AAG CG-3′ (SEQ ID NO:42); DE2-6: 5′-AGG UGA GCC CAA UAA UAU GGU UUG GGU UAG GAU AGG AAG UAG AGC CUU AAA CUC UCU AAG CG-3′ (SEQ ID NO:43); Squash: 5′-AGG UGA GCC CAA UAA UAC GGU UUG GGU UAG GAU AGG AAG UAG AGC CGU AAA CUC UCU AAG CG-3′ (SEQ ID NO:44). Constant sequences are indicated with gray dots. The sequences outside the randomized regions are shown in gray. Arrows indicate U→C and U→G mutations that were introduced to enhance the kissing loop interaction.

FIGS. 2A-2F demonstrate that Squash activates the fluorescence of DFHBI-1T and DFHO without utilizing a G-quadruplex. FIG. 2A is a schematic showing the secondary structure of Squash as predicted by mFOLD (Squash: 5′-GGC UAC AAG GUG AGC CCA AUA AUA CGG UUU GGG UUA GGA UAG GAA GUA GAG CCG UAA ACU CUC UAA GCG GUA GUC-3′ (SEQ ID NO:1)). Squash retained the main structural elements of add A-riboswitch aptamer including a 3-way junction and predicted kissing loop interactions (residues at positions 30, 31, 56, and 57, solid gray line). An additional base pair in the kissing loop between loop L2 and L3 (residues at positions 29 and 58, dotted gray line) improved fluorescence activation. FIG. 2B are spectra demonstrating that Squash binds and activates the fluorescence of both DFHBI-1T and DFHO. Shown are the excitation (Ex) and emission (Em) spectra of Squash bound to DFHBI-1T and DFHO (structures shown in inset). Spectra were measured using 20 μM RNA and 2 μM of the indicated fluorogenic dye. FIG. 2C is a bar graph showing that Squash shows similar fluorescence activation of DFHBI-1T as Broccoli but much higher activation of DFHO than Corn. Fluorescence activation was measured by incubating 200 nM dye and 10 μM RNA. By using a large excess of RNA compared to the fluorophore, it was ensured that the fluorescence observed is from 200 nM RNA-fluorophore complex. Data represent mean values±s.d. for n=3 independent experiments. The values inside the bars indicate fold activation. AU, arbitrary units. FIG. 2D is a graph showing the dissociation constant (K_{d}) between Squash and DFHO as measured by titration of 50 nM Squash with increasing concentration of DFHO, and fitted using a one-site saturation model. Data represent mean values±s.d. for n=3 independent experiments. FIG. 2E is a bar graph showing the results of an experiment in which Squash-DFHO fluorescence was measured in buffers containing exclusively the indicated cations. Squash was fluorescent in K⁺-free buffers suggesting that it lacks a G-quadruplex. Broccoli, which contains a G-quadruplex, exhibited markedly reduced fluorescence in the absence of K⁺. Data represent mean values±s.d. for n=3 independent experiments. FIG. 2F is a graph demonstrating that Squash-DFHO shows >80% fluorescence activation at 0.1 mM MgCl₂. Similar to the parental add A-riboswitch aptamer (see Lemay et al., “Folding of the Adenine Riboswitch,” Chem. Biol. 13:857-868 (2006), which is hereby incorporated by reference in its entirety), Squash-DFHO has a low magnesium requirement for fluorescence activation. Data represent mean values±s.d. for n=3 independent experiments.

FIGS. 3A-3D demonstrate that Squash shows efficient folding in vitro and in cells. FIG. 3A is a bar graph showing that Squash does not require the F30 folding scaffold. In vitro folding of Broccoli (with DFHBI-1T, left bars) is substantially improved when placed in F30 scaffold. By contrast, Squash (right bars) does not show increased folding when inserted into F30, suggesting that it already exhibits high folding. Data represent mean values±s.d. for n=3 independent experiments. FIG. 3B demonstrates that Squash-DFHO shows similar thermostabilty as Broccoli-BI, an aptamer-fluorophore pair evolved for improved binding and thermostability (see Li et al., “Fluorophore-Promoted RNA Folding and Photostability Enables Imaging of Single Broccoli-Tagged mRNAs in Live Mammalian Cells,” Angew. Chemie Int. Ed. 59:4511-4518 (2019), which is hereby incorporated by reference in its entirety). Melting temperature was measured using 1 μM RNA and 10 μM fluorophore. FIG. 3C are plots showing that F30 enhances Broccoli but not Squash fluorescence in HEK293T cells. Left column, flow cytometry analysis of DFHBI-1T-treated cells expressing linear unscaffolded Broccoli or linear F30-Broccoli (expressed using U6+27 promoter). Right column, DFHO-treated cells expressing linear Squash or linear F30-Squash. Cells were analyzed in the green (ex. 488 nm, em. 525±25 nm) or orange (ex. 488 nm, em. 570±20 nm) channel for Broccoli and Squash fluorescence, respectively. An auxiliary far-red channel (ex. 635 nm; em. 780±30) was used to measure cellular autofluorescence. F30 enhances the cellular fluorescence levels of Broccoli, but not Squash. FIG. 3D are images showing HEK293T cells expressing either 5S-Broccoli or 5S-F30-Broccoli incubated with 25 μM DFHBI-1T. The 5S-Broccoli appeared as faint dots while 5S-F30-Broccoli appeared as bright perinuclear puncta (see inset). Both 5S-Squash or 5S-F30-Squash appeared as bright perinuclear puncta (see inset). These data suggest that F30 is not needed to enhance Squash fluorescence. Scale bar, 20 μm.

FIGS. 4A-4G demonstrate the development of a ratiometric S-adenosyl-methionine (SAM) sensor from Squash. FIG. 4A is a schematic showing the design of the Squash-SAM sensor transducer library. The SAM-III aptamer (top) was fused to Squash (bottom) through a transducer helix (middle). The sensor was selected from a library of randomized transducer sequences generated using the sprouts/clips method. FIG. 4B is a bar graph showing the results of an experiment in which, after three rounds of SELEX, two Squash-SAM sensors, 5-1 and 4-2, were tested for SAM-induced fluorescence. Squash-SAM sensor RNAs (1 μM) were incubated with 10 μM DFHO and 0 or 0.1 mM SAM for 1 hour at 37° C. and then fluorescence was measured (ex. 495 nm; em. 562 nm). Data represent mean values±s.d. for n=3 independent experiments. FIGS. 4C-4D are plots showing the kinetics of fluorescence activation (FIG. 4C) and deactivation (FIG. 4D) of Squash-SAM sensors. Sensor activation was induced by adding 0.1 mM SAM to 1 μM sensor RNAs and 10 μM DFHO at 37° C. Deactivation was measured after removal of SAM by gel filtration. Comparison of the Squash-SAM sensors with Corn-SAM sensor (see Kim and Jaffrey, “A Fluorogenic RNA-Based Sensor Activated by Metabolite-Induced RNA Dimerization,” Cell Chem. Biol. 26:1725-1731 (2019), which is hereby incorporated by reference in its entirety) showed markedly faster activation kinetics of both Squash-based sensors compared to the Corn-based sensor. FIG. 4E is a schematic of the Squash-based ratiometric SAM sensor. The ratiometric sensor comprises Broccoli in one arm of F30 and the Squash-SAM sensor in the other arm. SAM binding activates the orange fluorescence by promoting Squash folding and subsequent binding with DFHO. The constitutive green fluorescence of Broccoli-BI is used for normalization, thus eliminating the confounding effect of cell-to-cell variation in sensor expression level, and different fluorescence measurements in thick vs. thin parts of the cell. FIG. 4F shows the orange to green (O/G) fluorescence ratio measured using the ratiometric sensor in individual HEK293T cells showed low cell-to-cell variation in SAM levels (also see next panel). Inhibition of SAM biosynthesis using cycloleucine showed marked reduction of SAM levels in all cells, as measured by the reduced O/G fluorescence ratio. The SAM levels were restored after withdrawal of cycloleucine. A control RNA with constitutive Squash fluorescence (see FIG. 12B) did not show any notable change in the O/G ratio upon cycloleucine treatment. FIG. 4G shows SAM trajectory plots of individual HEK293T cells upon cycloleucine treatment and withdrawal, measured by the ratiometric signal. Each trajectory plot (8 cells from three experiments) was generated based on the mean O/G fluorescence ratio (mean of O/G ratio of all the pixels comprising a cell) in single cells. The ratio was measured at 10 minute time intervals. Scale bar, 20 μm.

FIGS. 5A-5E demonstrate that metabolic origin of SAM in different cell types measured by the ratiometric sensor. FIG. 5A shows SAM levels in individual mES cells as determined using the O/G fluorescence ratio at 30 minute intervals after withdrawing threonine, methionine, or both. Threonine depletion did not lead to a reduction in SAM levels. The control constitutively fluorescent Squash was not affected by withdrawal of threonine and methionine. Scale bar, 20 μm. FIG. 5B shows SAM trajectory plots of O/G ratio in individual mES cells cultured in +2i upon withdrawal of methionine or both threonine and methionine. Overall, low cell-to-cell variation was seen (n=8 cells from three separate experiments). FIG. 5C shows SAM trajectory plots of O/G ratio in individual mES cells cultured in −2i upon withdrawal of methionine or both threonine and methionine shows high cell-to-cell variability. The rate of SAM drop was slower than the rate in cells cultured in +2i (n=8 cells from three separate experiments). FIG. 5D shows the effect of methionine withdrawal on SAM level in HCT116 cancer cells. SAM levels showed high cell-to-cell variability at baseline. SAM levels were measured at 20 minute time intervals after methionine withdrawal and after methionine (100 μM) addition. Cells indicated with a white arrow start with a very high level of SAM and show a slow drop in SAM levels over time, indicating low SAM utilization. Scale bar, 20 μm. FIG. 5E demonstrates that methionine withdrawal shows distinct metabolic subtypes based on SAM loss. Type I cells show high basal levels of SAM and a slow drop in SAM levels upon methionine depletion. Type II cells show intermediate levels of SAM and a rapid drop in SAM levels upon methionine depletion. Type III cells show low basal levels of SAM and a slow drop in SAM levels after methionine depletion. n=5 cells for each type, from three separate experiments.

FIGS. 6A-6H demonstrate the characterization of the fluorescence properties of different intermediates during development of Squash. FIG. 6A is a bar graph showing fluorescence activation of DFHO by Squash and its precursors. Fluorescence was measured with 200 nM dye and 10 μM RNA (ex: 495 nm, em: 562 nm, except for Corn; ex: 505 nm, em: 545 nm). Data represent mean values±s.d. for n=3 independent experiments. Squash showed >2-fold activation of DFHO compared to Corn. FIG. 6B is a bar graph showing fluorescence of initial hits after Round 7 of SELEX. In vitro transcribed RNAs from each hit (1 μM final) were mixed with DFHBI-1T (10 μM final) and the fluorescence was measured (ex: 452 nm, em: 503 nm, except for Broccoli; ex: 472 nm, em: 507 nm). Data represent mean values±s.d. for n=3 independent experiments. FIGS. 6C-6D are bar graphs showing fluorescence measurements of the hits with DFHBI-1T after first (FIG. 6C) and second (FIG. 6D) round of directed evolution, measured as in FIG. 6B. Data represent mean values±s.d. for n=3 independent experiments. FIG. 6E is a bar graph showing fluorescence measurements of hits using DFHO after the second round of directed evolution measured as in FIG. 6A. The fluorescence of the hits were normalized against Corn. Data represent mean values±s.d. for n=3 independent experiments. FIG. 6F is a graph showing normalized fluorescence. DE2-6 has one bulged nucleotide (U51) and one G·U pair based on mFold (FIG. 18D). Elimination of the bulge, the G·U pair, or both did not improve fluorescence of DE2-6. Fluorescence was measured as in FIG. 6A. Data represent mean values±s.d. for n=3 independent experiments. FIG. 6G is a bar graph showing that mutation (G30C) of the kissing loop of DE2-6 resulted in loss of fluorescence which was recovered by a complementary mutation (C57G) in the opposite loop of the predicted kissing loop. Similar results were also observed for the other kissing loop basepair. Two mutations (U29C, U58G) created an extra G·C basepair in the kissing loop compared to add A-aptamer and resulted in ˜20% increase in fluorescence of DE2-6. Further improvement of the kissing loop interaction cannot be achieved by further mutations (A28G+U59C in Squash). Data represent mean values±s.d. for n=3 independent experiments. FIG. 6H demonstrates that although Broccoli-BI and Squash-DFHO (20 μM RNA, 2 μM dye) excitation spectra overlap, their emission maxima show −57 nm separation. Structure of BI is shown in the inset.

FIGS. 7A-7H provide photophysical and biophysical characterization of Squash. FIG. 7A shows the absorbance spectra (50 μM RNA, 5 μM DFHBI-1T) of DFHBI-1T alone and in complex with Squash demonstrates a smaller red shift (Abs max: 450 nm) compared to Spinach (Abs max: 470 nm) (Paige and Jaffrey, “RNA Mimics of Green Fluorescent Protein,” Science 333:642-646 (2011), which is hereby incorporated by reference in its entirety) and Broccoli (Abs max: 469 nm) (Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014), which is hereby incorporated by reference in its entirety). Excess RNA was used to ensure binding of nearly all fluorophore. FIG. 7B demonstrates that DFHO shows a red-shifted absorbance spectrum upon binding Squash (50 μM RNA, 5 μM DFHO). FIG. 7C demonstrates that both Squash and Broccoli shows similar emission maxima. Squash also showed higher fluorescence (10 μM RNA, 1 μM DFHBI-1T, ex. 452 nm for Squash and 472 nm for Broccoli) intensity with DFHBI-1T compared to DE2-6. FIG. 7D demonstrates that fluorescence spectra (10 μM RNA, 1 μM DFHO, ex. 495 nm for Squash and 505 nm for Corn) showed a red-shifted Squash emission maxima (562 nm) compared to Corn (545 nm), consistent with a different mode of interaction between DFHO and Squash compared to Corn. Squash also showed more than two-fold higher fluorescence intensity with DFHO compared to Corn. FIG. 7E shows the results of an experiment in which K_{d} was measured by titration of 50 nM RNA with DFHBI-1T and then fitting the data using a one-site saturation model. Data represent mean values±s.d. for n=3 independent experiments. FIG. 7F shows the results of an experiment in which binding kinetics were performed as reported previously (Han et al., “Understanding the Photophysics of the Spinach-DFHBI RNA Aptamer-Fluorogen Complex to Improve Live-Cell RNA Imaging,” J. Am. Chem. Soc. 135:19033-19038 (2013), which is hereby incorporated by reference in its entirety). The fluorescence signal trace was fitted with a monoexponential curve to extract k_{obs}. k_{obs} was plotted as a function of total RNA (50 nM RNA) and fluorophore concentration. kₒₙ and k_{off} were extracted as the slope and intercept, respectively. Squash k_{off} (0.014±0.008 s⁻¹) is very similar to that of Corn (0.018±0.002 s⁻¹). kon for Squash-DFHO (162300±8100 M⁻¹ s⁻¹) is seven-times higher than Corn-DFHO (23000±3000 M⁻¹ s⁻¹) which could be due to the high folding of Squash. FIGS. 7G-7H provide K_{d} measurements for Broccoli and Squash binding to BI (FIG. 7G) and DFHO (FIG. 7H) were performed as in FIG. 7E, using 50 nM RNA and then fitting the data using a one-site saturation model. Data represent mean values±s.d. for n=3 independent experiments.

FIGS. 8A-8D compare the in cell photostability of Squash with other fluorogenic aptamers. FIG. 8A shows images comparing in cell photostability of Squash-DFHBI-1T pair with Broccoli-BI. Compared to DFHBI-1T, BI shows improved photostability when bound to Broccoli (Li et al., “Fluorophore-Promoted RNA Folding and Photostability Enables Imaging of Single Broccoli-Tagged mRNAs in Live Mammalian Cells,” Angew. Chemie Int. Ed. 59:4511-4518 (2019), which is hereby incorporated by reference in its entirety). The photostability of Squash-DFHBI-1T was compared with Broccoli-BI. Aptamers were expressed as circular RNAs (Litke and Jaffrey, “Highly Efficient Expression of Circular RNA Aptamers in Cells using Autocatalytic Transcripts,” Nat. Biotechnol. 37:667-675 (2019), which is hereby incorporated by reference in its entirety) and aptamer-expressing cells were incubated with dyes (10 μM). Continuous images (100 ms per frame) were taken for 10 seconds total while the cells were continuously illuminated. The highest available light power in the microscope was used since previously lower light levels were used and Broccoli-BI photobleaching was not readily detected (Li et al., “Fluorophore-Promoted RNA Folding and Photostability Enables Imaging of Single Broccoli-Tagged mRNAs in Live Mammalian Cells,” Angew. Chemie. Int. Ed. 59:4511-4518 (2019), which is hereby incorporated by reference in its entirety). Shown are every other frame starting from 0 ms to 1000 ms. Scale bar, 20 μm. FIG. 8B shows images comparing in cell photostability of Squash-DFHO with Corn-DFHO. Corn aptamer exhibits high photostability with DFHO compared to Spinach or Broccoli with DFHBI-1T23. The photostability of Squash-DFHO was compared with tCorn (Corn in the tRNA scaffold)-DFHO. The images were taken in as in FIG. 8A except the exposure time was 500 ms per frame. Shown are frame between 0 and 2500 ms. Scale bar, 20 μm. FIG. 8C shows quantification of in cell photostability for green emitting aptamers. Cellular mean fluorescence intensity was calculated and normalized to maximum intensity at 0 seconds. The normalized cellular fluorescence intensities were plotted against time. Both Broccoli-BI and Squash-DFHBI-1T showed a similar initial rate of drop in fluorescence, which reflects the rate of light-induced photoisomerization of the fluorophore to the low-fluorescence trans-isomer (Li et al., “Fluorophore-Promoted RNA Folding and Photostability Enables Imaging of Single Broccoli-Tagged mRNAs in Live Mammalian Cells,” Angew. Chemie Int. Ed 59:4511-4518 (2019), which is hereby incorporated by reference in its entirety). The higher plateau associated with Squash-DFHBI-1T likely reflects a higher on-rate for fluorophore binding since the plateau reflects the balance between light-induced isomerization/trans-fluorophore dissociation and cis-fluorophore rebinding (Li et al., “Fluorophore-Promoted RNA Folding and Photostability Enables Imaging of Single Broccoli-Tagged mRNAs in Live Mammalian Cells,” Angew. Chemie Int. Ed. 59:4511-4518 (2019), which is hereby incorporated by reference in its entirety). FIG. 8D shows quantification of in cell photostability for yellow-orange emitting aptamers. Assays were performed as in FIG. 8C. For comparison, mVenus, a fluorescent protein which has similar fluorescence emission properties as Corn-DFHO, was used.

FIGS. 9A-9C demonstrate that Squash exhibits higher cellular fluorescence than Corn when expressed as a circular RNA. FIG. 9A shows images comparing in-cell fluorescence intensity of circular Squash or circular tCorn (tRNA-scaffolded Corn). HEK293T cells expressing 5S-control (no aptamer), Tornado-tCorn or Tornado-Squash plasmids were imaged using 10 μM DFHO and the same microscope settings. Cells expressing circular Squash exhibited much brighter cellular fluorescence than cells expressing circular tCorn. Circular Squash showed mostly nucleus-excluded signal. Scale bar, 20 μm. FIG. 9B shows plots comparing cellular brightness of Squash and Corn using flow cytometry of HEK293T cells prepared as in FIG. 9A. Cells were analyzed in the yellow (ex 488 nm, em 545±17.5 nm) fluorescence channel. An auxiliary far-red channel (ex 635 nm; em 780±30) was used to measure cellular auto-fluorescence. Cells expressing circular Squash exhibited substantially more fluorescence. FIG. 9C shows the results of an experiment carried out to determine if the higher fluorescence of circular Squash in FIG. 9A and FIG. 10B was due to higher expression. Briefly, 10 g total RNA was isolated from HEK293T cells and analyzed by gel staining with DFHO and SYBR Gold as described previously (Filonov et al., “In-Gel Imaging of RNA Processing Using Broccoli Reveals Optimal Aptamer Expression Strategies,” Chem. Biol. 22:649-660 (2015), which is hereby incorporated by reference in its entirety). Bands corresponding to circular Squash and circular tCorn in SYBR Gold staining were identified by comparing them with the 5S-control lane and marked by white stars. Quantification of the band intensities indicates slightly higher expression of circular tCorn. Note, unlike Squash, two tCorn molecules are required to bind a single DFHO molecule. The higher fluorescence of circular Squash-expressing cells compared to tCorn is probably due to higher quantum yield and improved folding of Squash. Additionally, the DFHO fluorescence intensity of the band corresponding to circular Squash is much higher than that of the circular tCorn band, suggesting that Squash also folds better in the gel than Corn.

FIGS. 10A-10I demonstrate the development and characterization of Squash-SAM sensors. FIG. 10A is a bar graph showing the results of an experiment in which Squash was fused with the SAM-binding SAM-III aptamer through a sprouts and clips randomized transducer (NnNnNnNn for each strand). 2 μM of the RNA pool after each SELEX round was mixed with 10 μM DFHO and fluorescence was measured in the absence and presence of 0.1 mM SAM at 37° C. FIG. 10B shows the results of an experiment where, for each library member, 1 μM of the in vitro transcribed RNA was incubated with DFHO in the absence or presence of 0.1 mM SAM (37° C., 10 minutes) and put into separate tubes and imaged immediately (ex: 530±14 nm, em=605±25 nm). Squash-SAM sensors 4-2 and 5-1 are highlighted. FIG. 10C and FIG. 10F are bar graphs showing that Squash-SAM sensor 4-2 (FIG. 10C) and 5-1 (FIG. 10F) are activated by SAM and not related molecules. Briefly, 1 μM RNA was mixed with 10 μM DFHO and indicated molecules. Data represent mean values±s.d. for n=3 independent experiments. FIG. 10D and FIG. 10G show the measurement of K_{d} of Squash-SAM sensor 4-2 (FIG. 10D) and 5-1 (FIG. 10G) with DFHO were measured as in FIG. 7E. The K_{d}s are slightly weaker than that of Squash with DFHO (see FIG. 2D). Data represent mean values±s.d. for n=3 independent experiments. FIG. 10E and FIG. 10H show the measurement of K_{d} of Squash-SAM sensor 4-2 (FIG. 10E) and 5-1 (FIG. 10H) with SAM. Sensor RNA (1 μM) was titrated with SAM in presence of 10 μM DFHO (37° C.) and the resulting data was fitted using one-site saturation model. The K_{d} for the SAM-III riboswitch is ˜1 μM. Data represent mean values±s.d. for n=3 independent experiments. FIG. 10I is a bar graph showing that Broccoli-BI fluorescence (1 μM Broccoli, 10 μM BI) is not affected by SAM or related molecules making it an appropriate normalizer for ratiometric sensor. Data represent mean values±s.d. for n=3 independent experiments.

FIGS. 11A-11C demonstrate the detection of intracellular SAM levels using Squash-based SAM sensors. FIG. A shows the detection of intracellular SAM levels using the Squash-SAM sensor 4-2 and 5-1. HEK293T cells were transfected with the Tornado plasmids expressing the sensors or the constitutively fluorescent Squash aptamer (no SAM dependent fluorescence enhancement) as circular RNAs. Cells expressing the circular SAM biosensors or circular Squash were incubated with 10 μM DFHO and imaged every 10 minutes after treatment with 30 mM cycloleucine (an inhibitor of the major SAM biosynthesis enzyme, MAT2A) for 90 minutes total. Following this, cycloleucine was removed by replacing the imaging media with cycloleucine-free media. Images were taken again at 10 minute intervals for 90 minutes. Images are shown at specific intervals along with results for representative cells. Cells expressing the circular sensors showed drop in the cellular fluorescence during cycloleucine treatment and increase in cellular fluorescence during cycloleucine withdrawal. However, cell expressing circular Squash did not show any change in cellular fluorescence during this treatment. Scale bar, 20 μm. FIG. 11B and FIG. 11C show quantification of live-cell SAM levels based on Squash-SAM sensors 4-2 (FIG. 11B) and 5-1 (FIG. 11C), respectively. Cellular mean fluorescence intensity was calculated for each sensor and normalized to maximum intensity at time point 0. The normalized values of mean cellular fluorescence intensity were plotted against time at 10 minute intervals for six different cells. Each cell is represented using a different color. For each sensor, SAM decay was observed following addition of cycloleucine and SAM recovery is seen after withdrawal of cycloleucine. Interestingly for both the sensors, the SAM decay profile for each individual cell is very similar while the SAM recovery profiles are distinctly different.

FIGS. 12A-12F demonstrate that Broccoli-BI and Squash-DFHO can be used as an orthogonal pair of fluorogenic aptamers for two color imaging. FIG. 12A are images showing a lack of correlation of eqFP670 and Squash fluorescence expressed from the same plasmid (U6+27 promoter for Squash and PGK for eqFP670). Many HEK293T cells have high Squash fluorescence and low eqFP670 fluorescence (narrow white arrows). Some cells have correlated fluorescence (wide gray arrows). Flow cytometry also revealed poor correlation. Scale bar, 20 m. FIG. 12B is a schematic representation of the circular F30-Broccoli-Squash RNA used as a constitutive control for the ratiometric sensor (FIG. 4F). The RNA was generated by fusing Squash and Broccoli to each arm of an F30 scaffold. FIG. 12C demonstrates the high correlation between Squash and Broccoli fluorescence when expressed within a single RNA. The F30-Broccoli-Squash construct (FIG. 12B) was expressed as a circular RNA in HEK293T cells and imaged in both green and orange channels after incubation with 10 μM BI and 10 μM DFHO. Excellent correlation was observed for each cell (see FIG. 4F, bottom panel for O/G ratio). This was also confirmed by flow cytometry. FIG. 4D are images demonstrating that circular Broccoli-expressing cells showed negligible bleedthrough in the orange channel and circular Squash-expressing cells showed no bleedthrough in the green channel. Circular F30-Broccoli-Squash shows bright signal in both channels. FIG. 12E shows the results of an experiment carried out to determine if DFHO can compete with BI's ability to bind Broccoli. Briefly, HEK293T cells expressing circular Broccoli (10 μM BI) were imaged and green fluorescence before and after addition of DFHO (10 μM, 1 hour) was compared. The green fluorescence signal was not substantially affected. Similarly, BI does not affect Squash-DFHO fluorescence when circular Squash is expressed in HEK293T cells. FIG. 12F shows the results of an experiment carried out to test the possibility of FRET in the ratiometric sensor. Briefly, ratiometric Squash-SAM sensor 5-1 was expressed in HEK293T cells as a circle. After incubating the cells with 10 μM BI, an image was taken in the green channel. Then, DFHO (10 μM, 1 hour incubation) was added. No decrease in the Broccoli-BI (possible donor) green fluorescence was seen upon formation of the Squash-DFHO pair (possible acceptor), consistent with lack of FRET.

FIGS. 13A-13F demonstrate the quantification of cellular SAM concentration using ratiometric sensors. FIG. 13A is a standard curve measuring FRET signal (ex: 485 nm, em: 665 nm) in a biochemical assay to determine SAM concentration. Data represent mean values±s.d. for n=3 independent experiments. FIG. 13B shows the average cellular concentration of SAM measured from 1.2×10⁶ cells utilizing the standard curve in FIG. 13A. To calculate cellular volume, it was assumed that HEK293T cells are 17 μm-diameter spheres. The average SAM concentration was quantified at different time points during cycloleucine treatment and withdrawal. SAM concentrations were not affected by sensor expression. Data represent mean values±s.d. for n=3 independent experiments. FIGS. 13C-13D show the correlation between the ratiometric signal and biochemical SAM measurements for Squash-SAM sensors 5-1 (FIG. 13C) and 4-2 (FIG. 13D). To convert the sensor ratiometric signal into SAM concentration, the average ratiometric signal for HEK293T cells during cycloleucine treatment was calculated (n=6 cells, from FIG. 4G). Using the biochemical assay the average SAM concentration was determined at the same time points, allowing extrapolation of intracellular SAM concentrations from the O/G ratio. FIG. 13E shows the results of an experiment in which the average O/G fluorescence ratio was measured in 30 cells from three independent measurements for each indicated cell type and culture condition. HEK293T cells and mES cells cultured in +2i media showed a narrow distribution while HCT116 and mES cells cultured in −2i showed a wide range of O/G ratio. Sensors used for each cell type are indicated. Line indicates median, box shows the interquartile range, and whiskers are the minimum and maximum values. FIG. 13F is a box plot showing the average O/G fluorescence ratio for each cell in FIG. 13E converted into average cellular SAM concentration (based on FIG. 13C and FIG. 13D) and plotted for each condition. For HEK293T cells, the distribution of O/G fluorescence ratios was different for the two Squash-SAM sensors; however, when these O/G ratios are converted to SAM concentrations based on the specific O/G-to-SAM correlation for each sensor, the cellular SAM concentrations were similar regardless of the sensor. Line indicates median, box shows the interquartile range, and whiskers are the minimum and maximum values.

FIGS. 14A-14D demonstrate the effect of cycloleucine treatment and different amino acids depletion on SAM level in mESCs. FIG. 14A are images showing expression of Squash-SAM sensors 4-2 and 5-1 for ratiometric SAM imaging in mESCs. Unlike HEK293T cells, the SAM-dependent Squash signal (DFHO channel) was too dim for the 5-1 sensor. However, Squash-SAM sensor 4-2 gave much brighter cellular fluorescence signal in the DFHO channel. Thus, despite having lower dynamic range than Squash-SAM sensor 5-1, the Squash-SAM sensor 4-2 was used for ratiometric imaging of SAM in mESCs. Image acquisition time was 100 ms for BI channel and 500 ms for DFHO channel. Scale bar, 20 μm. FIG. 14B show the results of an experiment carried out to test whether MAT2A is the major SAM biosynthetic enzyme in mESCs. Briefly, Squash-SAM sensor 4-2 was expressed and cells were treated with cycloleucine (30 mM final), a MAT2A inhibitor. A rapid drop of cellular SAM levels was observed in 30 minutes for mESCs cultured in both +2i and −2i media. Note: mESCs cultured in −2i media exhibit a large flattened morphology, which accounts for their increased size relative to mESCs cultured in +2i. Scale bar, 20 μm. FIGS. 14C-14D demonstrate that the drop in SAM levels due to amino acid depletion in mESCs is reversible in cells cultured in either +2i (FIG. 14C) or −2i (FIG. 14D). In FIG. 5A, it was shown that depletion of methionine leads to a drop in SAM in mESCs. To see if reintroduction of the amino acids in the media leads to rise in SAM level, the amino acids were added back to the media. Images are shown at indicated time points. For most mESCs cultured in both +2i and −2i conditions, the SAM levels were observed to go back to the pre-treatment condition indicating the reversible nature and lack of cytotoxicity of this process. Scale bar, 20 μm.

FIGS. 15A-15E demonstrate the effect of different amino acids depletion and cycloleucine treatment on SAM level in HCT116 cells. FIG. 15A are images demonstrating that both Squash-SAM sensors 4-2 and 5-1 produced signal that is substantially higher than the background in both the channels. 5-1 was used because it has higher dynamic range. Scale bar, m. FIG. 15B are images demonstrating that threonine depletion does not change SAM levels in mESCs (see FIG. 5A). To test if this also holds in HCT116 cells, Squash-SAM sensor 5-1 was expressed in HCT116 cells and SAM level was monitored after threonine depletion. Most cells did not show any notable change in SAM levels, consistent with a lack of functional threonine dehydrogenase enzyme in these cells. FIG. 15C demonstrates that although threonine depletion does not change SAM levels (FIG. 15B), threonine could be important in methionine-depleted cells. To test this, Squash-SAM sensor 5-1 was expressed in HCT116 cells and SAM levels were monitored for 240 minutes after depleting both threonine and methionine. Threonine depletion did not exacerbate the drop in SAM levels, suggesting that threonine does not have a major role in SAM levels in these cells. FIG. 15D demonstrates that circular Squash-SAM sensor 5-1 expressed in HCT116 cells showed marked drop in SAM after cycloleucine (30 mM final) treatment, indicating MAT2A is required for SAM biosynthesis in these cells. A rapid drop in SAM levels was observed in 30 minutes for most cells. The cells indicated by white arrow (in FIG. 15C and FIG. 15D) start with a very high level of SAM and show slower drop in SAM level over time compared to other cells. FIG. 15E are graphs showing that HCT116 cells have three different cell populations based on baseline SAM levels (see FIG. 5D and FIG. 5E). The average cellular ratio of O/G fluorescence was quantified for these cells at 15 minute intervals after cycloleucine (30 mM final) treatment. All three populations showed slightly faster drop in SAM levels with cycloleucine treatment compared to depletion of methionine and threonine together.

FIGS. 16A-16F demonstrate the library diversity of the add A riboswitch aptamer-based sprouts and clips library and identification of fluorogenic aptamers from it. FIG. 16A is a graph showing that the sprouts and clips library contains many library members, but the relative frequency/probability of each library member is not equal. Shown is an analysis of the abundance of library members of the indicated length (on x axis) for a library containing a total of 22 ‘Nn’ sequences (see FIG. 1A) distributed over three junctional regions. A library member corresponding to ‘0’ on the x axis has same number of nucleotides as the parental add-A riboswitch aptamer (no sprouts or clips). Positive ‘x’ values are for sprouts and negative ‘x’ values are for clips. The abundance of library members of the indicated length was calculated with a constant clipping probability (93.3%) and varying sprouting probability. Theoretically possible number of mutants was calculated using the formula described in the Examples below. Blue triangles represent the number of all theoretically possible nucleotide sequences based on the length of the library members indicated on the x-axis. As sprouting probability increases, the average length of the library members compared to the parental sequence also increases. The colored circles are the predicted actual number of library members that have a length difference of x nucleotides compared to the parental sequence in a library assumed to contain 6×10¹⁴ total members. All numbers below 1 mean that number of molecules for those mutants is either 1 or 0, with the latter more likely for the mutants that are more towards the extremes of the library population. FIG. 16B is a graph showing the same analysis as in FIG. 1A, except that the sprouting frequency is fixed at 5.2%, and the clipping probability is varied as indicated. FIG. 16C is a bar graph showing fluorescence of RNA pools after each round of SELEX. In each experiment, 20 μM of the RNA pool after each round of SELEX was mixed with 10 μM of DFHBI-1T and fluorescence was measured (ex: 460 nm, em: 500 nm). Fluorescence activation was first detected in the RNA pool after the seventh round of SELEX. FIG. 16D is a FACS scatter plot showing the fluorescence distribution of E. coli cells transformed with the cloned plasmids generated from the RNA pool after round seven of SELEX. E. coli expressing RNA aptamers were incubated with 20 μM DFHBI-1T (in 1×PBS) and then sorted using the indicated gate. The position of each dot reflects RNA green fluorescence (ex: 488 nm, em: 525±25 nm, x-axis) and side scattering, SSC-A (y-axis). As can be seen, a small fraction of the library-expressing bacteria (green dots) exhibit significantly higher green fluorescence than most of the cells (yellow dots). FIG. 16E is a FACS scatter plot showing the fluorescence distribution of E. coli hits which were selected using the gate shown. 10×10⁶ E. coli cells which were transformed with the plasmid library encoding the RNA aptamers from round seven of SELEX were sorted. Screening was performed using same settings as in FIG. 16D, and the hits were collected. The hits have significantly higher green fluorescence than most of the other library members. FIG. 16F is an image of an agar plate showing the results of an experiment in which aptamer-expressing E. coli were screened. Briefly, aptamer-expressing E. coli were screened on agar plate supplemented with DFHBI-1T. FACS isolated cells were directly plated on LB-agar plate containing 10 μM DFHBI-1T. The next day, the resulting colonies were induced with IPTG (final concentration of 1 mM) for 3 hours at 37° C. The plate was imaged using a ChemiDoc MP imager. Fluorescence of the RNA aptamer-DFHBI-1T complexes in the colonies was detected using ex: 470±15 nm, em: 532±14 nm. The bright green colonies were picked and sequenced to identify three unique library members 9-1, 9-2, and 9-3 which showed fluorescence activation of DFHBI-1T.

FIGS. 17A-17F demonstrate the development of Squash aptamer by directed evolution. FIG. 17A is a FACS scatter plot showing the fluorescence distribution of E. coli hits during the first round of directed evolution. To identify mutations which could improve the green fluorescence of 9-1 aptamer, a doped 9-1 RNA library was subjected to four rounds of SELEX (see Table 2 for detailed conditions). Next, the RNA library was cloned into an expression plasmid and transformed into E. coli. 10×10⁶ E. coli transformed with the round four RNA library, were screened for green fluorescence in presence of 20 μM DFHBI-1T (ex: 488 nm, em: 525±25 nm) using the gate shown, and the hits were collected. The collected cells are represented by the dots within the gate. The grey dots are E. coli cells which have lower green fluorescence and only used here for comparison. FIG. 17B shows screening of aptamer-expressing E. coli on agar plate supplemented with DFHBI-1T after first directed evolution. FACS isolated cells were plated on LB-agar plate containing 10 μM DFHBI-1T. The next day resulted colonies were induced with IPTG (final concentration of 1 mM) for 3 hours at 37° C. The plate was imaged using a ChemiDoc MP imager. Fluorescence of the RNA-DFHBI-1T complexes in the colonies was detected using ex: 470±15 nm, em: 532±14 nm. The bright green colonies (based on normalization of the green signal to far red auto-fluorescence) were picked and sequenced to identify three unique library members DE1-1, DE1-2 and DE1-3 which showed improved fluorescence activation of DFHBI-1T compared to 9-1. FIG. 17C is a FACS scatter plot showing the green fluorescence distribution of E. coli hits during the second round of directed evolution. To identify mutations which could improve the green fluorescence of DE1-2 aptamer, a doped DE1-2 RNA library was subjected to SELEX (see Table 3 for detailed conditions). After four rounds of SELEX, the RNA library was cloned and analyzed as in FIG. 17A. The collected cells are represented by dots within the right gate. The dots in the left gate are E. coli cells which have lower green fluorescence and only used here for comparison. FIG. 17D shows screening of aptamer-expressing E. coli on agar plate supplemented with DFHBI-1T after the second directed evolution. The FACS-isolated cells were plated on LB-agar plate containing 10 μM DFHBI-1T as described in FIG. 17B and treated as described in FIG. 17B. The brightest green colonies were picked and sequenced to identify six unique library members DE2-1 to DE2-6 which showed improved fluorescence activation of DFHBI-1T compared to DE1-2. FIG. 17E is a FACS scatter plot showing the DFHO-induced yellow fluorescence distribution of E. coli hits during the second directed evolution. To identify mutations which could improve the yellow fluorescence of DE1-2 aptamer, the doped DE1-2 RNA library (same as in FIG. 17C) was subjected to SELEX using DFHO-agarose beads. After four rounds of SELEX, the RNA library was transformed into E. coli. 10×10⁶ transformed E. coli cells were screened for yellow fluorescence in presence of 10 μM DFHO (ex: 488 nm, em: 545±17.5 nm) using the gate shown, and the hits were collected. The collected cells are represented by the yellow dots. The blue dots are E. coli cells which have lower green fluorescence and only used here for comparison. FIG. 17F shows screening of aptamer-expressing E. coli on agar plate supplemented with DFHO after second directed evolution. FACS isolated cells were plated on LB-agar plate containing 10 μM DFHO. The next day, the resultant colonies were induced with IPTG (final concentration of 1 mM) for 3 hours at 37° C. The plate was imaged using a ChemiDoc MP imager. Fluorescence of the RNA-DFHO complexes in the colonies was detected using ex: 530±14 nm, em: 605±25 nm. The bright yellow colonies were picked and sequenced to identify six unique library members. These hits had the same sequence as DE2-1 to DE2-6 which were identified using DFHBI-1T based screening (see FIG. 17D). The aptamer DE2-6 was further modified by a pair of mutations which resulted in the final Squash aptamer.

FIGS. 18A-18E show the mFOLD-predicted structure of the different aptamer intermediates during development of Squash. FIGS. 18A-18E show the mFold-predicted secondary structure of add A-riboswitch aptamer (FIG. 18A; add A-riboswitch aptamer: 5′-GGC UUC AUA UAA UCC UAA UGA UAU GGU UUG GGA GUU UCU ACC AAG AGC CUU AAA CUC UUG AUU AUG AAG UC-3′ (SEQ ID NO:46), 9-1 aptamer (FIG. 18B; 9-1 aptamer: 5′-GGC UUC AAG GUG GCC CAA UGA UAU GGU UUG GGU UAG GAU AGG AAU AAG AGC CUU AAA CUC UUC AAA GCG GAA GUC-3′ (SEQ ID NO:47), DE1-2 aptamer (FIG. 18C; DE1-2 aptamer: 5′-GGC UUC AAG GUG AGC CCA AUA AUA UGG UUU GGG UUA GGA UAG GAA GAA GAG CCU UAA ACU CUC UAA GCG GAA GUC-3′ (SEQ ID NO:48), DE2-6 aptamer (FIG. 18D; DE2-6 aptamer: 5′-GGC UAC AAG GUG AGC CCA AUA AUA UGG UUU GGG UUA GGA UAG GAA GUA GAG CCU UAA ACU CUC UAA GCG GUA GUC-3′ (SEQ ID NO:49), and Squash aptamer (FIG. 18E; Squash aptamer: 5′-GGC UAC AAG GUG AGC CCA AUA AUA CGG UUU GGG UUA GGA UAG GAA GUA GAG CCG UAA ACU CUC UAA GCG GUA GUC-3′ (SEQ ID NO:1). Importantly all the DFHBI-1T and DFHO binding aptamers shown here (FIGS. 18B-18E) have a four-nucleotide expansion of the binding pocket compared to add A-riboswitch aptamer (see FIG. 1E). The kissing loop interaction denoted by grey lines in FIG. 18D and FIG. 18E are essential for fluorescence activation (see FIG. 6G). The bulged nucleotide U51 (nucleotide at position 51) and the G·U pair (nucleotides at positions 18 and 38) shown in FIG. 18D are also important for fluorescence activation of DE2-6 (see FIG. 6F). FIG. 18E is exactly the same as FIG. 2A.

FIGS. 19A-19B demonstrate that F30 enhances Broccoli but not Squash fluorescence in E. coli. FIG. 19A demonstrates that F30 enhances the green fluorescence of Broccoli but not Squash in E. coli when used with DFHBI-1T dye. In FIG. 3C it was shown that F30 enhances the fluorescence of Broccoli when expressed as linear RNA in HEK293T cells. To test whether this also holds in E. coli, flow cytometry analysis was performed to compare E. coli cells expressing either Broccoli or F30-Broccoli and Squash or F30-Squash. All of these were linear RNAs. The aptamers were expressed from a T7 promoter using a pET28c-based expression plasmid. An empty pET28c plasmid which does not produce any aptamer was used as a negative control. After overnight culture, cells were induced with 1 mM IPTG for 3 hours at 37° C. Then the cells were incubated with 25 μM DFHBI-1T and used for flow analysis. The position of each dot reflects RNA green fluorescence (ex: 488 nm, em: 525±25 nm, x-axis) and side scattering, SSC-A (y-axis). F30-Broccoli expressing cells exhibited markedly higher fluorescence than Broccoli-expressing cells. This is consistent with the known ability of F30 to enhance the folding of Broccoli. However Squash and F30-Squash expressing cells exhibited similar fluorescence intensity. This is consistent with the possibility that Squash is already well-folded and thus cannot benefit from F30. FIG. 19B demonstrates that F30 does not enhance Squash fluorescence in E. coli when used with the DFHO dye. Similar to FIG. 19A, flow cytometry analysis was performed on E. coli transformed with the pET28c plasmid expressing either linear Squash or linear F30-Squash. After induction with 1 mM IPTG for 3 hours at 37° C., cells were incubated with 10 μM DFHO and used for flow analysis. The position of each dot reflects RNA orange fluorescence (ex: 488 nm, em: 570±20 nm, x-axis) and side scattering, SSC-A (y-axis). As can be seen, F30 was again unable to enhance the fluorescence intensity of Squash in E. coli. Taken together, these data suggest that Squash is highly folded when expressed as linear RNA in E. coli and F30 could not further enhance its folding.

FIGS. 20A-20B demonstrate the difference between cellular fluorescence of unscaffolded aptamer and F30 scaffolded aptamer is not due to different expression level. FIG. 20A demonstrates that 5S-Broccoli and 5S-F30-Broccoli are expressed at similar levels in HEK293T cells. In FIG. 3D it was shown that the 5S-F30-Broccoli formed puncta which are much brighter than those formed by 5S-Broccoli. One possible reason for this observation could be that 5S-F30-Broccoli is expressed at much higher level than 5S-Broccoli in HEK293T cells. To test this, total cellular RNA was isolated from HEK293T cells transfected with plasmids expressing 5S-Empty (no aptamer), 5S-Broccoli, or 5S-F30-Broccoli and analyzed them by staining with DFHBI-1T and SYBR Gold as described previously (Filonov et al., “In-Gel Imaging of RNA Processing Using Broccoli Reveals Optimal Aptamer Expression Strategies,” Chem. Biol. 22:649-660 (2015), which is hereby incorporated by reference in its entirety). 10 μg of total RNA for each sample was separated by 6% denaturing PAGE (7 M urea) and the gel was stained with 10 μM DFHBI-1T after removing the urea from the gel (by washing with water). After imaging the gel for green fluorescence (left), the gel was destained with water to remove DFHBI-1T and then stained with SYBR Gold. Then another image was taken (right). The bands which correspond to the 5S-Broccoli and 5S-F30-Broccoli in SYBR Gold staining were identified by comparing them with the 5S-Empty lane and marked by white stars. As can be seen in the images, the 5S-Broccoli and 5S-F30-Broccoli bands have similar intensity in the SYBR Gold scan suggesting similar expression levels of these two RNAs. Interestingly, the DFHBI-1T fluorescence intensity of the band corresponding to 5S-F30-Broccoli band is higher than that of 5S-Broccoli probably due to higher in vitro folding promoted by the F30-scaffold. FIG. 20B demonstrates that 5S-Squash and 5S-F30-Squash are expressed at similar level in HEK293T cells. To test if the expression levels of Squash and F30 Squash are similar, the total cellular RNA from HEK293T cells were analyzed similarly as in FIG. 20A except 10 μM DFHO was used for the first stain instead of DFHBI-1T. As can be seen, the 5S-Squash and 5S-F30-Squash bands have similar intensity in the SYBR Gold scan suggesting similar expression levels of these two RNAs. The DFHO fluorescence intensity of the bands corresponding to 5S-F30-Squash and 5S-Squash are also similar probably due to similar in vitro folding (see FIG. 3A).

FIG. 21 is a schematic representation of the SELEX approach to identify transducer domains for the Squash-SAM sensor. To make a Squash-SAM sensor, a SELEX-based approach was used to identify transducer domains that would allow the SAM aptamer to control folding of Squash. As seen in FIG. 4A, to generate a transducer library, the transducer region was randomized using the sprouts and clips method which resulted in sequences where the transducer strands are not only variable in composition but also in size. The different color of the transducer helix in the different library members shown here represents both the unique sequence and size of the transducer helix. To remove the members of the transducer library which showed SAM-independent constitutive folding of Squash, a negative selection was performed by incubating library members with DFHO-agarose beads. Then the unbound library members were recovered, and incubated with SAM. During this step, the potential SAM sensors in the library will undergo a SAM-dependent folding of Squash. To capture these potential sensors, the RNA library (with SAM) was incubated with DFHO-agarose beads. The non-binders which do not fold Squash in presence of SAM (blue and yellow) were removed by washing the beads with a buffer containing SAM. Finally the winners were eluted from the beads using a SAM-free buffer. In the absence of SAM, the transducer helix of the winners will become unstable which would lead to unfolding of Squash and detachment from the beads. Lastly, the winning library members were amplified using RT-PCR to generate a dsDNA library. This dsDNA library was transcribed and the resulting RNA library was used for the next round of SELEX.

FIGS. 22A-22B provide an example of gating strategies for FACS sorting and flow cytometry. FIG. 22A shows gating strategies for sorting of bacterial cells to identify fluorogenic aptamers. Cells were initially gated according to FSC-A and SSC-A. Then cells were gated according to FSC—W and FSC—H, selecting only singlets. Then a gate (Spinach+) was chosen such that the hits will have significant higher fluorescence than the negative controls. A second gate (P1) was chosen to record a small part of the negative events during the actual sorting of ˜10 million cells. FIG. 22B shows gating strategies for flow cytometry with HEK293T cells. Cells were initially gated according to FSC-A and SSC-A. Then cells were gated according to SSC—H and SSC—W, selecting only singlets. Then cells were gated using DAPI stain to select for live cells.

FIG. 23 shows unprocessed gel images corresponding to FIG. 20A (top) and FIG. 20B (bottom).

FIG. 24 is a table showing sequence analysis of 20 randomly chosen members from the sprouts and clips library (SEQ ID NOS: 15, 18, 22, 23, 24, 37, 39, 45, 50, and 51-59). Only the sequences of the mutagenized regions are shown. The number in the bracket indicates the number of nucleotides present in that region in the parental add A-riboswitch aptamer both in the column titles and experimentally determined sequences listed in the columns.

FIG. 25 is a Table showing sequences used for in vitro selection (SEQ ID NOS: 60-63), hits from the first SELEX (SEQ ID NOS: 9, 64, and 65), sequences for the first directed evolution (SEQ ID NOS: 66-68), hits from the first directed evolution (SEQ ID NOS: 69, 10, 70), sequences for the second directed evolution (SEQ ID NOS: 71-73), hits from the second directed evolution (SEQ ID NOS: 74-78 and 11), sequences of RNAs used for in vitro studies (SEQ ID NOS: 12 and 79-82), sequences for Squash-SAM sensor transducer library (SEQ ID NOS: 83-85), sequences of the Squash-SAM sensors (SEQ ID NOS: 26, 86, and 87), and sequences of circular ratiometric SAM sensors (SEQ ID NO: 88 and SEQ ID NO: 89) described herein.

FIGS. 26A-26B show the fluorescence properties of the Beetroot aptamer bound to the DFAME fluorophore. FIG. 26A shows the structures of the fluorophore DFHO (left) and DFAME (right). DFAME contains a more extended π-electron conjugation system compared to DFHO. Extension of the π-electron conjugation system typically red shifts the fluorescence emission and excitation spectra in fluorescent dyes. FIG. 26B shows the excitation and emission spectra of the Beetroot-DFAME complex. Spectra were measured using 20 μM RNA and 2 μM DFAME.

FIGS. 27A-27C demonstrate that Beetroot is a dimer in vitro. FIG. 27A is a sequence alignment of Corn (Corn: 5′-CGA GGA AGG AGG UCU GAG GAG GUC ACU G-3′ (SEQ ID NO:103) and Beetroot (Beetroot: 5′-GUU AGG CAG AGG UGG GUG GUG UGG AGG AGU AUC UGU C-3′ (SEQ ID NO:96)). The sequence alignment of Beetroot with Corn shows high similarity and 7 and 2-nt extensions on the 5′ and 3′ ends of Beetroot, respectively. Conserved nucleotides between Corn and Beetroot are highlighted in grey. G residues that form the G-quadruplex in Corn (nucleotides at positions 8, 9, 11, 12, 18, 19, 21, and 22 of SEQ ID NO:103) are in orange and are completely conserved in Beetroot (nucleotides at positions 15, 16, 18, 19, 23, 24, 26, and 27 of SEQ ID NO:96). FIG. 27B is an image showing that Beetroot is a dimer in vitro. To test this, nondenaturing electrophoretic analysis was performed on a wild-type Beetroot and a mutant Beetroot with mutations in the G-quadruplex that are expected to disrupt any putative dimer formation. In-gel staining with DFAME confirmed that only the putative dimeric Beetroot band binds DFAME, while the putative monomer does not bind or activate the fluorescence of DFAME. Furthermore, SYBR-Gold staining showed that wild-type Beetroot migrates at a higher molecular weight compared to the putative monomer under nondenaturing conditions. The RNA band marked with an asterisk (*) reflects an RNA side product from in vitro transcription. FIG. 27C are images showing that Beetroot and Corn do not form heterodimers with each other in vitro. To test whether Beetroot and Corn can form heterodimers in vitro, nondenaturing electrophoretic analysis was performed on Beetroot, Corn, and a Beetroot-Corn equimolar mixture to measure heterodimer formation. If Beetroot (107 nt) and Corn (250 nt) are orthogonal dimers, two distinct molecular species would be expected: dimeric Beetroot and dimeric Corn. On the other hand, if Beetroot-Corn heterodimers are formed, a third molecular species, reflecting a Beetroot-Corn heterodimer, with a molecular weight in between dimeric Beetroot and dimeric Corn, would be expected. The nondenaturing electrophoretic data showed that only the homodimers form. In-gel staining with DFAME and DFHO also confirmed the identity of the putative dimeric species. The RNA band marked with an asterisk (*) reflects an RNA side product from in vitro transcription. A final concentration of 350 nM RNA was used in each lane.

FIG. 28 are images showing the genetic encoding of simple macromolecular RNA assemblies in cells using Corn. To test whether Corn can be used to form genetically encoded RNA assemblies in cells, circular RNAs containing 1, 3, and 5 copies of Corn were expressed in HEK293T cells. Clusters of these Corn-containing RNAs as large intracellular puncta, with an increased tendency of these Corn-based RNA assemblies as the number of Corn aptamers increases in each circular RNA were observed. Notably, when circular RNA containing five Corn monomers (5× Corn) was expressed, most yellow fluorescence was concentrated in the Corn-based RNA assemblies with minimal diffused yellow fluorescence in the cytosol compared to 1× Corn and 3× Corn. Scale bar, 10 μm.

FIGS. 29A-29B show genetically encoded Corn- and Beetroot-protein assemblies in cells. FIG. 29A are images demonstrating that corn-based RNA assemblies can recruit and cluster proteins in cells. To test whether Corn-based assemblies can recruit specific proteins to form RNA-protein assemblies, a 5× Corn circular RNA, which contained an MS2 hairpin (5× Corn-MS2), was expressed. The MS2 hairpin binds to the MS2 coat protein (MCP) that is fused to mCherry (MCP-mCherry). Colocalization of the red fluorescence from MCP-mCherry to the yellow fluorescence from 5×Corn-MS2 was observed upon DFHO incubation with the cells. Scale bar, 10 μm. FIG. 29B are images showing Beetroot-based RNA-protein assemblies in cells. To test whether Beetroot can be used to form genetically encoded RNA assemblies and whether such can be used assemblies to recruit proteins, a 5× Beetroot circular RNA, which contained a boxB hairpin (5× Beetroot-boxB), was expressed. The boxB hairpin binds to a short peptide sequence, called N-peptide, which is fused to GFP (GFP-N-peptide). Green-fluorescent Beetroot-based assemblies were observed in the cell. After incubation with DFAME, red fluorescence was also observed from DFAME-Beetroot, which colocalized with the green fluorescence from GFP. This likely reflects the concentrated red fluorescence from DFAME-Beetroot in the RNA assemblies. Scale bar, 10 μm.

FIG. 30 are images demonstrating that Beetroot and Corn form simultaneous orthogonal RNA-protein assemblies in cells. To test whether Beetroot and Corn can form orthogonal assemblies in the same cell, circular 5× Corn-MS2 and 5× Beetroot-boxB were expressed in the same cells, and MCP-mCherry and GFP-N-peptide were coexpressed to visualize these RNA assemblies. Distinct green- and red-fluorescent assemblies, likely corresponding to the Beetroot- and Corn-based assemblies, were observed. Notably, overlap between these fluorescent assembly species was not observed. Scale bar, 10 μm.

FIGS. 31A-31B demonstrate that DFAME does not exhibit fluorescence activation or cytotoxicity when applied in mammalian cells. FIG. 31A is a graph showing the results of an experiment evaluating fluorescence of DFAME when mixed with cells. An important criteria for a fluorogenic dye is that it must have very low fluorescence when mixed with cells. To test this, 10 μM DFAME was incubated with cultured HEK293T cells and the fluorescence intensity of the DFAME-incubated cells was measured. It was found that the DFAME-incubated cells resulted in measurable, but low levels of red fluorescence. Although the background fluorescence in cells induced by DFAME (n=48 cells) was higher than DFHO (n=47 cells) or DFHBI-1T (n=44 cells), it was reasoned that DFAME could be used if enough fluorogenic aptamer is used in cells. Data were collected from three independent cell cultures. Values are means±s.d. FIG. 31B is a graph showing the cytotoxicity of DFAME. To apply a fluorogenic dye in cells, it is critical that this dye has minimal cytotoxicity. To test the cytotoxicity of DFAME, HEK293T cells were first incubated with 10 μM DFAME, then illuminated for 10 minutes using a Nikon Intensilight C-HGFIE 130 W mercury lamp with the TRITC filter set. Control cells were illuminated under identical conditions either with 0.2% DMSO (illuminated with the TRITC filter set) or 10 μM malachite green (illuminated with the Cy5 filter set). Cells were then washed once with media to remove fluorophores and assayed for mitochondrial activity with MTT (3-[4,5-dimethylthiazol-2-yl]-2,5-diphenyltetrazolium bromide) based cell proliferation kit (Roche). MTT absorbance readings were performed at 550 nm with 650 nm absorbance used for background subtraction. The results showed that DFAME exhibited low cytotoxicity in HEK293T cells. Data were collected from three technical replicates. Values are means±s.d.

FIGS. 32A-32D demonstrate the fluorescence activation of different fluorogenic dyes by Beetroot and Corn. FIG. 32A shows the fluorescence emission spectrum of DFHO and DFHO in complex with Beetroot. To test whether Beetroot can activate DFHO, the fluorescence emission spectrum of the Beetroot-DFHO complex was measured. In brief, 20 μM Beetroot and 2 μM DFHO were mixed, and the fluorescence emission of the Beetroot-DFHO complex was measured. It was found that Beetroot can only weakly bind and activate the fluorescence of DFHO. FIG. 32B shows the fluorescence emission spectrum of DFHBI-1T and DFHBI-1T and Beetroot. To test whether Beetroot can activate DFHBI-1T, the fluorescence emission spectrum of the Beetroot-DFHBI-1T complex was measured using the same protocol described in FIG. 32A. It was found that Beetroot cannot bind and activate the fluorescence of DFHBI-1T. FIG. 32C shows the fluorescence emission spectrum of BI and BI+Beetroot. To test whether Beetroot can activate BI, the fluorescence emission spectrum of the Beetroot-BI complex was measured using the same protocol described in FIG. 32A. It was found that Beetroot cannot bind and activate the fluorescence of BI. FIG. 32D shows the fluorescence emission spectrum of DFAME and DFAME+Corn. To test whether Corn can activate DFAME, the fluorescence emission spectrum of the Corn-DFAME complex was measured using the same protocol described in FIG. 32A. It was found that corn can only weakly bind and activate the red fluorescence of DFAME.

FIGS. 33A-33F shows characterizations of Corn and Beetroot. FIGS. 33A-33C provide the dissociation constant (K_{d}) of Beetroot homodimer. To determine the K_{d} of the Beetroot homodimer, serial dilution of Beetroot was performed to yield total RNA concentrations of 1000 nM, 333 nM, 111 nM, 37 nM, 12.3 nM, 4.1 nM, and 1.4 nM, respectively. These RNAs were incubated at 20° C. for 12 hours and then analyzed using non-denaturing gel electrophoresis (FIG. 33A). The fraction of Beetroot monomer (FIG. 33B) and Beetroot dimer (FIG. 33C) were quantified after SYBR Gold staining. These results indicate that no dissociation of the dimer into monomer was detected as the Beetroot concentration was diluted from 1000 nM to 1.4 nM. This suggests that K_{d} of Beetroot homodimer is likely less than 1 nM. The RNA band marked with an asterisk (*) reflects an RNA side product from in vitro transcription. The same side product band was also observed in FIGS. 27B-27C. Quantification shown in (FIG. 27B) and (FIG. 27C) is based on two independent experiments. FIG. 33D are images showing genetic encoding of simple macromolecular RNA assemblies in cells using Corn when circular RNAs containing 3 and 5 copies of Corn were expressed in HEK293T cells. Clusters of these Corn-containing RNAs were observed as large intracellular puncta. Data shown here is a representative image from three independent cell cultures. Scale bar, 20 μm. FIG. 33E is a graph showing the quantification of the fraction of Corn and Beetroot in the cell that accumulates in puncta. As the valency of Corn increases from three to five, the fraction of Corn participating in the RNA assembly increases from 28±12% to 57±14%. Compared to 5× Corn, a fraction of 27±11% of 5× Beetroot are participating in the Beetroot RNA assembly. Values are means±s.d. FIG. 33F is a graph showing quantification of RNA assembly size in cells. Shown are the size distribution of RNA assemblies created using the indicated multivalent Corn or Beetroot RNA. As the valency of Corn increases from three to five, an increase in the median RNA assembly size was noticed from 0.88±0.37 μm to 1.0±0.54 μm. Compared to 5× Corn, 5× Beetroot assembly has a size of 0.68±0.25 μm. Values are means±s.d.

FIGS. 34A-34C shows genetically encoded Corn- and Beetroot-protein assemblies in cells. FIG. 34A are images showing that Corn-based RNA assemblies can recruit and cluster proteins in cells. 5× Corn-MS2 (the MS2 hairpin binds to MCP-mCherry) was expressed in cells. Colocalization of the red fluorescence from MCP-mCherry to the yellow fluorescence from 5×Corn-MS2 was observed upon DFHO incubation with the cells. Data shown here is a representative image from three independent cell cultures. Scale bar, 20 μm. FIG. 34B are images showing Beetroot-based RNA-protein assemblies in cells. 5× Beetroot-boxB (the boxB hairpin binds to GFP-N-peptide) was expressed in cells. Green-fluorescent Beetroot-based assemblies were observed in the cell. After incubation with DFAME, red fluorescence was also observed from DFAME-Beetroot, which colocalized with the green fluorescence from GFP. Data shown here is a representative image from three independent cell cultures. Scale bar, 20 μm. FIG. 34C are fluorescence images from FIG. 34A with enhanced contrast in the Corn-DFHO channel to show some of the RNA assemblies with dim fluorescence (as indicated by white arrows). Scale bar, 10 μm.

FIGS. 35A-35B show imaging Beetroot in HEK293T cells. To test whether Beetroot could be imaged in HEK293T cells, Beetroot was expressed in Tornado. FIG. 35A shows that Beetroot was tested with either the tRNA (Ponchon and Dardel, “Recombinant RNA technology: The tRNA Scaffold,” Nat. Methods. 4:571-576 (2007), which is hereby incorporated by reference in its entirety)(tRNA-Beetroot: 5′-gcc cgg ata get cag tcg gta gag cag cgg ccg GTT AGG CAG AGG TGG GTG GTG TGG AGG AGT ATC TGT Ccg gcc gcg ggt cca ggg ttc aag tcc ctg ttc ggg c-3′ (SEQ ID NO:104), where nucleotides in lowercase are tRNA and nucleotides in uppercase correspond to Beetroot) or F30 (Filonov et al., “In-Gel Imaging of RNA Processing Using Broccoli Reveals Optimal Aptamer Expression Strategies,” Chem. Biol. 22: 649-660 (2015), which is hereby incorporated by reference in its entirety) (F30-tRNA-Beetroot: 5′-TTG CCA TGT GTA TGT GGG ACG CGT TGC CAC GTT TCC CAC ATA CTC TGA TGA TCC GCT AGC gcc cgg ata get cag tcg gta gag cag cgg ccg GTT AGG CAG AGG TGG GTG GTG TGG AGG AGT ATC TGT Ccg gcc gcg ggt cca ggg ttc aag tcc ctg ttc ggg cGG TAC CGG ATC ATT CAT GGC AA-3′ (SEQ ID NO:105), where nucleotides in bold uppercase are F30, nucleotides in lowercase are tRNA and nucleotides in uppercase correspond to Beetroot) aptamer folding scaffold. FIG. 35B are images showing HEK293T cells which were transiently transfected with Beetroot-expressing plasmids. About 36 hours after transfection, these transfected HEK293T cells were incubated with 10 μM DFAME and 5 mM MgCl₂ for 1 hour prior to imaging. Detectable levels of red fluorescence were not observed in HEK293T cells. Data shown here is a representative image from three independent cell cultures. Scale bar, 20 m.

FIG. 36 demonstrates that Beetroot and Corn form simultaneous orthogonal RNA-protein assemblies in cells. To test whether Beetroot and Corn can form orthogonal assemblies in the same cell, circular 5× Corn-MS2 and 5× Beetroot-boxB were expressed in the same cells, and MCP-mCherry and GFP-N-peptide were coexpressed to visualize these RNA assemblies. Distinct green and red fluorescent assemblies, likely corresponding to the Beetroot and Corn-based assemblies, respectively, were observed. Notably, overlap between these fluorescent assembly species was not observed. Data shown here is a representative image from three independent cell cultures. Scale bar, 20 μm.

FIGS. 37A-37D show the synthesis and characterization of methyl (Z)-4-(3,5-difluoro-4-hydroxybenzylidene)-1-methyl-5-oxo-4,5-dihydro-1H-imidazole-2-carboxylate (DFAME). FIG. 37A shows a scheme for the synthesis of DFAME. DFHBI (50.4 mg, 0.2 mmol, 1.0 equiv.), and selenium dioxide (22.2 mg, 0.2 mmol, 1.0 equiv.), and anhydrous dioxane (1 mL) were stirred at reflux for 1 hour, then the solution was filtered by vacuum while hot. Next, the volatiles were removed in vacuo, the crude product was dissolved in dichloromethane. Methyl triphenylphosphoranylidene)acetate (56.8 mg, 0.17 mmol, 0.9 equiv.) was added at 0° C., after stirring for 16 hours at room temperature, the volatiles were removed in vacuo and the residue was purified by column chromatography (silica gel, 1:1 petroleum ether:ethyl acetate) to afford DFAME as a red solid (25.0 mg, 39%). FIG. 37B shows the ¹H NMR spectrum of DFAME. ¹H NMR (500 MHz, DMSO) δ 11.17 (s, 1H), 8.04 (d, 2H), 7.45 (d, J=15.7 Hz, 1H), 7.14 (s, 1H), 7.11 (d, J=15.7 Hz, 1H), 3.80 (s, 3H), 3.23 (s, 3H) (FIG. 37B). FIG. 37C shows the ¹³C NMR spectrum of DFAME. ¹³C NMR (126 MHz, DMSO) δ 169.30, 165.23, 158.25, 151.95 (d, J=241.9 Hz), 151.89 (d, J=241.9 Hz), 138.19, 136.98 (t, J=16.4 Hz), 129.57, 127.93, 126.83 (t, J=3.2 Hz), 124.29 (t, J=8.8 Hz), 116.04 (d, J=6.2 Hz), 115.89 (d, J=6.2 Hz), 52.21, 26.45 (FIG. 37C). FIG. 37D shows the HRMS of DFAME. ESI-HR calcd for C₁₅H₁₁F₂N₂O₄([M−H]⁻) 321.0692, found 321.0689.

### DETAILED DESCRIPTION

The present disclosure relates to novel nucleic acid aptamers that can bind selectively to conditionally fluorescent molecules (referred to herein as “fluorophores”) to enhance the fluorescence signal of the fluorophore upon exposure to radiation of suitable wavelength. Molecular complexes formed between the novel aptamers and fluorophores, sensors comprising such novel nucleic acid aptamers optimized for metabolite-induced conformational changes when fused to a metabolite-binding sequence, and uses of those novel materials are also disclosed.

The aptamer and molecular complexes disclosed herein are useful for a wide variety of purposes, both in vitro and in vivo, including monitoring the location or degradation of RNA molecules in vivo and monitoring and quantifying the amount of a target molecule in an in vitro or in vivo system. Importantly, the fluorophores are non-toxic, unlike other dyes. The detection procedures can be implemented using existing optical detection devices amenable to high-throughput microarrays or drug screening. Moreover, the generation of RNA-based small molecule sensors demonstrates that it is possible to vastly increase the number molecules that can be detected in cells beyond what is possible using current protein-based FRET sensors. The present disclosure provides a rapid, simple, and general approach to obtain sensors for any small molecule. These sensors should immediately find use as simple fluorometric reagents to measure small molecules, thereby simplifying assays, and permitting high-throughput fluorescence-based screens.

Fluorophores and their Synthesis

In some embodiments, the fluorophores used in the present disclosure are characterized by a low quantum yield at a desired wavelength in the absence of aptamer binding. In certain embodiments, the quantum yield of the fluorophore, in the absence of specific aptamer binding, is less than about 0.01, or less than about 0.001, or less than about 0.0001.

The fluorophores are substantially unable to exhibit increases in quantum yield upon binding or interaction with molecules other than the aptamer(s) that bind specifically to them. This includes other molecules in a cell or sample besides those aptamer molecules having a polynucleotide sequence that was selected for binding to the fluorophore.

In some embodiments, the fluorophores are water soluble, non-toxic, and cell permeable. In some embodiments, the fluorophores are soluble in an aqueous solution at a concentration of 0.1 μM, 1 μM, 10 μM, 50 μM, or higher. In some embodiments, incubating a cell with these concentrations of the fluorophore does not affect the viability of the cell. The fluorophores are, in some embodiments, capable of migrating through a cell membrane or cell wall into the cytoplasm or periplasm of a cell by either active or passive diffusion. In some embodiments, the fluorophores are able to migrate through both the outer and inner membranes of gram-negative bacteria, the cell wall and membrane of gram-positive bacteria, both the cell wall and plasma membrane of plant cells, cell wall and membrane of fungi and molds (e.g., yeast), the capsid of viruses, the plasma membrane of an animal cell, and/or through the GI tract or endothelial cell membranes in animals.

As used herein, the terms “enhance the fluorescence signal” or “enhanced signal” (i.e., upon specific aptamer binding) refer to an increase in the quantum yield of the fluorophore when exposed to radiation of appropriate excitation wavelength, a shift in the emission maxima of the fluorescent signal (relative to the fluorophore emissions in ethanol glass or aqueous solution), an increase in the excitation coefficient, or two or more of these changes. In some embodiments, the increase in quantum yield is at least about 1.5-fold, at least about 5 to 10-fold, at least about 20 to 50-fold, or at least about 100 to about 200-fold. Fold increases in quantum yield exceeding 500-fold and even 1000-fold have been achieved.

The radiation used to excite the fluorophore may be derived from any suitable source, such as any source that emits radiation within the visible spectrum or infrared spectrum. The radiation may be directly from a source of radiation (e.g., a light source) or indirectly from another fluorophore (e.g., a FRET donor fluorophore). The use of FRET pairs is discussed more fully hereinafter.

In some embodiments, fluorophores that can be used in accordance with the present disclosure include those according to formula I below:

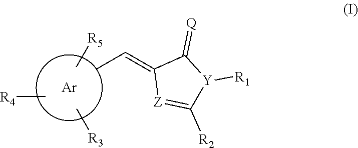

where,

Q is S or O,

Y is O or N,

Z is N or C(R₁₀),

Ar is an aromatic or hetero-aromatic ring system comprising one or two rings;

R₁ is present when Y is N, and is a C₁₋₈ hydrocarbon or —(CH₂)ₙ—R₆ where n is an integer greater than or equal to 1;

R₂ is methyl, a mono-, di-, or tri-halo methyl, an aldoxime, an O-methyl-aldoxime, iminomethyl, carboxylic acid, thioic acid, (thio)amido, alkyl(thio)amido, unsubstituted or substituted phenyl with up to three substituents (R₇-R₉), (meth)acrylate, C₂₋₈ unsaturated hydrocarbon optionally terminated with an amine, amide, carboxylic acid, ester, enone, oxime, O-methyl-oxime, imine, nitromethane, nitrile, ketone, mono-, di-, tri-halo, nitro, cyano, acrylonitrile, acrylonitrile-enoate, acrylonitrile-carboxylate, acrylonitrile-amide, alkylester, or a second aromatic or hetero-aromatic ring;

R₃-R₅ are independently selected from H, hydroxy, alkyl, alkoxy, fluoro, chloro, bromo, a mono-, di-, or tri-halo alkoxy, amino, alkylamino, dialkylamino, (thio)amido, alkyl(thio)amido, alkylthio, cyano, mercapto, nitro, and mono-, di-, or tri-halo methyl, ketone, carboxylic acid, thioc acid, alkylester, a surface-reactive group, a solid surface, or a functional group that can be linked to a reactive group on the solid surface;

R₆ is H, hydroxy, alkyl, alkoxy, fluoro, chloro, bromo, a mono-, di-, or tri-halo alkoxy, amino, alkylamino, dialkylamino, (thio)amido, alkyl(thio)amido, alkylthio, cyano, mercapto, nitro, and mono-, di-, or tri-halo methyl, ketone, carboxylic acid, alkylester, a surface-reactive group, a solid surface, or a functional group that can be linked to a reactive group on the solid surface; and

R₇-R₁₀ are independently selected from H, hydroxy, alkyl, alkoxy, fluoro, chloro, bromo, amino, alkylamino, dialkylamino, (thio)amido, alkyl(thio)amido, alkylthio, cyano, mercapto, nitro, and mono-, di-, or tri-halo methyl, ketone, carboxylic acid, thioic acid, and alkylester.

As used herein, alkyl substituents are C₁ to C₆ alkyls, in some embodiments methyl or ethyl groups. In the various substituents, an optional thio-derivative identified using, e.g., (thio)amido, is intended to encompass both amido and thioamido groups.

As used in the definition of R₃-R₆, the solid surface can be any solid surface, including glass, plastics, metals, semiconductor materials, ceramics, and natural or synthetic polymers (e.g., agarose, nitrocellulose). The solid surface can be an optically transparent material.

By surface-reactive group, it is intended that the group is a carboxylic acid (which can be modified by a carbodiimide to react with amines or alcohols), NHS ester, imidoester, PFP ester, p-nitrophenyl ester, hydroxymethyl phosphine, maleimide, haloacetyl group, haloacetamide group, vinyl sulfone, hydrazide, isocyanate, oxirane, epoxide, thiol, amine, alkyne, azide, anhydride, sulfonyl chloride, acyl chloride, ethylenimine, mixed disulfides, activated disulfides, or thiosulfinate.

By functional group that can be linked to a reactive group on a solid surface, it is intended that the group is any reactive group including, without limitation, carboxyl, amine, sulfhydryl, aldehyde, hydroxyl, thiol, or any of the groups listed as suitable for the surface-reactive group.

Suitable fluorophores may also encompass salts, including phenolate salts of compounds, including the compounds of formula (I).

Other known compounds within the scope of formula (I) include those where Ar is phenyl, Z and Y are both N, and either (i) R₃-R₅ are all H; (ii) R₁ and R₂ are methyl, R₄ and R₅ are H, and R₃ is hydroxy, methoxy, or dimethylamino; and (iii) R₁ is methyl, R₄ and R₅ are H, R₃ is hydroxy, and R₂ is a conjugated hydrocarbon chain. Other such compounds of formula I include those disclosed in He et al., “Synthesis and Spectroscopic Studies of Model Red Fluorescent Protein Chromophores,” Org. Lett. 4(9):1523-26 (2002); You et al., “Fluorophores Related to the Green Fluorescent Protein and Their Use in Optoelectornic Devices,” Adv. Mater. 12(22):1678-81 (2000); and Bourotte et al., “Fluorophores Related to the Green Fluorescent Protein,” Tetr. Lett. 45:6343-6348 (2004), each of which is hereby incorporated by reference in its entirety). In some embodiments, these previously known compounds are excluded from the scope of the invention.

Subclasses of these fluorophores, including oxazolithiones, pyrrolinthiones, imidazolithiones, and furanthiones, as well as those possessing an oxazolone ring, imidazolone ring, furanone ring, or pyrrolinone ring, are shown and/or described in PCT Application Publ. No. WO 2010/096584 to Jaffrey and Paige, which is hereby incorporated by reference in its entirety. In some embodiments, these previously known compounds are excluded from the scope of the invention.

Further diversification of fluorophore compounds can be achieved by conversion of an R₂ methyl group in compounds of formula (I) into an aldehyde using selenium dioxide (with dioxane under reflux). The resulting aldehyde can be converted into a C₂₋₈ unsaturated hydrocarbon, preferably a conjugated hydrocarbon, using the Wittig reaction. Basically, the resulting aldehyde is reacted with a triphenyl phosphine (e.g., Ph₃P=R₁₀ where R₁₀ is the unsaturated hydrocarbon) in the presence of strong base. The unsaturated hydrocarbon that is present in the Wittig reactant is optionally terminated with any desired functional group, preferably an amine, amide, carboxylic acid, (meth)acrylate, ester, enone, oxime, O-methyl-oxime, imine, nitromethane, nitrile, ketone, mono-, di-, tri-halo, nitro, cyano, acrylonitrile, acrylonitrile-enoate, acrylonitrile-carboxylate, acrylonitrile-amide, or a second aromatic or hetero-aromatic ring. These reactants are commercially available or readily synthesized by persons of skill in the art. Alternatively, the resulting aldehyde can be reacted with hydroxylamine or methoxyamine derivative according to the procedure of Maly et al., “Combinatorial Target-guided Ligand Assembly: Identification of Potent Subtype-selective c-Src Inhibitors,” Proc. Natl. Acad. Sci. U.S.A. 97(6): 2419-24 (2002), which is hereby incorporated by reference in its entirety) (see compounds of formulae IIIa, IIIb below). The aldehyde can also be reacted with nitromethane to form acrylonitro groups according to established protocols (see Muratore et al., “Enantioselective Bronsted Acid-catalyzed N-acyliminium Cyclization Cascades,” J. Am. Chem. Soc. 131(31):10796-7 (2009); Crowell and Peck, J. Am. Chem. Soc. 75:1075 (1953), each of which is hereby incorporated by reference in its entirety). Additionally, aldehydes can be reacted with nucleophilic cyano-containing molecules such as 2-cyanoacetamide, malononitrile methylcyanoacetate, cyano acetic acid, etc., in a Knoevenagel condensation reaction to produce acrylonitrile groups with different functional groups (Cope et al., J. Am. Chem. Soc. 63:3452 (1941), which is hereby incorporated by reference in its entirety).

Alternatively, the R₂ methyl can be replaced with a mono-, di-, or tri-halomethyl group. Halo-substituted acetamides are readily available, and are sufficiently reactive with the arylaldehydes.

In the compounds of formula (I), Ar can be any single or multiple (including fused) ring structure, except as noted above when Ar is phenyl. In some embodiments, Ar groups include substituted phenyl, naphthalenyl pyridinyl, pyrimidinyl, pyrrolyl, furanyl, benzofuranyl, thiophene-yl, benzothiophene-yl, thiazolyl, benzothiazolyl, imidizolyl, benzoimidizolyl, oxazolyl, benzoxazolyl, purinyl, indolyl, quinolinyl, chromonyl, or coumarinyl groups. The substituents of these Ar groups can be one or more of hydrogen, hydroxy, alkyl, alkoxy, fluoro, chloro, bromo, a mono-, di-, or tri-halo alkoxy, amino, alkylamino, dialkylamino, (thio)amido, alkyl(thio)amido, alkylthio, cyano, mercapto, nitro, and mono-, di-, or tri-halo alkyl, ketone, carboxylic acid, and thioc acid. The aromatic or hetero-aromatic group terminating the R₂ group can also be any one or the Ar groups identified above.

Other suitable subclasses of these compounds are the tri-substituted benzylidene imidazolones of formulae II, IIIa, and IIIb as described in PCT Application Publ. Nos. WO 2010/096584 and WO 2013/016694, both to Jaffrey et al., which are hereby incorporated by reference in their entirety.

Exemplary fluorophores identified in the above-referenced PCT Application Publ. Nos. WO 2010/096584 and WO 2013/016694 to Jaffrey et al. include, without limitation: 4-(3,4,5-trimethoxybenzylidene)-1,2-dimethyl-imidazol-5-one (“TMBI”); 4-(4-hydroxy-3,5-dimethoxybenzylidene)-1,2-dimethyl-imidazol-5-one (“DMHBI”); 4-(3,5-difluoro-4-hydroxybenzylidene)-1,2-dimethyl-imidazol-5-one (“DFHBI”); (E)-4-(3,5-difluoro-4-hydroxybenzylidene)-1-methyl-5-oxo-4,5-dihydro-1H-imidazole-2-carbaldehyde O-methyl oxime (“DFHBI-methyloxime”); 4-(3,5-dichloro-4-hydroxybenzylidene)-1,2-dimethyl-imidazol-5-one; 4-(3,5-dibromo-4-hydroxybenzylidene)-1,2-dimethyl-imidazol-5-one; 4-(2-hydroxybenzylidene)-1,2-dimethyl-imidazol-5-one (“o-HIBI”); 4-(2-methoxybenzylidene)-1,2-dimethyl-imidazol-5-one; 4-(3-fluoro-4-hydroxy-5-methoxybenzylidene)-1,2-dimethyl-imidazol-5-one; 4-(4-(dimethylamino)benzylidene)-1,2-dimethyl-imidazol-5-one (“DMABI”); 4-(4-(t-butylthio)benzylidene)-1,2-dimethyl-imidazol-5-one; 4-(4-(methylthio)benzylidene)-1,2-dimethyl-imidazol-5-one; 4-(4-cyanobenzylidene)-1,2-dimethyl-imidazol-5-one; 4-(3,5-difluoro-4-acetate)benzylidene-1,2-dimethyl-imidazol-5-one; 4-(4-hydroxy-3-nitrobenzylidene)-1,2-dimethyl-imidazol-5-one; 4-(4-hydroxy-3-methoxy-5-nitrobenzylidene)-1,2-dimethyl-imidazol-5-one; 4-(4-methoxy-3-nitrobenzylidene)-1,2-dimethyl-imidazol-5-one; 4-(4-bromobenzylidene)-1,2-dimethyl-imidazol-5-one; 4-(4-chlorobenzylidene)-1,2-dimethyl-imidazol-5-one; 4-(4-hydroxybenzylidene)-1,2-dimethyl-imidazol-5-one (“p-HBI”); 4-((indol-7-yl)methylene)-1,2-dimethyl-imidazole-5-one; 4-((indol-3-yl)methylene)-1,2-dimethyl-imidazole-5-one; 4-((indol-3-yl)methylene)-1-methyl-2-phenyl-imidazole-5-one; 4-(4-hydroxy-3,5-dimethoxybenzylidene)-1-methyl-2-phenyl-imidazole-5-one; 4-(4-(dimethylamino)benzylidene)-1-methyl-2-phenyl-imidazole-5-one; 4-(4-hydroxybenzylidene)-2-acetyl-1-methyl-imidazole-5-one; 4-(4-hydroxybenzylidene)-1-methyl-2-prop-1-enyl-imidazole-5-one; 3-(4-(4-hydroxybenzylidene)-4,5-dihydro-1-methyl-5-oxo-imidazol-2-yl)acrylamide; 3-(4-(4-hydroxybenzylidene)-4,5-dihydro-1-methyl-5-oxo-imidazol-2-yl)acrylic acid; and methyl 3-(4-(4-hydroxybenzylidene)-4,5-dihydro-1-methyl-5-oxo-imidazol-2-yl)acrylate. Of these, DFHBI and DFHBI-methyloxime have distinct emission maxima and high quantum yield and, therefore, may in some embodiments be particularly desirable.

Additional conditional fluorophores include, without limitation:

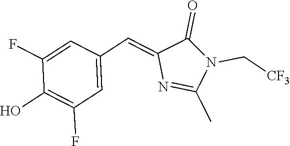 [SMILES: CC1=N/C(=C\c2cc(F)c(O)c(F)c2)C(=O)N1CC(F)(F)F]

(Z)-4-(3,5-difluoro-4-hydroxybenzylidene)-2-methyl-1-(2,2,2-trifluoroethyl)-1H-imidazol-5(4H)-one) (“DFHBI-1T”)

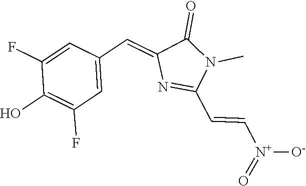 [SMILES: CN1C(=O)/C(=C/c2cc(F)c(O)c(F)c2)N=C1/C=C/[N+](=O)[O-]]

4-(3,5-difluoro-4-hydroxybenzylidene)-1-methyl-2-((E)-2-nitrovinyl)-1H-imidazol-5(4H)-one (“DFAN”)

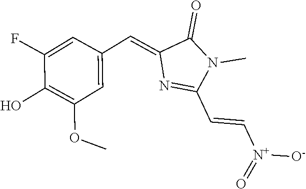 [SMILES: COc1cc(/C=C2N=C(/C=C/[N+](=O)[O-])N(C)C\2=O)cc(F)c1O]

4-(3-fluoro-4-hydroxy-5-methoxybenzylidene)-1-methyl-2-((E)-2-nitrovinyl)-1H-imidazol-5(4H)-one;

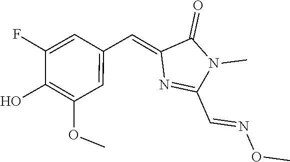 [SMILES: CO/N=C/C1=NC(=C\c2cc(F)c(O)c(OC)c2)/C(=O)N1C]

4-(3-fluoro-4-hydroxy-5-methoxybenzylidene)-1-methyl-5-oxo-4,5-dihydro-1H-imidazole-2-carbaldehyde O-methyl oxime;

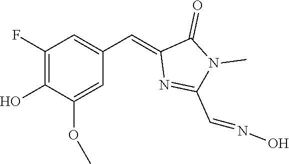 [SMILES: COc1cc(/C=C2N=C(/C=N/O)N(C)C\2=O)cc(F)c1O]

4-(3-fluoro-4-hydroxy-5-methoxybenzylidene)-1-methyl-5-oxo-4,5-dihydro-1H-imidazole-2-carbaldehyde oxime (“MFHO”);

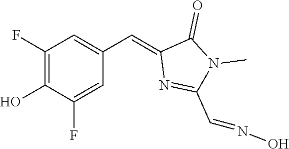 [SMILES: CN1C(=O)/C(=C/c2cc(F)c(O)c(F)c2)N=C1/C=N/O]

4-(3,5-difluoro-4-hydroxybenzylidene)-1-methyl-5-oxo-4,5-dihydro-1H-imidazole-2-carbaldehyde oxime (“DFHO”);

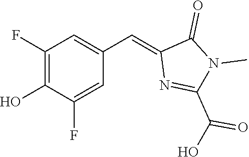 [SMILES: CN1C(=O)/C(=C/c2cc(F)c(O)c(F)c2)N=C1C(=O)O]

4-(3,5-difluoro-4-hydroxybenzylidene)-1-methyl-5-oxo-4,5-dihydro-1H-imidazole-2-carboxylic acid;

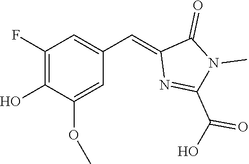 [SMILES: COc1cc(/C=C2\N=C(C(=O)O)N(C)C2=O)cc(F)c1O]

4-(3-fluoro-4-hydroxy-5-methoxybenzylidene)-1-methyl-5-oxo-4,5-dihydro-1H-imidazole-2-carboxylic acid;

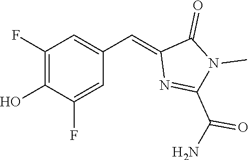 [SMILES: CN1C(=O)/C(=C/c2cc(F)c(O)c(F)c2)N=C1C(N)=O]

4-(3,5-difluoro-4-hydroxybenzylidene)-1-methyl-5-oxo-4,5-dihydro-1H-imidazole-2-carboxamide;

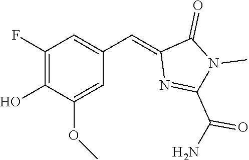 [SMILES: COc1cc(/C=C2\N=C(C(N)=O)N(C)C2=O)cc(F)c1O]

4-(3-fluoro-4-hydroxy-5-methoxybenzylidene)-1-methyl-5-oxo-4,5-dihydro-1H-imidazole-2-carboxamide;

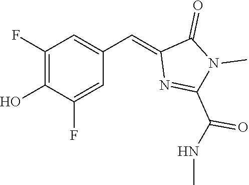 [SMILES: CNC(=O)C1=N/C(=C\c2cc(F)c(O)c(F)c2)C(=O)N1C]

4-(3,5-difluoro-4-hydroxybenzylidene)-N,1-dimethyl-5-oxo-4,5-dihydro-1H-imidazole-2-carboxamide;

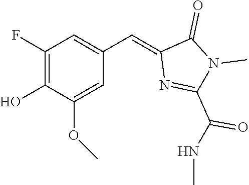 [SMILES: CNC(=O)C1=N/C(=C\c2cc(F)c(O)c(OC)c2)C(=O)N1C]

4-(3-fluoro-4-hydroxy-5-methoxybenzylidene)-N,1-dimethyl-5-oxo-4,5-dihydro-1H-imidazole-2-carboxamide;

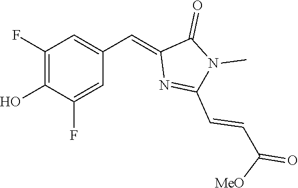 [SMILES: COC(=O)/C=C/C1=NC(=C\c2cc(F)c(O)c(F)c2)/C(=O)N1C]

methyl 3-((Z)-4-(3,5-difluoro-4-hydroxybenzylidene)-1-methyl-5-oxo-4,5-dihydro-1H-imidazol-2-yl)acrylate (“DFAME”);

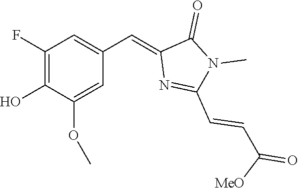 [SMILES: COC(=O)/C=C/C1=NC(=C\c2cc(F)c(O)c(OC)c2)/C(=O)N1C]

methyl 3-(4-(3-fluoro-4-hydroxy-5-methoxybenzylidene)-1-methyl-5-oxo-4,5-dihydro-1H-imidazol-2-yl)acrylate, and

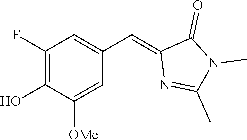 [SMILES: COc1cc(/C=C2\N=C(C)N(C)C2=O)cc(F)c1O]

4-(3-fluoro-4-hydroxy-5-methoxybenzylidene)-1,2-dimethyl-1H-imidazol-5(4H)-one (“MFHBI”). Of these, DFAN, DFAME, DFHO, MFHO, and MFHBI may be particularly desirable because of their distinct emission maxima, relative to DFHBI, DFHBI-1T, and DFHBI-methyloxime, and their high quantum yield. DFHBI-1T is also desirable because of its improved properties relative to DFHBI.

If cell permeability is a problem for some fluorophores, then acylation of phenolic moieties may improve the cell permeability without impacting fluorophore activity, as these acyl moieties are rapidly cleaved by intracellular esterases (see Carrigan et al., “The Engineering of Membrane-permeable Peptides,” Anal. Biochem. 341:290-298 (2005), which is hereby incorporated by reference in its entirety). For fluorophores with low cell permeability, their O-acyl esters can be trivially made by reacting the fluorophores with the appropriate acid chloride, e.g., myristoyl, octanoyl, or butanoyl chloride. To the extent that these acyl moieties are not rapidly cleaved, these may in fact improve the fluorescence of the various RNA-fluorophore complexes.

Another aspect of the present disclosure relates to a compound having a structure methyl (Z)-4-(3,5-difluoro-4-hydroxybenzylidene)-1-methyl-5-oxo-4,5-dihydro-1H-imidazole-2-carboxylate (DFAME).

A further aspect of the present disclosure relates to a method of making a compound having a structure methyl (Z)-4-(3,5-difluoro-4-hydroxybenzylidene)-1-methyl-5-oxo-4,5-dihydro-1H-imidazole-2-carboxylate (DFAME). Synthesis of methyl (Z)-4-(3,5-difluoro-4-hydroxybenzylidene)-1-methyl-5-oxo-4,5-dihydro-1H-imidazole-2-carboxylate (DFAME) is described infra in the Examples. In some embodiments, methyl (Z)-4-(3,5-difluoro-4-hydroxybenzylidene)-1-methyl-5-oxo-4,5-dihydro-1H-imidazole-2-carboxylate (DFAME) is synthesized from DFHBI, which is commercially available. DFHBI, selenium dioxide, and anhydrous dioxane are stirred at reflux and the solution is filtered by vacuum while hot. Volatiles are removed in vacuo and the crude product is dissolved in dichloromethane. Methyl (triphenylphosphoranylidene)acetate is added and stirred. The volatiles are removed in vacuo and the residue is purified by column chromatography to afford DFAME.

### Aptamers

The present disclosure also relates to nucleic acid molecules that are known in the art as aptamers. Aptamers are single-stranded nucleic acid molecules that have a secondary structure that may possess one or more stems (i.e., base-paired regions) as well as one or more non base-paired regions along the length of the stem. These non-base-paired regions can be in the form of a bulge or loop (e.g., internal loop) along the length of the stem(s) and/or a loop at the end of the one or more stem(s) (e.g., hairpin loop). These nucleic acid aptamers possess specificity in binding to a particular target molecule, and they noncovalently bind their target molecule through an interaction such as an ion-ion force, dipole-dipole force, hydrogen bond, van der Waals force, electrostatic interaction, stacking interaction or any combination of these interactions.

Identifying useful nucleic acid aptamers involves selecting aptamers that bind a particular target molecule with sufficiently high affinity (e.g., K_{d}<500 nM) and specificity from a pool or library of nucleic acids containing a random region of varying or predetermined length. For example, identifying useful nucleic acid aptamers can be carried out using an established in vitro selection and amplification scheme known as SELEX. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk and Gold, “Systematic Evolution of Ligands by Exponential Enrichment: RNA Ligands to Bacteriophage T4 DNA Polymerase,” Science 249:505-510 (1990), each of which is hereby incorporated by reference in their entirety. An established template-primer system (Bartel et al., “HIV-1 Rev Regulation Involves Recognition of Non-Watson-Crick Base Pairs in Viral RNA,” Cell 67:529-536 (1991), which is hereby incorporated by reference in its entirety) can be adapted to produce RNA molecules having a stretch of about 38-40 random bases sandwiched between 5′ and 3′ constant regions.

The synthetic oligonucleotide templates can be amplified by polymerase chain reaction (“PCR”) and then transcribed to generate the original RNA pool. Assuming that ten percent of the RNA molecules are free of chemical lesions that prevent second-strand synthesis and transcription, this pool would contain more than 3×10¹³ different sequences. Because filter binding is applicable for most protein targets, it can be used as the partitioning device, although other suitable schemes can be used. The selected primary RNA aptamers can be cloned into any conventional subcloning vector and sequenced using any variation of the dideoxy method. Next, the secondary structure of each primary RNA aptamer can be predicted by computer programs such as MulFold or mFOLD (Jaeger et al., “Improved Predictions of Secondary Structures for RNA,” Proc. Natl. Acad. Sci. U.S.A. 86:7706-7710 (1989), and Zuker, “On Finding All Suboptimal Foldings of an RNA Molecule,” Science 244:48-52 (1989), each of which is hereby incorporated by reference in its entirety). Mutational studies can be conducted by preparing substitutions or deletions to map both binding sites on the RNA aptamer and its target molecule, as well as to further enhance aptamer binding affinity.

Aptamers generated from SELEX experiments can be optimized to produce second generation aptamers with improved properties (Eaton et al., “Post-SELEX Combinatorial Optimization of Aptamers,” Bioorg. Med. Chem. 5:1087-1096 (1997), which is hereby incorporated by reference in its entirety). Through successive rounds of affinity maturation of a primary SELEX clone, it is possible to obtain aptamers that possess improved fluorescence and higher quantum yield characteristics than the original clone. Therefore, prior to using aptamers in cell-based experiments, each aptamer can be optimized using the following considerations:
Find the minimal aptamer sequence within the SELEX clone to identify the domain to subject to affinity maturation. This will lead to more desirable, smaller aptamers, which should be better for tagging RNAs with aptamers.
It is important to know if the aptamers are selective for their intended fluorophore or if they bind other fluorophores that are intended to bind to other aptamers. In dual color imaging experiments involving two RNA-fluorophore complexes, cross-reactive fluorophores would be problematic.
The fluorescence of the aptamer-fluorophore complexes may be optimized by affinity maturation. This may avoid unwanted interference or FRET.
Additionally, tagging the target molecule with multiple tandem aptamers rather than a single aptamer will increase the fluorescence of a tagged target molecule. Tagging of the aptamers should be possible without impacting the aptamer ability to bind specifically to a particular fluorophore or target molecule of interest.

If any cross-reactivity is observed, then a doped library can be prepared and subjected to “negative selection,” also called “counter-SELEX.” The ability of negative selection to generate aptamers with high degrees of selectivity, even among closely related molecules is known (Tuerk et al., “Using the SELEX Combinatorial Chemistry Process to Find High Affinity Nucleic Acid Ligands to Target Molecules,” Methods Mol. Biol. 67:219-230 (1997); Rink et al., “Creation of RNA Molecules that Recognize the Oxidative Lesion 7,8-dihydro-8-hydroxy-2′-deoxyguanosine (8-oxodG) in DNA,” Proc. Natl. Acad. Sci. U.S.A. 95:11619-11624 (1998); Haller et al., “In vitro Selection of a 7-Methyl-guanosine Binding RNA that Inhibits Translation of Capped mRNA Molecules,” Proc. Natl. Acad. Sci. U.S.A. 94:8521-8526 (1997); Edwards et al., “DNA-oligonucleotide Encapsulating Liposomes as a Secondary Signal Amplification Means,” Anal. Chem. 79:1806-1815 (1997), each of which is hereby incorporated by reference in its entirety). To perform negative selection, RNAs bound to dye-agarose are subjected to a washing step in which the buffer contains other fluorophores. This results in the elution of aptamers that have undesirable cross-reactivity. The RNAs that remain bound to the agarose beads are then eluted with the fluorophore of interest, and amplified as in the classic SELEX procedure. This process is repeated until clones are generated which do not bind and activate the fluorescence of inappropriate fluorophores.

Optimization of aptamers can also be achieved during re-selection by using rigorous washing conditions in all steps, including the use of high temperature (37° C. or 45° C.) washing buffers, mild denaturants, and low salt and high salt washes, etc. Since the quantum yield may reflect the efficiency of the RNA to conformationally restrict the photoexcited fluorophores, RNA aptamers that bind more tightly to the fluorophore may improve the quantum yield, and thereby the fluorescence of the RNA-fluorophore complexes. The proposed stringent washing conditions are intended to select for aptamers that bind more tightly to the fluorophore, and thereby improve the quantum yield. An additional benefit of generating RNA aptamers that bind with higher affinity to the fluorophore is that lower concentrations of fluorophore will be needed for live-cell experiments, which may reduce potential off-target or cytotoxic effects of the fluorophore. Since most aptamers that bind to small molecules bind with modest affinity, i.e., a K_{d} of >100 nM (Famulok et al., “Nucleic Acid Aptamers-from Selection in vitro to Applications in vivo,” Accounts Chem. Res. 33:591-599 (2000), which is hereby incorporated by reference in its entirety), it is expected that this high affinity will not affect the resistance to photobleaching.

Another method to use during optimization is the use of a smaller bias during doping. For example, the library can be doped with a 2:1:1:1 ratio instead of 5:1:1:1. This will result in more library members being substantially different from the parent aptamer.

The SELEX procedure can also be modified so that an entire pool of aptamers with binding affinity can be identified by selectively partitioning the pool of aptamers. This procedure is described in U.S. Patent Application Publication No. 2004/0053310 to Shi et al., which is hereby incorporated by reference in its entirety.

Single stranded DNA aptamers have advantages for in vitro settings due to their ease of synthesis and greater stability. Recent studies have argued that proper buffer conditions and certain RNA sugar modifications can lead to highly stable RNAs (Osborne et al., “Aptamers as Therapeutic and Diagnostic Reagents: Problems and Prospects,” Curr. Opin. Chem. Biol. 1:5-9 (1997); Faria et al., “Sugar Boost: When Ribose Modifications Improve Oligonucleotide Performance,” Curr. Opin. Mol. Ther. 10:168-175 (2008), each of which is hereby incorporated by reference in its entirety). Additionally, microarrays of RNAs have been shown to be stable in the presence of tissue lysates when suitable RNAase inhibitors are added (Collett et al., “Functional RNA Microarrays for High-throughput Screening of Antiprotein Aptamers,” Anal. Biochem. 338:113-123 (2005), which is hereby incorporated by reference in its entirety). Moreover, as part of the optimization and stabilization process, stabilizing hairpins can be added which markedly enhance aptamer levels in cells (Blind et al., “Cytoplasmic RNA Modulators of an Inside-out Signal-transduction Cascade,” Proc. Natl. Acad. Sci. U.S.A. 96:3606-3610 (1999), which is hereby incorporated by reference in its entirety). Regardless, DNA aptamer sequences that switch on fluorophores described herein would be inexpensive to synthesize and provide additional assurance of sensor stability in solution phase or microarray-based assays.

Another approach for optimization of the SELEX procedure, particularly with respect to the in vivo activity of aptamers in binding to an inducing fluorescence of conditionally fluorescent molecules of the type described herein, includes FACS sorting of recombinant cells that express the aptamer and exhibit fluorescence in the presence of both a properly folded aptamer and an appropriately selected conditionally fluorescent molecule. Briefly, SELEX is carried out until the RNA pool exhibits the capacity to bind to the conditional fluorophore of interest. At this point, the RNA pool is reverse transcribed and cloned into a bacterial expression plasmid to prepare an aptamer expression library. In some embodiments, the aptamer is cloned so that it is transcribed fused to a suitable aptamer-folding scaffold, e.g., tRNA^{Lys}₃ (Ponchon et al., “Recombinant RNA Technology: the tRNA Scaffold,” Nat Methods 4(7): 571-6 (2007); Paige et al, “RNA Mimics of Green Fluorescent Protein,” Science 333(6042): 642-6 (2011); and Strack et al., “A Superfolding Spinach2 Reveals the Dynamic Nature of Trinucleotide Repeat-Containing RNA,” Nat Methods 10(12):1219-24 (2013), which are hereby incorporated by reference in their entirety).

After transformation of the library into bacterial host cells and transcription induction, bacteria are then sorted by FACS in presence of the conditional fluorophore to identify those aptamers that exhibit the highest fluorescence. In certain embodiments, the plasmid may also contain a separate promoter for expressing a far-red fluorescent protein which allows the aptamer fluorescence to be normalized to cell volume. Sorted bacteria are recovered and grown on agar dishes and imaged in presence of the fluorophore. Plasmid DNA from the brightest colonies can be isolated, sequenced and transcribed into RNA for further characterization. This process can be repeated for more than one round.

A further approach for optimization of the SELEX procedure involves the generation of random libraries (see, e.g., Hall et al., “Design, Synthesis, and Amplification of DNA Pools for In Vitro Selection,” Curr. Protoc. Mol. Biol 24:24.2.1-24.2.27 (2009), which is hereby incorporated by reference in its entirety) containing (i) shortened members which comprise fewer nucleotides relative to a template sequence (termed “clips”) and/or (ii) extended members which comprise nucleotide insertion(s) relative to a template sequence (termed “sprouts”).

Overall, the protocols described infra offer rapid and efficient methods to isolate fluorescent aptamers from the large initial random library.

SELEX can be performed as readily with DNA as with RNA (Breaker, “DNA Aptamers and DNA Enzymes,” Curr. Opin. Chem. Biol. 1:26-31 (1997), which is hereby incorporated by reference in its entirety). The absence of a 2′-OH does not substantially impair the ability of DNA to fold or adopt structures. Indeed, SELEX has been used to identify DNAs that bind both small molecules and proteins, with structures that are reminiscent of RNA aptamers. Thus, DNA aptamers can be developed and subjected to analogous mutagenesis and truncation studies to identify entry points and analyte sensors as described herein.

As used herein, “nucleic acid” includes both DNA and RNA, in both D and L enantiomeric forms, as well as derivatives thereof (including, but not limited to, 2′-fluoro-, 2′-amino, 2′O-methyl, 5′iodo-, and 5′-bromo-modified polynucleotides). Nucleic acids containing modified nucleotides (Kubik et al., “Isolation and Characterization of 2′fluoro-, 2′amino-, and 2′fluoro-amino-modified RNA Ligands or Human IFN-gamma that Inhibit Receptor Binding,” J. Immunol. 159:259-267 (1997); Pagratis et al., “Potent 2′-amino, and 2′-fluoro-2′-deoxy-ribonucleotide RNA Inhibitors of Keratinocyte Growth Factor,” Nat. Biotechnol. 15:68-73 (1997), each which is hereby incorporated by reference in its entirety) and the L-nucleic acids (sometimes termed Spiegelmers©), enantiomeric to natural D-nucleic acids (Klussmann et al., “Mirror-image RNA that Binds D-adenosine,” Nat. Biotechnol. 14:1112-1115 (1996) and Williams et al., “Bioactive and nuclease-resistant L-DNA Ligand of Vasopressin,” Proc. Natl. Acad. Sci. U.S.A. 94:11285-11290 (1997), each which is hereby incorporated by reference in its entirety), and non-natural bases are used to enhance biostability. In addition, the sugar-phosphate backbone can be replaced with a peptide backbone, forming a peptide nucleic acid (PNA), other natural or non-natural sugars can be used (e.g., 2′-deoxyribose sugars), or phosphothioate or phosphodithioate can be used instead of phosphodiester bonds. The use of locked nucleic acids (LNA) is also contemplated.

In some embodiments, the nucleic acid molecule includes a domain—an aptamer—that binds specifically to a fluorophore having a methyne bridge between a substituted aromatic ring system and a substituted imidazol(thi)one, oxazol(thi)one, pyrrolin(thi)one, or furan(thi)one ring. In some embodiments, the fluorophore is a compound according to any formulae recited in PCT Application Publ. Nos. WO 2010/096584 and WO 2013/016694 to Jaffrey et al., which are briefly described above. These nucleic acid aptamers, upon binding to the fluorophore, induces the fluorophore to adopt a conformation whereby the fluorescent emission spectrum is substantially enhanced upon exposure to radiation of suitable wavelength.

The nucleic acid aptamer molecules disclosed herein may bind to fluorophores described herein to induce fluorescence in the, e.g., red, orange, yellow, or green region of the visible spectrum.

### I. Squash Aptamers

In some embodiments, the nucleic acid aptamer molecules according to the present disclosure bind to the fluorophore 4-(3,5-difluoro-4-hydroxybenzylidene)-1-methyl-5-oxo-4,5-dihydro-1H-imidazole-2-carbaldehyde oxime (“DFHO”); (Z)-4-(3,5-difluoro-4-hydroxybenzylidene)-2-methyl-1-(2,2,2-trifluoroethyl)-1H-imidazol-5(4H)-one (“DFHBI-1T”); (Z)-5-(3,5-difluoro-4-hydroxybenzylidene)-2,3-dimethyl-3,5-dihydro-4H-imidazol-4-one (“DFHBI”); (Z)-3-amino-5-(3,5-difluoro-4-hydroxybenzylidene)-2-thioxothiazolidin-4-one (“NRD5”); (Z)-5-(3,5-difluoro-4-hydroxybenzylidene)-3-methyl-2-(trifluoromethyl)-3,5-dihydro-4H-imidazol-4-one (“DFHBI-2T”); and/or the fluorophore (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”).

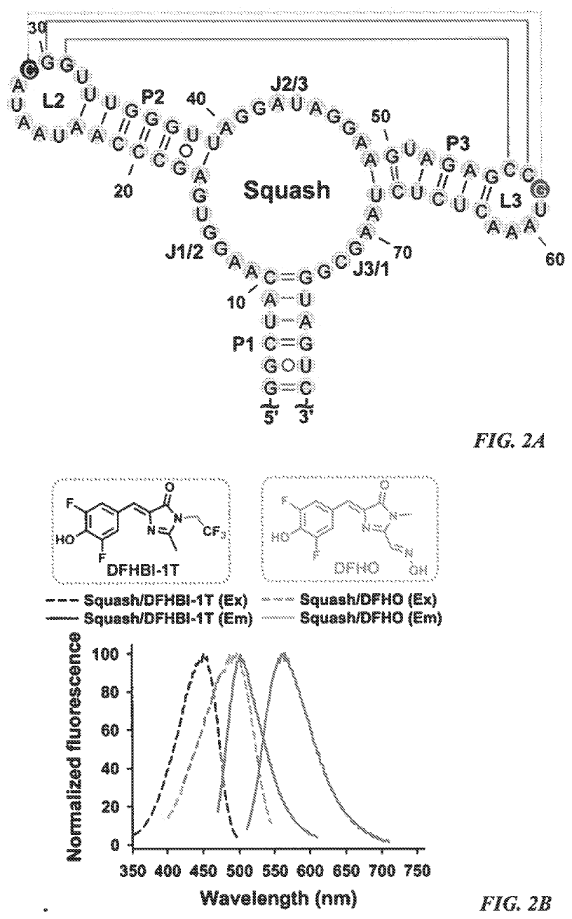

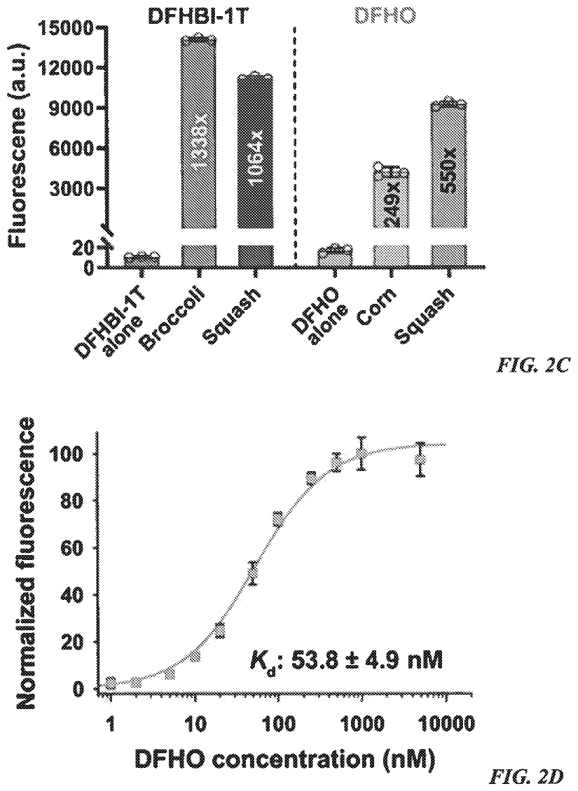

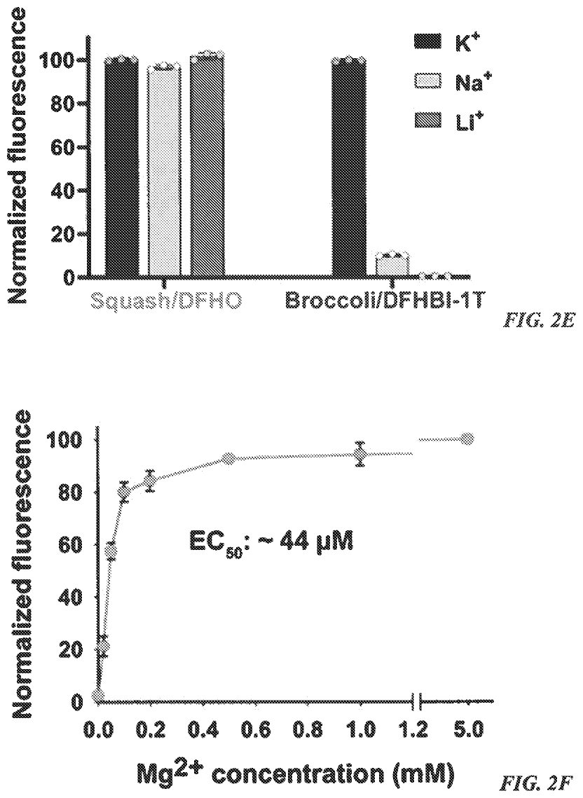

Such nucleic acid aptamer molecules, upon binding to the fluorophore, induce the fluorophore to adopt a conformation whereby the fluorescent emission spectrum is substantially enhanced upon exposure to radiation of suitable wavelength. Alternatively, the nucleic acid aptamers may suppress fluorophore conformations or conformational changes that lead to nonradiative decay of the photon-exicted fluorophore. An additional alternate mechanism is that the nucleic acid aptamers may alter the electronic properties to favor fluorescence emission over non-fluorescent decay pathways. Exemplary nucleic acid aptamer molecules include Squash nucleic acid aptamer molecules which, upon folding, form a three-way junction comprising helices P1, P2, and P3; loops L2 and L3, as well as junctional strands J1/2, J2/3, and J3/1, as shown in FIG. 2A. Such Squash nucleic acid aptamer molecules comprise base pairs within L2 and L3 which form kissing loop interactions, as shown in FIG. 2A between residues at positions 30 and 57 of SEQ ID NO: 1, residues at positions 29 and 58 of SEQ ID NO: 1, as well as residues at positions 31 and 56 of SEQ ID NO: 1.

Thus, according to one aspect of the present disclosure, the Squash nucleic acid aptamer molecule includes the nucleotide sequence of:
(i) GGC UAC AAG GUG AGC CCA AUA AUA CGG UUU GGG UUA GGA UAG GAA GUA GAG CCG UAA ACU CUC UAA GCG GUA GUC (SEQ ID NO: 1);
(ii) GGC UAC AAG GUG AGC CCA AUA AUA GGG UUU GGG UUA GGA UAG GAA GUA GAG CCC UAA ACU CUC UAA GCG GUA GUC (SEQ ID NO: 2);
(iii) GGC UAC AAG GUG AGC CCA AUA AUA NNN UUU GGG UUA GGA UAG GAA GUA GAG NNN UAA ACU CUC UAA GCG GUA GUC (SEQ ID NO: 3), where N at positions 25 and 54 are complementary to each other, N at positions 26 and 53 are complementary to each other, and N at positions 27 and 52 are complementary to each other, optionally where the complementary positions base pair with each other; or
(iv) GGC UAC AAG GUG NNN NNN NUA AUA NNN UNN NNN NNA GGA UAG GAN NNN NNN NNN UAA ANN NNN NAA GCG GUA GUC (SEQ ID NO: 4),
   where N at positions 25 and 54 are complementary to each other,
   N at positions 26 and 53 are complementary to each other,
   N at positions 27 and 52 are complementary to each other,
   N at positions 13 and 35 are complementary to each other,
   N at positions 14 and 34 are not complementary to each other,
   N at positions 15 and 33 are complementary to each other,
   N at positions 16 and 32 are complementary to each other,
   N at positions 17 and 31 are complementary to each other,
   N at positions 18 and 30 are complementary to each other,
   N at positions 19 and 29 are complementary to each other
   N at positions 45 and 64 are complementary to each other,
   N at positions 46 and 63 are complementary to each other,
   N at positions 48 and 62 are complementary to each other,
   N at positions 49 and 61 are complementary to each other,
   N at positions 50 and 60 are complementary to each other, and/or
   N at positions 51 and 59 are complementary to each other, optionally where the complementary positions base pair with each other.

Additional exemplary nucleic acid molecules include core Squash nucleic acid aptamers molecules, which may form a two-way junction comprising helices P2 and P3; loops L2 and L3, as well as junctional strand J2/3, as shown in FIG. 2A. Such core Squash nucleic acid aptamer molecules comprise base pairs within L2 and L3 which form kissing loop interactions, as shown in FIG. 2A.

Thus, according to another aspect of the present disclosure, the core Squash nucleic acid aptamer molecule includes the nucleotide sequence of:
(i) AAG GUG AGC CCA AUA AUA CGG UUU GGG UUA GGA UAG GAA GUA GAG CCG UAA ACU CUC UAA GCG (SEQ ID NO: 5);
(ii) AAG GUG AGC CCA AUA AUA GGG UUU GGG UUA GGA UAG GAA GUA GAG CCC UAA ACU CUC UAA GCG (SEQ ID NO: 6);
(iii) AAG GUG AGC CCA AUA AUA NNN UUU GGG UUA GGA UAG GAA GUA GAG NNN UAA ACU CUC UAA GCG (SEQ ID NO: 7), where N at positions 19 and 48 are complementary to each other, N at positions 20 and 47 are complementary to each other, and N at positions 21 and 46 are complementary to each other, optionally where the complementary positions base pair with each other; or
(iv) AAG GUG NNN NNN NUA AUA NNN UNN NNN NNA GGA UAG GAN NNN NNN NNN UAA ANN NNN NAA GCG (SEQ ID NO: 8), where N at positions 19 and 48 are complementary to each other,
   N at positions 20 and 47 are complementary to each other,
   N at positions 21 and 46 are complementary to each other,
   N at positions 7 and 29 are complementary to each other,
   N at positions 8 and 28 are not complementary to each other,
   N at positions 9 and 27 are complementary to each other,
   N at positions 10 and 26 are complementary to each other,
   N at positions 11 and 25 are complementary to each other,
   N at positions 12 and 24 are complementary to each other,
   N at positions 13 and 23 are complementary to each other,
   N at positions 39 and 58 are complementary to each other,
   N at positions 40 and 57 are complementary to each other,
   N at positions 41 and 56 are complementary to each other,
   N at positions 43 and 55 are complementary to each other,
   N at positions 44 and 54 are complementary to each other, and/or
   N at positions 45 and 53 are complementary to each other, optionally where the complementary sequences base pair with each other.

In some embodiments of this and other aspects of the present disclosure, key binding interactions are in the J regions. In some embodiments, P and L sequences can be changed without affecting the fluorophore-activating function.

Additional exemplary nucleic acid aptamer molecules include extended Squash nucleic acid aptamer molecules which, upon folding, form a three-way junction comprising helices P1, P2, and P3; loops L2 and L3, as well as junctional strands J1/2, J2/3, and J3/1, as shown in FIG. 2A, and which include additional nucleotides positioned 3′ and 5′ to the P1 helix. Such extended Squash nucleic acid aptamer molecules comprise base pairs within L2 and L3 which form kissing loop interactions, as shown in FIG. 2A between residues at positions 30 and 57 of SEQ ID NO: 1, residues at positions 29 and 58 of SEQ ID NO: 1, as well as residues at positions 31 and 56 of SEQ ID NO: 1.

Thus, according to another aspect of the present disclosure, the extended Squash nucleic acid aptamer molecule includes the nucleotide sequence of:

(i)
(SEQ ID NO: 9) (also referred to herein as 9-1)
GCC UAG GCU UCA AGG UGG CCC AAU GAU AUG GUU
UGG GUU AGG AUA GGA AUA AGA GCC UUA AAC UCU
UCA AAG CGG AAG UCU AGG C;
(ii)
(SEQ ID NO: 10) (also referred to herein as
DE1-2)
GCC UAG GCU UCA AGG UGA GCC CAA UAA UAU GGU
UUG GGU UAG GAU AGG AAG AAG AGC CUU AAA CUC
UCU AAG CGG AAG UCU AGG C;
(iii)
(SEQ ID NO: 11) (also referred to herein as
DE2-6)
GCC UAG GCU ACA AGG UGA GCC CAA UAA UAU GGU
UUG GGU UAG GAU AGG AAG UAG AGC CUU AAA CUC
UCU AAG CGG UAG UCU AGG C;
or
(iv)
(SEQ ID NO: 12) (also referred to herein
as Squash)
GGU AGG CUA CAA GGU GAG CCC AAU AAU ACG GUU
UGG GUU AGG AUA GGA AGU AGA GCC GUA AAC UCU
CUA AGC GGU AGU CUA CC.

According to another aspect of the present disclosure, the consensus Squash nucleic acid aptamer molecule includes the nucleotide sequence of:
(i) GGU AGG CUA CNN NNN NAG CCC AAU AAU ACG GUU UGG GUU NNN NNN NNN AGU AGA GCC GUA AAC UCU CUN NNN NGU AGU CUA CC (SEQ ID NO: 13), where N at each of positions 11-16, 40-48, and 69-73 can be any single nucleotide insertion of any length;
(ii) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 8 forms a stem loop, n at position 18 forms a stem loop, n at positions 1 and 24 form a stem;
(iii) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 1 has the sequence of CUA C, n at position 8 has the sequence of AGC CCA AUA AUA CGG UUU GGG UU (SEQ ID NO: 16), n at position 18 has the sequence of AGU AGA GCC GUA AAC UCU CU (SEQ ID NO: 17), and n at position 24 has the sequence of GUA G;
(iv) CUAC AAGGUG AGCCCAAUAAUACGGUUUGGGUU AGGAUAGGA AGUAGAGCCGUAAACUCUCU AAGCG GUAG (SEQ ID NO: 19);
(v) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 1 has the sequence of GGU CCA GAU GCC UUG UAA CCG AAA GGG ACA C (SEQ ID NO: 20), n at position 8 has the sequence of AGC CCA AUA AUA CGG UUU GGG U (SEQ ID NO: 21), n at position 18 has the sequence of AGU AGA GCC GUA AAC UCU CU (SEQ ID NO: 17), and n at position 24 has the sequence of GUG UCG AAA GGA UGG ACC (SEQ ID NO: 25);
(vi) GGU CCA GAU GCC UUG UAA CCG AAA GGG ACA CAA GGU GAG CCC AAU AAU ACG GUU UGG GUU AGG AUA GGA AGU AGA GCC GUA AAC UCU CUA AGC GGU GUC GAA AGG AUG GAC C (SEQ ID NO: 26);
(vii) nNA GNU GAN NNN NNN NNN NNN NNN NNN NNU AGG AUA GGA ANN NNN NNN NNN NNN NNN NUN AGC Gn (SEQ ID NO: 27), where n at positions 1 and 65 can be a nucleotide insertion of 1-200 nucleotide bases, N at positions 2, 5, 9-29, 41-58, and 60 is any nucleotide base, N at positions 9-29 forms a step loop, and N at positions 41-58 forms a stem loop comprising a bulge in the stem, optionally where n at positions 1 and 65 form a stem; (viii) nNA GNU GAN UAG GAU AGG AAN UNA GCG n (SEQ ID NO: 28), where n at positions 1 and 28 can be a nucleotide insertion of 1-200 nucleotide bases, N at positions 2, 5, and 23 are any nucleotide base, N at position 9 is a nucleotide insertion of 1-500 nucleotide bases where the insertion forms a stem loop, and N at position 21 is a nucleotide insertion of 1-500 nucleotide bases where the insertion forms a stem loop comprising a bulge in the stem, optionally where n at positions 1 and 28 form a stem;
(ix) nNA GNU GAN NNN NNN NNN NNN NNN NNN NNU AGG AUA GGA ANN NNN NNN NNN NNN NNN UNA GCG n (SEQ ID NO: 29), where n at positions 1 and 64 can be a nucleotide insertion of 1-200 nucleotide bases, N at positions 2, 5, 9-29, 41-57, and 59 is any nucleotide base, N at positions 9-29 forms a step loop, and N at positions 41-57 forms a stem loop, optionally where n at positions 1 and 64 form a stem;
(x) nNA GNU GAN UAG GAU AGG AAN UNA GCG n (SEQ ID NO: 30), where n at positions 1 and 28 can be a nucleotide insertion of 1-200 nucleotide bases, N at positions 2, 5, and 23 are any nucleotide base, N at position 9 is a nucleotide insertion of 1-500 nucleotide bases where the insertion forms a stem loop, and N at position 21 is a nucleotide insertion of 1-500 nucleotide bases where the insertion forms a stem loop, optionally where n at positions 1 and 28 form a stem; or
(xi) nAA GGU GAG CCC AAU AAU ACG GUU UGG GUU AGG AUA GGA AGU AGA GCC GUA AAC UCU CUA AGC Gn (SEQ ID NO: 31), where n at positions 1 and 65 can be a nucleotide insertion of 1-200 nucleotide bases, optionally where n at positions 1 and 65 forms a stem.

According to some embodiments, a nucleic acid aptamer molecule includes the nucleotide sequence of: GGU AGG CUA CAA GGU GAG CCC AAU AAU ACG GUU UGG GUU AGG AUA GGA AGU AGA GCC GUA AAC UCU CUA AGC GGU AGU CUA CC (SEQ ID NO: 12) (also known as Squash). This aptamer sequence can be preceded or followed by additional nucleotide sequences at its 5′ and 3′ ends that do not materially affect the relevant structure or binding activity.

The nucleic acid aptamer molecules according to the present disclosure may induce at least a 100-fold, at least a 200-fold, at least a 300-fold, at least a 400-fold, at least a 500-fold, at least a 600-fold, at least a 700-fold, at least an 800-fold, at least a 900-fold, at least a 1,000-fold, or more enhancement in the fluorescence of a fluorophore, such as the fluorophore 4-(3,5-difluoro-4-hydroxybenzylidene)-1-methyl-5-oxo-4,5-dihydro-1H-imidazole-2-carbaldehyde oxime (“DFHO”) and/or the fluorophore (Z)-4-(3,5-difluoro-4-hydroxybenzylidene)-2-methyl-1-(2,2,2-trifluoroethyl)-1H-imidazol-5(4H)-one (DFHBI-1T).

The nucleic acid aptamer molecules according to the present disclosure may, in some embodiments, comprise a secondary structure including a multi-branched loop such as a 3-way junction. In accordance with such embodiments, the nucleic acid aptamer molecules may comprise a first hairpin loop and a second hairpin loop, where at least one, two, or three nucleic acid residues in the first hairpin loop may base pair with one, two, or three nucleic acid residues in the second hairpin loop, respectively.

In some embodiments, the nucleic acid aptamers according to the present disclosure do not bind to the fluorophore through a G-quadruplex.

### II. Beetroot Aptamers

In some embodiments, the nucleic acid aptamer molecules according to the present disclosure bind to the fluorophore 3,5-difluoro-4-hydroxybenzylidene imidazolinone-2-acrylate methyl (DFAME) and/or the fluorophore 4-(3,5-difluoro-4-hydroxybenzylidene)-1-methyl-5-oxo-4,5-dihydro-1H-imidazole-2-carbaldehyde oxime (“DFHO”). Such nucleic acid aptamer molecules, upon binding to the fluorophore, induce the fluorophore to adopt a conformation whereby the fluorescent emission spectrum is substantially enhanced upon exposure to radiation of suitable wavelength. Exemplary nucleic acid aptamer molecules include Beetroot nucleic acid aptamer molecules which, upon folding, form a helix and a loop.

Thus, according to another aspect of the present disclosure, the Beetroot nucleic acid aptamer molecule includes the nucleotide sequence of:
(i) GGU GGG UGG UGU GGA GGA GUA (SEQ ID NO: 32) (also referred to herein as a core Beetroot nucleic acid aptamer sequence);
(ii) nGG UGG GUG GUG UGG AGG AGU An (SEQ ID NO: 33), where n at positions 1 and 23 is a nucleotide insertion of 1-200 nucleotide bases, optionally where n at position 1 forms a stem with n at position 23;
(iii) NNN NNN GGU GGG UGG UGU GGA GGA GUA NNN NNN (SEQ ID NO: 34), where N at positions 1 and 33 are complementary to each other and form a base pair,
   N at positions 2 and 32 are complementary to each other and form a base pair,
   N at positions 3 and 31 are complementary to each other and form a base pair,
   N at positions 4 and 30 are complementary to each other and form a base pair,
   N at positions 5 and 29 are complementary to each other and form a base pair, and/or
   N at positions 6 and 28 are complementary to each other and form a base pair, optionally where N at positions 1-6 forms a stem with N at positions 28-33; or
(iv) nnn nNN NNN NGG UGG GUG GUG UGG AGG AGU ANN NNN N (SEQ ID NO: 35), where n at positions 1-4 is any nucleotide base,
   N at positions 5 and 37 are complementary to each other and form a base pair,
   N at positions 6 and 36 are complementary to each other and form a base pair,
   N at positions 7 and 35 are complementary to each other and form a base pair,
   N at positions 8 and 34 are complementary to each other and form a base pair,
   N at positions 9 and 33 are complementary to each other and form a base pair, and/or
   N at positions 10 and 32 are complementary to each other and form a base pair, optionally where N at positions 5-10 forms a stem with N at positions 32-37.

In some embodiments, the Beetroot nucleic acid aptamer molecule comprises a core Beetroot nucleic acid aptamer sequence (i.e., SEQ ID NO: 32).

In some embodiments, the Beetroot nucleic acid aptamer comprises a helix. In accordance with such embodiments, the Beetroot nucleic acid aptamer sequence is selected from the group consisting of SEQ ID NO: 33, SEQ ID NO: 34, and SEQ ID N: 35).

In some embodiments, the Beetroot nucleic acid aptamer includes additional nucleotides positioned 5′ or 3′ to the helix. In accordance with such embodiments, the Beetroot nucleic acid aptamer sequence is SEQ ID NO: 35.

In accordance with this aspect of the disclosure, the nucleic acid aptamer molecules may form dimers, e.g., homodimers. In some embodiments, a nucleic acid homodimer may form a G-quadruplex.

Nucleic acid aptamer molecules (i.e., aptamers) described herein may include both monovalent aptamers that contain a single first domain for binding to the fluorophore, as well as multivalent aptamers that contain more than one aptamer domain.

In some embodiments, the nucleic acid aptamer molecule can include a plurality of first domains for binding to multiple identical fluorophore compounds per molecule. These can be in the form of concatemers of a single type of aptamer that binds to a single fluorophore. Examples of these concatemers that are useful for expanding the fluorescent emissions per molecule include 2-mers, 3-mers, 4-mers, 5-mers, 6-mers, 7-mers, 8-mers, 9-mers, 10-mers, 11-mers, 12-mers, 13-mers, 14-mers, 15-mers, 16-mers, 17-mers, 18-mers, 19-mers, 20-mers, 21-mers, 22-mers, 23-mers, 24-mers, 25-mers, 26-mers, 27-mers, 28-mers, 29-mers, 30-mers, 31-mers, and 32-mers. In forming these concatemers, the plurality of aptamer domains can be separated by linker regions of a suitable length (e.g., about 30 to about 100 nts) that prevents steric or folding interference between the distinct aptamer domains, allowing each to properly fold and bind to their target fluorophores. Alternatively, the concatemers can contain multiple types of aptamers that bind to a several different fluorophores, and collectively achieve a blended emission profile.

In many of these aptamer constructs, where a single fluorophore binding domain is used, the single fluorophore binding domain can be replaced with a concatemer containing multiple fluorophore binding domains. For example, multiple fluorophore binding sequences, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or more, can be linked together in series with adjacent fluorophore binding sequences separated by a spacer sequence that is sufficiently long (e.g., 2 to 100 nucleotides) so as to inhibit interference between adjacent fluorophore binding sequences. In certain embodiments, the fluorophore binding sequences can be slightly different from one another (or at least relative to immediately adjacent fluorophore binding sequences) to ensure that each aptamer sequence self-hybridizes to fold properly rather than hybridize with other aptamer sequences. Because each individual aptamer sequence within the concatemer is capable of binding to its fluorophore, use of the concatemer is expected to increase the fluorescence per aptamer construct. In this way, it is possible to design aptamer constructs where as few as a single molecule can be detected.

Nucleic acid aptamer molecules described herein can also be directed to specific cellular locations by creating nucleic acid fusion with a nucleic acid sequence that is targeted to specific domains in the cells due to intrinsic sequence properties, because they bind biomolecules or proteins that are at these cellular locations.

In some embodiments, a nucleic acid aptamer construct described herein includes one or more first domains that bind specifically to multiple identical fluorophore compounds per molecule, and a second domain that includes a random nucleotide sequence.

By “random,” it is contemplated that the entirety of the second domain, or merely a portion thereof, contains a nucleotide sequence that is not known a priori, but rather is generated randomly. Thus, a portion of the second domain may contain a known sequence, but the entirety of the second domain sequence is not known. Multivalent aptamer constructs of this type are prepared as “turn-on” sensors, as described above, and are useful for de novo screening and identification of aptamers having affinity for a target molecule of interest. These multivalent nucleic acid aptamer constructs can be generated during a modified SELEX process as described hereinafter. Thus, in some embodiments, the present disclosure relates to a library of these multivalent nucleic acid aptamer constructs. In the library, each member of the initial library preferably contains a unique or substantially unique random sequence (i.e., shared by few, if any, other initial library members).

### Sensors

Another aspect of the present disclosure relates to an RNA-based metabolite sensor comprising (i) a metabolite-binding aptamer portion and (ii) a regulated aptamer portion comprising the nucleic acid aptamer molecule according to the present disclosure and a transducer domain, where the regulated aptamer portion is linked to the metabolite-binding aptamer portion by the transducer domain.

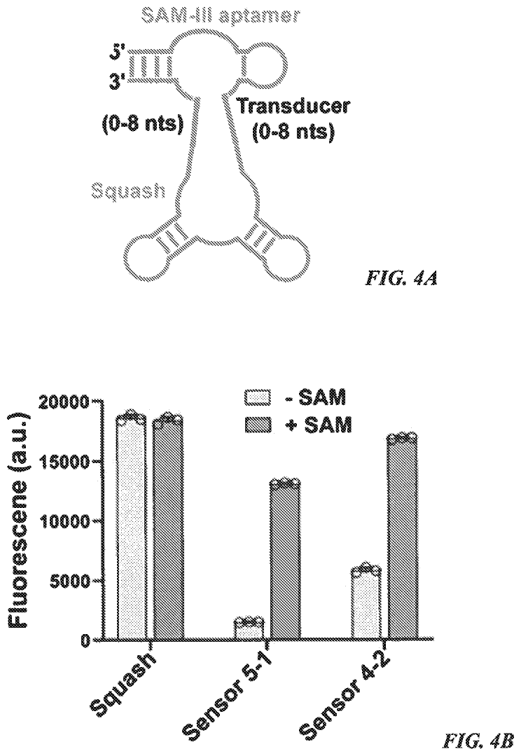

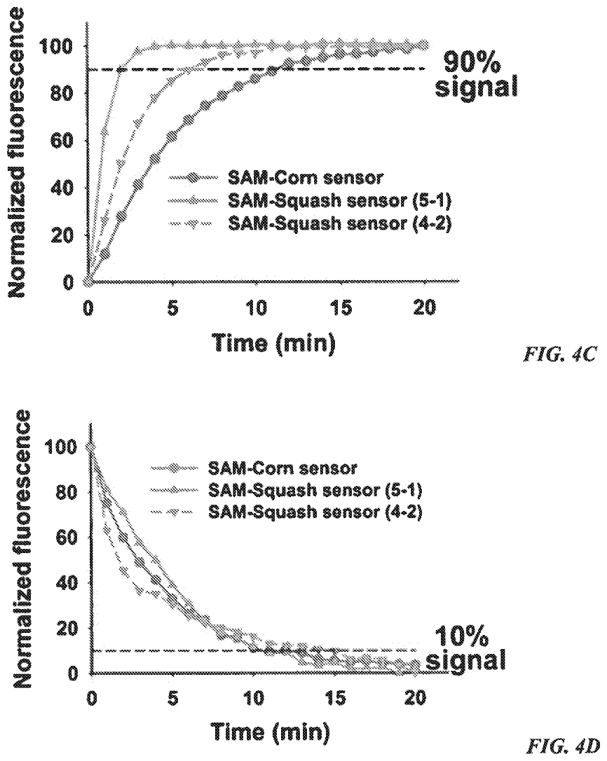

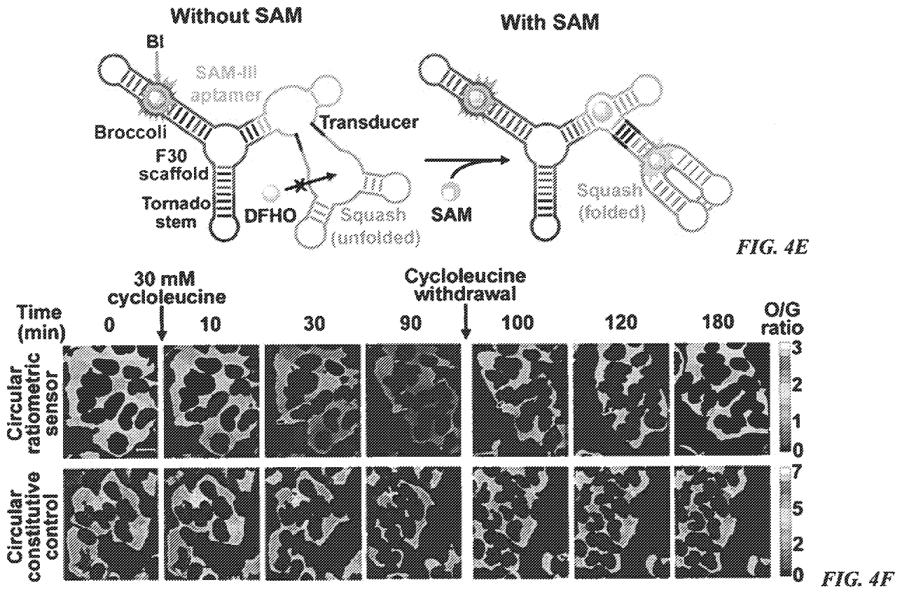

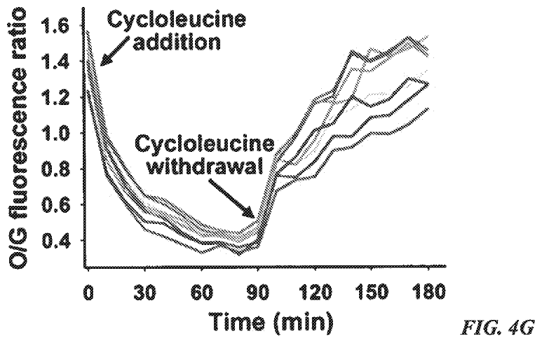

An exemplary RNA-based metabolite sensor according to the present disclosure is shown in the embodiment illustrated in FIG. 4A. In accordance with this embodiment, a metabolite-binding aptamer (i.e., the SAM-III aptamer) was fused to a regulated aptamer portion comprising a nucleic acid molecule according to the present disclosure (i.e., Squash) through a transducer helix.

In accordance with this aspect of the disclosure, the regulated aptamer portion may comprise one or more nucleic acid aptamer molecules according to the present disclosure that bind specifically to multiple identical fluorophore compounds per molecule.

In accordance with this aspect of the disclosure, the metabolite-binding portion binds specifically to a target molecule of interest (i.e., one that is distinct of the fluorophore). In some embodiments, the metabolite-binding aptamer portion comprises multiple target binding sites.

Also contemplated herein are concatemers of such RNA-based metabolite sensors, having the structure (metabolite-binding aptamer portion-regulated aptamer portion)m, where m is an integer greater than 1. In these concatemers, the metabolite-binding aptamer portion of each functional RNA-based metabolite sensor can be the same or different. Likewise, the regulated aptamer portion of each RNA-based metabolite sensor can be the same or different. In another embodiment, the concatemer includes a single metabolite-binding aptamer portion that binds specifically to the target molecule of interest and a plurality of regulated aptamer portions, which can be the same or different but which bind to the same fluorophore.

The target molecule of interest can be any biomaterial or small molecule including, without limitation, proteins, nucleic acids (RNA or DNA), lipids, oligosaccharides, carbohydrates, small molecules, hormones, cytokines, chemokines, cell signaling molecules, metabolites, organic molecules, and metal ions. The target molecule of interest can be one that is associated with a disease state or pathogen infection.

In some embodiments, the metabolite-binding aptamer portion binds specifically to a target molecule, e.g., a target nucleic acid via hybridization (e.g., Watson-Crick base-pairing). Thus, the metabolite-binding aptamer portion may comprise a nucleotide sequence that is sufficiently complementary to its target molecule so as to hybridize under appropriate conditions with the target molecule that is physiologically found within a cell or within a biological sample. Upon hybridization between the metabolite-binding aptamer portion and the target molecule, and the binding of the nucleic acid aptamer molecule according to the present disclosure (e.g., a Squash nucleic acid aptamer molecule or a Beetroot nucleic acid aptamer molecule) to a fluorophore (introduced to the sample or cell), the target molecule is effectively labeled by the fluorophore. Presence of the target molecule therefore can be detected based on the presence of fluorescence by the particular fluorophore employed.

Protein or polypeptide targets can be any length, and can include, without limitation, phosphoproteins, lipid-modified proteins, nitrosylated proteins, sulfenated proteins, acylated proteins, methylated proteins, demethylated proteins, C-terminal amidated proteins, biotinylated proteins, formylated proteins, gamma-carboxylated proteins, glutamylated proteins, glycylated proteins, iodinated proteins, hydroxylated proteins, isoprenylated proteins, lipoylated proteins (including prenylation, myristoylation, farnesylation, palmitoylation, or geranylation), proteins covalently linked to nucleotides such as ADP ribose (ADP-ribosylated) or flavin, oxidated proteins, proteins modified with phosphatidylinositol groups, proteins modified with pyroglutamate, sulfated proteins, selenoylated proteins, proteins covalently linked to another protein (including sumoylation, neddylation, ubiquitination, or ISGylation), citrullinated proteins, deamidated proteins, eliminylated proteins, disulfide bridged proteins, proteolytically cleaved proteins, proteins in which proline residues have been racemized, any peptides sequences that undergo the above mentioned modifications, and proteins which undergo one or more conformational changes. In addition, proteins or peptides that possess a mutation can be distinguished from wildtype forms. Complexes of two or more molecules include, without limitation, complexes having the following interactions: protein-protein, protein-cofactor, protein-inhibiting small molecules, protein-activating small molecules, protein-small molecules, protein-ion, protein-RNA, protein-DNA, DNA-DNA, RNA-DNA, RNA-RNA, modified nucleic acids-DNA or RNA, aptamer-aptamer. In addition, nucleic acids that possess a mutation can be distinguished from wildtype forms.

Nucleic acid targets can be any type of nucleic acid including, without limitation, DNA, RNA, LNA, PNA, genomic DNA, viral DNA, synthetic DNA, DNA with modified bases or backbone, mRNA, noncoding RNA, PIWI RNA, termini-associated RNA, promoter-associated RNA, tRNA, rRNA, microRNA, siRNA, post-transcriptionally modified RNA, synthetic RNA, RNA with modified bases or backbone, viral RNA, bacteria RNA, RNA aptamers, DNA aptamers, ribozymes, and DNAzymes.

Lipid targets include, without limitation, phospholipids, glycolipids, mono-, di-, tri-glycerides, sterols, fatty acyl lipids, glycerolipids, glycerophospholipids, sphingolipids, sterol lipids, prenol lipids, saccharolipids, polyketides, eicosanoids, prostaglandins, leukotrienes, thromboxanes, N-acyl ethanolamine lipids, cannabinoids, anandamides, terpenes, and lipopolysaccharides.

Small molecule targets include, without limitation, carbohydrates, monosaccharides, polysaccharides, galactose, fructose, glucose, amino acids, peptides, nucleic acids, nucleotides, nucleosides, cyclic nucleotides, polynucleotides, vitamins, drugs, inhibitors, single atom ions (such as magnesium, potassium, sodium, zinc, cobalt, lead, cadmium, etc.), multiple atom ions (such as phosphate), radicals (such as oxygen or hydrogen peroxide), and carbon-based gases (carbon dioxide, carbon monoxide, etc.).

Targets can also be whole cells or molecules expressed on the surface of whole cells. Exemplary cells include, without limitation, cancer cells, bacterial cells, or normal cells. Targets can also be viral particles.

A number of aptamers for these classes of target biomolecules have been identified previously, and can be incorporated into the multivalent nucleic acid aptamer constructs of the present disclosure. For example, other known RNA aptamers include, without limitation, RNA ligands of T4 DNA polymerase, RNA ligands of HIV reverse transcriptase, RNA ligands of bacteriophage R17 coat protein, RNA ligands for nerve growth factor, RNA ligands of HSV-1 DNA polymerase, RNA ligands of Escherichia coli ribosomal protein S1, and RNA ligands of HIV-1 Rev protein (U.S. Pat. No. 5,270,163 to Gold et al., which is hereby incorporated by reference in its entirety); RNA ligands of Bacillus subtilis ribonuclease P (U.S. Pat. No. 5,792,613 to Schmidt et al., which is hereby incorporated by reference); RNA ligands of ATP and RNA ligands of biotin (U.S. Pat. No. 5,688,670 to Szostak et al., which is hereby incorporated by reference in its entirety); RNA ligands of prion protein (Weiss et al., “RNA Aptamers Specifically Interact with the Prion Protein PrP,” J. Virol. 71(11):8790-8797 (1997), which is hereby incorporated by reference in its entirety); RNA ligands of hepatitis C virus protein NS3 (Kumar et al., “Isolation of RNA Aptamers Specific to the NS3 Protein of Hepatitis C Virus from a Pool of Completely Random RNA,” Virol. 237(2):270-282 (1997); Urvil et al., “Selection of RNA Aptamers that Bind Specifically to the NS3 Protein of Hepatitis C Virus,” Eur. J. Biochem. 248(1):130-138 (1997); Fukuda et al., “Specific RNA Aptamers to NS3 Protease Domain of Hepatitis C Virus,” Nucleic Acids Symp. Ser. 37:237-238 (1997), each of which is hereby incorporated by reference in its entirety); RNA ligands of chloramphenicol (Burke et al., “RNA Aptamers to the Peptidyl Transferase Inhibitor Chloramphenicol,” Chem. Biol. 4(11):833-843 (1997), which is hereby incorporated by reference in its entirety); RNA ligands of the adenosine moiety of S-adenosyl methionine (Burke and Gold, “RNA Aptamers to the Adenosine Moiety of S-Adenosyl Methionine: Structural Inferences from Variations on a Theme and the Reproducibility of SELEX,” Nucleic Acids Res. 25(10):2020-2024 (1997), which is hereby incorporated by reference in its entirety); RNA ligands of protein kinase C (Conrad et al., “Isozyme-Specific Inhibition of Protein Kinase C by RNA Aptamers,” J. Biol. Chem. 269(51):32051-32054 (1994); Conrad and Ellington, “Detecting Immobilized Protein Kinase C Isozymes with RNA Aptamers,” Anal. Biochem. 242(2):261-265 (1996), each which is hereby incorporated by reference in its entirety); RNA ligands of subtilisin (Takeno et al., “RNA Aptamers of a Protease Subtilisin,” Nucleic Acids Symp. Ser. 37:249-250 (1997), which is hereby incorporated by reference in its entirety); RNA ligands of yeast RNA polymerase II (Thomas et al., “Selective Targeting and Inhibition of Yeast RNA Polymerase II by RNA Aptamers,” J. Biol. Chem. 272(44):27980-27986 (1997), which is hereby incorporated by reference in its entirety); RNA ligands of human activated protein C (Gal et al., “Selection of a RNA Aptamer that Binds to Human Activated Protein C and Inhibits its Protein Function,” Eur. J. Biochem. 252(3):553-562 (1998), which is hereby incorporated by reference in its entirety); and RNA ligands of cyanocobalamin (Lorsch and Szostak, “In vitro Selection of RNA Aptamers Specific for Cyanocobalamin,” Biochem. 33(4):973-982 (1994), which is hereby incorporated by reference in its entirety). Additional RNA aptamers are continually being identified and isolated by those of ordinary skill in the art, and these, too, can be incorporated into the multivalent aptamer constructs of the present invention.

In some embodiments, the RNA-based metabolite sensor includes a nucleic acid aptamer molecule according to the present disclosure (e.g., a Squash nucleic acid aptamer molecule or a Beetroot nucleic acid aptamer molecule) that binds to the fluorophore substantially only after the metabolite-binding aptamer portion binds to the target molecule. In RNA-based metabolite sensors of this type, the metabolite-binding aptamer portion possesses a stable structure and is capable of binding to the target molecule, whereas the regulated aptamer portion possess a structure that is substantially incapable of binding the fluorophore (or does so with reduced affinity). Upon binding of the target molecule by the metabolite-binding aptamer portion, however, the secondary structure of the regulated aptamer portion is altered and adopts a structure that is capable of binding the fluorophore with sufficiently high affinity. As a consequence of target molecule binding, the fluorophore becomes bound by the regulated aptamer portion and upon exposure to radiation of appropriate wavelength emits a fluorescent emission signal. RNA-based metabolite sensor of this type can be used as “turn-on” sensors.

Thus, in some embodiments of the RNA-based ratiometric sensor according to the present disclosure, the transducer domain is stabilized upon specific binding of the metabolite. In accordance with such embodiments, binding of the metabolite induces folding of the regulated aptamer portion.

To facilitate the ability of these sensors to “turn-on” in the presence of the target molecule, the metabolite-binding aptamer portion can be coupled at its 5′ and 3′ ends to the nucleic acid molecule according to the present disclosure via the transducer domain. The transducer domain may include a pair of antiparallel stem-forming sequences, one coupled by phosphodiester bond between a first portion of the fluorophore-specific aptamer and a 5′ end of the target-binding aptamer, and the other coupled by phosphodiester bond between a second portion of the fluorophore-specific aptamer and a 3′ end of the target-binding aptamer. The transducer domain may preferably include one or more mismatched base pairs or an overall low number of base pairs (e.g., one or two base pairs) such that stem formation of the transducer domain is thermodynamically unfavorable in the absence of target molecule binding to the metabolite-binding aptamer portion, and thermodynamically favorable after target molecule binding to the metabolite-binding aptamer portion.

In some embodiments of the RNA-based ratiometric sensor according to the present disclosure, the transducer domain is a thermodynamically unstable helix.

As described in PCT Application Publ. Nos. WO 2010/096584 and WO 2013/016694, both to Jaffrey et al., which are hereby incorporated by reference in their entirety, RNA-based metabolite sensors have been developed that are specific for the biomolecules ADP, adenosine, guanine, GTP, SAM, and streptavidin.

In some embodiments, the RNA-based metabolite sensor includes a regulated aptamer portion that binds to the fluorophore substantially only in the absence of the metabolite-binding aptamer portion binding to the target molecule. In RNA-based metabolite sensors of this type, the metabolite-binding aptamer portion a stable structure and is capable of binding to the target molecule, and the regulated aptamer portion or regions of the RNA-based metabolite sensor adjacent to the regulated aptamer portion possess a structure that is capable of binding the fluorophore with sufficiently high affinity. Upon binding of the target molecule by the metabolite-binding aptamer portion, however, the secondary structure of the regulated aptamer portion is altered and adopts a structure that is substantially incapable of binding the fluorophore with high affinity. As a consequence of target molecule binding, the fluorophore dissociates from the regulated aptamer portion and despite exposure to radiation of appropriate wavelength the fluorophore will no longer emit a fluorescent emission signal (or emits only a substantially diminished level of fluorescent emissions). RNA-based metabolite sensors of this type can be used as “turn-off” sensors.

In some embodiments, the RNA-based metabolite sensors described herein can be used as sensors for tracking the presence, location, or quantity of a fused nucleic acid molecule of interest in a cell or an in vitro sample; for determining the presence, location, or quantity of a target molecule of interest in a cell or an in vitro sample; for high throughput screening assays to assess the ability of an agent to modulate certain cellular functions, such as transcription levels or splicing, or for modulating the activity or availability of a target molecule; for microarray detection of analytes or genes of interest; and de novo screening of sensor molecules for particular targets of interest using a modified SELEX.

An exemplary “turn-on” sensor for SAM has the nucleotide sequence according to SEQ ID NO: 26 as follows:

5′-GGU CCA GAU GCC UUG UAA CCG AAA GGG aca cAA GGU
GAG CCC AAU AAU ACG GUU UGG GUU AGG AUA GGA AGU
AGA GCC GUA AAC UCU CUA AGC Ggu guC GAA AGG AUG
GAC C-3′
[Nucleotides in bold are Squash; nucleotides in
italic are SAM-III aptamer; transducer sequence
is shown in lowercase.]

Another exemplary “turn-on” sensor for SAM has the nucleotide sequence according to SEQ ID NO: 86 as follows:

5′-GGU CCA GAU GCC UUG UAA CCG AAA GGG aau cAA GGU
GAG CCC AAU AAU ACG GUU UGG GUU AGG AUA GGA AGU
AGA GCC GUA AAC UCU CUA AGC Gga uuC GAA AGG AUG
GAC C-3′
[Nucleotides in bold are Squash; nucleotides in
italic are SAM-III aptamer; transducer sequence
is shown in lowercase.]

This SAM sensors of SEQ ID NO: 26 and SEQ ID NO: 86 comprises the SAM-binding aptamer portion of the SAM-III riboswitch (Lu et al., “Crystal Structures of the SAM-III/SMK Riboswitch Reveal the SAM-Dependent Translation Inhibition Mechanism,” Nat. Struct. Mol. Biol. 15:1076-1083 (2008), which is hereby incorporated by reference in its entirety) fused to a fluorogenic aptamer via a transducer domain. The transducer domain is a thermodynamically unstable helix, which is stabilized upon SAM binding, thus allosterically inducing the folding of the fluorogenic aptamer (Kim and Jaffrey, “A Fluorogenic RNA-Based Sensor Activated by Metabolite-Induced RNA Dimerization,” Cell Chem. Biol. 26:1725-1731 (2019) and Litke and Jaffrey, “Highly Efficient Expression of Circular RNA Aptamers in Cells Using Autocatalytic Transcripts,” Nat. Biotechnol. 37:667-675 (2019), which are hereby incorporated by reference in their entirety).

Another aspect of the present disclosure relates to an RNA-based ratiometric metabolite sensor comprising:
(i) a regulated fluorescence activating aptamer comprising the RNA-based metabolite sensor according to the present disclosure and
(ii) a constitutive fluorescence activating aptamer.

Suitable RNA-based metabolite sensors are described in detail supra.

In some embodiments of the RNA-based ratiometric metabolite sensor according to the present disclosure, the sensor is circular.

In some embodiments of the RNA-based ratiometric metabolite sensor according to the present disclosure, the sensor comprises a first arm, a second arm, and a third arm.

In some embodiments of the RNA-based ratiometric metabolite sensor according to the present disclosure, the first arm comprises the regulated fluorescence activating aptamer, the second arm comprises the constitutive fluorescence activating aptamer; and a third arm comprises a Tornado stem. For example, the regulated fluorescence activating aptamer may be a Squash-SAM sensor according to SEQ ID NO: 26 or SEQ ID NO: 86 and the constitutive fluorescence activating aptamer may be Broccoli.

In some embodiments of the RNA-based ratiometric metabolite sensor according to the present disclosure, the sensor comprises an F30 scaffold.

In some embodiments of the RNA-based ratiometric metabolite sensor according to the present disclosure, the constitutive fluorescence activating aptamer is Broccoli.

In some embodiments of the RNA-based ratiometric metabolite sensor according to the present disclosure, the constitutive fluorescence activating aptamer is Corn.

An exemplary RNA-based ratiometric metabolite sensor according to the present disclosure has the nucleotide sequence according to SEQ ID NO: 88 as follows:

AAC CAU GCC GAC UGA UGG CAG UUG CCA UGU GUA UGU GGC CAG AUG CCU UGU AAC CGA AAG GGA CAC AAG GUG AGC CCA AUA AUA CGG UUU GGG UUA GGA UAG GAA GUA GAG CCG UAA ACU CUC UAA GCG GUG UCG AAA GGA UGG CCA CAU ACU CUG AUG AUC GAG ACG GUC GGG UCC AGA UAU UCG UAU CUG UCG AGU AGA GUG UGG GCU CGA UCA UUC AUG GCA ACU GCC AUC AGU CGG CGU GGA CUG UAG [Tornado Stem: nucleotides 1-21 and 227-252; F30 Scaffold: nucleotides 22-38, 145-162, and 212-226; Squash: nucleotides 67-129; Broccoli: nucleotides 163-211; SAM-III aptamer: nucleotides 39-62 and 134-144; Transducer sequence: nucleotides 63-66 and 130-133; This is a circular RNA and therefore does not comprise 5′ or 3′ ends.]

Another aspect of the present disclosure relates to a system comprising: the RNA-based ratiometric metabolite sensor according to the present disclosure; a first fluorophore molecule; and a second fluorophore molecule. Suitable RNA-based ratiometric metabolite sensors, first fluorophore molecules, and second fluorophore molecules are described infra.

In some embodiments of the system according to the present disclosure, the first fluorophore molecule is 4-(3,5-difluoro-4-hydroxybenzylidene)-1-methyl-5-oxo-4,5-dihydro-1H-imidazole-2-carbaldehyde oxime (“DFHO”), (Z)-5-(3,5-difluoro-4-hydroxybenzylidene)-3-methyl-2-(trifluoromethyl)-3,5-dihydro-4H-imidazol-4-one (“DFHBI-2T”), (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”), (Z)-3-amino-5-(3,5-difluoro-4-hydroxybenzylidene)-2-thioxothiazolidin-4-one (“NRD5”), or methyl (E)-3-(4-((Z)-3,5-difluoro-4-hydroxybenzylidene)-1-methyl-5-oxo-4,5-dihydro-1H-imidazol-2-yl)acrylate (“DFAME”).

In some embodiments of the system according to the present disclosure, the second fluorophore molecule is (Z)-3-((1H-benzo[d]imadazol-4-yl)methyl)-5-(3,5-difluoro-4-hydroxybenzylidene)-2-methyl-3,5-dihydro-4H-imidazol-4-one (“BI”).

### Molecular Complexes

Another aspect of the present disclosure is directed to a molecular complex comprising a fluorophore molecule comprising a methyne bridge between a substituted aromatic ring system and a substituted imidazol(thi)one, oxazol(thi)one, pyrrolin(thi)one, or furan(thi)one; and the nucleic acid aptamer molecule according to the present disclosure (e.g., a Squash nucleic acid aptamer molecule or a Beetroot nucleic acid aptamer molecule) specifically to the fluorophore molecule; where the fluorophore molecule has substantially enhanced fluorescence, in comparison to the fluorophore molecule prior to specific binding, upon exposure to radiation of suitable wavelength.

Suitable exemplar fluorophore molecules are described in more detail supra.

In some embodiments, the fluorophore molecule is selected from the group consisting of 4-(3,5-difluoro-4-hydroxybenzylidene)-1-methyl-5-oxo-4,5-dihydro-1H-imidazole-2-carbaldehyde oxime (“DFHO”), (Z)-5-(3,5-difluoro-4-hydroxybenzylidene)-3-methyl-2-(trifluoromethyl)-3,5-dihydro-4H-imidazol-4-one (“DFHBI-2T”), (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”), (Z)-3-amino-5-(3,5-difluoro-4-hydroxybenzylidene)-2-thioxothiazolidin-4-one (“NRD5”), or methyl (E)-3-(4-((Z)-3,5-difluoro-4-hydroxybenzylidene)-1-methyl-5-oxo-4,5-dihydro-1H-imidazol-2-yl)acrylate (“DFAME”).

In some embodiments, when the Squash nucleic acid aptamer molecule according to the present disclosure comprises the nucleic acid sequence of:
(1) GGC UAC AAG GUG AGC CCA AUA AUA CGG UUU GGG UUA GGA UAG GAA GUA GAG CCG UAA ACU CUC UAA GCG GUA GUC (SEQ ID NO: 1);
(2) GGC UAC AAG GUG AGC CCA AUA AUA GGG UUU GGG UUA GGA UAG GAA GUA GAG CCC UAA ACU CUC UAA GCG GUA GUC (SEQ ID NO: 2);
(3) GGC UAC AAG GUG AGC CCA AUA AUA NNN UUU GGG UUA GGA UAG GAA GUA GAG NNN UAA ACU CUC UAA GCG GUA GUC (SEQ ID NO: 3), where N at positions 25 and 54 are complementary to each other, N at positions 26 and 53 are complementary to each other, and N at positions 27 and 52 are complementary to each other, optionally where the complementary positions base pair with each other;
(4) GGC UAC AAG GUG NNN NNN NUA AUA NNN UNN NNN NNA GGA UAG GAN NNN NNN NNN UAA ANN NNN NAA GCG GUA GUC (SEQ ID NO: 4),
   where N at positions 25 and 54 are complementary to each other,
   N at positions 26 and 53 are complementary to each other,
   N at positions 27 and 52 are complementary to each other,
   N at positions 13 and 35 are complementary to each other,
   N at positions 14 and 34 are not complementary to each other,
   N at positions 15 and 33 are complementary to each other,
   N at positions 16 and 32 are complementary to each other,
   N at positions 17 and 31 are complementary to each other,
   N at positions 18 and 30 are complementary to each other,
   N at positions 19 and 29 are complementary to each other
   N at positions 45 and 64 are complementary to each other,
   N at positions 46 and 63 are complementary to each other,
   N at positions 48 and 62 are complementary to each other,
   N at positions 49 and 61 are complementary to each other,
   N at positions 50 and 60 are complementary to each other, and/or
   N at positions 51 and 59 are complementary to each other, optionally where the complementary positions base pair with each other;
(5) AAG GUG AGC CCA AUA AUA CGG UUU GGG UUA GGA UAG GAA GUA GAG CCG UAA ACU CUC UAA GCG (SEQ ID NO: 5);
(6) AAG GUG AGC CCA AUA AUA GGG UUU GGG UUA GGA UAG GAA GUA GAG CCC UAA ACU CUC UAA GCG (SEQ ID NO: 6);
(7) AAG GUG AGC CCA AUA AUA NNN UUU GGG UUA GGA UAG GAA GUA GAG NNN UAA ACU CUC UAA GCG (SEQ ID NO: 7), where N at positions 19 and 48 are complementary to each other, N at positions 20 and 47 are complementary to each other, and N at positions 21 and 46 are complementary to each other, optionally where the complementary positions base pair with each other;
(8) AAG GUG NNN NNN NUA AUA NNN UNN NNN NNA GGA UAG GAN NNN NNN NNN UAA ANN NNN NAA GCG (SEQ ID NO: 8),
   where N at positions 19 and 48 are complementary to each other,
   N at positions 20 and 47 are complementary to each other,
   N at positions 21 and 46 are complementary to each other,
   N at positions 7 and 29 are complementary to each other,
   N at positions 8 and 28 are not complementary to each other,
   N at positions 9 and 27 are complementary to each other,
   N at positions 10 and 26 are complementary to each other,
   N at positions 11 and 25 are complementary to each other,
   N at positions 12 and 24 are complementary to each other,
   N at positions 13 and 23 are complementary to each other
   N at positions 39 and 58 are complementary to each other,
   N at positions 40 and 57 are complementary to each other,
   N at positions 41 and 56 are complementary to each other,
   N at positions 43 and 55 are complementary to each other,
   N at positions 44 and 54 are complementary to each other, and/or
   N at positions 45 and 53 are complementary to each other, optionally where the complementary sequences base pair with each other;
(9) GCC UAG GCU UCA AGG UGG CCC AAU GAU AUG GUU UGGG UUA GGA UAG GAA UAA GAG CCU UAA ACU CUU CAA AGC GGA AGU CUA GGC (SEQ ID NO: 9) [9-1];
(10) GCC UAG GCU UCA AGG UGA GCC CAA UAA UAU GGU UUG GGU UAG GAU AGG AAG AAG AGC CUU AAA CUC UCU AAG CGG AAG UCU AGG C (SEQ ID NO: 10) [DE1-2];
(11) GCC UAG GCU ACA AGG UGA GCC CAA UAA UAU GGU UUG GGU UAG GAU AGG AAG UAG AGC CUU AAA CUC UCU AAG CGG UAG UCU AGG C (SEQ ID NO: 11) [DE2-6];
(12) GGU AGG CUA CAA GGU GAG CCC AAU AAU ACG GUU UGG GUU AGG AUA GGA AGU AGA GCC GUA AAC UCU CUA AGC GGU AGU CUA CC (SEQ ID NO: 12) [Squash];
(13) GGU AGG CUA CNN NNN NAG CCC AAU AAU ACG GUU UGG GUU NNN NNN NNN AGU AGA GCC GUA AAC UCU CUN NNN NGU AGU CUA CC (SEQ ID NO: 13), where N at each of positions 11-16, 40-48, and 69-73 can be any single nucleotide insertion of any length;
(14) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 8 forms a stem loop, n at position 18 forms a stem loop, n at positions 1 and 24 form a stem;
(15) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 1 has the sequence of CUA C, n at position 8 has the sequence of AGC CCA AUA AUA CGG UUU GGG UU (SEQ ID NO: 16), n at position 18 has the sequence of AGU AGA GCC GUA AAC UCU CU (SEQ ID NO: 17), and n at position 24 has the sequence of GUA G;
(16) CUA CAA GGU GAG CCC AAU AAU ACG GUU UGG GUU AGG AUA GGA AGU AGA GCC GUA AAC UCU CUA AGC GGU AG (SEQ ID NO: 19);
(17) nNA GNU GnA GGA UAG GAn NAG CGn (SEQ ID NO: 14), where n at positions 1, 8, 18, and 24 can be a nucleotide insertion of 1-200 nucleotide bases and N at positions 2, 5, and 19 is any nucleotide base; optionally, where n at position 1 has the sequence of GGU CCA GAU GCC UUG UAA CCG AAA GGG ACA C (SEQ ID NO: 20), n at position 8 has the sequence of AGC CCA AUA AUA CGG UUU GGG U (SEQ ID NO: 21), n at position 18 has the sequence of AGU AGA GCC GUA AAC UCU CU (SEQ ID NO: 17), and n at position 24 has the sequence of GU GUC GAA AGG AUG GAC C (SEQ ID NO: 25);
(18) GGU CCA GAU GCC UUG UAA CCG AAA GGG ACA CAA GGU GAG CCC AAU AAU ACG GUU UGG GUU AGG AUA GGA AGU AGA GCC GUA AAC UCU CUA AGC GGU GUC GAA AGG AUG GAC C (SEQ ID NO: 26);
(19) nNA GNU GAN NNN NNN NNN NNN NNN NNN NNU AGG AUA GGA ANN NNN NNN NNN NNN NNN NUN AGC Gn (SEQ ID NO: 27), where n at positions 1 and 65 can be a nucleotide insertion of 1-200 nucleotide bases, N at positions 2, 5, 9-29, 41-58, and 60 is any nucleotide base, N at positions 9-29 forms a step loop, and N at positions 41-58 forms a stem loop comprising a bulge in the stem, optionally where n at positions 1 and 65 form a stem;
(20) nNA GNU GAN UAG GAU AGG AAN UNA GCG n (SEQ ID NO: 28), where n at positions 1 and 28 can be a nucleotide insertion of 1-200 nucleotide bases, N at positions 2, 5, and 23 are any nucleotide base, N at position 9 is a nucleotide insertion of 1-500 nucleotide bases where the insertion forms a stem loop, and N at position 21 is a nucleotide insertion of 1-500 nucleotide bases where the insertion forms a stem loop comprising a bulge in the stem, optionally where n at positions 1 and 28 form a stem;
(21) nNA GNU GAN NNN NNN NNN NNN NNN NNN NNU AGG AUA GGA ANN NNN NNN NNN NNN NNN UNA GCG n (SEQ ID NO: 29), where n at positions 1 and 64 can be a nucleotide insertion of 1-200 nucleotide bases, N at positions 2, 5, 9-29, 41-57, and 59 is any nucleotide base, N at positions 9-29 forms a step loop, and N at positions 41-57 forms a stem loop, optionally where n at positions 1 and 64 form a stem;
(22) nNA GNU GAN UAG GAU AGG AAN UNA GCG n (SEQ ID NO: 30), where n at positions 1 and 28 can be a nucleotide insertion of 1-200 nucleotide bases, N at positions 2, 5, and 23 are any nucleotide base, N at position 9 is a nucleotide insertion of 1-500 nucleotide bases where the insertion forms a stem loop, and N at position 21 is a nucleotide insertion of 1-500 nucleotide bases where the insertion forms a stem loop, optionally where n at positions 1 and 28 form a stem; or
(23) nAA GGU GAG CCC AAU AAU ACG GUU UGG GUU AGG AUA GGA AGU AGA GCC GUA AAC UCU CUA AGC Gn (SEQ ID NO: 31), where n at positions 1 and 65 can be a nucleotide insertion of 1-200 nucleotide bases, optionally where n at positions 1 and 65 forms a stem, the fluorophore is 4-(3,5-difluoro-4-hydroxybenzylidene)-1-methyl-5-oxo-4,5-dihydro-1H-imidazole-2-carbaldehyde oxime (“DFHO”) and/or the fluorophore (Z)-4-(3,5-difluoro-4-hydroxybenzylidene)-2-methyl-1-(2,2,2-trifluoroethyl)-1H-imidazol-5(4H)-one (DFHBI-1T).

In some embodiments, when the nucleic acid aptamer molecule according to the present disclosure comprises the Beetroot nucleic acid aptamer sequence of:
(1) GGU GGG UGG UGU GGA GGA GUA (SEQ ID NO: 32);
(2) nGG UGG GUG GUG UGG AGG AGU An (SEQ ID NO: 33), where n at positions 1 and 23 is a nucleotide insertion of 1-200 nucleotide bases, optionally where n at position 1 forms a stem with n at position 23;
(3) NNN NNN GGU GGG UGG UGU GGA GGA GUA NNN NNN (SEQ ID NO: 34), where N at positions 1 and 33 are complementary to each other and form a base pair,
   N at positions 2 and 32 are complementary to each other and form a base pair,
   N at positions 3 and 31 are complementary to each other and form a base pair,
   N at positions 4 and 30 are complementary to each other and form a base pair,
   N at positions 5 and 29 are complementary to each other and form a base pair, and/or
   N at positions 6 and 28 are complementary to each other and form a base pair, optionally where N at positions 1-6 forms a stem with N at positions 28-33; or
(4) nnn nNN NNN NGG UGG GUG GUG UGG AGG AGU ANN NNN N (SEQ ID NO: 35), where n at positions 1-4 is any nucleotide base,
   N at positions 5 and 37 are complementary to each other and form a base pair,
   N at positions 6 and 36 are complementary to each other and form a base pair,
   N at positions 7 and 35 are complementary to each other and form a base pair,
   N at positions 8 and 34 are complementary to each other and form a base pair,
   N at positions 9 and 33 are complementary to each other and form a base pair, and/or
   N at positions 10 and 32 are complementary to each other and form a base pair, optionally wherein N at positions 5-10 forms a stem with N at positions 32-37, the fluorophore is 5-difluoro-4-hydroxybenzylidene imidazolinone-2-acrylate methyl (DFAME) and/or 4-(3,5-difluoro-4-hydroxybenzylidene)-1-methyl-5-oxo-4,5-dihydro-1H-imidazole-2-carbaldehyde oxime (“DFHO”).

In some embodiments, the molecular complex is formed by a nucleic acid aptamer molecule comprising a plurality of first domains for binding to multiple identical fluorophore compounds per molecule and one or more fluorescent compounds that are bound to at least one, and optionally all, of the first domains present in the nucleic acid aptamer molecule. These molecular complexes can exist in vitro, in isolated form, or in vivo following introduction of the nucleic acid aptamer molecule (or a genetic construction or expression system encoding the same) into a host cell.

In some embodiments, the nucleic acid aptamer molecule includes one or more first domains and a second domain that binds specifically to a target molecule of interest. The molecular complex, therefore, can include the nucleic acid aptmer molecule, the target molecule (bound specifically by the second domain), and one or more fluorescent compounds that are bound to the first domain(s). These molecular complexes can exist in vitro, in isolated form or tethered to a substrate such as on an arrayed surface, or in vivo following introduction of the nucleic acid molecule (or a genetic construction or expression system encoding the same) into a host cell.

In some embodiments, the nucleic acid aptamer molecule includes a plurality of aptamer sensor concatemers, each monomer including a first domain and a second domain. The molecular complex, therefore, can include the nucleic acid aptamer molecule, a plurality of target molecules (bound specifically by the plurality of second domains), and a plurality of fluorescent compounds that are bound to the plurality of first domain(s). These molecular complexes can exist in vitro, in isolated form or tethered to a substrate such as on an arrayed surface, or in vivo following introduction of the nucleic acid molecule (or a genetic construction or expression system encoding the same) into a host cell.

In some embodiments, the nucleic acid aptamer molecule includes an aptamer sequence linked to a hybridization probe sequence that is complementary to a target nucleic acid molecule. The molecular complex, therefore, can include the nucleic acid aptamer molecule hybridized to the target nucleic acid molecule, and one or more fluorophores bound specifically to the fluorophore-specific aptamer domain. These molecular complexes can exist in vitro, in isolated form or tethered to a substrate such as on an arrayed surface, or in vivo following introduction of the nucleic acid molecule (or a genetic construction or expression system encoding the same) into a host cell. In some embodiments, these complexes can exist in fixed cells or on histologic tissue sections in the manner of an in situ hybridization protocol.

Although in vitro host cells are described herein, it should be appreciated to skilled artisans that the host cells can be present in a whole organism, preferably a non-human organism.

For formation of the molecular complex inside a cell, the fluorophore is introduced into the cell where it can interact with (and be bound by) the aptamer that specifically binds to it. According to one approach, the cell or the sample is contacted with the fluorophore by incubating the cell or the sample with the fluorophore. The fluorophore will be taken up by the cell, where it may freely diffuse throughout the cell. According to another approach, the fluorophore is injected into the cell or administered to a plant, embryo, mammal, or transgenic animal including the cell.

Accordingly, another aspect of the present disclosure is directed to a host cells comprising a molecular complex according to the present disclosure.

### Genetic Constructs

While the nucleic acid aptamer molecules described herein can be synthesized from chemical precursor, they also can be prepared either in vitro or in vivo using recombinant templates or constructs, including transgenes, that encode the nucleic acid aptamer molecules. Whether using in vitro transcription or transgenes suitable for expression in vivo, these genetic constructs can be prepared using well known recombinant techniques.

Accordingly, another aspect of the present disclosure relates to a constructed DNA molecule encoding the nucleic acid aptamer molecule (e.g., a Squash nucleic acid aptamer molecule or a Beetroot nucleic acid aptamer molecule) according to the present disclosure.

In some embodiments, the constructed DNA molecule includes a first region encoding one or more nucleic acid aptamer molecules according to the present disclosure. Where multiple nucleic acid aptamer molecules according to the present disclosure are present, they can be separated by a linker sequence.

In some embodiments, the constructed DNA molecule encodes an RNA fusion product. Such a product is formed by joining together one piece of DNA encoding an RNA molecule of interest and a second piece of DNA encoding a nucleic acid aptamer molecule (e.g., a Squash nucleic acid aptamer molecule or a Beetroot nucleic acid aptamer molecule) that binds specifically to a fluorophore of the invention. As described above, the nucleic acid aptamer molecule can be in the form of a concatemer that contains multiple fluorophore-binding domains.

In some embodiments, the constructed DNA molecule encodes a molecular sensor, which is formed by joining together one piece of DNA encoding an RNA aptamer molecule that is specific for a target molecule and a second piece of DNA encoding a nucleic acid aptamer molecule (e.g., a Squash nucleic acid aptamer molecule or a Beetroot nucleic acid aptamer molecule) that binds specifically to a fluorophore of the invention, and optionally a third piece of DNA encoding the transducer molecule. The conjoined RNA sequences can cooperate in the manner described above, so as to achieve a “turn-on” sensor or “turn-off” sensor.

In some embodiments, an empty construct can be prepared for preparation of an RNA fusion product. Such an empty construct includes a DNA sequence encoding a nucleic acid aptamer molecule (e.g., a Squash nucleic acid aptamer molecule or a Beetroot nucleic acid aptamer molecule) that binds specifically to a fluorophore of the invention, along with appropriate regulatory sequences (discussed below), and a restriction enzyme insertion site that can be used for subsequent insertion of a desired DNA molecule (encoding an RNA molecule of interest). As described above, the nucleic acid aptamer molecule can include a concatemer of fluorophore-binding domains. The restriction enzyme insertion site can include one or more enzymatic cleavage sites to facilitate insertion of virtually any DNA coding sequence as desired. The restriction enzyme insertion site is preferably located between the promoter sequence and the aptamer-encoding DNA sequence.

In some embodiments, the constructed DNA molecule encodes an RNA aptamer, however, within the region encoding the RNA aptamer, an intron is positioned therein. This spatially segregates the RNA aptamer-encoding regions, whereby transcription in the absence of a proper spliceosome will not afford a functional aptamer molecule. In the presence of a proper spliceosome, excision of the intron from a transcript of the constructed DNA molecule affords the RNA aptamer molecule. This will allow the RNA aptamer to bind to the fluorophore to induce fluorescence.

In some embodiments, the sequences within the intron contribute to the fluorophore-binding aptamer, whereby prior to splicing the RNA molecule is capable of exhibiting fluorescence when bound to the fluorophore. However, in the presence of a proper spliceosome, splicing of the RNA molecule destroys the fluorophore-binding aptamer, thereby inhibiting fluorescence.

Preparation of the DNA molecule can be carried out by well-known methods of DNA ligation. DNA ligation utilizes DNA ligase enzymes to covalently link or ligate fragments of DNA together by catalyzing formation of a phosphodiester bond between the 5′ phosphate of one strand of DNA and the 3′ hydroxyl of another. Typically, ligation reactions require a strong reducing environment and ATP. The commonly used T4 DNA ligase is an exemplary DNA ligase in preparing the DNA molecule described herein. Once the DNA molecule of the present disclosure has been constructed, it can be incorporated into host cells as described infra.

Transcription of the DNA molecule of the present disclosure is often dependent upon the presence of a promoter, which is a DNA sequence that directs the binding of RNA polymerase and thereby promotes RNA synthesis. Accordingly, the DNA molecule of the present disclosure may include a promoter operably coupled to the first region to control expression of the RNA aptamer. Because not all polymerases require promoters, the promoter sequence is optional.

The DNA sequences of eukaryotic promoters differ from those of prokaryotic promoters. Furthermore, eukaryotic promoters and accompanying genetic signals may not be recognized in or may not function in a prokaryotic system and, further, prokaryotic promoters are not recognized and do not function in eukaryotic cells.

Promoters vary in their “strength” (i.e., their ability to promote transcription). Depending on the application, it may be desirable to use strong promoters to obtain a high level of transcription. For instance, when used simply as a label high expression levels may be preferred, whereas to assess transcript behavior it may be desirable to obtain lower levels of expression that allow the cell to process the transcript.

Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

As described above, one type of regulatory sequence is a promoter located upstream or 5′ to the coding sequence of the DNA molecule. Depending upon the desired activity, it is possible to select the promoter for not only in vitro production of the RNA aptamer, but also in vivo production in cultured cells or whole organisms, as described below. Because in vivo production can be regulated genetically, another suitable class of promoters is an inducible promoter which induces transcription of the DNA molecule in response to specific conditions, thereby enabling expression of the RNA aptamer as desired (i.e., expression within specific tissues, or at specific temporal and/or developmental stages). The various promoter types can be driven by RNA polymerases I, II, or III.

Suitable promoters for use with the constructed DNA molecule of the present disclosure include, without limitation, a T7 promoter, a SUP4 tRNA promoter, an RPR1 promoter, a GPD promoter, a GAL1 promoter, an hsp70 promoter, an Mtn promoter, a UAShs promoter, and functional fragments thereof. The T7 promoter is a well-defined, short DNA sequence that can be recognized and utilized by T7 RNA polymerase of the bacteriophage T7. The T7 RNA polymerase can be purified in large scale and is commercially available. The transcription reaction with T7 promoter can be conducted in vitro to produce a large amount of the molecular complex of the present invention (Milligan et al., “Oligoribonucleotide Synthesis Using T7 RNA Polymerase and Synthetic DNA Templates,” Nucleic Acids Res. 15(21):8783-8798 (1987), which is hereby incorporated by reference in its entirety). The T7 RNA polymerase can also be used in mammalian and bacterial cells to produce very high levels of RNA. The SUP4 tRNA promoter and RPR1 promoter are driven by RNA polymerase III of the yeast Saccharomyces cerevisiae, and suitable for high level expression of RNA less than 400 nucleotides in length (Kurjan et al., Mutation at the Yeast SUP4 tRNA^{tyr} Locus: DNA Sequence Changes in Mutants Lacking Suppressor Activity,” Cell 20:701-709 (1980); Lee et al., “Expression of RNase P RNA in Saccharomyces cerevisiae is Controlled by an Unusual RNA Polymerase III Promoter,” Proc. Natl. Acad. Sci. USA 88:6986-6990 (1991), each of which is hereby incorporated by reference in its entirety). The glyceraldehyde-3-phosphate dehydrogenase (GPD) promoter in yeast is a strong constitutive promoter driven by RNA polymerase II (Bitter et al., “Expression of Heterologous Genes in Saccharomyces cerevisiae from Vectors Utilizing the Glyceraldehyde-3-phosphate Dehydrogenase Gene Promoter,” Gene 32:263-274 (1984), which is hereby incorporated by reference in its entirety). The galactokinase (GAL1) promoter in yeast is a highly inducible promoter driven by RNA polymerase II (Johnston and Davis, “Sequences that Regulate the Divergent GAL1-GAL10 Promoter in Saccharomyces cerevisiae,” Mol. Cell. Biol. 4:1440-1448 (1984), which is hereby incorporated by reference in its entirety). The heat shock promoters are heat inducible promoters driven by the RNA polymerase II in eukaryotes. The frequency with which RNA polymerase II transcribes the major heat shock genes can be increased rapidly in minutes over 100-fold upon heat shock. Another inducible promoter driven by RNA polymerase II that can be used in the present invention is a metallothionine (Mtn) promoter, which is inducible to the similar degree as the heat shock promoter in a time course of hours (Stuart et al., “A 12-Base-Pair Motif that is Repeated Several Times in Metallothionine Gene Promoters Confers Metal Regulation to a Heterologous Gene,” Proc. Natl. Acad. Sci. USA 81:7318-7322 (1984), which is hereby incorporated by reference in its entirety).

Initiation of transcription in mammalian cells requires a suitable promoter, which may include, without limitation, β-globin, GAPDH, β-actin, actin, Cstf2t, SV40, MMTV, metallothionine-1, adenovirus Ela, CMV immediate early, immunoglobulin heavy chain promoter and enhancer, and RSV-LTR. Termination of transcription in eukaryotic genes involves cleavage at a specific site in the RNA which may precede termination of transcription. Also, eukaryotic termination varies depending on the RNA polymerase that transcribes the gene. However, selection of suitable 3′ transcription termination regions is well known in the art and can be performed with routine skill.

Spatial control of an RNA molecule can be achieved by tissue-specific promoters, which have to be driven by the RNA polymerase II. The many types of cells in animals and plants are created largely through mechanisms that cause different genes to be transcribed in different cells, and many specialized animal cells can maintain their unique character when grown in culture. The tissue-specific promoters involved in such special gene switching mechanisms, which are driven by RNA polymerase II, can be used to drive the transcription templates that code for the molecular complex of the present invention, providing a means to restrict the expression of the molecular complex in particular tissues. Any of a variety of tissue-specific promoters can be selected as desired.

For gene expression in plant cells, suitable promoters may include, without limitation, nos promoter, the small subunit ribulose bisphosphate carboxylase genes, the small subunit chlorophyll A/B binding polypeptide, the 35S promoter of cauliflower mosaic virus, and promoters isolated from plant genes, including the Pto promoter itself (see Vallejos et al., “Localization in the Tomato Genome of DNA Restriction Fragments Containing Sequences Homologous to the rRNA (45S), the major chlorophyll_{A/B} Binding Polypeptide and the Ribulose Bisphosphate Carboxylase Genes,” Genetics 112: 93-105 (1986) (disclosing the small subunit materials), which is hereby incorporated by reference in its entirety). The nos promoter and the 35S promoter of cauliflower mosaic virus are well known in the art.

In addition, the constructed DNA molecule may also include an operable 3′ regulatory region, selected from among those which are capable of providing correct transcription termination and polyadenylation of mRNA for expression in plant cells. A number of 3′ regulatory regions are known to be operable in plants. Exemplary 3′ regulatory regions include, without limitation, the nopaline synthase 3′ regulatory region (Fraley et al., “Expression of Bacterial Genes in Plant Cells,” Proc. Nat'l. Acad. Sci. USA, 80:4803-4807 (1983), which is hereby incorporated by reference in its entirety) and the cauliflower mosaic virus 3′ regulatory region (Odell et al., “Identification of DNA Sequences Required for Activity of the Cauliflower Mosaic Virus 35S Promoter,” Nature, 313(6005):810-812 (1985), which is hereby incorporated by reference in its entirety). Virtually any 3′ regulatory region known to be operable in plants would suffice for proper expression of the coding sequence of the constructed DNA molecule of the present invention.

Another type of regulatory sequence is known as an enhancer. Enhancer elements do not need to be located immediately upstream of the promoter or the sequence which encodes the transcript that will be made. Enhancers can, in fact, be located very far away. Nevertheless, they can also serve as regulatory elements, and could potentially be regulated by signaling molecules and thereby influence the expression of a target RNA inside a cell. Exemplary enhancer elements include, without limitation, the well-known SV40 enhancer region and the 35S enhancer element.

Once the DNA molecule of the present invention has been constructed, it can be incorporated into cells using conventional recombinant DNA technology. Generally, this involves inserting the DNA molecule into an expression system to which the DNA molecule is heterologous (i.e., not normally present). The heterologous DNA molecule is inserted into the expression system or vector in proper sense orientation. The vector contains the necessary elements for their persistent existence inside cells and for the transcription of an RNA molecule that can be translated into the molecular complex of the present disclosure.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and transfection, and replicated in cultures including prokaryotic organisms and eukaryotic cells grown in tissue culture.

Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/−(see “Stratagene Cloning Systems” Catalog (1993) from Stratagene, La Jolla, Calif, which is hereby incorporated by reference in its entirety), pQE, pIH821, pGEX, pET series (see Studier et al., “Use of T7 RNA Polymerase to Direct Expression of Cloned Genes,” Gene Expression Technology, vol. 185 (1990), which is hereby incorporated by reference in its entirety), pIIIEx426 RPR, pIIIEx426 tRNA (see Good and Engelke, “Yeast Expression Vectors Using RNA Polymerase III Promoters,” Gene 151:209-214 (1994), which is hereby incorporated by reference in its entirety), p426GPD (see Mumberg et al., “Yeast Vectors for the Controlled Expression of Heterologous Proteins in Different Genetic Background,” Gene 156:119-122 (1995), which is hereby incorporated by reference in its entirety), p426GAL1 (see Mumberg et al., “Regulatable Promoters of Saccharomyces cerevisiae: Comparison of Transcriptional Activity and Their Use for Heterologous Expression,” Nucl. Acids Res. 22:5767-5768 (1994), which is hereby incorporated by reference in its entirety), pUAST (see Brand and Perrimon, “Targeted Gene Expression as a Means of Altering Cell Fates and Generating Dominant Phenotypes,” Development 118:401-415 (1993), which is hereby incorporated by reference in its entirety), and any derivatives thereof. Suitable vectors are continually being developed and identified.

A variety of host-vector systems may be utilized to express the DNA molecule. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, adeno-associated virus, retroviral vectors, etc.); insect cell systems infected with virus (e.g., baculovirus); and plant cells infected by bacteria or transformed via particle bombardment (i.e., biolistics).

Accordingly, another aspect of the present disclosure relates to an expression system comprising an expression vector into which is inserted a DNA molecule according to the present disclosure. The expression elements of these vectors vary in their strength and specificities. Depending upon the host-vector system utilized, any one of a number of suitable transcription elements can be used.

Another aspect of the present disclosure relates to a transgenic host cell comprising the expression system according to the present disclosure. Once the constructed DNA molecule has been cloned into an expression system, it is ready to be incorporated into a host cell. Such incorporation can be carried out by the various forms of transformation, depending upon the vector/host cell system such as transformation, transduction, conjugation, mobilization, or electroporation. The DNA sequences are cloned into the vector using standard cloning procedures in the art, as described by Maniatis et al., Molecular Cloning: A Laboratory Manual, Cold Springs Laboratory, Cold Springs Harbor, New York (1982), which is hereby incorporated by reference in its entirety. Suitable host cells include, but are not limited to, bacteria, yeast, mammalian cells, insect cells, plant cells, and the like. The host cell is preferably present either in a cell culture (ex vivo) or in a whole living organism (in vivo). In some embodiments, the host cell is either isolated, non-human, or both isolated and non-human.

Mammalian cells suitable for carrying out the present disclosure include, without limitation, COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, NS-1 cells, embryonic stem cells, induced pluripotent stem cells, and primary cells recovered directly from a mammalian organism. With regard to primary cells recovered from a mammalian organism, these cells can optionally be reintroduced into the mammal from which they were harvested or into other animals.

The expression of high levels of functional RNA aptamers within cells can be complicated by several factors including RNA stability, short half-life, and difficulties in cellular targeting. Nonetheless, substantial progress has been achieved over the last several years. The first demonstration of aptamer function in live cells involved nuclear targets (Klug et al., “In Vitro and In Vivo Characterization of Novel mRNA Motifs that Bind Special Elongation Factor SelB,” Proc. Natl. Acad. Sci. U.S.A. 94:6676-6681 (1997); Shi et al., “RNA Aptamers as Effective Protein Antagonists In a Multicellular Organism,” Proc. Natl. Acad. Sci. U.S.A. 96:10033-10038 (1999); Thomas et al., “Selective Targeting and Inhibition of Yeast RNA Polymerase II by RNA Aptamers,” J. Biol. Chem. 272: 27980-27986 (1997), which are hereby incorporated by reference in their entirety). Aptamer function within the nucleus of mammalian cells has also been demonstrated (Symensma et al., “Polyvalent Rev Decoys Act as Artificial Rev-Responsive Elements,” J. Virol. 73:4341-4349 (1999), which is hereby incorporated by reference in its entirety). More recently, effective strategies for cytoplasmic targeting of aptamer have also been developed. For example, the human tRNA initiator sequence, which mediates highly efficient nuclear export to deliver functional chimeric RNA aptamers to the cytosol has been used (Chaloin et al., “Endogenous Expression of a High-Affinity Pseudoknot RNA Aptamer Suppresses Replication of HIV-1,” Nucl. Acids Res. 30:4001-4008 (2002), which is hereby incorporated by reference in its entirety). Functional RNA aptamers have also been directly delivered to the cytoplasm by lipofection (Theis et al., “Discriminatory Aptamer Reveals Serum Response Element Transcription Regulated by Cytohesin-2,” Proc. Natl. Acad. Sci. U.S.A. 101:11221-11226 (2004), which is hereby incorporated by reference in its entirety). Finally, most recently, very high levels of aptamer expression (1×10⁷ molecules per cell) have been achieved by fusion with a highly stable transcript (Choi et al., “Intracellular Expression of the T-cell Factor-1 RNA Aptamer as an Intramer,” Mol. Cancer Ther. 5:2428-2434 (2006), which is hereby incorporated by reference in its entirety).

Plant tissues suitable for transformation include leaf tissue, root tissue, meristems, zygotic and somatic embryos, and anthers. It is particularly preferred to utilize embryos obtained from anther cultures. The expression system of the present invention can be used to transform virtually any plant tissue under suitable conditions, and the transformed cells can be regenerated into whole plants.

One approach to transforming plant cells and/or plant cell cultures, tissues, suspensions, etc. with a DNA molecule of the present disclosure is particle bombardment (also known as biolistic transformation) of the host cell. This technique is disclosed in U.S. Pat. Nos. 4,945,050, 5,036,006, and 5,100,792, all to Sanford, et al., which are hereby incorporated by reference in their entirety. Another method of introducing DNA molecules into a host cell is fusion of protoplasts with other entities, either minicells, cells, lysosomes, or other fusible lipid-surfaced bodies that contain the DNA molecule (Fraley et al., “Expression of Bacterial Genes in Plant Cells,” Proc. Natl. Acad. Sci. U.S.A. 80:4803-4807 (1983), which is hereby incorporated by reference in its entirety). The DNA molecule of the present disclosure may also be introduced into the plant cells and/or plant cell cultures, tissues, suspensions, etc. by electroporation (Fromm et al., “Expression of Genes Transferred into Monocot and Dicot Plant Cells by Electroporation,” Proc. Natl. Acad. Sci. U.S.A. 82:5824 (1985), which is hereby incorporated by reference in its entirety).

In producing transgenic plants, the DNA construct in a vector described above can be microinjected directly into plant cells by use of micropipettes to transfer mechanically the recombinant DNA (Crossway, “Integration of Foreign DNA Following Microinjection of Tobacco Mesophyll Protoplasts,” Mol. Gen. Genetics 202:179-85 (1985), which is hereby incorporated by reference in its entirety). The genetic material may also be transferred into the plant cell using polyethylene glycol (Krens et al., “In Vitro Transformation of Plant Protoplasts with Ti-Plasmid DNA,” Nature 296:72-74 (1982), which is hereby incorporated by reference in its entirety). Alternatively, genetic sequences can be introduced into appropriate plant cells by means of the Ti plasmid of A. tumefaciens or the Ri plasmid of A. rhizogenes, which is transmitted to plant cells on infection by Agrobacterium and is stably integrated into the plant genome (Schell, “Transgenic Plants as Tools to Study the Molecular Organization of Plant Genes,” Science 237:1176-83 (1987), which is hereby incorporated by reference in its entirety). After transformation, the transformed plant cells must be regenerated, and this can be accomplished using well known techniques as described in Evans et al., Handbook of Plant Cell Cultures, Vol. 1, MacMillan Publishing Co., New York (1983); and Vasil (ed.), Cell Culture and Somatic Cell Genetics of Plants, Acad. Press, Orlando, Vol. I (1984) and Vol. III (1986), each of which is hereby incorporated by reference in its entirety.

### Methods of Use

Another aspect of the present disclosure relates to a method of detecting a target molecule. This method involves forming a molecular complex according to the present disclosure; exciting the fluorophore molecule with radiation of appropriate wavelength; and detecting fluorescence by the fluorophore molecule, whereby fluorescence by the fluorophore identifies presence of the target molecule.

Suitable nucleic acid aptamer molecules (i.e., aptamers), target molecules, and fluorophores for use in the methods according to the present disclosure are described in detail supra.

In some embodiments, the fluorophore molecule is 4-(3,5-difluoro-4-hydroxybenzylidene)-1-methyl-5-oxo-4,5-dihydro-1H-imidazole-2-carbaldehyde oxime (“DFHO”), (Z)-5-(3,5-difluoro-4-hydroxybenzylidene)-3-methyl-2-(trifluoromethyl)-3,5-dihydro-4H-imidazol-4-one (“DFHBI-2T”), (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”), (Z)-3-amino-5-(3,5-difluoro-4-hydroxybenzylidene)-2-thioxothiazolidin-4-one (“NRD5”), or methyl (E)-3-(4-((Z)-3,5-difluoro-4-hydroxybenzylidene)-1-methyl-5-oxo-4,5-dihydro-1H-imidazol-2-yl)acrylate (“DFAME”).

In some embodiments, said forming the molecular complex is carried out in a cell. The methods may be carried out in vitro, in vivo, or ex vivo.

In the various methods of use, the formation of molecular complexes of the present disclosure (e.g., fluorophore:nucleic acid aptamer molecule complexes (i.e., fluorophore: aptamer complexes) or fluorophore:nucleic acid aptamer molecule:target complexes (i.e., fluorophore:aptamer:target)) can be identified, quantified, and monitored for various purposes, as discussed more fully below. Detection of molecular complex formation, through the fluorescent output of the fluorophore or a FRET partner (e.g., donor or acceptor), can be used to detect complex formation in a cell-free sample (e.g., cell extracts, fractions of cell extracts, or cell lysates), histological or fixed samples, tissues or tissue extracts, bodily fluids, serum, blood and blood products, environmental samples, or in whole cells. Thus, detection and quantification can be carried out in vivo by fluorescence microscopy or the like, or detection and quantification can be carried in vitro on any of the above extracts or on a sample obtained via in vitro mixing of sample materials and reagents.

The genetic constructs can be introduced into living cells using infective or non-infective transformation procedures that are well known in the art.

Regardless of the intended use, a suitable radiation source is used to illuminate the fluorophore after exposing the fluorophore and aptamer to one another. The radiation source can be used alone or with optical fibers and any optical waveguide to illuminate the sample. Suitable radiation sources include, without limitation, filtered, wide-spectrum light sources (e.g., tungsten, or xenon arc), laser light sources, such as gas lasers, solid state crystal lasers, semiconductor diode lasers (including multiple quantum well, distributed feedback, and vertical cavity surface emitting lasers), dye lasers, metallic vapor lasers, free electron lasers, and lasers using any other substance as a gain medium. Common gas lasers include Argon-ion, Krypton-ion, and mixed gas (e.g., Ar Kr) ion lasers, emitting at 455, 458, 466, 476, 488, 496, 502, 514, and 528 nm (Ar ion); and 406, 413, 415, 468, 476, 482, 520, 531, 568, 647, and 676 nm (Kr ion). Also included in gas lasers are Helium Neon lasers emitting at 543, 594, 612, and 633 mn. Typical output lines from solid state crystal lasers include 532 nm (doubled Nd:YAG) and 408/816 nm (doubled/primary from Ti:Sapphire). Typical output lines from semiconductor diode lasers are 635, 650, 670, and 780 rnm. Infrared radiation sources can also be employed.

Excitation wavelengths and emission detection wavelengths will vary depending on both the fluorophore and the nucleic acid aptamer molecule that are being employed. Examples of different aptamer:fluorophore combinations are described in PCT Application Publ. Nos. WO 2010/096584 and WO 2013/016694, both to Jaffrey et al., which are hereby incorporated by reference in their entirety. As demonstrated therein, several different aptamer molecules can differently affect the emission spectrum of a single fluorophore, affording very distinct emission patterns.

Detection of the emission spectra can be achieved using any suitable detection system. Exemplary detection systems include, without limitation, a cooled CCD camera, a cooled intensified CCD camera, a single-photon-counting detector (e.g., PMT or APD), dual-photon counting detector, spectrometer, fluorescence activated cell sorting (FACS) systems, fluorescence plate readers, fluorescence resonance energy transfer, and other methods that detect photons released upon fluorescence or other resonance energy transfer excitation of molecules.

In some embodiments, the detector is optically coupled to receive the output emissions of the fluorophore:aptamer complex through a lens system, such as in an optical microscope. In some embodiments, a fiber optic coupler is used, where the input to the optical fiber is placed in close proximity to the substrate surface of a biosensor, either above or below the substrate. In some embodiments, the optical fiber provides the substrate for the attachment of nucleic acid sensor molecules and the biosensor is an integral part of the optical fiber.

In some embodiments, the interior surface of a glass or plastic capillary tube provides the substrate for the attachment of the fluorophore or the sensor molecule (or molecular complex). The capillary can be either circular or rectangular in cross-section, and of any dimension. The capillary section containing the biosensors can be integrated into a microfluidic liquid-handling system which can inject different wash, buffer, and analyte-containing solutions through the sensor tube. Spatial encoding of the fluorophore or nucleic acid sensor molecules can be accomplished by patterning them longitudinally along the axis of the tube, as well as radially, around the circumference of the tube interior. Excitation can be accomplished by coupling a laser source (e.g., using a shaped output beam, such as from a VCSEL) into the glass or plastic layer forming the capillary tube. The coupled excitation light will undergo TIR at the interior surface/solution interface of the tube, thus selectively exciting fluorescently labeled biosensors attached to the tube walls, but not the bulk solution. In some embodiments, detection can be accomplished using a lens-coupled, or proximity-coupled large area segmented (pixelated) detector, such as a CCD. In some embodiments, a scanning (i.e., longitudinal/axial and azimuthal) microscope objective lens/emission filter combination is used to image the biosensor substrate onto a CCD detector. In some embodiments, a high resolution CCD detector with an emission filter in front of it is placed in extremely close proximity to the capillary to allow direct imaging of the fluorophore:nucleic acid aptamer complexes. In some embodiments, highly efficient detection is accomplished using a mirrored tubular cavity that is elliptical in cross-section. The sensor tube is placed along one focal axis of the cavity, while a side-window PMT is placed along the other focal axis with an emission filter in front of it. Any light emitted from the biosensor tube in any direction will be collected by the cavity and focused onto the window of the PMT.

In some embodiments, the optical properties of a molecular complex are analyzed using a spectrometer (e.g., such as a luminescence spectrometer). The spectrometer can perform wavelength discrimination for excitation and detection using either monochromators (i.e., diffraction gratings), or wavelength bandpass filters. In some embodiments, the fluorophores of the molecular complexes are excited at absorption maxima appropriate to the fluorophore being used and fluorescence intensity is measured at emission wavelengths appropriate for the complexes being detected. Given that the intensity of the excitation light is much greater than that of the emitted fluorescence, even a small fraction of the excitation light being detected or amplified by the detection system will obscure a weak biosensor fluorescence emission signal. In some embodiments, the biosensor molecules are in solution and are pipetted (either manually or robotically) into a cuvette or a well in a microtiter plate within the spectrometer. In some embodiments, the spectrometer is a multifunction plate reader capable of detecting optical changes in fluorescence or luminescence intensity (at one or more wavelengths), time-resolved fluorescence, fluorescence polarization (FP), absorbance (epi and transmitted), etc., such as the Fusion multifunction plate reader system (Packard Biosciences, Meriden, Conn.). Such a system can be used to detect optical changes in biosensors either in solution, bound to the surface of microwells in plates, or immobilized on the surface of solid substrate (e.g., a microarray on a glass substrate). This type of multiplate/multisubstrate detection system, coupled with robotic liquid handling and sample manipulation, is particularly amenable to high-throughput, low-volume assay formats.

In some embodiments where the sensor molecules or fluorophores are attached to substrates, such as a glass slide or in microarray format, it is desirable to reject any stray or background light in order to permit the detection of low intensity fluorescence signals. In some embodiments, a small sample volume (about 10 nl) is probed to obtain spatial discrimination by using an appropriate optical configuration, such as evanescent excitation or confocal imaging. Furthermore, background light can be minimized by the use of narrow-bandpass wavelength filters between the sample and the detector and by using opaque shielding to remove any ambient light from the measurement system.

In some embodiments, spatial discrimination of a molecular complex (fluorophore:nucleic acid aptamer complexes or fluorophore:nucleic acid aptamer:target molecule complexes) attached to a substrate in a direction normal to the interface of the substrate is obtained by evanescent wave excitation. This is illustrated in PCT Application Publ. No. WO 2010/096584 to Jaffrey and Paige, which is hereby incorporated by reference in its entirety. Evanescent wave excitation utilizes electromagnetic energy that propagates into the lower-index of refraction medium when an electromagnetic wave is totally internally reflected at the interface between higher and lower-refractive index materials. In some embodiments a collimated laser beam is incident on the substrate/solution interface (at which the fluorophore:nucleic acid aptamer complexes or fluorophore:nucleic acid aptamer:target molecule complexes are immobilized) at an angle greater than the critical angle for total internal reflection (TIR). This can be accomplished by directing light into a suitably shaped prism or an optical fiber. In the case of a prism, the substrate is optically coupled (via index-matching fluid) to the upper surface of the prism, such that TIR occurs at the substrate/solution interface on which the molecular complexes are immobilized. Using this method, excitation can be localized to within a few hundred nanometers of the substrate/solution interface, thus eliminating autofluorescence background from the bulk analyte solution, optics, or substrate. Target recognition is detected by a change in the fluorescent emission of the molecular complex, whether a change in intensity or polarization. Spatial discrimination in the plane of the interface (i.e., laterally) is achieved by the optical system.

In some embodiments, a TTRF evanescent wave excitation optical configuration is implemented using a detection system that includes a universal fluorescence microscope. Any fluorescent microscope compatible with TTRF can be employed. The TTRF excitation light or laser can be set at either an angle above the sample shining down on the sample, or at an angle through the objective shining up at the sample. Effective results can been obtained with immobilization of either the aptamer or the fluorophore using NHS-activated glass slides. The fluorophore containing a free amine (at the R₁ position) can be used to react with the NHS-slide. RNA can be modified with a 5′ amine for NHS reactions by carrying out T7 synthesis in the presence of an amine modified GTP analog (commercially available).

In some embodiments, the output of the detection system is preferably coupled to a processor for processing optical signals detected by the detector. The processor can be in the form of personal computer, which contains an input/output (I/O) card coupled through a data bus into the processor. CPU/processor receives and processes the digital output signal, and can be coupled to a memory for storage of detected output signals. The memory can be a random access memory (RAM) and/or read only memory (ROM), along with other conventional integrated circuits used on a single board computer as are well known to those of ordinary skill in the art. Alternatively or in addition, the memory may include a floppy disk, a hard disk, CD ROM, or other computer readable medium which is read from and/or written to by a magnetic, optical, or other reading and/or writing system that is coupled to one or more processors. The memory can include instructions written in a software package (for image processing) for carrying out one or more aspects of the present invention as described herein.

In addition to their specificity in binding to fluorophores, a number of the aptamers have demonstrated that their affinity for the target fluorophore can be modulated by environmental conditions.

In some embodiments, the affinity of the aptamer for the fluorophore is partially or entirely ion dependent, i.e., any mono or divalent ion. For example, PCT Application Publ. No. WO/2010/096584 to Jaffrey and Paige, which is hereby incorporated by reference in its entirety, describes aptamers that are responsive to Mg²⁺ or K⁺. Others have identified aptamers that bind specifically to other ions, and can be incorporated into the sensors of the present invention. These include, without limitation, aptamers specific to zinc (Rajendran et al., “Selection of Fluorescent Aptamer Beacons that Light Up in the Presence of Zinc,” Anal. Bioanal. Chem. 390(4):1067-1075 (2008), which is hereby incorporated by reference in its entirety), cobalt (Breaker et al., “Engineered Allosteric Ribozymes as Biosensor Components,” Curr. Op. in Biotech 13(1):31-39 (2002), which is hereby incorporated by reference in its entirety), and lead (Brown et al., “A Lead-dependent DNAzyme with a Two-step Mechanism,” Biochem. 42(23):7152-7161 (2003), which is hereby incorporated by reference in its entirety).

In some embodiments, the affinity of the aptamer for the fluorophore is temperature dependent. Thus, a titration exists where at very high temperatures, no binding will occur, but at lower temperatures the highest degree of binding will occur. Based on the profile of a particular aptamer-fluorophore pair, the temperature within a system can be determined based on the measured fluorescence output. Aptamers that possess this property can be used as a sensor (discussed below) to determine the temperature of the environment.

In some embodiments, the affinity of the aptamer for the fluorophore is partially pH dependent. The aptamers are fairly stable near neutral pH, but at higher or lower pH, the folding of the aptamer or the interaction between fluorophore/aptamer is disrupted such that changes in fluorescence can be measured as the pH varied away from neutral. Aptamers that possess this property can be used as a sensor (discussed below) to determine the pH of the environment.

The multivalent aptamers having first and second domains can be used for detection of a target molecule in a medium or sample. This is carried out by exposing the nucleic acid aptamer molecule of the invention to a medium suspected to contain the target molecule under conditions effective to allow the second domain to bind specifically to the target molecule, if present, and also exposing the nucleic acid aptamer molecule and medium to a fluorophore of the invention under conditions effective to allow the first domain to bind specifically to the fluorophore after binding of the target molecule by the second domain, thereby inducing the fluorophore to adopt a conformation that exhibits enhanced fluorescent emissions. Detection of molecular complex formation is then achieved by exciting the fluorophore (or FRET partner) with radiation of appropriate wavelength and detecting fluorescence by the fluorophore (or FRET partner), whereby the detection of fluorescence emissions by the fluorophore indicates binding of the nucleic acid molecule to the target molecule and, hence, its presence.

This can be carried out in whole cells either by introducing the nucleic acid aptamer molecule into the whole cell, or by transforming the whole cell with a transgene encoding the nucleic acid aptamer molecule. The fluorophore can be introduced into the environment of the whole cell, where it is readily taken up. This can also be carried out in vitro, i.e., in a cell free environment. An image of the detection process can also be acquired or generated using the detection systems described above.

The present disclosure is particularly adaptable to a microarray format, where the nucleic acid aptamer molecules are tethered at discrete locations on a substrate surface, i.e., solid support. The solid support used to form the microarray surface can include, without limitation, glass, metal, and ceramic supports. Tethering of the nucleic acid aptamer molecules can be carried out using a 5′ biotin to streptavidin-coated glass (ArrayIt, Inc). Alternatively, the sensor molecules of the present disclosure can be provided with an extraneous sequence at its 5′ end, where the extraneous sequence allows for tethering the sensor molecule to a hybridization partner tethered to the array surface using standard techniques. The hybridization partners can be printed onto the array surface, and the sensor molecules allowed to hybridize prior to or after exposing the sensor to the sample. In these array systems, fluorophore is in solution and is recruited to the glass surface only if the target molecule binds the second domain of the surface-bound aptamer, thereby creating a fluorophore:aptamer:target complex that can be detected, e.g., using TIRF. The sensors can be spotted in an array format, i.e., an array of microspots, or configured in other shapes or designs on surfaces, so that the sensors are positioned in a spatially defined manner. This will allow one or a series of sensors that are specific to distinct target molecules to be assayed following contact with a mixture that contains one or more of the target molecules at known or unknown concentrations. The fluorescence intensity can be used to determine the concentrations if suitable solutions containing known amounts of target analytes are used to calibrate the fluorescence signals.

Detection assays can also be carried out using the aptamer constructs that include a first domain that contains the fluorophore-binding aptamer and a second domain that is a hybridization probe has a nucleotide sequence complementary to a target nucleic acid molecule. For example, to detect viral RNA present in a sample, the hybridization probe will contain a nucleotide sequence complementary to the viral RNA. After attaching any nucleic acid in a sample to a substrate (e.g., glass surface), the sample is exposed to the fluorophore and the aptamer construct under conditions to allow hybridization to occur. Subsequent detection of the molecular complex (fluorophore:aptamer construct:complementary viral RNA target), as measured by the fluorescent emissions by the fluorophore on the substrate via TIRF, indicates presence of the viral RNA target. This same assay can be carried out using an aptamer construct that possess a second domain, which instead of being a hybridization probe, includes either an aptamer sequence or a non-aptamer sequence that binds to a specific protein (e.g., MS2 sequence binds the MS2 protein or a fusion protein containing the same), in which case binding of the protein to the substrate (e.g., in an ELISA format) will also allow for detection.

Alternatively, detection assays can be carried out using these same types of aptamer constructs using a fixed cell sample or histologic tissue sample. Where ever the target molecule is present in these samples, the aptamer construct can be bound to the sample and the fluorophore will identify its presence.

While microarrays for monitoring the transcriptome are commonplace and have revolutionized biology, similar approaches are not available to study the proteome. The system and method of the present disclosure allow the production of a protein-sensing microarray. This platform for protein detection has the potential to dramatically speed up the analysis of proteins for innumerable applications. For example, these arrays can be used to assay a set of specific proteins, such as clinically relevant biomarkers, or large sections of the proteome, such as proteins of specific functional classes. Current microarray technologies that utilize a panel of antibodies requires labeling of the proteins in biological samples with fluorescent dyes, such as Cy5-NHS, in order for the protein to be detected after binding to the antibodies. This is problematic, because this labeling procedure may affect the epitope recognized by the antibody. In contrast, the sensor arrays of the present invention do not require target labeling because the sensor will only bind to the fluorophore (at its first domain) after that target molecule has been bound by its second domain. The microarray format of the present invention also overcomes a number of challenges that plagued antibody arrays due to: (1) the low cost of the aptamer sensor molecule; (2) the ease with which oligonucleotides can be coupled to microarray surfaces; (3) the ability to reliably synthesize homogeneous preparations of oligonucleotides, which is a challenge with antibodies; (4) the increased stability of oligonucleotides compared to antibodies; (5) the highly specific nature of aptamer-protein interactions, which typically involve large surfaces (Stoltenburg et al., “SELEX—A Revolutionary Method to Generate High-affinity Nucleic Acid Ligands,” Biomolecular Engineering 24:381-403 (2007); Hermann and Patel, “Adaptive Recognition by Nucleic Acid Aptamers,” Science 287:820-825 (2000), each of which is hereby incorporated by reference in its entirety) rather than short epitopes as with antibodies; and (6) the ease of sample preparation, as the fluorescent signaling obtained using these protein sensors does not require the sample processing step of fluorescent dye tagging. Instead, binding of the target protein to the sensor is sufficient to elicit a fluorescent signal (in the presence of the solution phase fluorophore), thereby dramatically simplifying the analysis of protein mixtures.

Thus, upon exposure to the target and fluorophore, the molecular complex will form and the fluorophore, upon illumination, will exhibit emission patterns from the discrete location on the array surface. Using appropriate mapping software, the presence of the fluorescent emission signal will positively identify the target molecule as being present in the sample being queried. As noted above, quantification can be carried out if reliable calibration is performed.

Yet another aspect of the present disclosure involves a method for detecting nucleic acid molecules using a gel separation technique. RNA or DNA molecules to be detected can be recovered from cells using well known techniques, or collected following in vitro synthesis. First, the recovered nucleic acid molecules are separated on a gel using known procedures and techniques, and thereafter the separated nucleic acid molecules can optionally be transferred to a solid substrate. Regardless, the separated nucleic acid molecules are then exposed to a conditionally fluorescent fluorophore of the type described herein. The gel or substrate (containing the separated nucleic acid molecules and fluorophores, whether present in the form of a molecular complex or not) is illuminated with light of a wavelength suitable to induce fluorescence emissions by the fluorophore that is bound by a nucleic acid molecule (i.e., in the form of a molecular complex). Detection of fluorescent emissions of the fluorophore indicates the location of the nucleic acid molecule on the gel or substrate.

A further aspect of the disclosure involves using an RNA-based metabolite sensor according to the present disclosure. In some embodiments, the sensor comprises a metabolite-binding aptamer portion and a regulated aptamer portion comprising a nucleic acid molecule according to the present disclosure that binds a specific fluorophore and a transducer domain, where the regulated aptamer portion is linked to the metabolite-binding aptamer portion by the transducer domain. In accordance with such embodiments, the metabolite-binding aptamer portion binds specifically to a target molecule for determining the location of a target molecule, particularly within a whole cell. This aspect involves forming a molecular complex (fluorophore:RNA-based metabolite sensor:target molecule), exciting the fluorophore with light of an appropriate wavelength, and then detecting fluorescence by the fluorophore, whereby fluorescence by the fluorophore identifies presence of the target molecule. In whole cells, this embodiment can be carried out by introducing the RNA-based metabolite sensor into the whole cell, or by transforming the whole cell with a transgene encoding the RNA-based metabolite sensor. Once inside the cell, the RNA-based metabolite sensor will bind specifically to the target molecule via its metabolite-binding aptamer domain. The fluorophore can be introduced into the environment of the whole cell, where it is readily taken up. An image of the detection process can also be acquired or generated using the detection systems described above.

A DNA construct encoding one or more nucleic acid aptamer molecules or RNA-based metabolite sensors according to the present disclosure can be used to measure the transcription by a promoter of interest in a cell. This can be carried out by introducing a DNA construct or transgene encoding the RNA one or more nucleic acid aptamer molecules or RNA-based metabolite sensors according to the present disclosure into a cell, introducing the fluorophore into the cell, and then determining whether a molecular complex comprising (i) the one or more nucleic acid aptamer molecules or RNA-based metabolite sensors according to the present disclosure and (ii) the fluorophore forms, as measured by the amount of fluorescence detected within the cell.

This aspect of the disclosure can be used to screen agents for their ability to modulate transcription of the DNA construct and, thus, native genes that contain the same promoter as the DNA construct. When screening an agent, the agent is introduced to the cell, preferably prior to introducing the fluorophore. After a suitable time delay (to allow for transcription of the nucleic acid aptamer to occur), the fluorophore can be introduced to the cell. The detection of an increase or decrease in fluorescence by the molecular complex within the cell, relative to an otherwise identical but untreated control cell, indicates that the agent altered the level of transcription by the promoter.

In some embodiments, the same DNA construct can be used in an in vitro detection procedure, whereby the DNA construct and agent are both introduced into a cell and the fluorophore may or may not be introduced to the cell. In some embodiments, RNA transcripts are recovered from the cell (using known cell lysis and RNA collection procedures) after exposure to the fluorophore. In some embodiments, RNA transcripts are first recovered from the cell, and then the fluorophore is introduced to the recovered RNA transcripts. The fluorophore can be bound to a solid surface of a suitable detection device, such as TIRF system or other detectors of the type described above. The detection of an increase or decrease in fluorescence by the fluorophore:aptamer complex within the recovered RNA transcripts, relative to the RNA transcripts recovered from an otherwise identical but untreated control cell, indicates that the agent altered the level of transcription by the promoter.

In some embodiments, the entire transcription and detection process can be carried out in vitro in the presence of the agent. This can be used to monitor the production of transcripts, and the effects of the agents on those transcripts.

In some embodiments, the agent can be, without limitation, a genetic or transgenic condition unique to a particular cell type, a drug (small molecule), amino acid, protein, peptide, polypeptide, vitamin, metal, carbohydrate, lipid, a polymer, or RNAi that influences transcription levels.

A further aspect of the present disclosure relates to the monitoring an RNA molecule within a cell. This aspect of the disclosure involves the use of a DNA construct of the disclosure that expresses an RNA fusion that includes an RNA aptamer of the invention joined to an RNA molecule of interest. After introducing the DNA construct into a cell and allowing for transcription to occur, the fluorophore of the invention can be introduced to the cell. Alternatively, the RNA molecule can be expressed or synthesized in vitro and later introduced into the cell. Regardless of the approach, this will allow the RNA aptamer portion of the RNA fusion molecule to bind specifically to the fluorophore (forming an aptamer:fluorophore complex) and enhance its fluorescence emissions. Detection of the RNA fusion molecule (including its location, its quantitation, or its degradation) can be carried out by exposing the cell to radiation of a wavelength suitable to induce fluorescence emissions by the fluorophore within the molecular complex; and then measuring the fluorescent emissions of the fluorophore or a FRET partner. The (sub)cellular location of the fluorescence emissions indicates the location of the transcript. Also, any decrease in the fluorescence emissions over time indicates degradation of the transcript. The latter can be confirmed by recovering RNA transcripts and measuring for the RNA fusion using, e.g., RT-PCR. Finally, the level of fluorescence correlates to the quantity of the RNA fusion molecule that is present.

In some embodiments, the RNA product to be monitored can be any of a variety of RNA molecules having diverse functions. These include, without limitation, pre-mRNA, mRNA encoded a native or non-native expression product, pre-rRNA, rRNA, tRNA, hnRNA, snRNA, miRNA, siRNA, shRNA, long noncoding RNA, PIWI RNA, termini-associated RNA, noncoding RNAs, promoter-associated RNAs, viral RNAs, ribozyme, a stabilizing RNA molecule, an RNA sequence that binds a protein such as a MS2 protein-binding RNA, a targeting element that can localize the fusion nucleic acid molecule to a specific localization in the cell. The RNA product can be fused to either the 5′ end or the 3′ end of the aptamer molecule of the present disclosure.

The monitoring of the RNA can also be carried out by exposing the cell to an extracellular RNA molecule that includes an aptamer of the present disclosure, and cellular uptake of the RNA molecule can be observed via microscopy or measurement of the fluorescent emissions upon exposure to the fluorophore (either before or after cell uptake).

Thus, this aspect can used to monitor the effects of an experimental treatment on RNA localization, trafficking, expression levels, rate of degradation, etc., where the experimental treatment can be exposing the cell or organism to an agent such as a drug (small molecule), amino acid, protein, peptide, polypeptide, vitamin, metal, carbohydrate, lipid, a polymer, or RNAi that influences the target molecule or the expression level of another protein in a pathway influenced by the target RNA molecule, expression of a native or foreign gene in the cell or organism, or exposing the cell or organism to a change in environmental conditions (e.g., temperature, hypoxic or hyperoxic conditions, atmospheric pressure, pH, etc.). These treatments can be carried out directly on a transformed cell or cell population. Alternatively, these treatments can be performed on an organism that contains one or more cells transformed with a DNA construction encoding the fusion RNA molecule of interest.

To enhance the fluorescent signal, it is possible to tailor the number of fluorophores that can be bound to a single RNA transcript by using a concatemer of RNA aptamers. In addition, this aspect of the disclosure is particularly adaptable to assessing the trafficking or degradation of multiple RNA molecules simultaneously. This is possible due to the tailored emission spectra of different aptamer:fluorophore complexes. Thus, this aspect can include introducing a second DNA construct into a cell, where the second DNA construct encoding a distinct RNA fusion molecule that includes a distinct RNA aptamer of the invention (or a concatemer thereof) joined to a distinct RNA molecule of interest. After introducing the DNA construct into the cell or organism, and allowing for transcription to occur, a second fluorophore of the invention can be introduced to the cell or organism, i.e., one that is bound specifically by the aptamer present in the second RNA fusion molecule but not the first, and vice versa. This will allow the fluorophore-specific aptamer portion of the RNA to bind specifically to the fluorophore (forming an aptamer:fluorophore complex) and enhance its fluorescence emissions. Detection of fluorescence can be carried out as described above. Simultaneous detection of separate emission peaks will allow for detecting localization or co-localization of both complexes.

In a related aspect, the disclosure materials can be used to assess RNA folding, unfolding, or folding-unfolding kinetics by monitoring changes in fluorescence after exposing the RNA fusion protein to a fluorophore of the present invention (to form a molecular complex). The unfolding or folding event can be produced by exposing the molecular complex to an agent such as a protein (e.g., enzyme such as helicase), chemical (e.g., a small organic molecule, vitamin, amino acid, antibiotic, protein, lipid, carbohydrate, polymer, nucleotide, RNA-binding protein, or RNA-binding molecule), ribozyme, or environmental changes (e.g., temperature, hypoxic or hyperoxic conditions, atmospheric pressure, pH, etc.). The RNA aptamer can be the target of the folding or unfolding, or the RNA aptamer can be fused to the target of the folding or unfolding and, as such, incidentally be subject to its folding or unfolding. For the fusion RNA molecule, this aspect of the disclosure can be practiced in vivo in which case the folding or unfolding event can be affected by the expression of a gene within a cell or organism where the gene encodes a protein, an RNA, a non-coding RNA, an RNAi molecule (e.g., siRNAi, shRNA). Detection of unfolding can be measured by a decrease in fluorescence, and detection of folding can be measured by an increase in fluorescence, following exposure of the in vitro system or cell to radiation of a wavelength suitable to induce fluorescence emissions by the fluorophore within the molecular complex; and then measuring the fluorescent emissions of the fluorophore or a FRET partner.

In a related aspect, the disclosure materials can be used to assess RNA binding to another moiety by observing the proximity of the fluorescence signal generated by the RNA aptamer (or RNA fusion) to a moiety. The moiety can be an RNA sequence (e.g., mRNA encoding a protein or noncoding RNA of the types described above), DNA or modified nucleic acid molecule. The RNA aptamer can be the target of the binding event, or the RNA aptamer can be fused to the target of the RNA binding event and, as such, incidentally be subject to structural changes following the binding event. For the fusion RNA molecule, this aspect of the disclosure can be practiced in vivo in which case the RNA binding event can be carried by the expression of a transgene encoding the RNA fusion molecule within a cell or organism. Detection of RNA binding can be measured by a decrease in fluorescence, and a decrease in RNA binding can be measured by an increase in fluorescence, following exposure of the in vitro system or cell to radiation of a wavelength suitable to induce fluorescence emissions by the fluorophore within the molecular complex; and then measuring the fluorescent emissions of the fluorophore or a FRET partner.

A further aspect of the disclosure relates to monitoring a target molecule in a cell. This aspect of the disclosure can be carried out using a RNA-based metabolite sensor that includes a metabolite-binding aptamer portion and a regulated aptamer portion, as described above, where the regulated aptamer portion binds specifically to the fluorophore only after the metabolite-binding aptamer portion binds specifically to the target molecule. Both the nucleic acid aptamer molecules and a fluorophore according to the present disclosure may be introduced into a cell, allowing the fluorophore:RNA-based metabolite sensor:target complex to form in the presence of the target molecule and enhancing the fluorescence emissions by the fluorophore. Upon exposure of the cell to radiation of suitable wavelength to induce fluorescence emissions by the fluorophore that is bound in the complex or a FRET partner; and then measuring the fluorescent emissions of the fluorophore or FRET partner to monitor the target molecule. In this manner, the cellular location of the fluorescence emissions indicates the location of the target molecule, a decrease in the fluorescence emissions over time indicates degradation of the target molecule, and an increase in the fluorescence emissions over time indicates accumulation of the target molecule. Quantitation of the target molecule can be correlated to the level of fluorescence measured.

The target molecule in this aspect of the disclosure can be any protein, lipid, carbohydrate, hormone, cytokine, chemokine, cell signaling molecule, metabolite, organic molecule, or metal ion, as described above.

This aspect of the disclosure can be carried by introducing the nucleic acid aptamer molecule directly into the cell or, alternatively, by introducing into the cell a gene that encodes the nucleic acid aptamer molecule.

Another aspect of the present disclosure relates to a method of screening a drug that modifies gene expression. This aspect can be carried out using a transgene that encodes an nucleic acid aptamer molecule or RNA-based metabolite sensor of the present disclosure. The transgene can be provided with a promoter of interest whose activity is being monitored with respect to the drug being screened. After introducing the transgene into a cell, the cell is exposed to the drug and a fluorophore of the invention, effectively introducing these compounds into the cell. Thereafter, the level of RNA aptamer transcription is measured by exposing the cell to radiation of a wavelength suitable to induce fluorescence emissions by the fluorophore that is bound by the RNA aptamer molecule or a FRET partner, and the fluorescent emissions of the fluorophore or FRET partner are measured, as described above. A reduction or absence of fluorescent emissions, relative to an otherwise identical control cell that is not exposed to the drug, indicates that the drug inhibits expression of the transgene. An increase of fluorescent emissions, relative to an otherwise identical control cell that is not exposed to the drug, indicates that the drug promotes expression of the transgene.

Another aspect of the present disclosure relates to a method of screening a drug that modifies RNA splicing. This aspect can be carried out using a transgene that encodes an nucleic acid aptamer molecule of the present disclosure, where the RNA transcript of the transgene includes an intron that, with proper splicing, will result in a mature RNA molecule that is a functional fluorophore-binding RNA aptamer of the invention. This method is carried out by introducing the transgene into a cell and exposing the cell to a drug, and allowing transcription to occur such that both the immature transcript and the drug will both be present in the cell when splicing is to occur. A fluorophore of the invention is also introduced into the cell, whereby the mature RNA aptamer, if properly spliced, will be able to bind specifically to the fluorophore to enhance its fluorescence emissions. Detection of whether proper splicing occurred (or not) can be carried out by exposing the cell to radiation of a wavelength suitable to induce fluorescence emissions by the fluorophore (that is bound by the mature RNA aptamer molecule), or its FRET partner, and then measuring the fluorescent emissions of the fluorophore or FRET partner. A reduction or absence of fluorescent emissions, relative to an otherwise identical control cell that is not exposed to the drug, indicates that the drug inhibits proper splicing of the transcript. An increase of fluorescent emissions, relative to the otherwise identical control cell that is not exposed to the drug, indicates that the drug promotes proper splicing of the transcript.

This aspect of the disclosure can also be carried out in vitro. Basically, a medium is provided that contains the immature RNA transcript (with intron), a spliceosome including an appropriate splicing enzyme, a drug to be screened, and the fluorophore. As noted above, the immature RNA transcript includes first and second exons having an intervening intron region, and the first and second exons, upon excision of the intron, form an RNA aptamer molecule of the present disclosure. Upon exposing the medium to radiation of a wavelength suitable to induce fluorescence emissions by the fluorophore that is bound by the RNA aptamer molecule (or a FRET partner), any fluorescent emissions of the fluorophore (or FRET partner) are measured. A reduction or absence of fluorescent emissions, relative to an otherwise identical medium that lacks the drug, indicates that the drug inhibits proper splicing of the transcript. An increase of fluorescent emissions, relative to an otherwise identical medium that lacks the drug, indicates that the drug promotes proper splicing of the transcript.

In some embodiments, as an alternative to exposing the cell or organism to a drug, the cell or organism can be exposed to a protein or polypeptide, modifying the expression level of a gene with the cell or organism where the gene encodes a protein, an RNA, a non-coding RNA, a shRNA, or other RNA, introducing a transgene into the cell or organism where the transgene expresses and RNAi molecule, or exposing the cell or organism to a change in environmental conditions of the types described above.

Yet another aspect of the disclosure relates to a method of screening a drug for activity on a target molecule (i.e., either enhancing or diminishing activity of the target molecule). This process is carried out by introducing or expressing within a cell an RNA-based metabolite sensor of the present disclosure that includes a metabolite-binding aptamer portion and a regulated aptamer portion, as described above, where the regulated aptamer portion binds specifically to the fluorophore only after the metabolite-binding aptamer portion binds specifically to the target molecule. A fluorophore of the type described above is also introduced into the cell, where the fluorophore is bound specifically to the regulated aptamer portion of the nucleic acid molecule when the target molecule is bound by the metabolite-binding aptamer portion, thereby enhancing fluorescent emissions by the fluorophore. Upon exposure of the cell to radiation of suitable wavelength to induce fluorescence emissions by the fluorophore that is bound in the complex or a FRET partner, and then measuring the fluorescent emissions of the fluorophore or FRET partner, it is possible to determine whether the activity of the target molecule is modified by the drug. Where a difference exists in the fluorescent emissions by the fluorophore or FRET partner, relative to an otherwise identical cell that lacks the drug, then this will indicates that the drug modifies the activity of the target molecule.

A further aspect of the disclosure relates to the de novo creation of aptamer-based sensor molecules for a particular target, without any prior knowledge of the aptamer for the particular target. This process is achieved using a modified SELEX procedure, where the nucleic acid molecules of the pool each contain a partially destabilized aptamer molecule that contains a first domain that binds specifically to a fluorophore of the present invention, and a second domain that comprises a wholly or partly random sequence. By partially destabilizing the first domain, only after binding of the second domain to the target molecule is first domain capable of binding specifically to the fluorophore.

SELEX is carried out by exposing the pool of nucleic acid molecules to a target molecule and the fluorophore (whereby fluorescence emissions by the fluorophore are enhanced by the binding of the first domain to the fluorophore). Illuminating the fluorophore with light of a wavelength suitable to induce fluorescence emissions by the fluorophore that is bound by the first domain molecule, and measuring the fluorescent emissions of the fluorophore provide an indication as to whether any members of the pool bound to the target molecule (via their second domain).

RNAs members of the pool can be “precleared” by passing the RNAs over fluorophore-bound to agarose. This will remove all library members that retain constitutive fluorophore-binding activity (i.e., even in the absence of a functional second domain that binds to the target). In the next step, the pool is exposed to the fluorophore-bound agarose, except that this time the target will be added to the incubation buffer. All washes will also contain target. After washing, the elution will occur in the same buffer, except that no target will be present. Thus, any RNAs whose binding to the fluorophore is dependent on target will elute. These RNAs will be recovered and used for subsequent rounds of SELEX to enrich for target-regulated sensors. The fluorescence of each pool will be tested as above in the presence of the fluorophore with or without the target of interest, and individual clones that exhibit target-dependent fluorescence can be isolated.

A negative selection can also be used to ensure that the sensors do not respond to structurally related molecules. To do this, the structurally related molecules can also be introduced in the elution buffer, so that if they promote fluorophore binding they will be retained on the agarose (whereas sensor constructs that are unaltered by these structurally related molecules will elute).

Fundamentally, this same approach can be used to screen drugs for binding to a target nucleic acid molecule of interest. RNA sequences of interest that have no known drug to target the same can be screened against a library, for instance a chemical library, to find new molecules that would bind to this RNA sequence of interest. Because binding of drugs typically stabilizes RNA sequences, the sensor can be a turn-on sensor of the type described above. Rather than using a random nucleotide sequence for the second domain, the RNA sequence of interest is used as the second domain and it is fused to the fluorophore-binding aptamers of the invention (a first domain). Upon drug binding to the second domain, the nucleic acid molecule will adopt a stabilized conformation that allows the first domain to bind and induce fluorescence of a fluorophore. Thus, the chemical library can be screened based on whether or not the test molecule increases the overall fluorescence. This will allow for the rapid screening of chemical libraries for the discovery of new drugs that bind to known RNA sequences of interest.

In a further aspect of the disclosure, a transgene of the present disclosure can be inserted into a viral genome and then packaged to form an infective delivery vehicle, or the transgene can be inserted into a virus like particle to form a pseudovirion. Infection of a cell by the virus or pseudovirus can be detected by measuring expression of the transgene encoding the RNA aptamer or RNA fusion. Expression of the transgene can be detected by exposing the cell to the fluorophore and then exposing the cell to radiation of a wavelength suitable to induce fluorescence emissions by the fluorophore that is bound by the RNA aptamer molecule (or a FRET partner). Any fluorescent emissions of the fluorophore (or FRET partner) reflect transgene expression and, hence, viral or pseudoviral infection of the cell. In contrast, the absence of fluorescence indicates that the virus or pseudovirus did not infect the cell. This aspect of the disclosure can be used to screen putative therapeutic agents for their ability to inhibit viral infection. Additionally, viral particles themselves can be quantified by fluorescence if the viral particle contains single-stranded RNA containing the aptamer sequence and the fluorophore.

### Kits

A further aspect of the present disclosure relates to various kits that can be used for practicing the present disclosure. The kit components can vary depending upon the intended use, and any reagents identified in this application can be included in the one or more kits. The kits can be packaged with components in separate containers or as mixtures, as noted below. Instructions for use may also be provided.

For example, according to one embodiment, the kit may comprising a fluorophore comprising a methyne bridge between a substituted aromatic ring system and a substituted imidazol(thi)one, oxazol(thi)one, pyrrolin(thi)one, or furan(thi)one ring; and a nucleic acid molecule according to the present disclosure. Accordingly, the kit can include one or more fluorophores of the type described above and one or more nucleic acid molecules according to the present disclosure or genetic constructs encoding those nucleic acid molecules. The genetic construct can be designed for RNA trafficking studies, or for expression of multivalent sensor molecules.

In some embodiments, the aptamer component that is responsible for binding to the fluorophore can be selected such that each of a plurality of nucleic acid aptamers causes a different emission profile by a single fluorophore. In this way, a single fluorophore can be used for multiple, simultaneous detections. In some embodiments, the plurality of nucleic acid aptamers can be supplied separately, e.g., in different containers, or they can be supplied as a mixture or as a range of mixtures, such that each mixture is characterized by a different blended fluorescent emission pattern with the same fluorophore. Suitable fluorophores are described in detail infra.

In some embodiments, the fluorophore molecule is 4-(3,5-difluoro-4-hydroxybenzylidene)-1-methyl-5-oxo-4,5-dihydro-1H-imidazole-2-carbaldehyde oxime (“DFHO”), (Z)-5-(3,5-difluoro-4-hydroxybenzylidene)-3-methyl-2-(trifluoromethyl)-3,5-dihydro-4H-imidazol-4-one (“DFHBI-2T”), (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”), (Z)-3-amino-5-(3,5-difluoro-4-hydroxybenzylidene)-2-thioxothiazolidin-4-one (“NRD5”), or methyl (E)-3-(4-((Z)-3,5-difluoro-4-hydroxybenzylidene)-1-methyl-5-oxo-4,5-dihydro-1H-imidazol-2-yl)acrylate (“DFAME”).

In some embodiments, the kit can include one or more fluorophores that are immobilized on a substrate to allow for SELEX. The substrate can be an FTIR suitable flow cell. The kit can also include one or more “turn-on” sensor molecules, which are matched for each of the one or more fluorophores, i.e., the fluorophore-specific domain of the sensor is specific for only one of the surface-bound fluorophores or elicits distinct emissions by two or more of the surface-bound fluorophores. This will allow for detection of the target molecule in a sample.

In some embodiments, the kit can include one or more nucleic acid aptamers that are immobilized on a support, which can be a surface of a substrate. Examples of suitable supports include, without limitation, another nucleotide sequence including RNA, DNA, PNA or modifications or mixtures of these oligonucleotides; a macromolecular structure composed of nucleic acid, such as DNA origami; a surface composed of glass, such as a glass slide; a surface formed of a plastic material such as plastic slides; a protein or polypeptide, such as an antibody; an oligosaccharide; a bead or resin. The substrate can be provided with a plurality of the nucleic acid aptamers that are positioned at discrete locations so as to form an array. The spots on the array where the nucleic acid aptamers are retained can have any desired shape or configuration.

In some embodiments, the kit can include a plurality of distinct fluorophores of the disclosure, and a plurality of distinct nucleic acid molecules of the invention which bind specifically to at least one of the plurality of fluorophores. Preferably, only a single monovalent or multivalent nucleic acid aptamer molecule is provided for each fluorophore. To enable their use together, each fluorophore:aptamer pair should be characterized by a distinct emission spectrum such that each can be detected independently. As demonstrated by the accompanying examples, a plurality of distinct aptamer/fluorophore complexes can achieve distinguishable emission spectra. The multiple colors will allow imaging of multiple RNAs simultaneously and allow the development of protein-RNA and RNA-RNA FRET systems.

For example, using multiple sensor molecules with distinct fluorophores that are compatible with FRET, detection of interactions of RNA or DNA with fluorescent proteins, RNAs, or other molecules can be achieved. FRET occurs if an appropriate acceptor fluorophore is sufficiently close to the acceptor fluorophore. Therefore, the interaction of a fluorescent protein, RNA, DNA, or other molecule with an RNA-fluorophore complex can be detected by measuring the FRET emission upon photoexcitation of the acceptor. Measurements like this can be used to measure the rate of binding of a fluorescent molecule to an RNA that is tagged with an RNA-fluorophore complex in both in vitro and in vivo settings. In a similar application, the RNA-fluorophore complex can serve as a donor and a fluorescent protein, RNA, DNA, other molecule can serve as the acceptor. In these cases, the RNA-fluorophore complex can be excited, and FRET emission can be detected to confirm an interaction. As used herein, a FRET partner refers to either a FRET acceptor or a FRET donor, which is used in combination with a fluorophore/aptamer complex of the disclosure.

In some embodiments, the kit can include an empty genetic construct of the invention, as described above, along with one or more of the following: one or more restriction enzymes, one or more fluorophore compounds of the invention (which are operable with the aptamer sequence encoded by the construct), and instructions for inserting a DNA molecule encoding an RNA molecule of interest into the restriction sites for formation of a genetic construct that encodes a transcript comprising the RNA molecule of interest joined to the RNA aptamer molecule.

### Methods of Generating a Randomized Aptamer Library

Another aspect of the present disclosure relates to a method of generating a randomized aptamer library. This method involves providing a DNA sequence encoding a riboswitch aptamer; modifying the DNA sequence encoding the riboswitch aptamer by introducing deletions, point mutations, and/or insertions or random nucleotides to generate a library comprising a plurality of modified sequences.

In some embodiments, the riboswitch aptamer is selected from the group consisting of add A-riboswitch, SAM-II-riboswitch, TPP-riboswitch, FMN-riboswitch, cyanocobalamin-riboswitch, etc.

The library may be modified to comprise spontaneous insertions (termed “sprouts”) and/or deletions (termed “clips”) at one, two, three, four, five, six, seven, eight, nine, ten, or more positions of the DNA sequence encoding the riboswitch aptamer. In some embodiments, the modified positions are in the ligand-binding pocket of the riboswitch aptamer. The generation of such a random sprouts and clips library is described in the Examples which follow.

In some embodiments, modifying the DNA sequence encoding the riboswitch aptamer is carried out by identifying one or more DNA sequences which encode a ligand binding region of the riboswitch aptamer (e.g., by identifying the region(s) which comprise the ligand binding pocket of the riboswitch aptamer). In accordance with such embodiments, the DNA sequences which encode the ligand binding region of the riboswitch aptamer are modified by introducing deletions, point mutations, and or insertions of random nucleotides.

To introduce point mutations, said modifications may be incorporated by chemically synthesizing the DNA sequence encoding the riboswitch aptamer with a phosphoramidite mixture that contains all four nucleotides for a given position within the identified ligand binding region of the riboswitch aptamer.

To introduce point mutations and deletions (i.e., shortening, also termed “clips”), said modifying may be incorporated by chemically synthesizing the DNA sequence encoding the riboswitch aptamer with a phosphoramidite mixture that contains all four nucleotides for a given position within the identified ligand binding region of the riboswitch aptamer and by carrying out the phosphoramidite coupling reaction for a given position within the identified ligand binding region of the riboswitch aptamer for 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, or 20 seconds. In some embodiments, deletions in the DNA sequence encoding the riboswitch aptamer are introduced by carrying out the phosphoramidite coupling reaction for a given position for 5 seconds, 4 seconds, 3 seconds, 2 seconds, or less. In some embodiments, deletions in the DNA sequence encoding the riboswitch aptamer are introduced by carrying out the phosphoramidite coupling reaction for a given position for 2 seconds.

To introduce insertions or random additions of nucleotides (i.e., expanded sequences, also termed “sprouts”), said modifications may be incorporated by chemically synthesizing the DNA sequence encoding the riboswitch aptamer with a phosphoramidite mixture that contains all four nucleotides at a concentration in the range of 0.1 to 1 mM (the standard concentration being 100 mM) for a given position within the identified ligand binding region of the riboswitch aptamer.

In accordance with all aspects of the methods of generating a randomized aptamer library, said methods does not involve a “capping step” during synthesis of the portion of the oligonucleotide that contains the randomized nucleotide sequences. This allows incorporation of either deletion or insertion of random nucleotides into the DNA library. All the other nucleotides in the sequence which has a specific identity (i.e., A, T, G or C) are synthesized with the capping step.

In some embodiments, the randomized aptamer library comprises approximately 7.3×10²⁶ possible different members.

The presently claimed method of generating a randomized aptamer library uses a novel DNA synthesis method to generate DNA libraries where both the sequence and the size are randomized. There are couple of methods that researchers have previously reported which can generate similar libraries. However, none of the previous methods can generate library diversity as large as the presently disclosed method of generating a randomized aptamber library.

In particular, the method described by Bartel et al., “HIV-1 Rev Regulation Involves Recognition of Non-Watson-Crick Base Pairs in Viral RNA,” Cell 67(3):529-536 (1991), which is hereby incorporated by reference in its entirety, generated a library by omitting the capping step. The capping step removes any growing strand that did not incorporate the phosphoramidite that is being coupled in that round of synthesis. Therefore omitting the capping step includes the strands which did not couple to the phosphoramidite, introducing point deletions (clips). However they used the natural inefficiency of the phosphoramidite coupling step (˜1-2% in each round) to generate these deletions. The method of generating a randomized aptamer library according to the present disclosure was developed by surveying many conditions and used more inefficient coupling conditions to generate deletions more frequently (˜7% in each round).

The presently claimed method of generating a randomized aptamer library allows for the insertion of nucleotides, which increase the size of the library members. This cannot be achieved by the method described by Bartel et al., “HIV-1 Rev Regulation Involves Recognition of Non-Watson-Crick Base Pairs in Viral RNA,” Cell 67(3):529-536 (1991), which is hereby incorporated by reference in its entirety.

Further, the method described by Giver et al., “Selective Optimization of the Rev-Binding Element of HIV-1,” Nucleic Acids Res. 21:5509-5516 (1993), which is hereby incorporated by reference in it is entirety, uses a cumbersome procedure to generate a library which is randomized in both sequence and size. To generate two stretches of 6-9 random nucleotides separated by a constant region, first four separate columns were used to generate a pool with random regions of 6, 7, 8, 9 nucleotides. The first column was synthesized with 6 random nucleotides, the second with 7 random nucleotides, etc.

Following the addition of the flanking constant sequence, the synthesis was stopped, the four columns were opened, and the resins from the four columns were mixed. The mixed resins were then equally re-divided into four new columns and the synthesis was resumed. The first column incorporated 6 positions, the second column 7 positions, etc. Thus, the first column contained oligonucleotides in which the first random segment was 6, 7, 8, or 9 residues long, and a second random segment that was uniformly 6 residues long. The second column contained oligonucleotides in which the first random segment was 6, 7, 8, or 9 residues long and a second random segment was uniformly 7 residues long, and so forth. Following the completion of all four syntheses, the reactions were combined to generate the final random sequence pool.

Although the method described in Giver et al., “Selective Optimization of the Rev-Binding Element of HIV-1,” Nucleic Acids Res. 21:5509-5516 (1993), which is hereby incorporated by reference in it is entirety, theoretically generated a pool that is similar to the presently described randomized aptamer library, it requires multiple mixing steps which could be tedious. Secondly, the method described in Giver et al., “Selective Optimization of the Rev-Binding Element of HIV-1,” Nucleic Acids Res. 21:5509-5516 (1993), which is hereby incorporated by reference in it is entirety, synthesized a pool where the random region can vary only between 6-9 nucleotides, while the presently described method of generating a random aptamer library can generate library where the number of random nucleotide can vary between 0 to 44 nucleotides. Lastly, incorporating multiple stretches of random regions flanked by constant regions, increases the number of mixing steps required. However, the presently described method of generating a randomized aptamer library does not use any manual mixing of the resin and the DNA library can be synthesized in one simple go making it much easier.

The method described by Porter et al., “Recurrent RNA Motifs as Scaffolds for Genetically Encodable Small-Molecule Biosensors,” Nat. Chem. Biol. 13(3):295-301(2017), which is hereby incorporated by reference in its entirety, used a method where a SELEX library was generated using previously reported riboswitch and ribozyme structures as scaffolds. The nucleotides which constitute the binding pockets of these RNAs were randomized while the overall structure where unperturbed. Although this library generated binding pockets of different sequence, their size mostly remained unchanged. The method of generating a randomized aptamer library according to the present disclosure, on the other hand, not only changes the sequence of the binding pocket, but also its size as well which can accommodate ligands of different sizes.

Another method described by Dixon et al., “Reengineering Orthogonally Selective Riboswitches,” Proc. Natl. Acad. Sci. U.S.A 107:2830-2835 (2010), which is hereby incorporated by reference in its entirety, earlier used a similar concept, but was carried out by mutagenizing a few residues that directly contact the ligand, thereby limiting the size of the library. Dixon et al., “Reengineering Orthogonally Selective Riboswitches,” Proc. Natl. Acad. Sci. U.S.A 107:2830-2835 (2010), which is hereby incorporated by reference in its entirety, used this library to find RNAs which binds molecules that are slightly different from the wild type ligand. The presently disclosed randomized aptamer library can be used to find aptamers for molecules which have no resemblance with the parental ligand.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

### EXAMPLES

The following examples are provided to illustrate embodiments of the present technology but are by no means intended to limit its scope.

### Materials and Methods for Examples 1-7

### Reagents and Equipment

Unless otherwise stated, all reagents were from Sigma-Aldrich except for cell culture reagents, which were from Invitrogen. These reagents were used without further purification. DFHBI-1T, DFHO, and BI fluorophores were obtained from Lucerna Technologies (New York, NY) or were synthesized as described previously (Song et al., “Plug-and-Play Fluorophores Extend the Spectral Properties of Spinach,” J. Am. Chem. Soc. 136:1198-1201 (2014); Song et al., “Imaging RNA polymerase III transcription using a Photostable RNA—Fluorophore Complex,” Nat. Chem. Biol. 13:1187-1194 (2017); and which are hereby incorporated by reference in their entirety). DNA libraries were ordered from the Keck Oligonucleotide Synthesis facility (Yale University) and primers were ordered from Integrated DNA Technologies (IDT). Absorbance spectra were recorded with a Thermo Scientific NanoDrop 2000 spectrophotometer with cuvette capability. ChemiDoc MP imager (Bio-Rad) was used to record bacterial colony fluorescence on agar plates as described previously (Filonov et al., “Broccoli: Rapid Selection of an RNA Mimic of Green Fluorescent Protein by Fluorescence-Based Selection and Directed Evolution,” J. Am. Chem. Soc. 136: 16299-16308 (2014), which is hereby incorporated by reference in its entirety) and images were collected using Image Lab (ver. 5.2.1). Fluorescence was measured on FluoroMax-4 fluorimeter (Horiba Scientific) using FluorEssence (ver. 3.5). Fluorescence was also measured on Spectramax iD3 plate reader (Molecular Devices) using SoftMax Pro (ver. 7.1). FACS experiments were performed using BD FACSAria II instrument (BD Biosciences) and flow cytometry was performed using BD LSRFortessa (BD Biosciences). Sorting and flow cytometry data was collected using BD FACSDiva (ver. 8.0.1) software and analyzed using FlowJo (ver. 10.7.1). Fluorescence imaging experiments were performed using an Eclipse Ti-E microscope (Nikon) and images were collected using NIS Elements (ver. 3.22.15). Data were plotted using Sigmaplot (ver. 10.0) and GraphPad Prism (ver. 9.2.0).

### Preparation of Affinity Matrix

DFHBI and DFHO affinity matrix were prepared as described before (Paige et al., “RNA Mimics of Green Fluorescent Protein,” Science 333:642-646 (2011) and Li et al., “Fluorophore-Promoted RNA Folding and Photostability Enables Imaging of Single Broccoli-Tagged mRNAs in Live Mammalian Cells,” Angew. Chemie Int. Ed. 59:4511-4518 (2019), which are hereby incorporated by reference in their entirety). Amine-functionalized DFHBI or DFHO (50 mM in DMSO) was diluted to 5 mM in 100 mM HEPES-KOH buffer (pH 7.5). 2 mL of the fluorophore solution was then added to 1 mL of NHS-activated Sepharose (GE Life Sciences) beads, which had been washed with 2×1 mL of cold reaction buffer. The beads were then incubated with amine-functionalized fluorophore solution overnight at 4° C. in the dark with gentle agitation. Then the beads were washed with the reaction buffer to remove the unreacted DFHBI or DFHO and incubated with 5 mL of 100 mM Tris HCl (pH 8.0) for 2 hours at room temperature to inactivate any remaining NHS-activated sites. After thorough washing with the reaction buffer and then with water, the beads were stored in 1:1 ethanol:0.1 M sodium acetate (pH 5.2) at 4° C. The efficiency of the coupling was calculated by quantifying the amount of free DFHBI or DFHO in the flow-through using absorbance. Using this approach, it was estimated that the Sepharose beads contain approximately 5 μmol of DFHBI or DFHO per mL of resin after the coupling reaction.

### SELEX Procedure

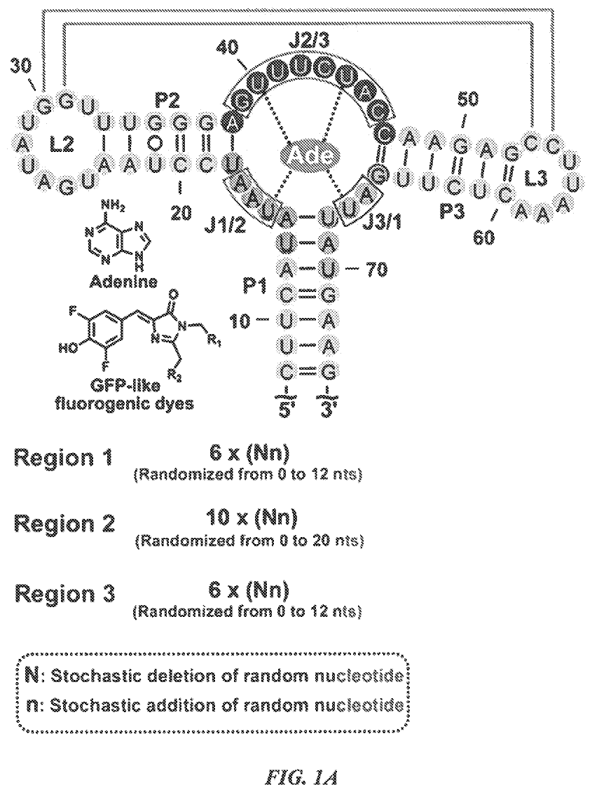

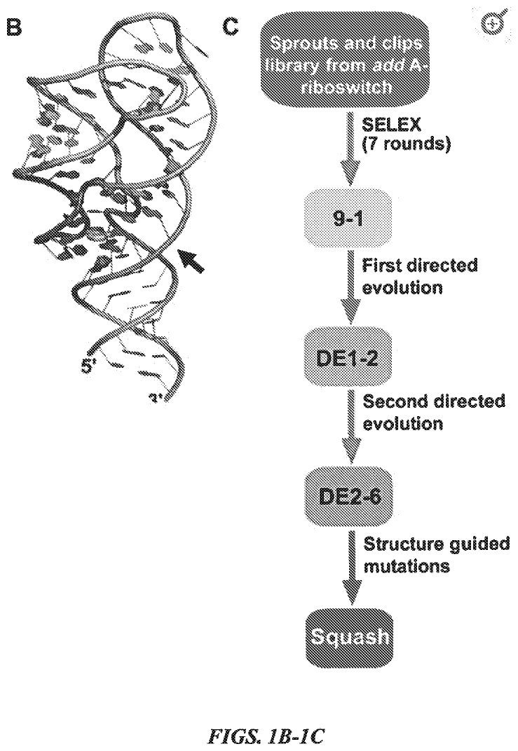

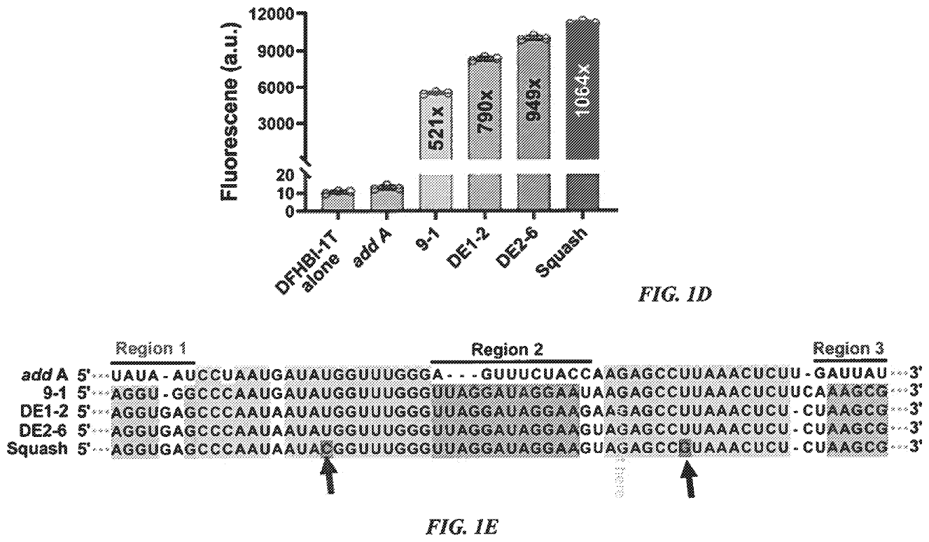

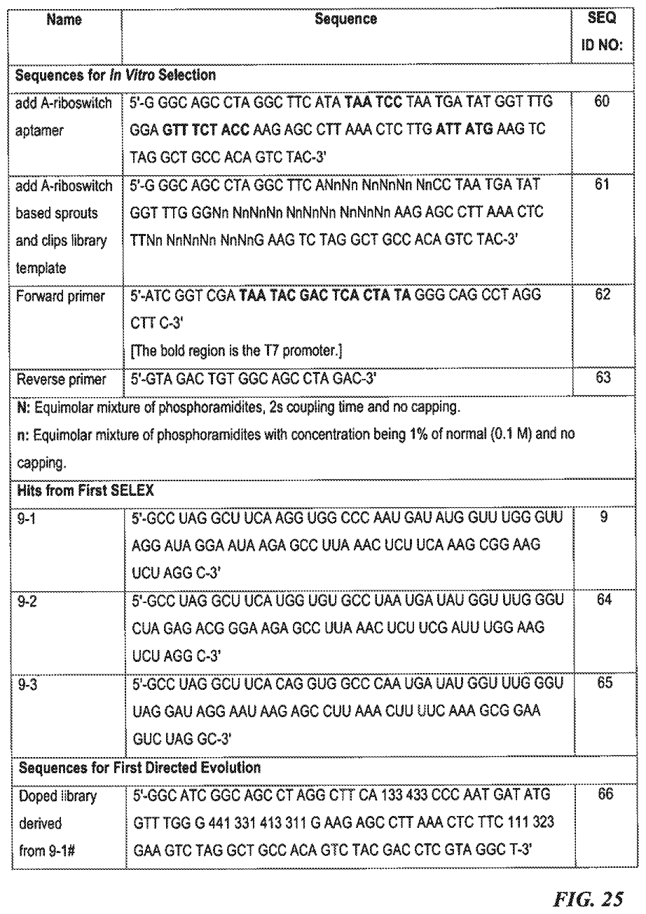

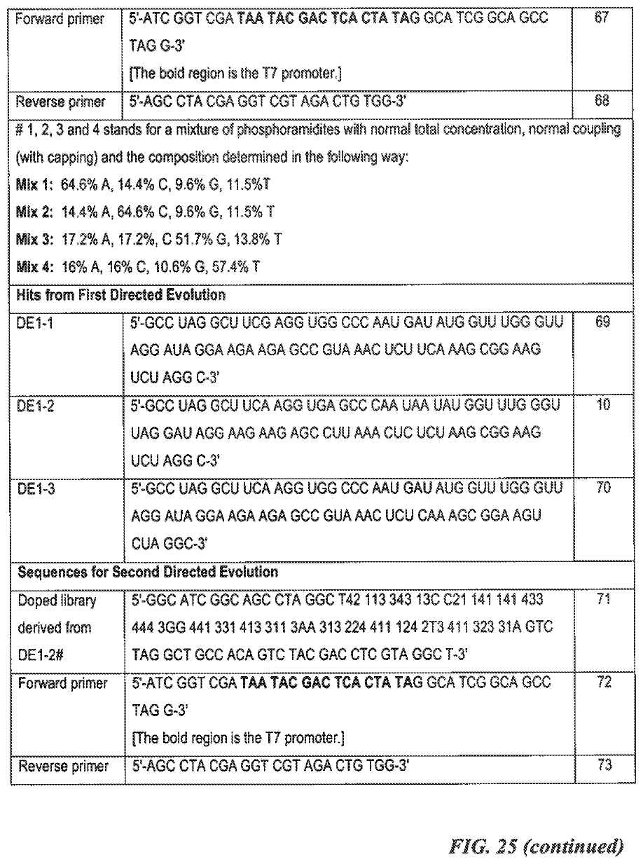

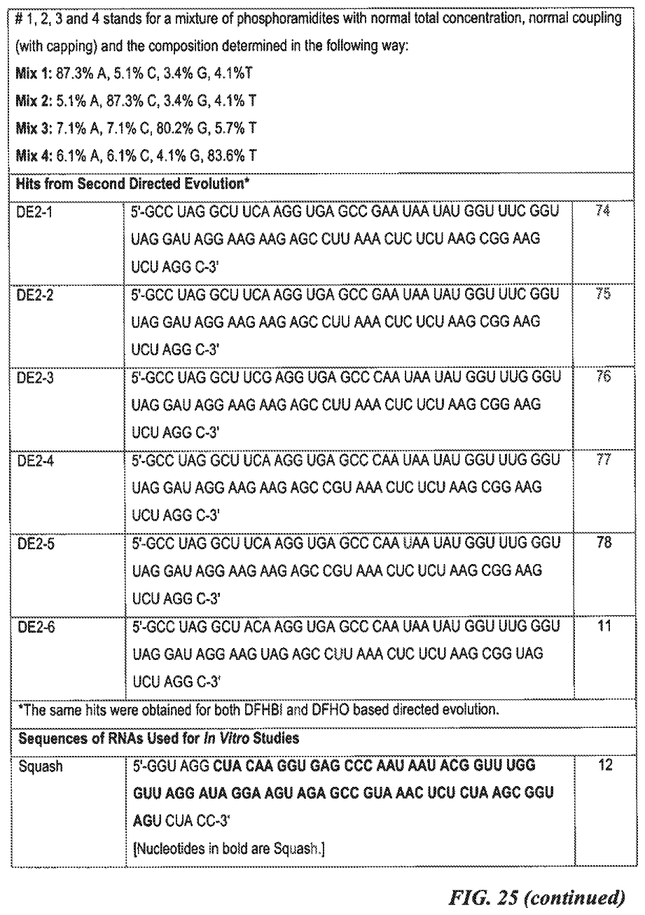

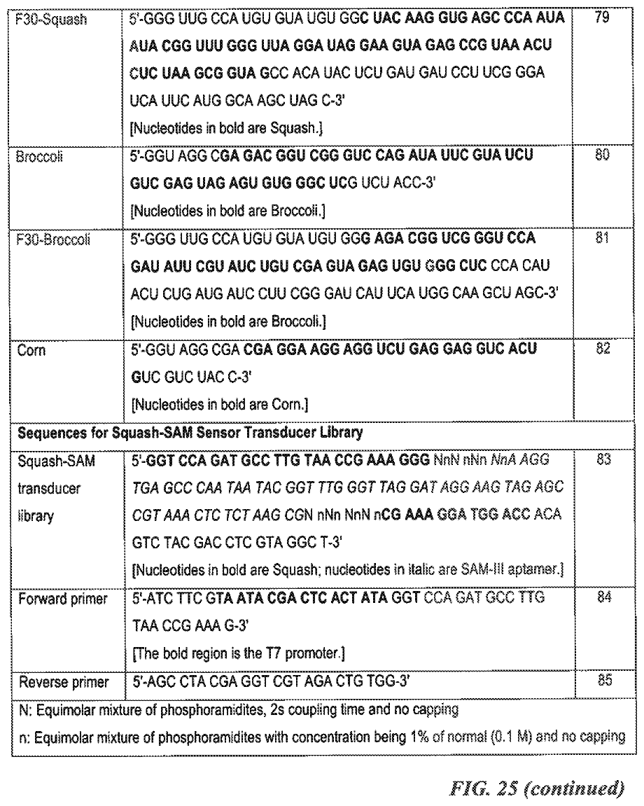

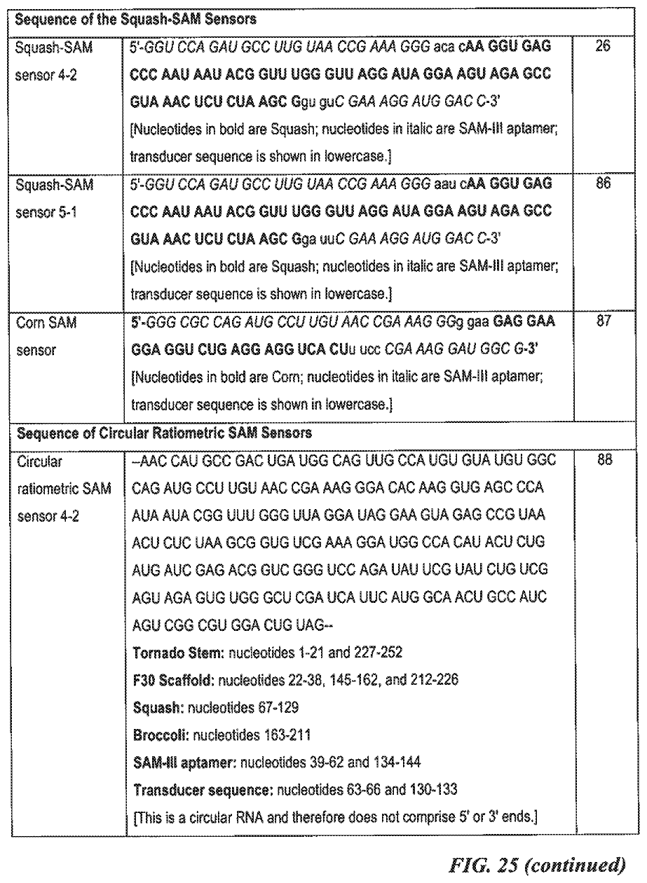

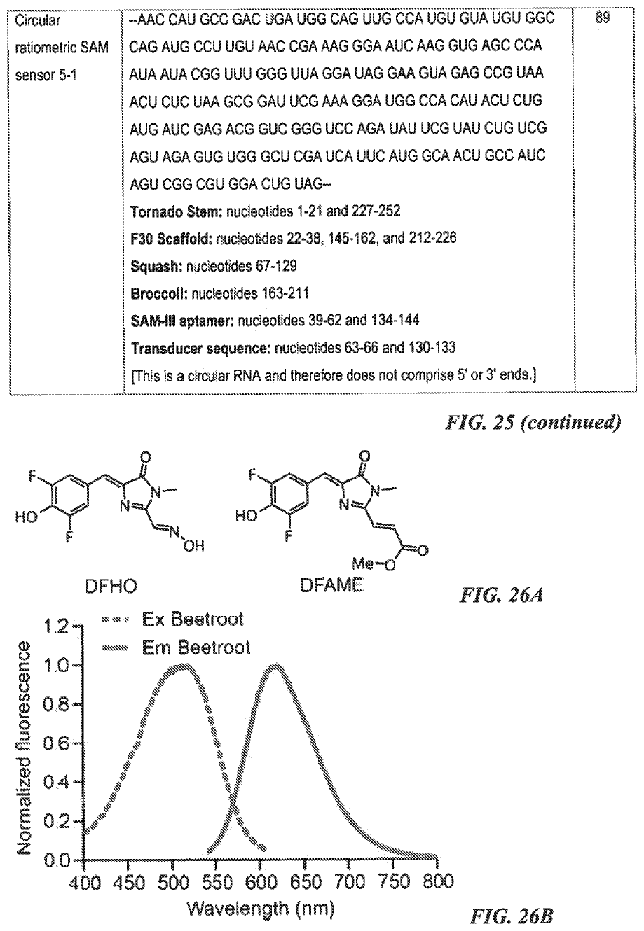

The single-stranded sprouts and clips random DNA library (FIG. 1A; FIG. 25 (sequences for in vitro selection)) was purified using denaturing PAGE (8%, 7 M urea). Approximately 1 nanomole of purified ssDNA library was amplified for five cycles in 10 mL final volume (divided in a 96 well plate) using Taq DNA polymerase with Thermopol buffer (New England Biolabs) to generate the dsDNA template for in vitro transcription.

Approximately 6×10¹⁴ different sequences of dsDNA template were transcribed in 250 μL T7 RNA polymerase transcription reaction using the AmpliScribe T7-Flash Transcription Kit (Lucigen). After treatment with DNase I (30 minutes at 37° C.), the RNA was purified using RNA Clean and Concentrator 100 columns (Zymo Research).

SELEX was performed essentially as described before (Paige et al., “RNA Mimics of Green Fluorescent Protein,” Science 333:642-646 (2011) and Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014), which are hereby incorporated by reference in their entirety) using a single selection buffer for every round (40 mM HEPES-KOH (pH 7.4), 100 mM KCl and 5 mM MgCl₂). First, library members capable of binding to the Sepharose beads were pre-cleared by incubation with “mock” beads consisting of aminohexyl-functionalized Sepharose. The mock beads (300 L) were washed and then equilibrated in the selection buffer. Approximately 2 nanomole of the library RNA was diluted in 500 μL of selection buffer containing 0.1 mg/mL of yeast transfer RNA (tRNA) and incubated with the mock beads for 30 minutes with gentle agitation. After collecting the flow-through, the mock beads were washed with 500 μL of selection buffer containing 0.1 mg/mL of yeast tRNA and the flow-through was collected again. The combined flow-through from both the steps was then incubated with pre-equilibrated DFHBI-functionalized beads (300 μL) for 30 minutes with gentle agitation. Then the DFHBI-functionalized beads were washed with 3×500 L of selection buffer. Finally, RNAs that specifically bound to DFHBI were eluted with 1 mM DFHBI-1T.

The eluted RNA was ethanol precipitated using NaOAc (0.3 M final), glycoblue (0.1 mg/mL final) and 75% ethanol (final). Then the RNA was pelleted by centrifugation at 20000×g for 30 minutes after overnight incubation at −20° C. Then the RNA was reverse-transcribed using Superscript IV reverse transcriptase (Invitrogen) following manufacturer's protocol in a total volume of 30 μL. The entire 30 μL reverse-transcription reaction was PCR-amplified in a total volume of 300 μL using standard Taq DNA polymerase conditions with Thermopol buffer (New England Biolabs). The PCR reaction was purified using QIAquick PCR Purification Kit (Qiagen) and used for in vitro transcription to generate the RNA pool for the next round. Selection pressure was exerted on the later rounds by more stringent washing (see Table 1 for SELEX conditions). The presence of fluorescent RNA species in each round was assessed by mixing 20 μM RNA pool and 10 μM DFHBI-1T and measuring fluorescence emission of this solution on a fluorimeter in comparison with the fluorophore alone. After round 7, the RNA library were cloned into bacterial expression plasmid for FACS-based screening (described below).

TABLE 1
SELEX Condition for add-A Riboswitch Aptamer
Derived Sprouts and Clips Library

| | | Volume of | | | |
|---|---|---|---|---|---|
| | RNA Amount | selection | Bead amount* | | |
| Round | (nmoles) | buffer | (μL) | Washes | Elution** |
| 1 | 2 | 500 μL | 300 | 3 × 500 μL | 2 × 300 μL |
| 2 | 0.5 | 200 μL | 100 | 4 × 500 μL | 2 × 300 μL |
| 3 | 0.2 | 200 μL | 40 | 5 × 500 μL | 2 × 200 μL |
| 4 | 0.1 | 200 μL | 20 | 6 × 300 μL | 2 × 200 μL |
| 5 | 0.1 | 200 μL | 20 | 7 × 300 μL | 2 × 200 μL |
| 6 | 0.1 | 200 μL | 10 | 8 × 300 μL | 2 × 200 μL |
| 7 | 0.1 | 200 μL | 10 | 10 × 300 μL | 2 × 200 μL |
| 8 | 0.1 | 200 μL | 10 | 12 × 300 μL | 2 × 200 μL |

*The amount of ‘mock’ bead used for each round is same as the amount of DFHBI-coupled bead used for that round; the DFHBI-coupled bead capacity was approximately 5 μmoles of DFHBI per mL of resin.
**Elution was done with 1 mM DFHBI-IT in the selection buffer at room temperature. Elute from the two different elution were combined and then precipitated together.

### Directed Evolution of Fluorogenic Aptamers

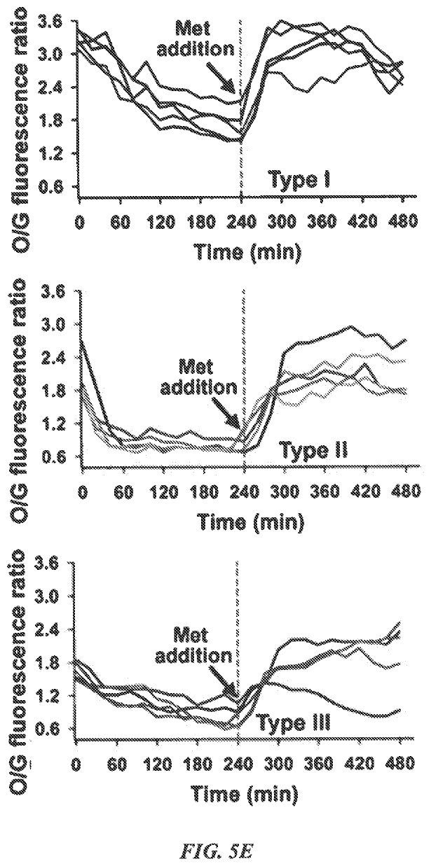

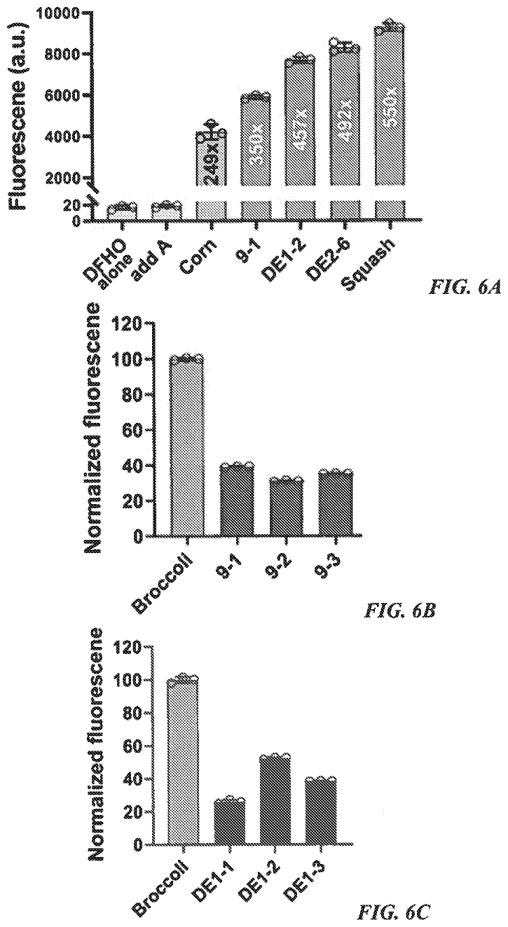

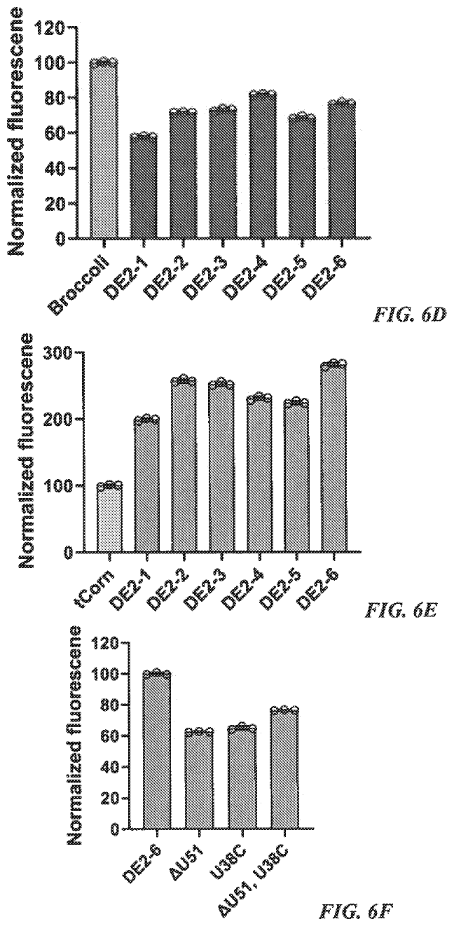

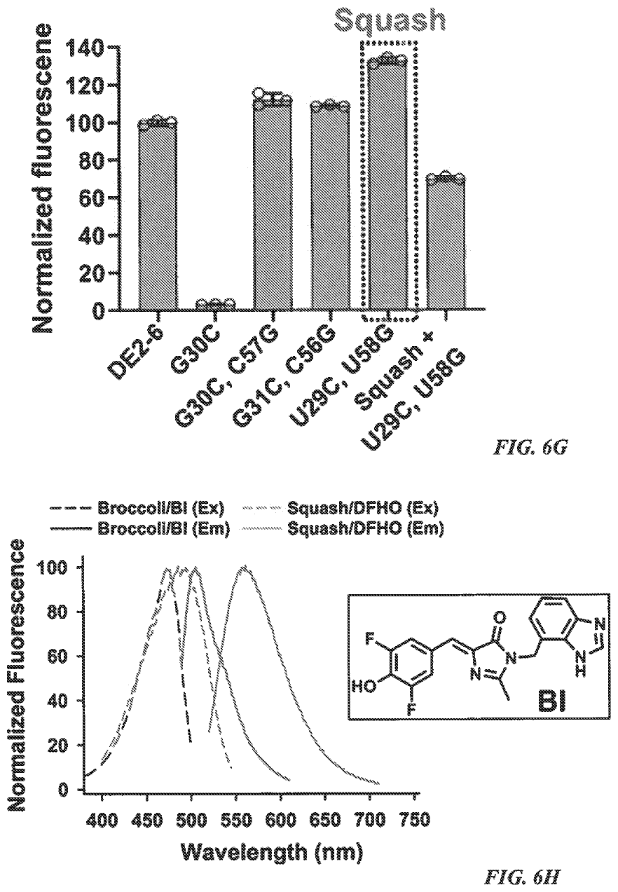

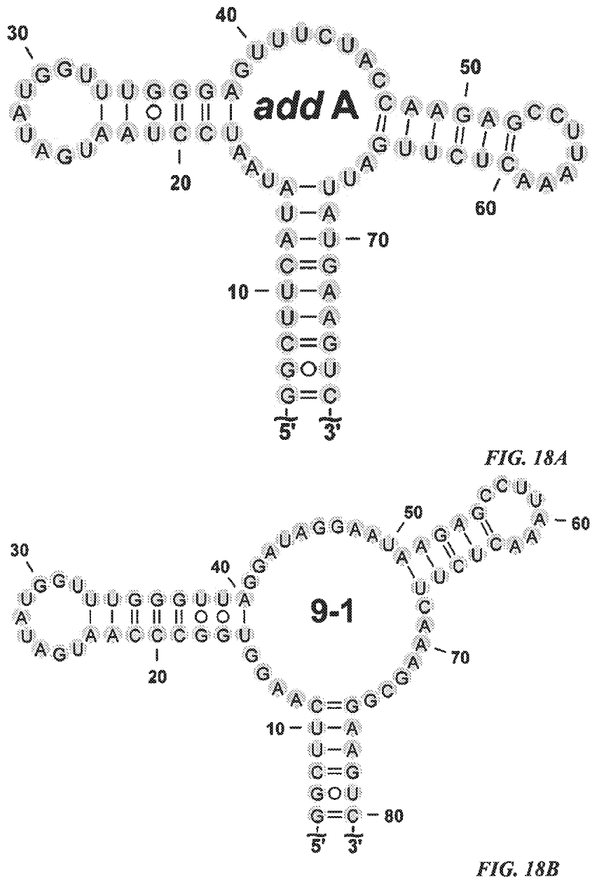

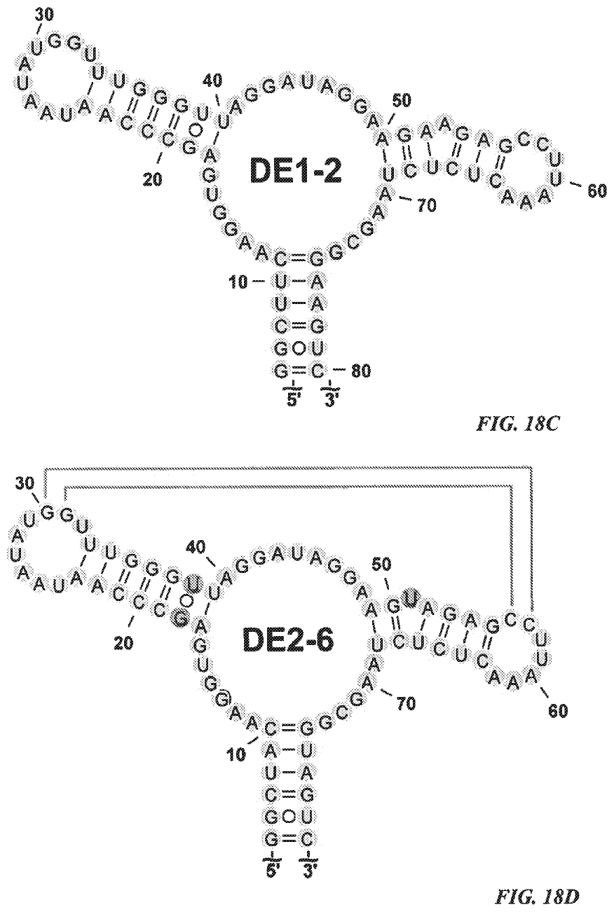

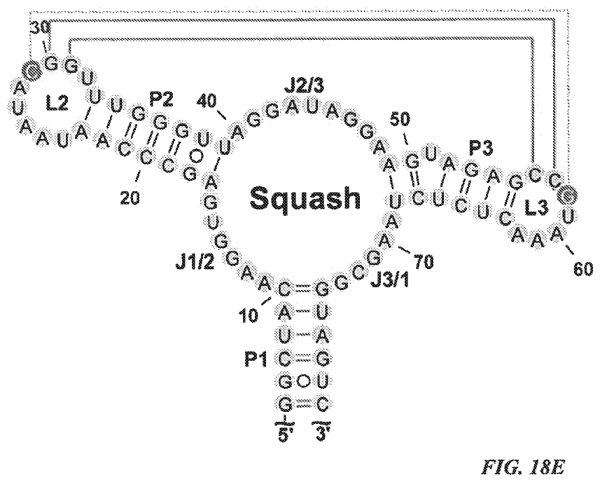

The directed evolution using doped library was conducted as described in Filonov et al., “Broccoli: Rapid Selection of an RNA Mimic of Green Fluorescent Protein by Fluorescence-Based Selection and Directed Evolution,” J. Am. Chem. Soc. 136: 16299-16308 (2014), which is hereby incorporated by reference in its entirety. In brief, these libraries were created in a way that each library member resembles the parental aptamer 9-1, except that there are on average eight mutations per sequence (FIG. 25 (Sequences for First Directed Evolution)). In order to obtain this library, every mutagenized position is chemically synthesized with a phosphoramidite mixture that contains primarily the original nucleotide at that position, but also contains a small fraction of the other three nucleotides (Filonov et al., “Broccoli: Rapid Selection of an RNA Mimic of Green Fluorescent Protein by Fluorescence-Based Selection and Directed Evolution,” J. Am. Chem. Soc. 136: 16299-16308 (2014), which is hereby incorporated by reference in its entirety). After each selection, the in vitro fluorescence of individual library members were measured to select the candidate for the next round of directed evolution (FIGS. 6B-6E; FIGS. 18A-18E). During the directed evolution, selection pressure was exerted by lowering the magnesium concentration in selection buffer and increasing the temperature of the wash buffer (Table 2). A second directed evolution was performed using a doped library based on DE1-2 aptamer (FIG. 25 (Sequences for Second Directed Evolution)) for both DFHBI and DFHO (Table 3). However, the same hits were obtained for both DFHBI- and DFHO-based directed evolution (FIG. 25 (Hits from Second Directed Evolution)). Finally the DE2-6 aptamer selected from the second directed evolution was further optimized by introducing rational structure-based mutations (FIGS. 6F-6G).

TABLE 2
SELEX Condition for the First Directed Evolution

| | RNA Amount | Bead amount* | Mg²⁺ Conc. | | Wash buffer temp | |
|---|---|---|---|---|---|---|
| Round | (nmoles) | (μL) | (mM) | Washes | (° C.) | Elution** |
| 1 | 1 | 100 | 1 | 6 × 300 μL | 37 | 2 × 200 μL |
| 2 | 0.3 | 20 | 0.5 | 8 × 300 μL | 40 | 2 × 200 μL |
| 3 | 0.1 | 20 | 0.2 | 10 × 300 μL | 43 | 2 × 200 μL |
| 4 | 0.1 | 10 | 0.1 | 12 × 300 μL | 46 | 2 × 200 μL |

*The amount of ‘mock’ bead used for each round is same as the amount of DFHBI-coupled bead used for that round.
**Elution was done with 1 mM DFHBI-IT or 1 mM DFHO in the selection buffer at 37° C.; elute from the two different elution were combined and then precipitated together.

TABLE 3
SELEX Condition for the Second Directed Evolution

| | RNA Amount | Bead amount* | Mg²⁺ Conc. | | Wash buffer temp | |
|---|---|---|---|---|---|---|
| Round | (nmoles) | (μL) | (mM) | Washes | (° C.) | Elution** |
| 1 | 1 | 100 | 0.2 | 6 × 300 μL | 37 | 2 × 200 μL |
| 2 | 0.2 | 20 | 0.1 | 8 × 300 μL | 40 | 2 × 100 μL |
| 3 | 0.1 | 20 | 0.1 | 10 × 300 μL | 43 | 2 × 100 μL |
| 4 | 0.1 | 10 | 0.1 | 12 × 300 μL | 46 | 2 × 100 μL |

*The amount of ‘mock’ bead used for each round is same as the amount of DFHBI-coupled bead used for that round; the DFHBI-agarose bead capacity was approximately 5 μmoles/mL of resin.
**Elution was done with 1 mM DFHBI-IT or 1 mM DFHO in the selection buffer at 37° C.; elute from the two different elution were combined and then precipitated together.

### Bacterial Library Generation and FACS Sorting

The RNA pool after seven rounds of SELEX on the ‘sprouts and clips’ random library, or the RNA pool after four rounds of SELEX on the doped library, were reverse transcribed and PCR amplified. Then these PCR products were cloned into pET28c F30-Broccoli plasmid using BglII and NheI sites using T4 DNA ligase (New England Biolabs).

The resulting ligation mixtures were purified and electroporated into BL21 DE3 E. coli (Lucigen). These cells then were grown in LB media with kanamycin. Typical bacterial libraries contain 5-20×10⁶ individual members. Next day the cells were diluted 1:10 in LB and induced for RNA expression with 1 mM IPTG for 3 hours. Cells were preincubated with either 20 μM DFHBI-1T or 10 μM DFHO (in 1×PBS) and then sorted on a FACSAria II instrument. The sample holder of the sorter was maintained at 37° C. to sort cells expressing thermostable aptamers. For DFHBI-1T-binding aptamers, cells were excited with the 488 nm laser and their emission was collected using 525±25 nm emission filter. To isolate yellow fluorescent aptamers, cells were excited with the 488 nm laser and emission was collected using 545±17.5 nm emission filter. Cells were sorted using arbitrary gates where roughly five hits are obtained in 10000 sorted cells. The hits from sorting were collected into 1 mL SOC media and recovered by shaking at 37° C. for 1 hour. Then the cells were plated on LB-agar supplemented with kanamycin and 10 μM DFHBI-1T or 5 μM DFHO.

The next day, the bacterial colonies were induced with 1 mM IPTG for 3 hours and imaged on a ChemiDoc MP imager using either green fluorescence (470±15 nm ex; 532±14 nm em), or yellow fluorescence (530±14 nm ex; 605±25 nm em). To normalize the colony size, autofluorescence signal from bacterial colonies were collected using the Cy5 channel (630±+15 nm ex; 697±22.5 nm emission). Images were processed and normalized in ImageJ (NIH) to identify colonies expressing the brightest aptamers.

### In Vitro Characterization of Aptamers

After sequencing the plasmids from the brightest colonies, dsDNA templates (containing T7 promoter) were generated using PCR amplification. To generate truncation, deletion and point mutants, dsDNA templates containing T7 promoter were PCR amplified from the appropriate ssDNA sequences (IDT). PCR products were then purified with QIAquick PCR Purification Kit (Qiagen) and transcribed using AmpliScribe T7-Flash Transcription Kit (Lucigen). After treatment with DNase I (30 min at 37° C.), the RNA was purified using RNA Clean and Concentrator 25 columns (Zymo Research).

All RNAs used for in vitro studies (except the initial hits from SELEX and the DE2-6 mutants) were purified using 8% denaturing PAGE (7M urea) and eluted in 0.3 M NaOAc (pH 5.2) overnight. The RNA was ethanol precipitated and quantified using absorbance in a NanoDrop 2000 spectrometer. All in vitro RNA properties were measured in 40 mM HEPES-KOH (pH 7.4), 100 mM KCl, 0.5 mM MgCl₂ buffer unless specified.

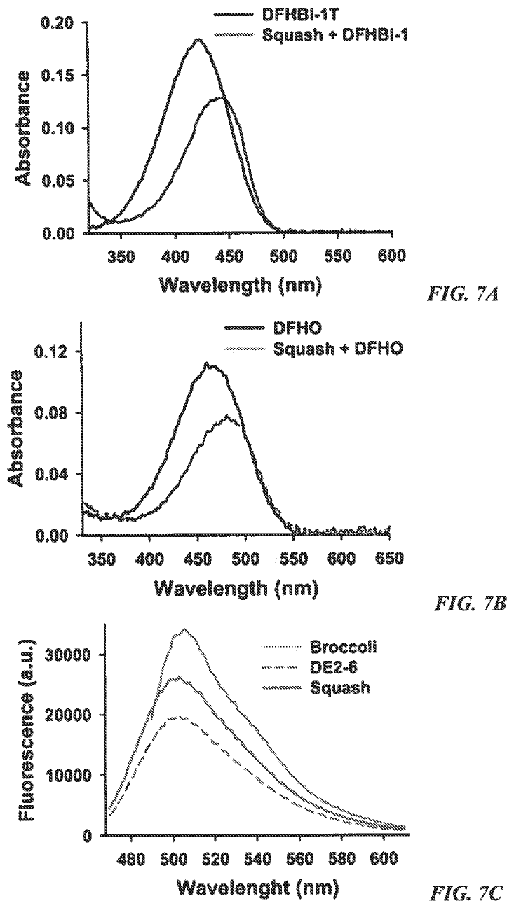

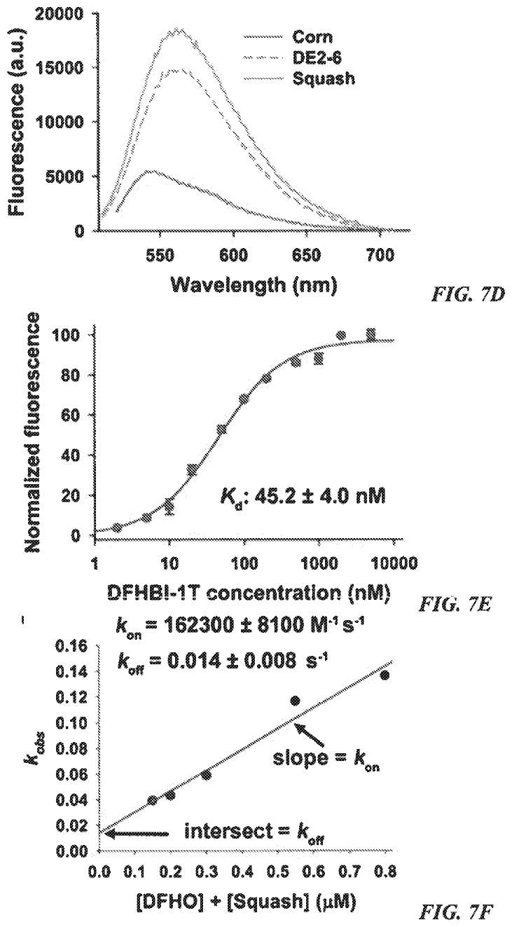

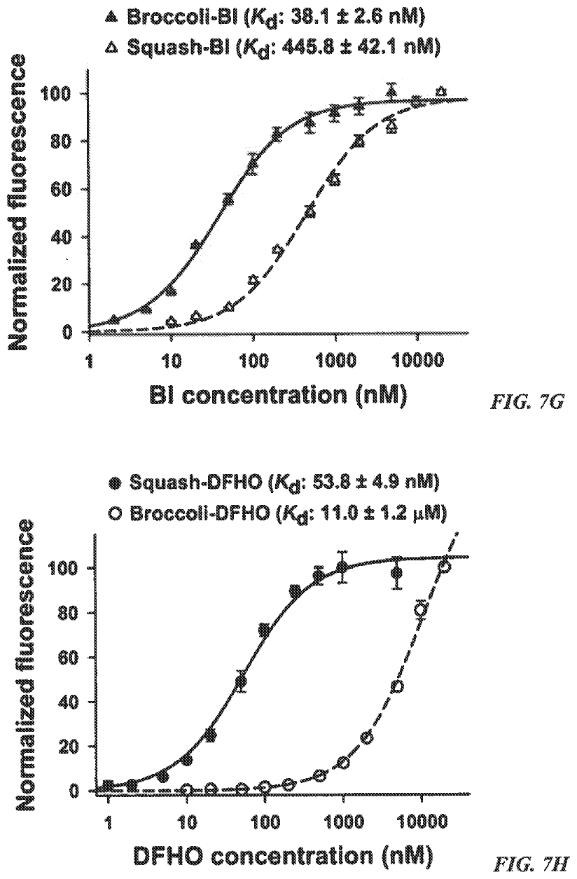

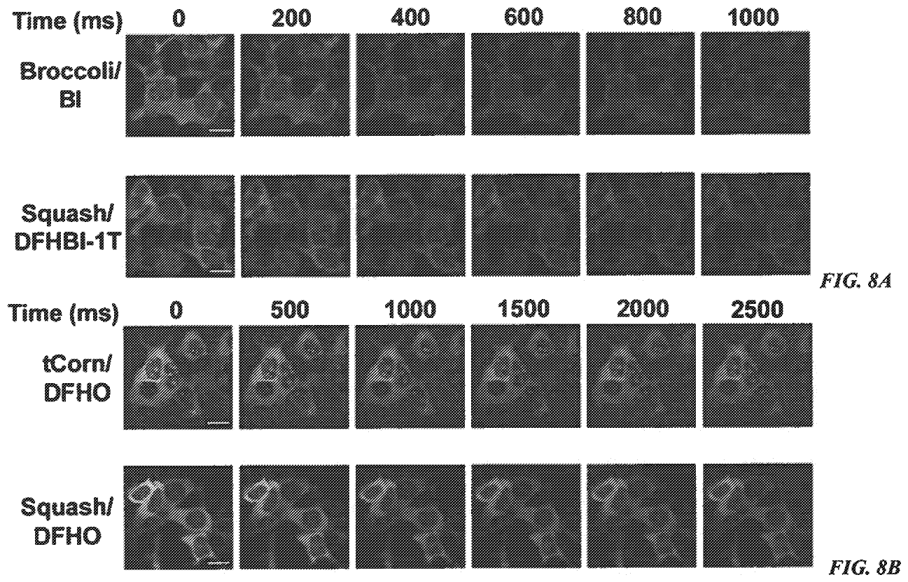

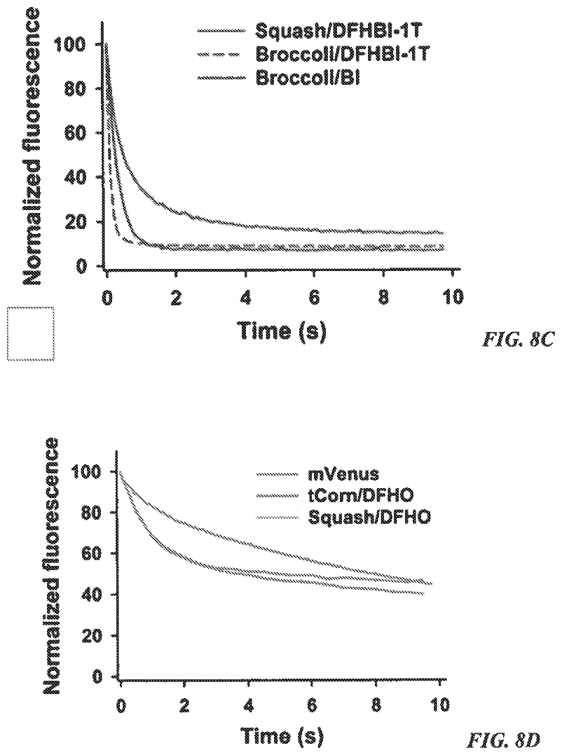

Absorption, excitation, and emission spectra were measured using conditions where the RNA is in excess and the fluorophore is limiting to ensure that no free fluorophore contributes to the absorbance or fluorescence signal (FIGS. 7A-7D). This approach also allowed a fixed concentration of RNA-fluorophore complex which is equal to the concentration of the fluorophore that was added (Strack et al., “A Superfolding Spinach2 Reveals the Dynamic Nature of Trinucleotide Repeat-Containing RNA,” Nat. Methods 10(12):1219-24 (2013), which is hereby incorporated by reference in its entirety).

For potassium-dependence assay, separate buffers were prepared for K⁺, Na⁺ and Li⁺. HEPES solution was neutralized with KOH, NaOH or LiOH to generate a 1 M solution (pH 7.4). RNA (1 μM final) was diluted in a buffer containing 40 mM HEPES (pH 7.4) and 0.5 mM MgCl₂. Then the salts (KCl, NaCl or LiCl) were added to a final concentration of 100 mM to the corresponding solutions. Finally, DFHBI-1T or DFHO was added to the RNA solution at a final concentration of 10 μM and incubated for 20 minutes. The fluorescence was measured on a FluoroMax-4 fluorimeter.

To measure magnesium dependence, 1 μM RNA was incubated with 10 μM DFHO in 40 mM HEPES-KOH (pH 7.4), 100 mM KCl buffer with different concentrations of MgCl₂ for 20 minutes and then fluorescence emission was measured on a on a FluoroMax-4 fluorimeter.

To measure the thermostability RNA-fluorophore complexes, 1 μM of RNA was incubated with 10 μM fluorophore. Then the fluorescence values were recorded in 3° C. increments from 19° C. to 61° C., with 5-minute incubation at each temperature to allow for equilibration.

All quantum yields were determined by comparing each RNA-fluorophore complex with Broccli-DFHBI-1T or Corn-DFHO. All measurements for quantum yield were taken in the presence of excess RNA (10 μM final) compared to the fluorophores (0.2 μM final) to avoid interference from unbound fluorophore. The integral of the emission spectra for each Squash-fluorophore complex was compared with the corresponding integrals for Broccoli or Corn to calculate the quantum yield.

### Affinity Measurements

Dissociation constants (K_{d}) for the RNA-fluorophore complexes were determined as described previously (Paige et al., “RNA Mimics of Green Fluorescent Protein,” Science 333:642-646 (2011) and Filonov et al., “Broccoli: Rapid Selection of an RNA Mimic of Green Fluorescent Protein by Fluorescence-Based Selection and Directed Evolution,” J. Am. Chem. Soc. 136: 16299-16308 (2014), which are hereby incorporated by reference in their entirety). In brief, the RNA aptamer at a fixed concentration (50 nM) was titrated with increasing fluorophore concentration and the resulting increase in fluorescence was recorded. For each fluorophore concentration, a background signal for fluorophore only solution was also measured separately and subtracted from the measured RNA-fluorophore signal. Data was fitted to a single site saturation model using nonlinear regression analysis in Sigmaplot software.

### In Vitro Folding of RNAs

Folding measurements were performed essentially as described before (Strack et al., “A Superfolding Spinach2 Reveals the Dynamic Nature of Trinucleotide Repeat-Containing RNA,” Nat. Methods 10(12):1219-24 (2013), which is hereby incorporated by reference in its entirety). Briefly, the fluorescence intensities of two solutions were compared: one having excess fluorophore and limiting RNA, and the other with excess RNA and limiting fluorophore. This allows calculation of the percent of the aptamer that is folded. For these experiments, 0.2 μM of fluorophore (or RNA) and 10 μM of RNA (or fluorophore) was used. The signal from the limiting RNA condition was divided by the signal from the limiting fluorophore condition to determine the fraction folded.

### Measurement of On and Off Rates for Squash-DFHO Pair

To measure on and off rates of Squash binding to DFHO, the same protocol that was reported previously to measure kinetic rates for Spinach-DFHBI was used (Han et al., “Understanding the Photophysics of the Spinach-DFHBI RNA Aptamer-Fluorogen Complex to Improve Live-Cell RNA Imaging,” J. Am. Chem. Soc. 135:19033-19038 (2013), which is hereby incorporated by reference in its entirety). Briefly, 50 nM RNA was mixed with different amounts of DFHO and binding kinetics was monitored on the fluorimeter as fluorescence signal increases. Each fluorescence signal trace was fitted with a monoexponential curve and the observed rate constant (k_{obs}) was extracted. Then k_{obs} values were plotted as a function of total RNA and fluorophore concentration. The resulting points were fitted with a line. This linear fit allowed extraction of the binding rate constant (kₒₙ) and the unbinding rate constant (k_{off}) as the slope and intercept, respectively (FIG. 7F). The k_{off} value calculated for Squash-DFHO (0.014±0.008 s⁻¹) is very similar to that of Corn-DFHO (0.018±0.002 s⁻¹). However, the association rate constant (kₒₙ) for Squash-DFHO pair (162300±8100 M⁻¹ s⁻¹) is seven times higher than that of Corn-DFHO pair (23000±3000 M⁻¹ s⁻¹) which could be due to the highly folded nature of Squash aptamer.

### SELEX for Generating Squash-SAM Sensors

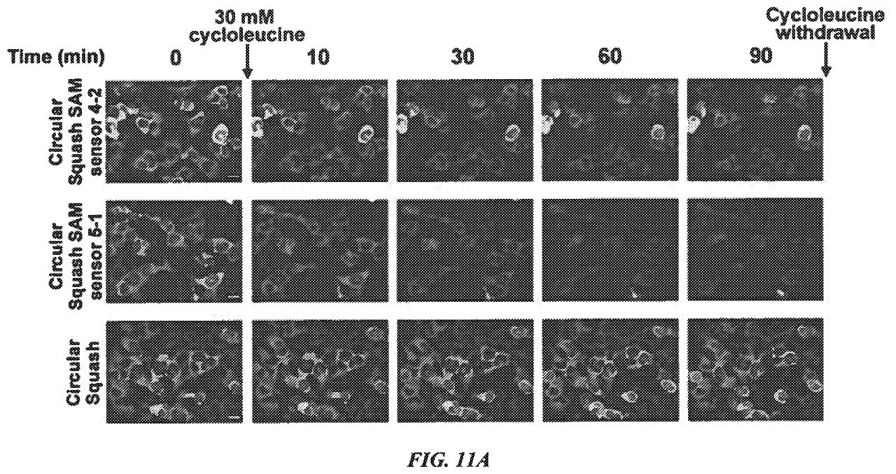

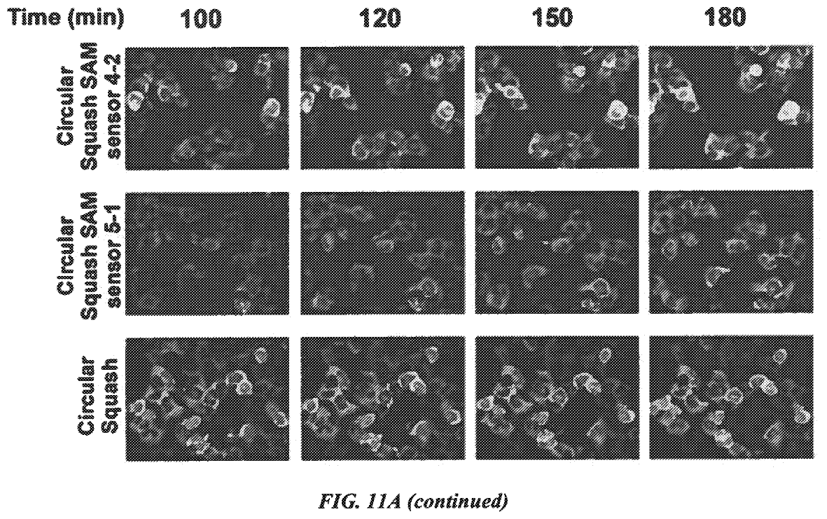

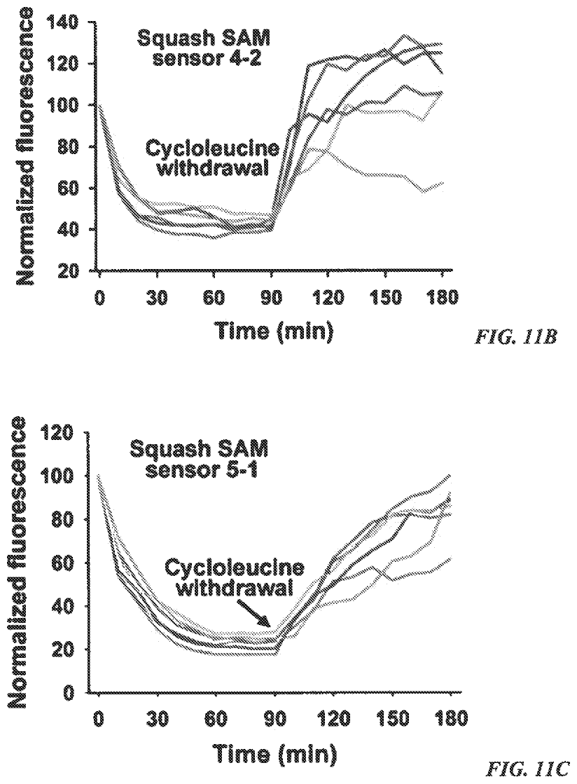

The single stranded sprouts and clips transducer DNA library (FIG. 25 (Sequences for Squash-SAM Sensor Transducer Library)) used for sensor SELEX was synthesized by the Keck Oligo Synthesis facility at Yale University. The RNA library was generated as described previously for regular SELEX. The SELEX procedure was conducted to isolate RNAs which shows conditional folding of Squash upon binding SAM (FIGS. 11A-11C). Similar methods are also described in the literature (Endoh and Sugimoto, “Selection of RNAs for Constructing ‘Lighting-UP’ Biomolecular Switches in Response to Specific Small Molecules,” PLoS One 8:e60222 (2013) and Endoh and Sugimoto, “Signaling Aptamer Optimization through Selection Using RNA-Capturing Microsphere Particles,” Anal. Chem. 92:7955-7963 (2020), which are hereby incorporated by reference in their entirety). The SELEX was performed using a single selection buffer for every round (40 mM HEPES-KOH (pH 7.4), 100 mM KCl and 0.5 mM MgCl₂). During the first step, RNA species capable of binding to the Sepharose-DFHO beads without SAM (constitutively fluorescent) were removed by incubation with the beads. Approximately 0.5 nanomole of the library RNA was diluted in 300 μL of selection buffer containing 0.1 mg/mL of yeast tRNA and incubated with pre-equilibrated Sepharose-DFHO beads (50 l) for 30 minutes with gentle agitation. After collecting the flow-through, the beads were washed with 300 μL of selection buffer containing 0.1 mg/mL of yeast tRNA and the flow-through was collected again. The combined flow-through from both the steps were mixed with SAM (0.1 mM final) and incubated for 10 minutes to induce folding of Squash. Then this solution was incubated with pre-equilibrated DFHO-functionalized beads (50 L) for 30 minutes with gentle agitation. Then the beads were washed gently with 3×300 μL of selection buffer containing 0.1 mM SAM. Finally, sensor RNA was eluted with SAM free selection buffer (2×200 μL).

The eluted RNA was treated as previously to generate the RNA pool for the next round. Selection pressure was exerted on the later rounds by more stringent washing (5×200 μL for 2^{nd} and 7×200 μL for 3^{rd} round) with SAM containing buffer. To monitor the progress of SELEX, 2 μM of the RNA pool after each round of SELEX was mixed with 10 μM of DFHO and fluorescence was measured (ex=495 nm, em=562 nm) in the absence and presence of 0.1 mM SAM. After three rounds, RNA library members were cloned using TOPO cloning and 48 colonies were picked in random from two agar plates.

dsDNA templates from the selected bacterial colonies were PCR amplified from purified plasmid and the RNA was generated by in vitro transcription. For each library member, 1 μM of the RNA was mixed with 10 μM of DFHO in the absence and presence of 0.1 mM SAM and put into separate PCR tubes. The solutions were heated at 37° C. for 10 minutes and immediately imaged using a BioRad ChemiDoc MP imager (ex: 530±14 nm, em=605±25 nm). Library members which showed substantial SAM dependent fluorescence enhancement were used for further characterization.

### In Vitro Characterization of the Sensors

For all in vitro measurements, the sensor RNAs (FIG. 25 (Sequence of the Squash-SAM sensors)) were purified using 8% denaturing PAGE (7 M urea) as described above. To test the ability of each indicated transducer sequence to mediate SAM-induced fluorescence, 1 μM of the RNA was mixed with 10 μM of DFHO in the absence or presence of 0.1 mM SAM in a buffer containing 40 mM HEPES-KOH (pH 7.4), 100 mM KCl and 0.5 mM MgCl₂. After 1 hour incubation at 37° C., fluorescence signal of each sample was measured at 37° C. using a Fluoromax-4 fluorimeter with 495 nm excitation and 562 nm emission. For both conditions, background signal (for 10 μM DFHO only) was deducted from the signal obtained for the sensor.

To measure the activation rate of the Squash-SAM sensors, a solution containing 1 μM sensor RNA and 10 μM DFHO (in buffer containing 40 mM HEPES-KOH (pH 7.4), 100 mM KCl and 0.5 mM MgCl₂) was incubated at 37° C. for 30 minutes. Then SAM (0.1 mM final) was quickly added to the stirring RNA solution, and fluorescence was recorded over 20 minutes at 1 minute intervals at 37° C. (495 nm ex; 562 nm em). The fluorescence signal was normalized to the intensity at 20 minutes (100) and intensity at 0 minutes (0).

To measure the deactivation rate of the sensors, 1 μM sensor RNA and 10 μM DFHO was incubated with 0.1 mM SAM (in buffer containing 40 mM HEPES-KOH (pH 7.4), 100 mM KCl and 0.5 mM MgCl₂) for 1 hour at 37° C. Then this solution was buffer-exchanged with the same buffer used above (without SAM) using a Micro Bio-Spin Column with Bio-Gel P-30 beads (Bio-Rad) to remove SAM. DFHO (10 μM final) was added to the collected flow-through and the fluorescence emission was recorded as with the activation experiments. The fluorescence measurement was normalized to the intensity at 0 min (100) and the intensity at 20 minutes (0).

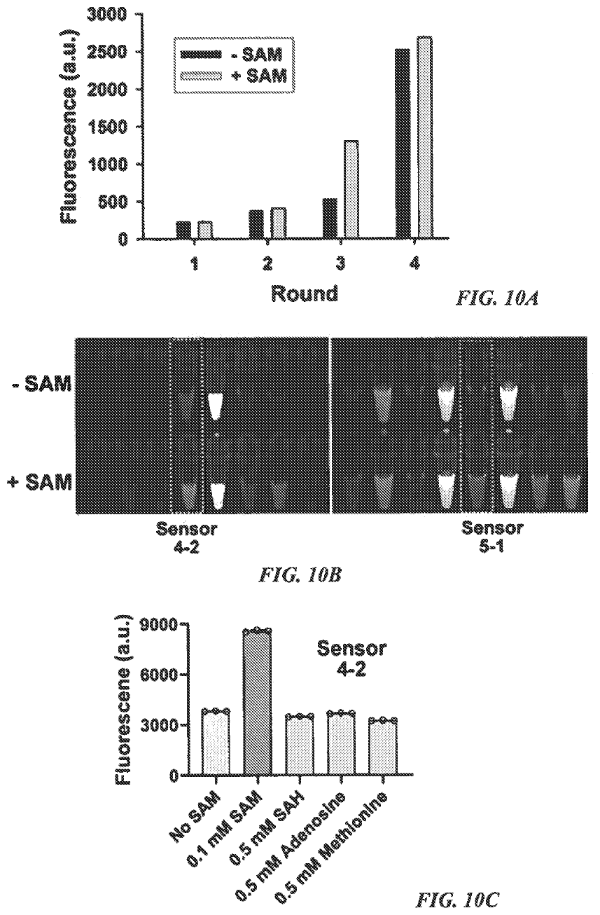

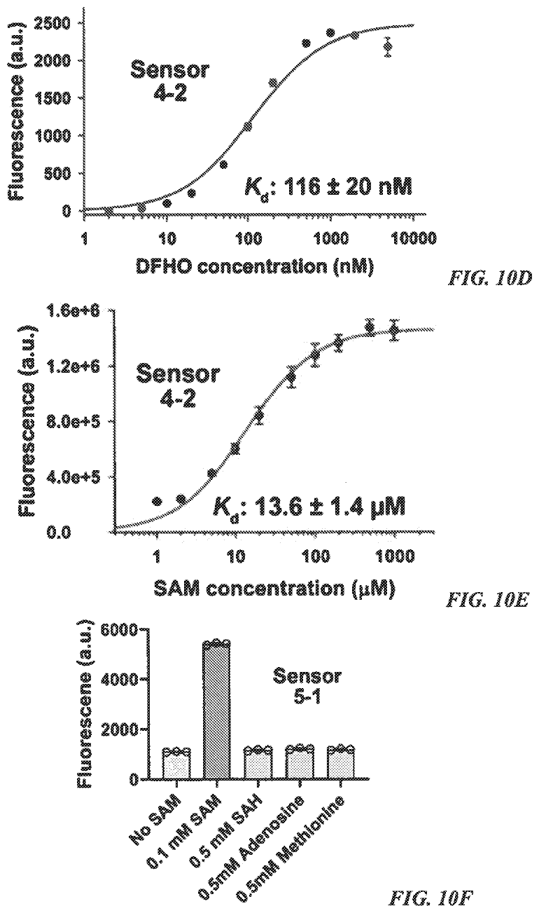

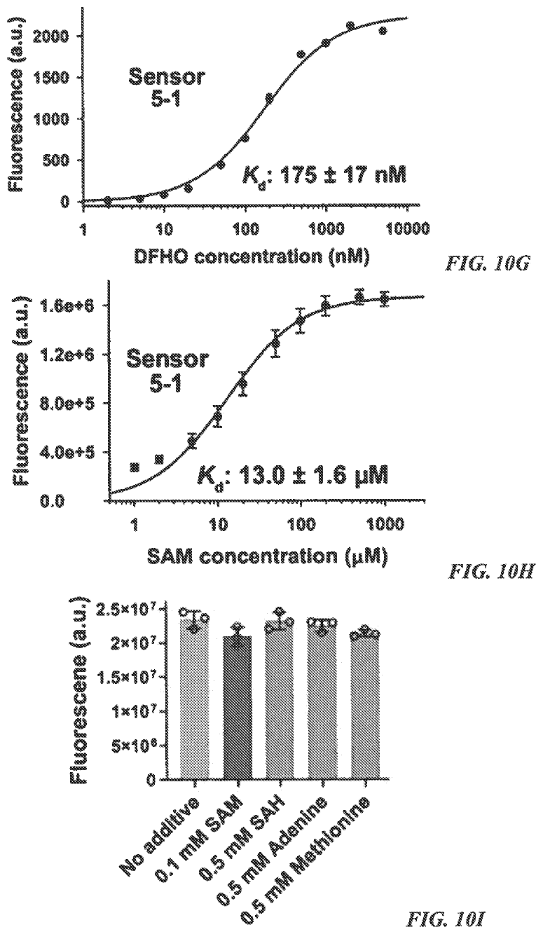

Dissociation constants (K_{d}) for the sensor complexes were determined as described previously (Kim and Jaffrey, “A Fluorogenic RNA-Based Sensor Activated by Metabolite-Induced RNA Dimerization,” Cell Chem. Biol. 26:1725-1731 (2019), which is hereby incorporated by reference in its entirety). For measurement of sensor-DFHO affinity, a solution containing the sensor RNA (100 nM final) and 0.1 mM SAM was titrated with increasing DFHO concentration and the resulting increase in fluorescence was recorded (FIG. 10D; FIG. 10G). For each DFHO concentration, a background signal for DFHO only solution was also measured separately and subtracted from the signal measured for RNA-DFHO signal. For measuring K_{d} of the sensors to SAM, a solution containing the sensor RNA (1 μM final) and DFHO (10 μM final) was titrated with increasing concentration of SAM and the resulting increase in fluorescence was recorded (FIG. 10E; FIG. 10H). The fluorescence was measured at 37° C. using Spectramax iD3 plate reader with 495 nm excitation and 562 nm emission. For each concentration of SAM measured, a background signal for no SAM sample was also measured separately and subtracted from the signal measured for RNA, DFHO and SAM together. In both cases, data was fitted to a single site saturation model using nonlinear regression analysis in Sigmaplot software.

### Cloning of the Plasmids for Mammalian Expression

U6 constructs were expressed from pAV-U6+27 plasmid, which expresses the 27 nt-leader sequence of the U6 small nucleolar RNA from the U6 promoter (Endoh and Sugimoto, “Signaling Aptamer Optimization through Selection Using RNA-Capturing Microsphere Particles,” Anal. Chem. 92:7955-7963 (2020), which is hereby incorporated by reference in its entirety). 5S constructs were expressed from pAV-5S plasmid, which expresses full length human 5S rRNA from its endogenous promoter. Different RNA constructs were fused to the 3′ end of either pAV-U6+27 or pAV-5S. The sequence encoding the constructs were amplified by PCR and then digested with XbaI and SalI and inserted into pAV-U6+27 or pAV-5S plasmids (digested with the same restriction enzymes) using T4 DNA ligase.

In the case of preparing pAV-Tornado aptamers and sensors, dsDNA templates containing the appropriate sequences were prepared with flanking NotI and SacII restriction sites. Then the appropriate sequences were inserted in a clonable Tornado expression cassette on a pAV-U6+27 vector through cloning using NotI and SacII restriction sites (Litke and Jaffrey, “Highly Efficient Expression of Circular RNA Aptamers in Cells Using Autocatalytic Transcripts,” Nat. Biotechnol. 37:667-675 (2019), which is hereby incorporated by reference in its entirety). For the ratiometric sensors, the Squash-SAM sensor was inserted into one arm of an F30 scaffold and Broccoli aptamer was inserted into the other arm (FIG. 25 (Sequences of Circular Ratiometric SAM Sensors)). dsDNA sequence for the ratiometric sensors flanked by NotI and SacII restriction sites were generated using PCR and cloned into the pAV-Tornado plasmid as described above.

### Cell Culture Conditions

All cell lines except mES cells were obtained directly from the American Type Culture Collection (ATCC). HEK293T (ATCC CRL-3216) were cultured in DMEM media (Life Technologies, no. 11995-065) with 10% FBS, 100 U/mL penicillin and 100 μg/mL of streptomycin under standard tissue culture conditions (at 37° C. and 5% CO₂). HCT116 cell s (ATCC CCL-247) were cultured RPMI 1640 media (Life Technologies, no. 11875-093) with 10% FBS, 100 U/mL penicillin and 100 μg/mL of streptomycin under standard tissue culture conditions. Cells were screened for mycoplasma contamination before passaging using Universal Mycoplasma Detection Kit (ATCC 30-1012K) according to ATCC recommendations.

Mouse embryonic stem cells (mES cells) were previously generated from C57BL/6×12954/SvJae F1 male embryos (Paul et al., “Localized Expression of Small RNA Inhibitors in Human Cells,” Mol. Ther. 7:237-247 (2003), which is hereby incorporated by reference in its entirety). After thawing the cells, they were cultured in gelatin-coated plates using proprietary Knockout DMEM formulation supplemented with 15% heat-inactivated FBS (Corning, 35-010-CV), lx GlutaMAX (Life Technologies, no. 35050-061), 1×Non-essential amino acids (Life Technologies no. 11140-050), β-ME (55 μM final), and 1000 units/mL of LIF (Millipore Sigma no. ESG1107). Because Knockout DMEM media is proprietary, it is not possible to generate customized media lacking certain ingredients. Therefore, after culturing the mES cells in Knockout DMEM for two passages, the mES cells were cultured in an experimental media (Paul et al., “Localized Expression of Small RNA Inhibitors in Human Cells,” Mol. Ther. 7:237-247 (2003), which is hereby incorporated by reference in its entirety) containing a 1:1 mix of glutamine-free DMEM (Life Technologies no. 11960-069) and glutamine-free Neurobasal medium (Life Technologies no. 21103-049) supplemented with 10% heat-inactivated FBS (Corning, 35-010-CV), lx GlutaMAX (Life Technologies, no. 35050-061), β-ME (55 μM final), and 1000 units/mL of LIF (Millipore Sigma no. ESG1107). This media described here is designated −2i media. To generate the +2i media, the −2i media was supplemented with a MEK inhibitor (PD0325901) and a GSK30 inhibitor (CHIR99021) with final concentration being 1 μM and 3 μM respectively. To dissociate the mES cells from plates during passages StemPro Accutase (Life Technologies, no. A1110501) was used.

### Media Preparation for Imaging

For imaging experiments media containing no phenol red were used to reduce background fluorescence. For imaging HEK293T cells, Flurobrite DMEM (Life Technologies, no. A1896701) supplemented with 10% FBS, lx GlutaMAX, 100 U/mL penicillin, and 100 mg/mL streptomycin was used. For imaging in HCT116 cells, RPMI 1640 media lacking amino acids, glucose, and glutamine (MyBioSource, no. MBS653421) was used as starting point (Palmer et al., “Design and Application of Genetically Encoded Biosensors,” Trends Biotechnol. 29:144-152 (2011), which is hereby incorporated by reference in its entirety). This media was supplemented with 1× Minimal Essential Media Non-essential Amino Acids (MEM NEAA), 5 mM glucose, 1× GlutaMAX, 10% dialyzed FBS (Gemini, no. 100-108), 100 U/mL penicillin, and 100 μg/mL streptomycin. Essential amino acids, except methionine and threonine, were added back at the same concentrations found in MEM amino acids solution to generate the methionine and threonine free media. Methionine (100 μM final) and threonine (400 μM final) were added to this media as required for the amino acid withdrawal experiments.

For mES cells, a custom version of the DMEM and Neurobasal media each lacking methionine, threonine and phenol red were prepared by the Media Preparation core at Sloan Kettering Institute. The −2i imaging media (without methionine and threonine) was prepared mixing these two media in 1:1 ratio and supplemented with 10% dialyzed FBS (Gemini, no. 100-108), 1× GlutaMAX (Life Technologies, no. 35050-061), β-ME (55 μM final), and 1000 units/mL of LIF (Millipore Sigma no. ESG1107). Methionine (200 μM final) and threonine (800 μM final) were added to this media as required for the amino acid withdrawal experiments. To generate the +2i imaging media (without methionine and threonine), the −2i imaging media was supplemented with the MEK inhibitor (PD0325901) and the GSK30 inhibitor (CHIR99021) with final concentration being 1 μM and 3 μM respectively. Again methionine (200 μM final) and threonine (800 μM final) were added to this media as required for the amino acid depletion experiments.

### Flow Cytometry of Mammalian Cell

HEK293T cells were plated in 12-well plates and transfected with the appropriate RNA expressing plasmid next day using FuGENE HD (Promega) following manufacturer's recommendation. After 36 h, the cells were washed with 1×PBS once, dissociated from the plate using TrypLE Express Enzyme (Life Technologies, no. 12604013), re-suspended in 4% FBS/1×PBS solution containing 10 μM DFHBI-1T or 5 μM DFHO as required, and kept on ice until analysis on the BD LSRFortessa instrument. Transfected cells were analyzed in green channels (ex=488 nm, em=525±25 nm) for DFHBI-1T and orange channel (ex=488 nm, em=570±20 nm) for DFHO. For Corn-DFHO, a yellow channel (ex 488 nm, em 545±17.5 nm) was used due to blue shifted emission of Corn. An auxiliary far-red channel (ex=635 nm, em=780±30 nm) was used to measures cellular auto-fluorescence. Processing and analysis of the data was performed in the FlowJo software.

### Imaging Aptamer-Tagged RNAs and SAM Sensors in HEK293T Cells

Cell imaging for HEK293T cell was carried out as described previously (Song et al., “Imaging RNA polymerase III transcription using a Photostable RNA—Fluorophore Complex,” Nat. Chem. Biol. 13:1187-1194 (2017), which is hereby incorporated by reference in its entirety). HEK293T cells were plated on poly-D-lysine and mouse laminin-coated glass-bottom 24-well plates (MatTek). The next day, cells were transfected with the appropriate plasmid using FuGENE HD reagent (Promega). After 36 hours, cells were washed with 1×PBS once and incubated with the imaging media described above containing appropriate fluorophore. After 30 minute incubation in the incubator, Hoechst 33342 was added to a final concentration of 0.1 μg/mL.

The 24-well plate was transferred to a Tokai Hit stage top incubator (37° C. and 5% CO2) and live fluorescence images were taken with a Nikon Eclipse Ti-E microscope fitted with a CoolSnap HQ2 CCD camera (Photometrics) through a 40× air objective (NA 0.75) and analyzed with the ImageJ software to detect Broccoli (470±20 nm ex; 495 nm dichroic; 525±25 nm em), Corn (500±12 nm ex; 520 nm dichroic; 542±13.5 nm em), and Squash (500±12 nm ex; 520 nm dichroic; 570±20 nm em), and Hoechst (350±25 nm ex; 400 nm dichroic; 460±25 nm em).

### Ratiometric Imaging of SAM in Mammalian Cells

For experiments in HEK293T cells, cell culture and transfection was performed as described above. HCT116 cells were plated on 24-well ibiTreat μ-Plate (ibidi GmbH, Germany) coated with poly-D-lysine and mouse laminin. For mES cells, cells were plated on 24-well ibiTreat μ-Plate coated with gelatin. For both of these cell lines, plasmids encoding RNA sensors were transfected using Lipofectamine Stem transfection reagent (Life Technologies, no. A1896701). Approximately 36 hours after transfection, cells were washed with 1×PBS once and then incubated in the appropriate imaging media described above supplemented with required fluorophore(s). For imaging with the ratiometric sensors, 10 μM DFHO and 5 μM BI were used.

After placing the plates on a Tokai Hit stage top incubator (37° C. and 5% CO2), live fluorescence images were taken with a Nikon Eclipse Ti-E microscope fitted with a CoolSnap HQ2 CCD camera (Photometrics) through a 40× air objective (NA 0.75). For live cell imaging over long time period, the perfect focus system (PFS) was used to avoid focal drifts. For Squash-SAM sensors a modified YFP filter cube (500±12 nm ex; 520 nm dichroic mirror; 570±20 nm em) was used. For the ratiometric sensors custom designed filter cubes were used to avoid bleed through of one channel into the other. For the green channel (Broccoli-BI), a filter cube with 460±7 nm excitation filter, 473 nm dichroic mirror, and 500±12 nm emission filter was used. For the orange channel (Squash-DFHO), a filter cube with 512±12.5 nm excitation filter, 532 nm dichroic mirror, and 575±29.5 nm emission filter was used. Hoechst-stained nuclei were imaged as described above. For ratiometric SAM imaging, 500 ms exposure time was used for the orange channel and 100 ms exposure time for the green channel.

### Image Analysis for Generating SAM Trajectories

Image analysis was performed in ImageJ (FIJI) ver. 1.53c. For the single-color SAM sensors, images analysis was performed as before (Kim and Jaffrey, “A Fluorogenic RNA-Based Sensor Activated by Metabolite-Induced RNA Dimerization,” Cell Chem. Biol. 26:1725-1731 (2019), which is hereby incorporated by reference in its entirety). Briefly, images obtained at each time points were background subtracted and the mean intensity of each cell at time 0 was defined as 100. The mean fluorescence intensity at any other time point is normalized to the value at time 0 and normalized mean fluorescence of each cell was plotted as a function of time.

For the ratiometric sensor, it is important to generate the orange to green (O/G) fluorescence ratio only for areas containing the cells. For each time points, images from both channels were background subtracted. Then the constitutively fluorescent green channel was used to create a binary mask to identify the cells. This mask was multiplied with the background subtracted images from each channel to generate masked images for each channel. Finally, images containing O/G fluorescence ratio was generated by dividing the masked orange channel image with masked green channel image. The ratio in the image is coded by pseudocolor.

### Quantification of SAM Using Biochemical Assay

SAM concentration in HEK293T cells were measured as described previously (Moon et al., “Naturally Occurring Three-Way Junctions can be Repurposed as Genetically Encoded RNA-Based Sensors,” Cell Chem. Biol. 28:1-12 (2021), which is hereby incorporated by reference in its entirety) using a commercially available kit (Bridge-It S-adenosylmethionine (SAM) fluorescence assay kit, Mediomics, USA). For cycloleucine treatment experiments, SAM was extracted from HEK293T cells at each time points using 80% methanol as described previously (Moon et al., “Naturally Occurring Three-Way Junctions can be Repurposed as Genetically Encoded RNA-Based Sensors,” Cell Chem. Biol. 28:1-12 (2021) and Mentch et al., “Histone Methylation Dynamics and Gene Regulation Occur through the Sensing of One-Carbon Metabolism,” Cell Metab. 22:861-873 (2015), which are hereby incorporated by reference in their entirety). After evaporating the liquid, the cell extract was dissolved in 25 μL of water. The SAM concentration was then determined according to the manufacturer's protocol. Briefly, a standard curve was generated using standard solutions of SAM supplied with the kit. Then the concentration of SAM in the cellular extract was determined at each time point using the standard curve. Finally, the cellular concentration of SAM was determined with the assumption that cells are spherical with approximate diameter of 17 μm giving us a specific volume for each cell.

### Statistics and Reproducibility

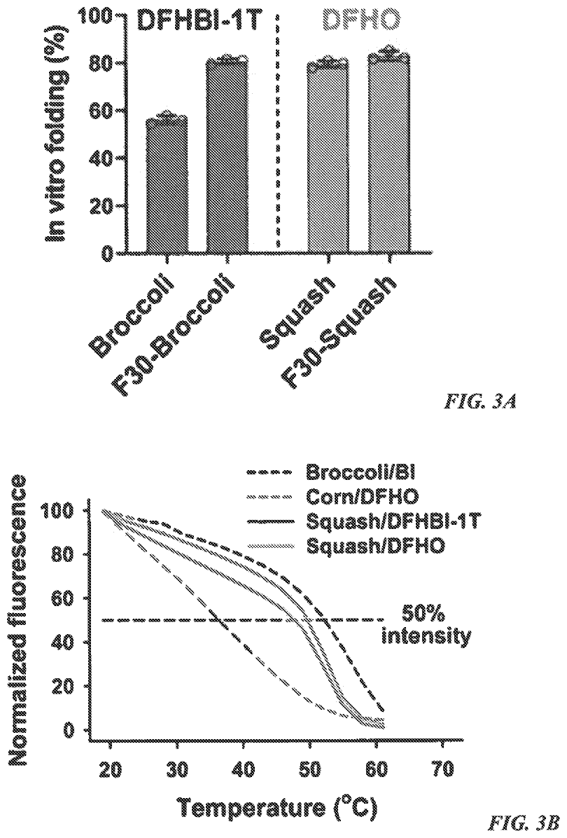

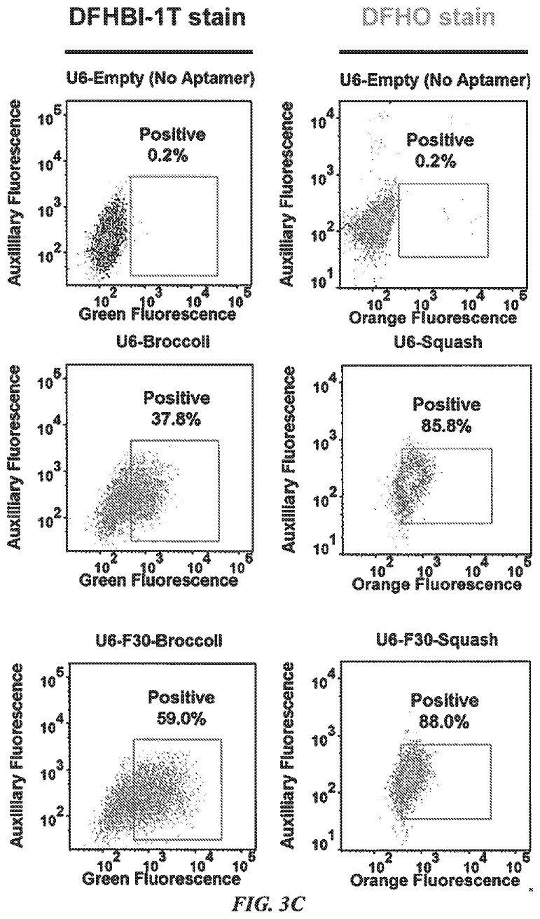

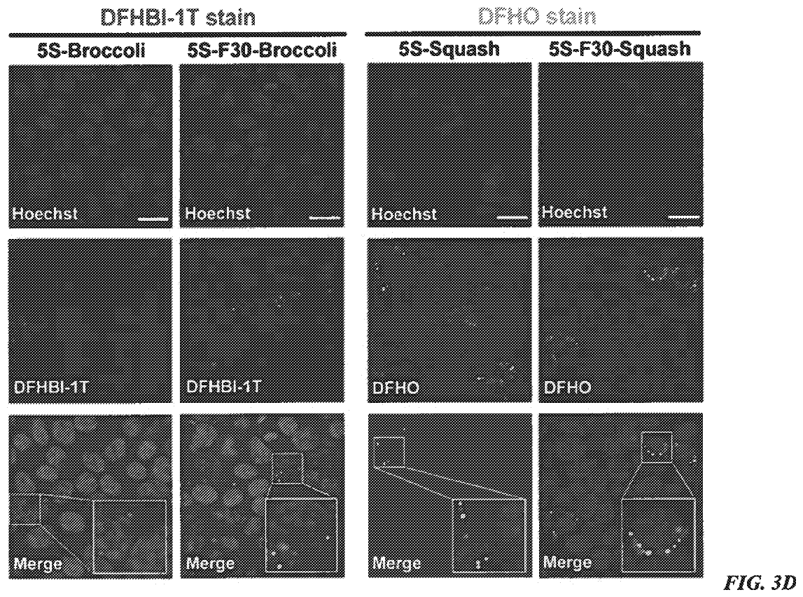

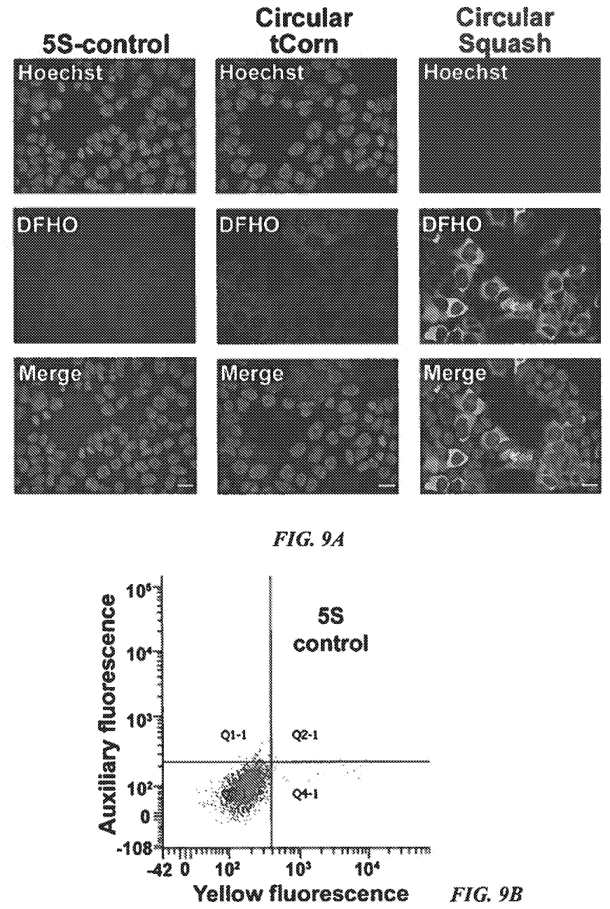

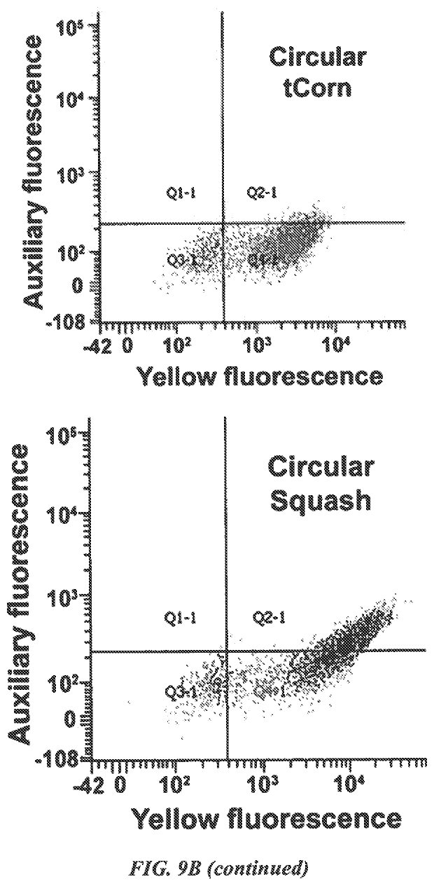

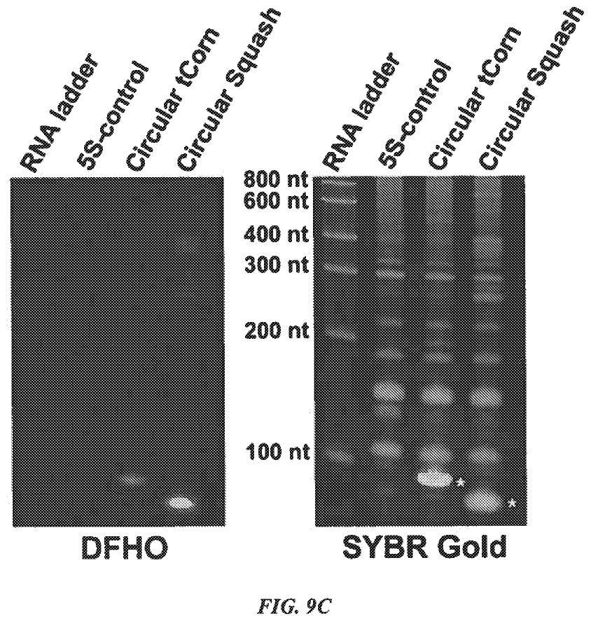

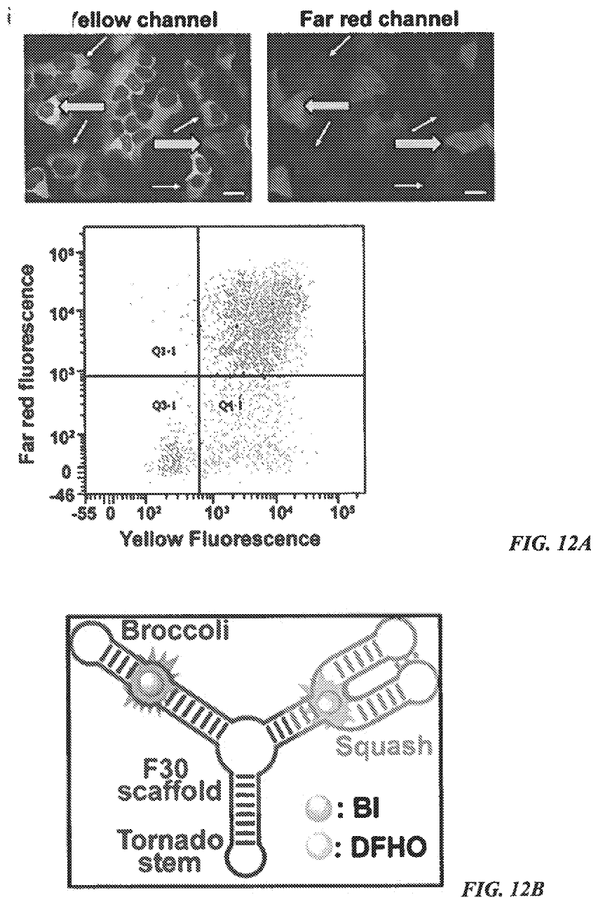

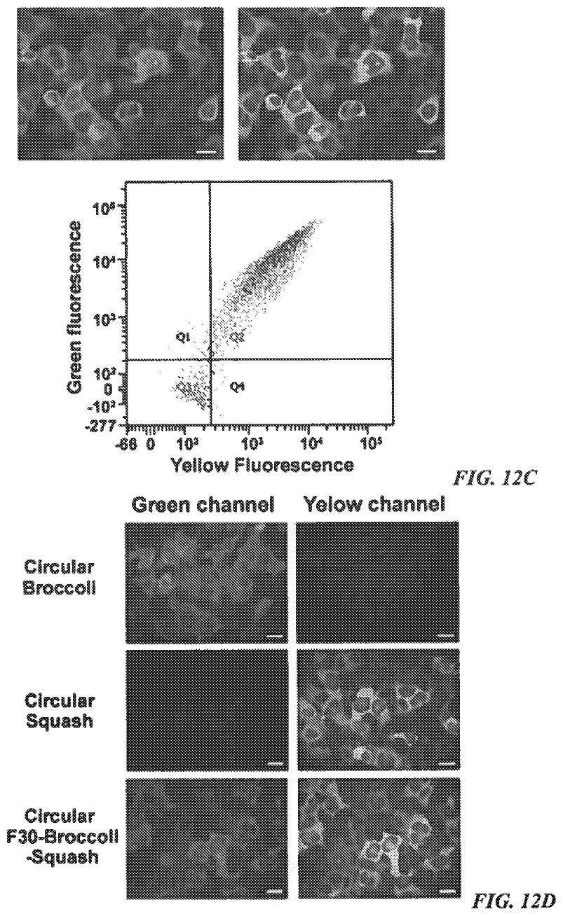

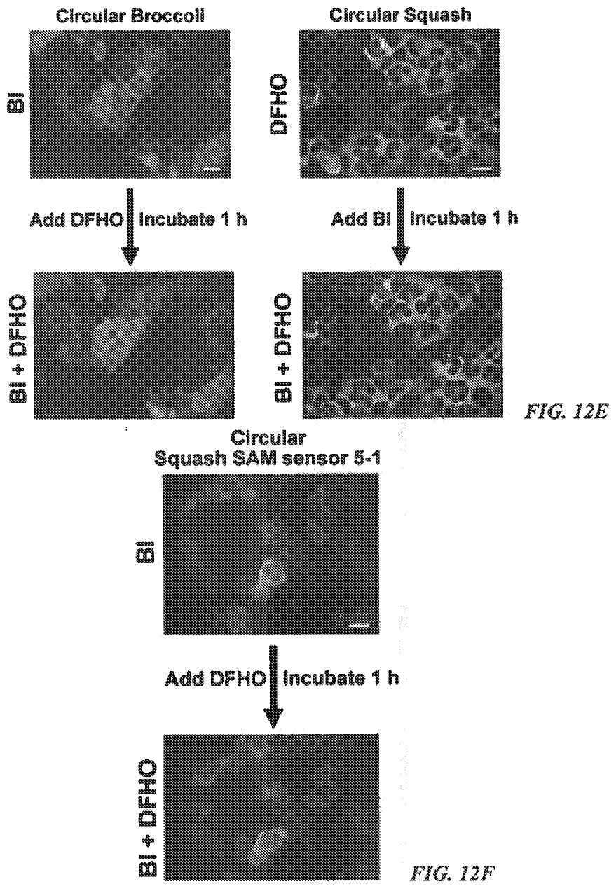

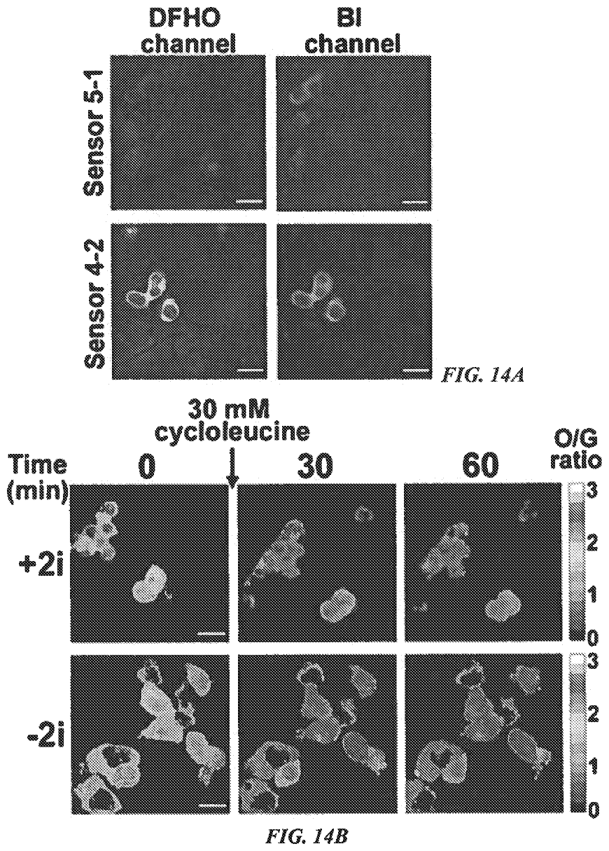

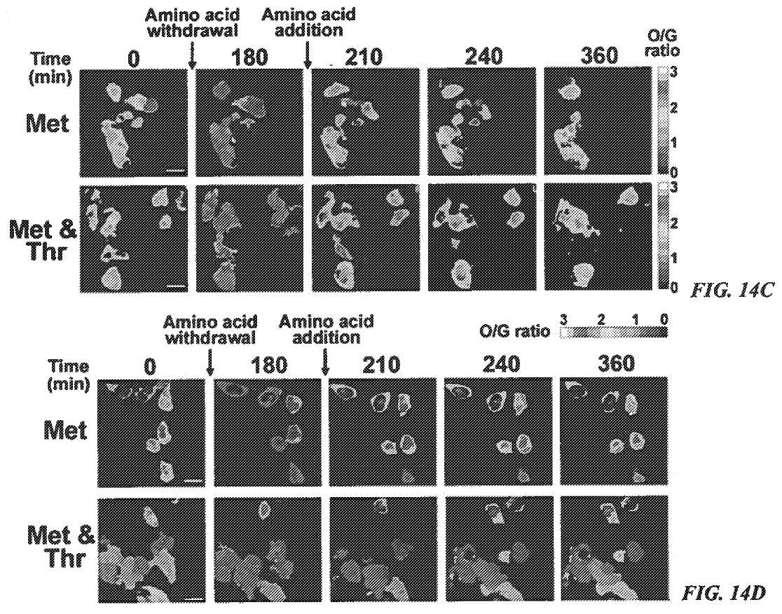

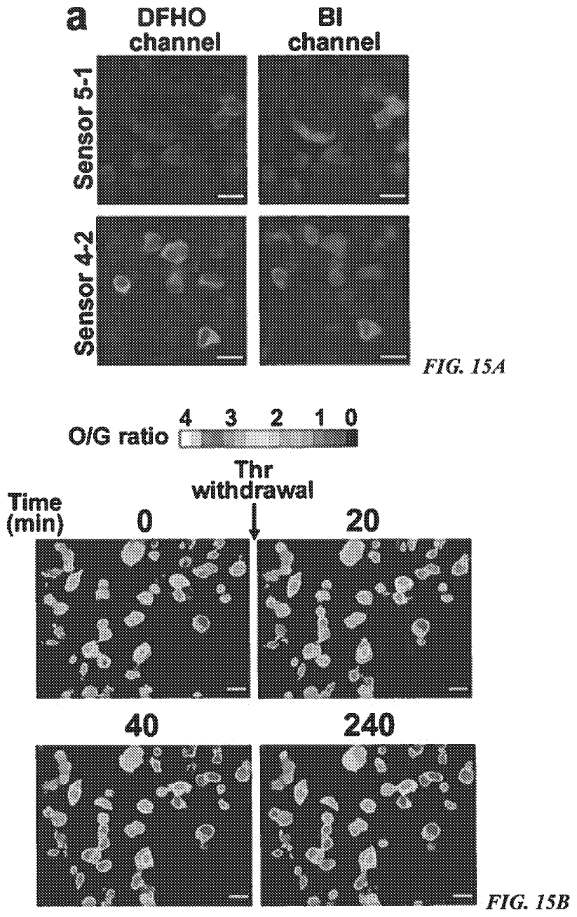

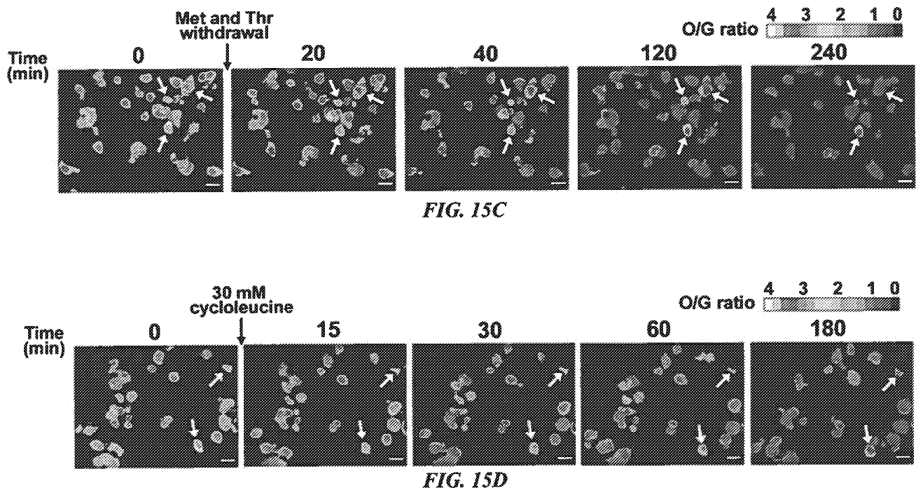

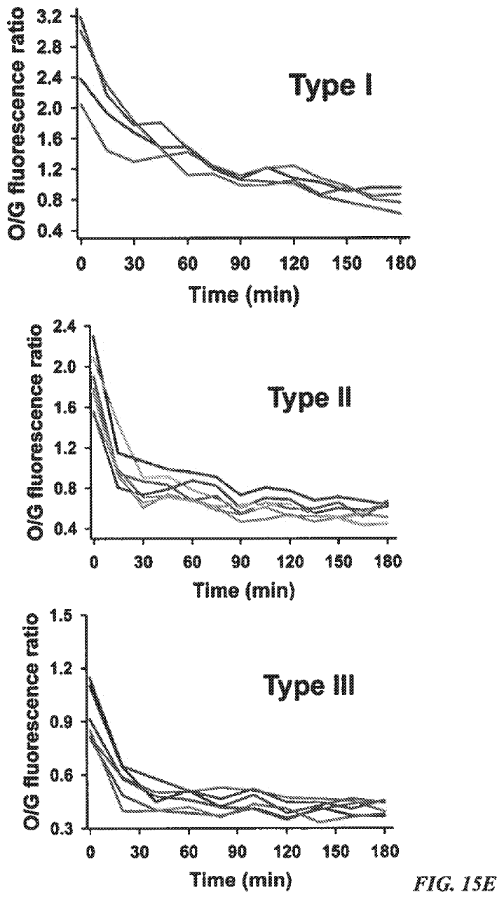

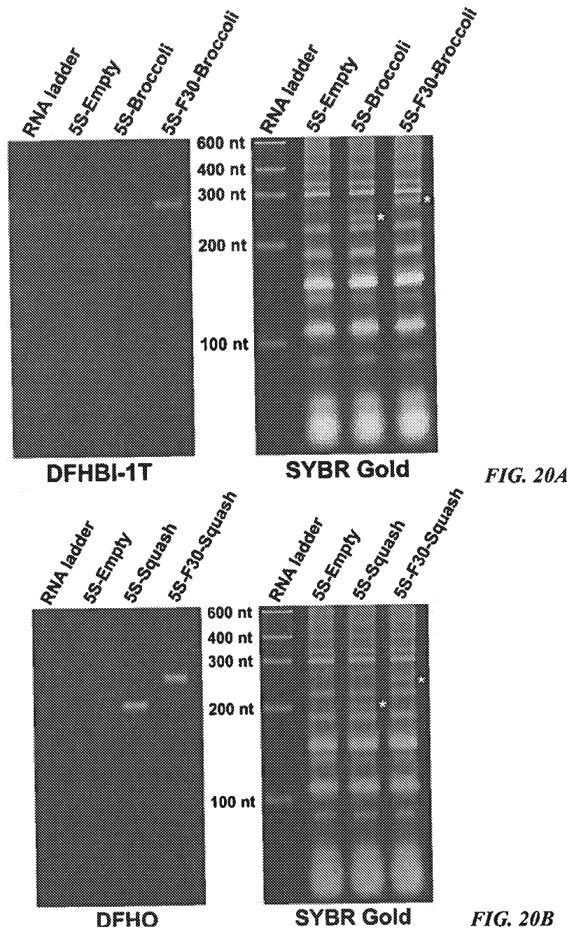

All data are expressed as means±s.d. with the number of independent experiments (n) listed for each experiment. Statistical analyses were performed using Excel (Microsoft) and Prism (GraphPad). Experiments which show micrographs were repeated independently at least thrice and showed similar results. This applies to FIG. 3C; FIG. 4G; FIG. 9A; FIG. 9C; FIGS. 12D-12F; FIGS. 14A-14D; FIGS. 15A-15D; FIGS. 20A-20B).

### Data Availability

The following plasmids generated in this study are available through Addgene: pAV-U6+27-Squash (ID 177913), pAV-5S-Squash (ID 177914), pAV-U6+27-Tornado-Squash (ID 177915), pAV-U6+27-Tornado-Squash-SAM-sensor 4-2 (ID 177916), pAV-U6+27-Tornado-Squash-SAM-sensor 5-1 (ID 177917), pAV-U6+27-Tornado-F30-Squash-Broccoli (ID 177918), pAV-U6+27-Tornado-F30-Broccoli-Squash-SAM-sensor 4-2 (ID 177919), pAV-U6±27-Tornado-F30-Broccoli-Squash-SAM-sensor 5-1 (ID 177920).

### Code Availability

The custom code used to analyze the “sprouts and clips” library is deposited in Bitbucket.

### Example 1—Design of an Expanding and Contracting RNA Library for SELEX

RNA aptamers that are selected in vitro often exhibit poor folding, unlike some naturally occurring riboswitch aptamers which can fold before mRNA transcription has been completed (Frieda and Block, “Direct Observation of Cotranscriptional Folding in an Adenine Riboswitch,” Science 338:397-401 (2012), which is hereby incorporated by reference in its entirety). It was reasoned that the ligand-binding domain of a naturally occurring high-folding riboswitch could be evolved to bind and activate a fluorogenic dye. The adenine-binding aptamer in the add A-riboswitch from V. vulnificus (Mandal and Breaker, “Adenine Riboswitches and Gene Activation by Disruption of a Transcription Terminator,” Nat. Struct. Mol. Biol. 11:29-35 (2004) and Serganov et al., “Structural Basis for Discriminative Regulation of Gene Expression by Adenine- and Guanine-Sensing mRNAs,” Chem. Biol. 11:1729-1741 (2004), which are hereby incorporated by reference in their entirety) (FIG. 1A, FIG. 1B), which folds in ˜2 seconds (Dalgarno et al., “Single-Molecule Chemical Denaturation of Riboswitches,” Nucleic Acids Res. 41:4253-4265 (2013), which is hereby incorporated by reference in its entirety), was chosen. This aptamer is unusual in that it folds in magnesium concentrations as low as 100 μM (Lemay et al., “Folding of the Adenine Riboswitch,” Chem. Biol. 13:857-868 (2006), which is hereby incorporated by reference in its entirety). This may be useful since the mammalian cytoplasm typically contains lower magnesium levels (Grubbs, R., “Intracellular Magnesium and Magnesium Buffering,” BioMetals 15:251-259 (2002) and Romani, A., “Magnesium Homeostasis in Mammalian Cells,” in Metallomics and the Cell (ed. Banci L) 69-118 (Springer, 2013), which are hereby incorporated by reference in their entirety) (0.2-1.0 mM) than the bacterial cytoplasm, which can be in the millimolar range (Tyrrell et al., “The Cellular Environment Stabilizes Adenine Riboswitch RNA Structure,” Biochemistry 52:8777-8785 (2013), which is hereby incorporated by reference in its entirety).

GFP-like fluorophores are useful for fluorogenic aptamers since they exhibit low cytotoxicity, high cell permeability, minimal fluorescence when incubated in cells, and can be fluorescently activated by RNA aptamers (Paige et al, “RNA Mimics of Green Fluorescent Protein,” Science 333(6042): 642-646 (2011); Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014); Song et al., “Plug-and-Play Fluorophores Extend the Spectral Properties of Spinach,” J. Am. Chem. Soc. 136:1198-1201 (2014); Song et al., “Imaging RNA polymerase III transcription using a Photostable RNA—Fluorophore Complex,” Nat. Chem. Biol. 13:1187-1194 (2017); and Steinmetzger et al., “A Multicolor Large Stokes Shift Fluorogen-Activating RNA Aptamer with Cationic Chromophores,” Chem. Eur. J. 25:1931-1935 (2019), which are hereby incorporated by reference in their entirety). It was reasoned that the ligand-binding domain of the adenine aptamer could be evolved to bind and activate these dyes. However, the ligand-binding pocket may be too small to accommodate GFP-like fluorophores such as DFHBI-1T ((Z)-4-(3,5-difluoro-4-hydroxybenzylidene)-2-methyl-1-(2,2,2-trifluoroethyl)-1H-imidazol-5(4H)-one) and DFHO ((Z)-4-(3,5-difluoro-4-hydroxybenzylidene)-1-methyl-5-oxo-1H-imidazole-2-carbaldehyde oxime) (FIG. 1A). The nucleotides that comprise the ligand-binding pocket of an aptamer can be randomized to create RNA libraries that can be used in SELEX (Tuerk and Gold, “Systematic Evolution of Ligands by Exponential Enrichment: RNA Ligands to Bacteriophage T4 DNA Polymerase,” Science 249:505-510 (1990) and Ellington and Szostak, “In vitro Selection of RNA Molecules that Bind Specific Ligands,” Nature 346:818-822 (1990), which are hereby incorporated by reference in their entirety) to discover new ligand-binding aptamers. These types of libraries were previously generated by either randomizing few residues (Dixon et al., “Reengineering Orthogonally Selective Riboswitches,” Proc. Natl. Acad. Sci. U.S.A. 107:2830-2835 (2010), which is hereby incorporated by reference in its entirety) or the entire binding pocket (Porter et al., “Recurrent RNA Motifs as Scaffolds for Genetically Encodable Small-Molecule Biosensors,” Nat. Chem. Biol. 13:295-301 (2017), which is hereby incorporated by reference in its entirety) in purine riboswitches. However, these libraries only change the sequence, not the size, of the ligand-binding pocket.

To develop a SELEX library in which the ligand-binding pocket varies in both sequence and size, the DNA synthesis protocol used to make random libraries was modified (Hall et al., “Design, Synthesis, and Amplification of DNA Pools for In Vitro Selection,” Curr. Protoc. Mol. Biol. Chapter 24: Unit 24.2 (2009), which is hereby incorporated by reference in its entirety). In conventional oligonucleotide library synthesis, a phosphoramidite mixture representing all four nucleotides is prepared (Hall et al., “Design, Synthesis, and Amplification of DNA Pools for In Vitro Selection,” Curr. Protoc. Mol. Biol. Chapter 24: Unit 24.2 (2009), which is hereby incorporated by reference in its entirety). This mixture is used for site-specific incorporation of a random nucleotide at specified positions in each growing oligonucleotide strand (Hall et al., “Design, Synthesis, and Amplification of DNA Pools for In Vitro Selection,” Curr. Protoc. Mol. Biol. Chapter 24: Unit 24.2 (2009), which is hereby incorporated by reference in its entirety). To induce spontaneous shortening of the SELEX library, the coupling time was reduced from 25 seconds to 2 seconds. This reduces the coupling efficiency from ˜100% to ˜93% (Table 4). As a result, approximately 7% of the strands lose a nucleotide every time this mixture is used. The loss of any nucleotide relative to the full-length sequence was designated as a “clip”.

TABLE 4
Analysis of the Deletion (Clip) Libraries

| | | Number of deletions, |
|---|---|---|
| Coupling | Sequence length | nt (probability |
| time (s) | analyzed, nt | of deletion, %) |
| 25 | 648 | 4 (0.6) |
| 10 | 648 | 19 (2.9) |
| 5 | 648 | 28 (4.3) |
| 2 | 312 | 21 (6.7) |

To create random additions of nucleotides, a new mixture containing all four phosphoramidites was created, but at 1% the normal concentration. This dilute mixture exhibits an approximately 5% coupling efficiency (Table 5), thus causing stochastic insertions at specific positions in the oligonucleotide. The appearance of a stochastically added nucleotide was designated as a “sprout” in the library.

TABLE 5
Analysis of the Insertion (Sprout) Libraries

| Concentration of | | Number of insertions, |
|---|---|---|
| phosphoramidites, % | Sequence length | nt (probability |
| from standard | analyzed, nt | of insertion, %) |
| 1 | 600 | 31 (5.2) |
| 0.1 | 600 | 5 (0.8) |

Importantly, the “capping step” during oligonucleotide synthesis was omitted. The capping step comprises 5′-OH acetylation of any oligonucleotide that fails to couple to a phosphoramidite (Bartel et al., “HIV-1 Rev Regulation Involves Recognition of Non-Watson-Crick Base Pairs in Viral RNA,” Cell 67:529-536 (1991), which is hereby incorporated by reference in its entirety). Removing this step is important since clips rely on stochastically inefficient coupling. Similarly, removing the capping step is important for sprouts, because coupling reactions typically do not occur when using the dilute phosphoramidite mixture for sprouts. Thus, removing the capping step is required for sprouts and clips.

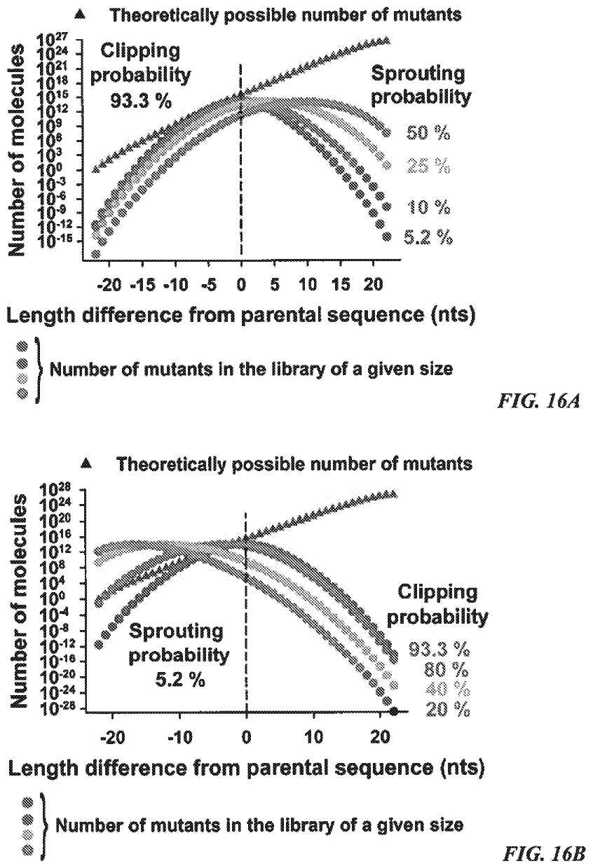

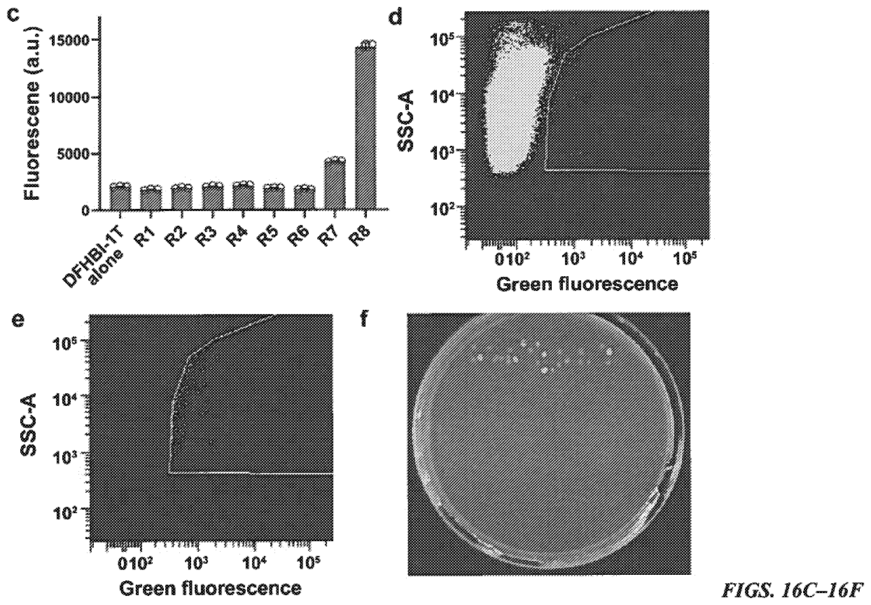

The adenine aptamer ligand-binding pocket comprises three strands at the center of a three-way junction (FIG. 1A, FIG. 1). Therefore, a sprouts and clips library in which the ligand-binding pocket was randomized for both sequence and size was created (FIG. 1A, FIG. 1i). Every nucleotide in the ligand-binding pocket was randomized sequentially using the standard phosphoramidite mixture with reduced coupling time, followed by the dilute phosphoramidite mixture (FIG. 1A). Thus, a 6 nt-long strand in the ligand-binding pocket can be transformed into as few as 0 and as many as 12 random nucleotides. Overall, the theoretically diversity of this sprouts/clips library is >7.3×10²⁶ members, which contrasts with a diversity of only 1.7×10¹³ if standard randomization is used (FIGS. 16A-16B). In this library, the overall structure of the aptamer should remain, while the ligand-pocket will vary.

### Example 2—In Vitro Evolution and Identification of Squash

To evolve the adenine aptamer into a fluorogenic aptamer, aptamers that bind to agarose-immobilized DFHBI were selected for using a SELEX procedure (Paige et al., “RNA Mimics of Green Fluorescent Protein,” Science 333:642-646 (2011); Filonov et al., “Broccoli: Rapid Selection of an RNA Mimic of Green Fluorescent Protein by Fluorescence-Based Selection and Directed Evolution,” J. Am. Chem. Soc. 136: 16299-16308 (2014); and Song et al., “Imaging RNA polymerase III transcription using a Photostable RNA—Fluorophore Complex,” Nat. Chem. Biol. 13:1187-1194 (2017), which are hereby incorporated by reference in their entirety) (FIG. 1C; Table 1). By the seventh round of SELEX, the pool exhibited fluorescence upon incubation with DFHBI-1T (FIG. 16C). At this point, the library was cloned into a bacterial expression vector, transformed into E. coli, and fluorescent aptamers were identified by sorting cells based on DFHBI-1T-induced fluorescence (FIGS. 16D-16F). After recovery of plasmids from the brightest cells, three library members which induced fluorescence activation of DFHBI-1T were found (FIG. 1D; FIG. 6B). These aptamers also activated the fluorogenic dye DFHO, inducing an orange fluorescence (FIG. 6A) suggesting that these aptamers could be used for different fluorescence applications depending on the fluorophore.

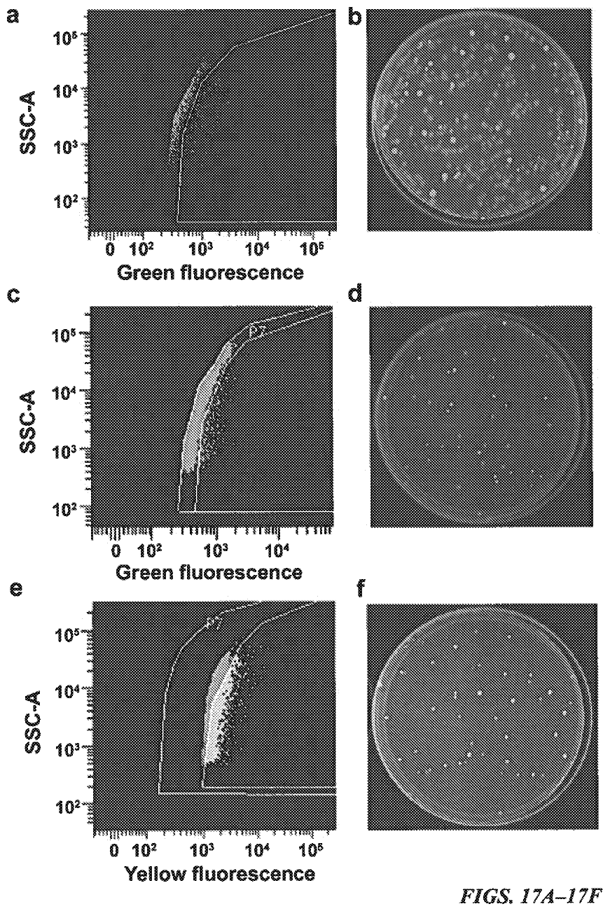

Notably, each aptamer contained an expanded ligand-binding pocket with sprouts in one or more of the randomized strands (FIG. 1E). The aptamer with the highest fluorescence activation (aptamer 9-1, ˜521-fold fluorescence activation) was selected for optimization by directed evolution (Filonov et al., “Broccoli: Rapid Selection of an RNA Mimic of Green Fluorescent Protein by Fluorescence-Based Selection and Directed Evolution,” J. Am. Chem. Soc. 136: 16299-16308 (2014) and Bartel et al., “HIV-1 Rev Regulation Involves Recognition of Non-Watson-Crick Base Pairs in Viral RNA,” Cell 67:529-536 (1991), which is hereby incorporated by reference in its entirety) (FIG. 1D; FIG. 6B). For directed evolution, a DNA library of 9-1 aptamer mutants was synthesized such that each nucleotide in the ligand-binding pocket has a controlled probability of being converted into one of the other three nucleotides (Paige et al., “RNA Mimics of Green Fluorescent Protein,” Science 333:642-646 (2011); Filonov et al., “Broccoli: Rapid Selection of an RNA Mimic of Green Fluorescent Protein by Fluorescence-Based Selection and Directed Evolution,” J. Am. Chem. Soc. 136: 16299-16308 (2014); and Sunbul et al., “Super-Resolution RNA Imaging using a Rhodamine-Binding Aptamer with Fast Exchange Kinetics,” Nat. Biotechnol. 39:686-690 (2021), which are hereby incorporated by reference in their entirety) (FIG. 25 (Sequences for First Directed Evolution)). Four rounds of SELEX were performed using this library to enrich for binding to DFHBI-agarose (Table 2). After expression in E. coli and FACS (FIGS. 17A-17B), the brightest aptamer (designated DE1-2) exhibited approximately 790-fold fluorescence enhancement of DFHBI-1T (FIG. 1D). A subsequent round of directed evolution resulted in aptamer DE2-6, which showed approximately 949-fold activation of DFHBI-1T and 492-fold activation of DFHO (FIG. 1D; FIG. 2C; FIGS. 17C-17F).

In certain bacteria, adenine riboswitches contain an extra G·U basepair in the kissing loop interaction compared to the two-basepair kissing loop in V. vulnificus (Frieda and Block, “Direct Observation of Cotranscriptional Folding in an Adenine Riboswitch,” Science 338:397-401 (2012), which is hereby incorporated by reference in its entirety). Therefore, the effect of an additional basepair was tested by replacing U residues in loops L2 and L3 with G and C, respectively. These mutations increased the fold activation of both DFHBI-1T and DFHO (949 fold to 1064 fold, and 492 fold to 550 fold, respectively) (FIG. 1D; FIG. 6A; FIG. 6G). This improved aptamer binds DFHBI-1T and DFHO (K_{d} of 45 nM and 54 nM, respectively) with high quantum yield (0.71 and 0.60 for DFHBI-1T and DFHO, respectively) (Table 6). This aptamer also shows high brightness and high photostability in mammalian cells (FIGS. 6A-6H; 7A-7H; 8A-8D; 9A-9C). The resulting aptamer was designated Squash, in keeping with previous nomenclature systems, and to reflect the multi-colored nature of Squash-fluorophore complexes (FIGS. 2A-2F).

TABLE 6
Comparison of Photophysical and Biochemical Properties
of Squash with Previous Fluorogenic Aptamers

| | Excitation | Emission | Extinction | Fluorescence | | | |
|---|---|---|---|---|---|---|---|
| | maximum | maximum | coefficient | quantum | | K_{d} | % folded |
| | (nm) | (nm) | (M⁻¹cm⁻¹) | yield | Brightness *** | (nM) | in vitro |
| DFHBI-1T | 426 | 495 | 19,900* | 0.00098 | 0.12 | — | — |
| Broccoli/DFHBI-1T | 472 | 507 | 29,600 | 0.94 | 100 | 360 | 55 |
| Squash/DFHBI-1T | 452 | 503 | 24,200 | 0.71 | 62 | 45 | 78 |
| DFHO | 473 | 561 | 27,000** | 0.0006 | 0.22 | — | — |
| Corn/DFHO | 505 | 545 | 29,000 | 0.25 | 100 | 70 | 52 |
| Squash/DFHO | 495 | 562 | 24,600 | 0.60 | 204 | 54 | 80 |

All in vitro measurements for Squash reported here were performed in 40 mM HEPES-KOH (pH 7.4), 100 mM KCl, and 0.5 mM MgCl₂ buffer. Folding experiments for all the aptamers were also performed in the buffer mentioned above. The photophysical properties of Broccoli/DFHBI-1T (Ellington and Szostak, “In Vitro Selection of RNA Molecules that Bind Specific Ligands,” Nature 346: 818-822 (1990), which is hereby incorporated by reference in its entirety) and Corn/DFHO (Porter et al., “Recurrent RNA Motifs as Scaffolds for Genetically Encodable Small-Molecule Biosensors,” Nat. Chem. Biol. 13: 295-301 (2017), which is hereby incorporated by reference in its entirety) are taken from previous reports.
*The extinction coefficient of DFHBI-1T and
**DFHO are reported here at 452 nm and 495 nm respectively.
*** Brightness (extinction coefficient × quantum yield) is reported relative to previous measurements for Broccoli (Ellington and Szostak, “In Vitro Selection of RNA Molecules that Bind Specific Ligands,” Nature 346: 818-822 (1990), which is hereby incorporated by reference in its entirety) and Corn (Porter et al., “Recurrent RNA Motifs as Scaffolds for Genetically Encodable Small-Molecule Biosensors,” Nat. Chem. Biol. 13: 295-301 (2017), which is hereby incorporated by reference in its entirety).

### Example 3—Squash Activates Fluorescence without a G-Quadruplex

Squash maintains sequence similarity to the parental adenine aptamer except for expansion and mutation of its ligand-binding pocket (FIG. 1E; FIG. 2A). The kissing loop interactions were confirmed by mutation of the G30·C57 basepair to C30·C57, which markedly impaired Squash-induced fluorescence activation of DFHO (FIG. 6G). A compensatory mutation (C30·G57) restored fluorescence. Although diverse fluorogenic aptamers bind fluorophores through a G-quadruplex (Truong and Ferre-D'Amaré, “From Fluorescent Proteins to Fluorogenic RNAs: Tools for Imaging Cellular Macromolecules,” Protein Sci. 28:1374-1386 (2019), which is hereby incorporated by reference in its entirety), Squash fluorescence is not dependent on potassium (FIG. 2E), suggesting a G-quadruplex-independent mechanism of fluorophore binding and activation. Similar to the parental add A-aptamer, Squash also has low Mg²⁺ requirement for folding (FIG. 2F).

Next, an effort was made to identify fluorophores that would allow Squash to be imaged in cells expressing Broccoli. Broccoli can be imaged with BI, a DFHBI-1T derivative that binds and enhances the folding and brightness of Broccoli (Lie et al., “Fluorophore-Promoted RNA Folding and Photostability Enables Imaging of Single Broccoli-Tagged mRNAs in Live Mammalian Cells,” Angew. Chemie. Int. Ed. 59:4511-4518 (2019), which is hereby incorporated by reference in its entirety). Importantly, BI binds Squash with much weaker affinity than Broccoli (FIG. 7G). For Squash, DFHO was used, since Squash-DFHO shows a markedly distinct excitation/emission spectra (Ex=495 nm; Em=562 nm) than Broccoli-BI (Ex=470 nm; Em=505 nm) (FIG. 2B; FIG. 6H). Additionally, DFHO binds very weakly to Broccoli (K_{d}˜11 μM) (FIG. 7H). Thus, Broccoli-BI and Squash-DFHO can be imaged together as parts of a ratiometric sensor without substantial spectral interference.

### Example 4—Squash Shows Efficient Folding In Vitro and in Cells

To determine if Squash exhibits improved folding, an in vitro folding assay was used (Strack et al., “A Superfolding Spinach2 Reveals the Dynamic Nature of Trinucleotide Repeat-Containing RNA,” Nat. Methods 10:1219-1224 (2013), which is hereby incorporated by reference in its entirety) where the first step is to determine the fluorescence of a fully folded Squash-fluorophore complex. To prepare this complex, 10 μM Squash is incubated with 0.2 μM DFHO. By having large excess of Squash, there is likely to be enough folded Squash to form 0.2 μM Squash-DFHO complex. Next, 0.2 μM Squash is incubated with excess (10 μM) DFHO. In this assay, the percent of folded Squash will determine the amount of Squash-DFHO complex that can form during the second step, up to a maximum of 0.2 μM (see Strack et al., “A Superfolding Spinach2 Reveals the Dynamic Nature of Trinucleotide Repeat-Containing RNA,” Nat. Methods 10:1219-1224 (2013), which is hereby incorporated by reference in its entirety) for more details). Using this approach, it was found that approximately 80% of Squash is folded (FIG. 3A), compared to 60% for Spinach2 (Strack et al., “A Superfolding Spinach2 Reveals the Dynamic Nature of Trinucleotide Repeat-Containing RNA,” Nat. Methods 10:1219-1224 (2013), which is hereby incorporated by reference in its entirety) and 55% for Broccoli (Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014), which is hereby incorporated by reference in its entirety).

It was next asked if Squash folding increases when it is inserted into a “folding scaffold.” The F30 folding scaffold is based off of a naturally occurring three-way junction packaging RNA in the 4|29 bacteriophage (Shu et al., “Programmable Folding of Fusion RNA In Vivo and In Vitro Driven by pRNA 3WJ Motif of phi29 DNA Packaging Motor,” Nucleic Acids Res. 42(2):e10 (2014), which is hereby incorporated by reference in its entirety). Aptamers are inserted via their helical stem regions into either arm of F30, which facilitates aptamer folding (Filonov et al., “In-Gel Imaging of RNA Processing Using Broccoli Reveals Optimal Aptamer Expression Strategies,” Chem. Biol. 22: 649-660 (2015), which is hereby incorporated by reference in its entirety). Although Broccoli folding increases after insertion into F30, Squash folding was unaffected by F30 (FIG. 3A), suggesting that Squash is already highly folded.

To further assess folding, the thermal stability of Squash-DFHO was measured. As a control Broccoli bound to BI, which markedly improves Broccoli thermal stability, was used (Li et al., “Fluorophore-Promoted RNA Folding and Photostability Enables Imaging of Single Broccoli-Tagged mRNAs in Live Mammalian Cells,” Angew. Chemie. Int. Ed. 59:4511-4518 (2019), which is hereby incorporated by reference in its entirety). Squash-DFHO exhibited a similar Tₘ (50.5° C.) as Broccoli-BI (52.5° C.) (FIG. 3B). Thus, Squash's thermostability is consistent with a highly folded structure.

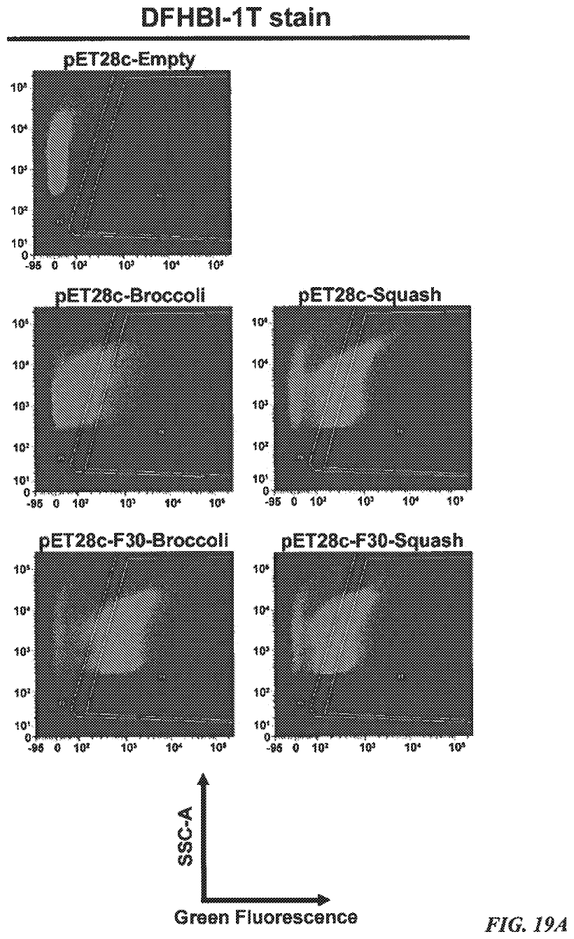

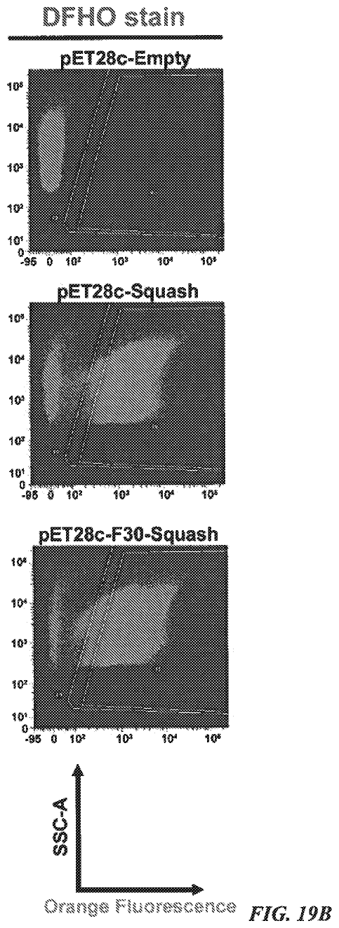

It was next asked whether Squash is highly folded in HEK293T cells. As observed previously (Filonov et al., “In-Gel Imaging of RNA Processing Using Broccoli Reveals Optimal Aptamer Expression Strategies,” Chem. Biol. 22: 649-660 (2015), which is hereby incorporated by reference in its entirety), cells expressing F30-Broccoli showed higher fluorescence than Broccoli without F30 (FIG. 3C; FIG. 19A). However, cells expressing Squash and F30-Squash showed similar cellular fluorescence (FIG. 3D; FIGS. 19A-19B). These results suggest that Squash folding is already so high that it cannot be further enhanced by F30.

Lastly, 5S rRNA was imaged in HEK293T cells. The 5S-Broccoli in DFHBI-1T-incubated cells appeared as faint dots using a 200 ms imaging time (FIG. 3D). This signal was markedly higher when 5S was tagged with F30-Broccoli (FIG. 3D). By contrast, 5S-Squash was readily detectable as a perinuclear punctate signal without using F30 (FIG. 3D). It was confirmed that the tagged 5S constructs were expressed at similar levels (FIGS. 20A-20B). Overall, these data suggest that Squash is well-folded in HEK293T cells.

### Example 5—Design of a Squash-Based SAM-Sensor

Squash was next converted into a sensor of SAM, a metabolite that influences cellular differentiation and cancer progression (Su et al., “Metabolic Control of Methylation and Acetylation,” Curr. Opin. Chem. Biol. 30:52-60 (2016), which is hereby incorporated by reference in its entirety). Non-ratiometric RNA-based SAM sensors were previously created using Broccoli, Red Broccoli and Corn (Kim and Jaffrey, “A Fluorogenic RNA-Based Sensor Activated by Metabolite-Induced RNA Dimerization,” Cell Chem. Biol. 26:1725-1731 (2019); Li et al., “Imaging Intracellular S-Adenosyl Methionine Dynamics in Live Mammalian Cells with a Genetically Encoded Red Fluorescent RNA-Based Sensor,” J. Am. Chem. Soc. 142:14117-14124 (2020); Litke and Jaffrey, “Highly Efficient Expression of Circular RNA Aptamers in Cells Using Autocatalytic Transcripts,” Nat. Biotechnol. 37:667-675 (2019); and Moon et al., “Naturally Occurring Three-Way Junctions can be Repurposed as Genetically Encoded RNA-Based Sensors,” Cell Chem. Biol. 28:1-12 (2021), which are hereby incorporated by reference in their entirety). The SAM sensors comprise the SAM-binding aptamer portion of the SAM-III riboswitch (Lu et al., “Crystal Structures of the SAM-III/SMK Riboswitch Reveal the SAM-Dependent Translation Inhibition Mechanism,” Nat. Struct. Mol. Biol. 15:1076-1083 (2008), which is hereby incorporated by reference in its entirety) fused to a fluorogenic aptamer via a transducer domain. The transducer domain is a thermodynamically unstable helix, which is stabilized upon SAM binding, thus allosterically inducing the folding of the fluorogenic aptamer (Kim and Jaffrey, “A Fluorogenic RNA-Based Sensor Activated by Metabolite-Induced RNA Dimerization,” Cell Chem. Biol. 26:1725-1731 (2019) and Litke and Jaffrey, “Highly Efficient Expression of Circular RNA Aptamers in Cells Using Autocatalytic Transcripts,” Nat. Biotechnol. 37:667-675 (2019), which are hereby incorporated by reference in their entirety).

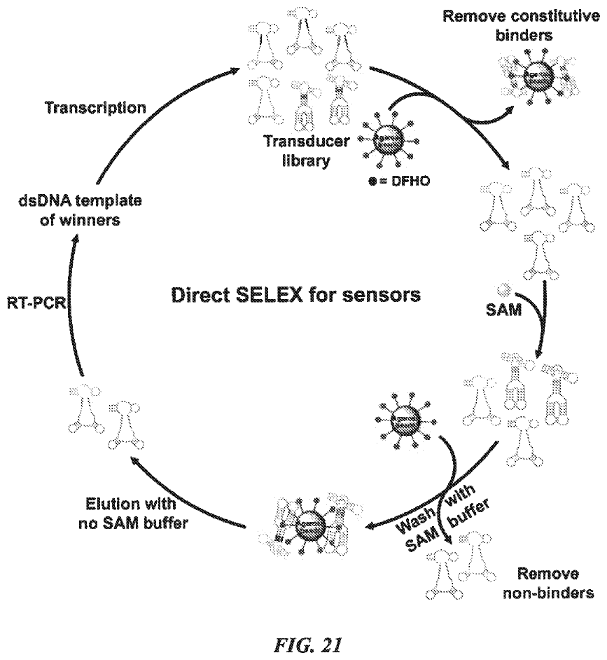

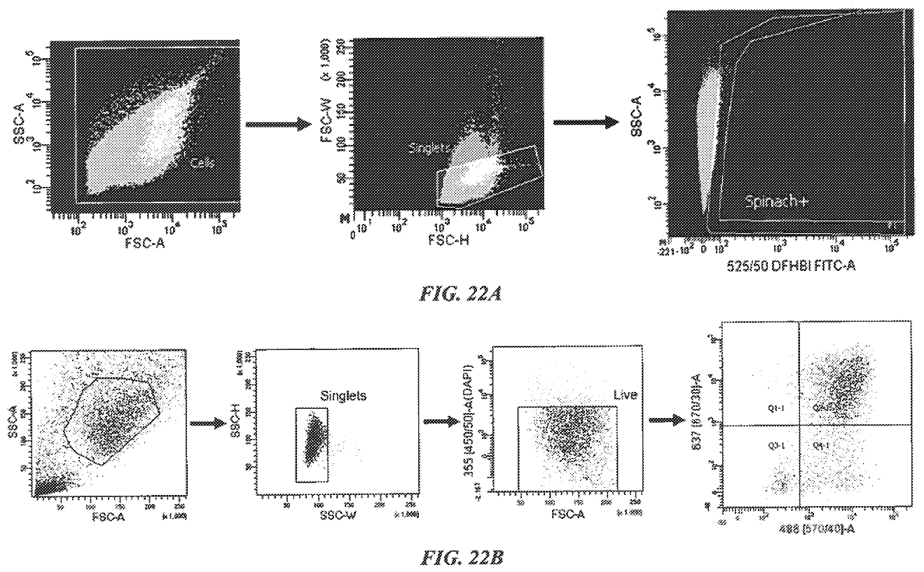

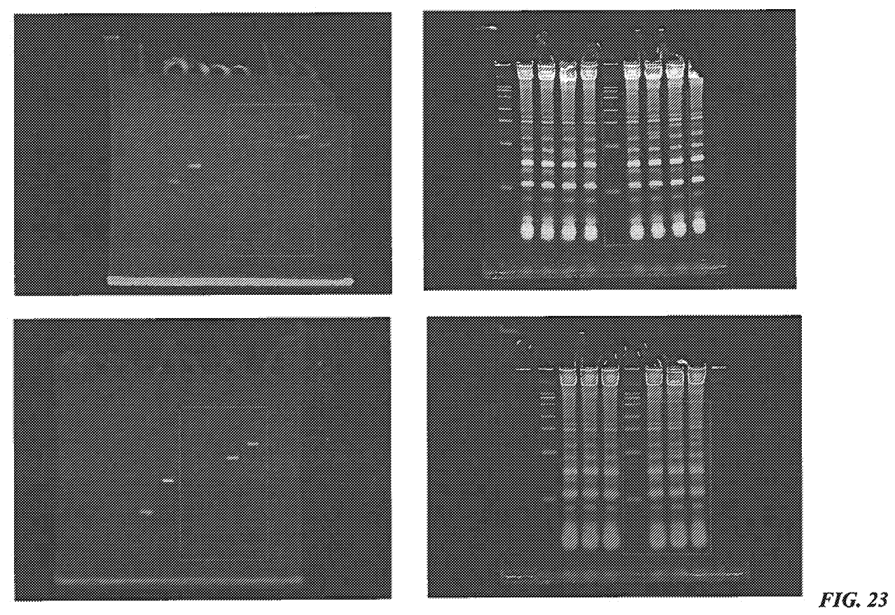

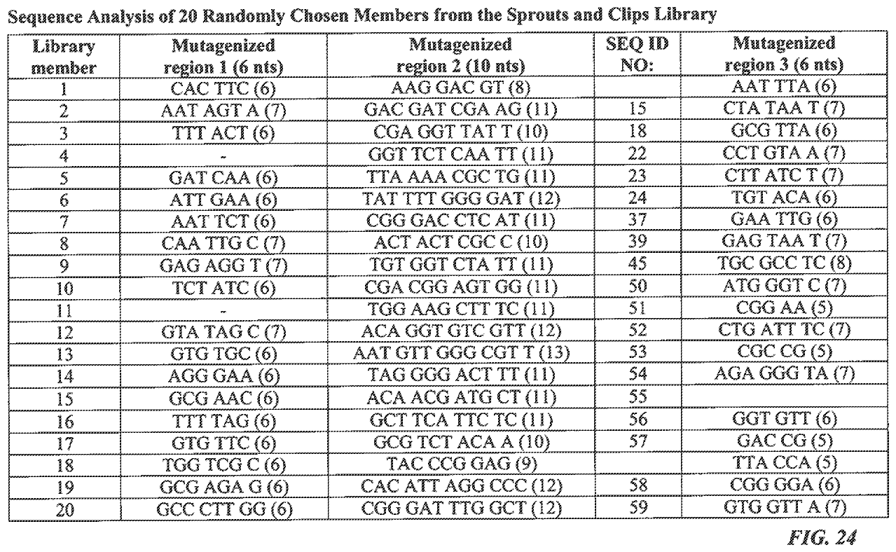

To identify an optimal transducer domain for the Squash-SAM sensor, a sprouts/clips DNA library was prepared by randomizing the transducer helix connecting the SAM aptamer and Squash (FIG. 4A; FIG. 25 (Sequences for Squash-SAM Sensor Transducer Library)). The library was transcribed into RNA, and library members that bind to DFHO-agarose beads were captured in the presence of 100 μM SAM (FIG. 21). After washing the beads with buffer containing 100 μM SAM, RNA was eluted by switching to a SAM-free buffer. After reverse transcription, the process was repeated for two more rounds, which resulted in an RNA pool that exhibited SAM-dependent fluorescence (FIG. 10A).

SAM-induced fluorescence of 48 individual library members was tested (FIG. 10B). Sensor 5-1 showed the highest fold increase in fluorescence (8.7-fold) upon incubation with SAM (FIG. 4B). Sensor 4-2 showed higher total fluorescence, but a lower fold activation due to a higher baseline signal (FIG. 4B). Sensor 5-1 is expected to give greater ability to detect different SAM levels, while Sensor 4-2 is expected to be useful if sensor RNA expression is low.

Both sensor 5-1 and 4-2 were highly selective for SAM over other related metabolites (FIG. 10C; FIG. 10F). Both Squash-SAM sensors showed rapid fluorescence activation upon addition of 100 μM SAM, with 90% fluorescence achieved in 2 minutes (Sensor 5-1) and 6 minutes (Sensor 4-2), compared with 11 minutes for the Corn-based SAM sensors, with similar deactivation kinetics (FIG. 4C; FIG. 4D).

To use the Squash-SAM sensor in HEK293T cells, each sensor was expressed as a circular RNA using the Tornado system, which enables RNA-based sensors to be expressed at high levels in mammalian cells (Litke and Jaffrey, “Highly Efficient Expression of Circular RNA Aptamers in Cells Using Autocatalytic Transcripts,” Nat. Biotechnol. 37:667-675 (2019), which is hereby incorporated by reference in its entirety) (FIG. 11A). For both sensors, a rapid decrease in fluorescence was observed within 30 minutes of treatment with cycloleucine, an inhibitor of SAM biosynthesis (Kim and Jaffrey, “A Fluorogenic RNA-Based Sensor Activated by Metabolite-Induced RNA Dimerization,” Cell Chem. Biol. 26:1725-1731 (2019); Li et al., “Imaging Intracellular S-Adenosyl Methionine Dynamics in Live Mammalian Cells with a Genetically Encoded Red Fluorescent RNA-Based Sensor,” J. Am. Chem. Soc. 142:14117-14124 (2020); and Litke and Jaffrey, “Highly Efficient Expression of Circular RNA Aptamers in Cells Using Autocatalytic Transcripts,” Nat. Biotechnol. 37:667-675 (2019), which are hereby incorporated by reference in their entirety) (FIGS. 11A-11C). Upon removal of cycloleucine, the fluorescence returned over 60 minutes, indicating restoration of SAM levels. Overall, these data indicate that sensors 5-1 and 4-2 detect SAM in mammalian cells.

### Example 6—A Ratiometric Sensor for SAM Imaging in Live Mammalian Cells

An RNA-based sensor could become ratiometric by co-expressing a fluorescent protein for normalization. However, it was found that RNA and protein expression were poorly correlated, even when expressed from the same plasmid (FIG. 12A). Thus, a single RNA that functions as a ratiometric sensor was developed. This RNA contains the Squash-SAM sensor in one arm and Broccoli in other arm of the F30 three-way junction (FIG. 4E). As a control, expression of an RNA with Squash in one arm and Broccoli in the other arm of F30 showed high correlation and minimal spectral overlap between the orange and green fluorescence signals using both microscopic imaging and flow cytometry (FIG. 4F; FIGS. 12B-12D).

Before using the sensor for ratiometric imaging, it was first confirmed that the fluorophores used to detect Squash and Broccoli are selective for their cognate aptamer and do not affect the fluorescence of the other aptamer. To test this, Squash fluorescence in Squash-expressing mammalian cells incubated with 10 μM DFHO was first measured. Subsequent application of 10 μM BI did not affect Squash fluorescence levels (FIG. 12E). Similarly, Broccoli fluorescence in BI-incubated cells expressing circular Broccoli was not affected by subsequent addition of 10 μM DFHO (FIG. 12E). These data confirm that these fluorophores are selective for their cognate aptamers. It was also verified that Broccoli and Squash do not exhibit FRET which would adversely affect fluorescence signals from the SAM sensor (FIG. 12F). Finally, the Broccoli-BI pair is not affected by SAM or structurally related molecules making it an ideal normalizer (FIG. 10I).

It was next asked whether the ratiometric sensor detects endogenous SAM. The ratiometric signal was markedly reduced in HEK293T cells within 30 minutes of cycloleucine treatment (FIGS. 4F-4G). Notably, the overall time course and magnitude in the drop of fluorescence was relatively homogeneous across all imaged cells (FIGS. 4F-4G). When cycloleucine was removed, the ratiometric signal returned to basal levels within 90 minutes (FIGS. 4F-4G). Importantly, the effect of cycloleucine is not due to cytotoxicity because a control Squash RNA is not affected by this treatment (FIG. 4F).

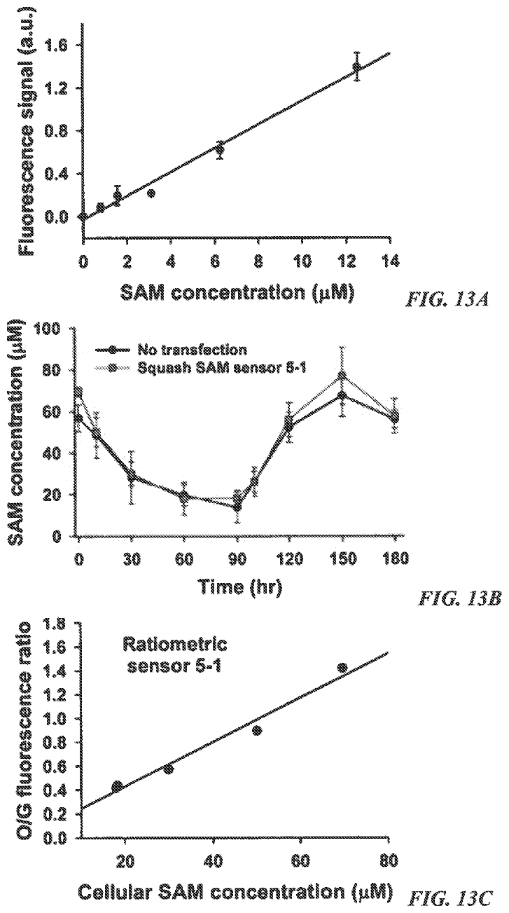

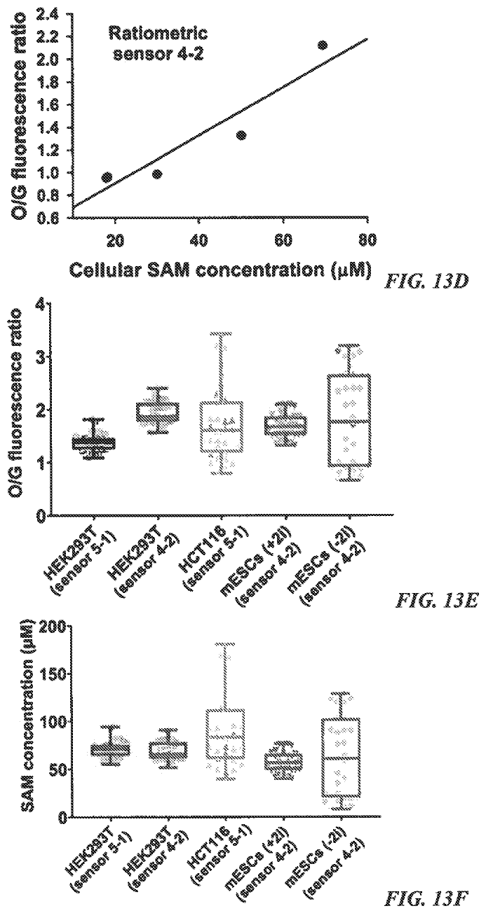

Biochemical measurements of SAM levels at each time point after cycloleucine treatment correlate with the average ratiometric signal in cells (FIGS. 13A-13D). Based on this correlation between cellular fluorescence and SAM levels, the average SAM concentration in HEK293T cells varies between 60 and 90 μM (FIGS. 13E-13F). Taken together, the ratiometric Squash-SAM sensor enables quantitative detection of SAM levels in cells.

### Example 7—Cell-to-Cell Heterogeneity and Metabolic Origins of SAM in Different Cell Types

Mouse embryonic stem cells (mES cells) contain enzymatic machinery to metabolize threonine to promote SAM biosynthesis (Wang et al., “Dependence of Mouse Embryonic Stem Cells on Threonine Catabolism,” Science 325:435-439 (2009) and Shyh-Chang et al., “Influence of Threonine Metabolism on S-Adenosylmethionine and Histone Methylation,” Science 339:222-226 (2013), which are hereby incorporated by reference in their entirety). Threonine is a precursor for 5-methyltetrahydrofolate, which is used to generate methionine from homocysteine (Sanderson et al., “Methionine Metabolism in Health and Cancer: A Nexus of Diet and Precision Medicine,” Nat. Rev. Cancer 19:625-637 (2019), which is hereby incorporated by reference in its entirety). However, bulk metabolic labeling studies using isotopically labeled threonine shows that only 5% of SAM is derived from threonine in mES cells (Shyh-Chang et al., “Influence of Threonine Metabolism on S-Adenosylmethionine and Histone Methylation,” Science 339:222-226 (2013), which is hereby incorporated by reference in its entirety). Therefore, it remains unclear to what extent threonine is required to maintain methionine levels for SAM biosynthesis in mES cells.

To test this, mES cells cultured in media containing serum and leukemia inhibitory factor, as well as inhibitors of mitogen-activated protein kinase and glycogen synthase kinase-3β were used. mES cells cultured in this media (designated +2i), are highly homogenous with low propensity for differentiation (Ying et al., “The Ground State of Embryonic Stem Cell Self-Renewal,” Nature 453:519-523 (2008), which is hereby incorporated by reference in its entirety). In contrast, mES cells cultured without the two kinase inhibitors (−2i media) exhibit more cellular heterogeneity and varying tendencies to differentiate (Chambers et al., “Nanog Safeguards Pluripotency and Mediates Germline Development,” Nature 450:1230-1234 (2007) and Filipczyk et al., “Biallelic Expression of Nanog Protein in Mouse Embryonic Stem Cells,” Cell Stem Cell 13:12-13 (2013), which are hereby incorporated by reference in their entirety).

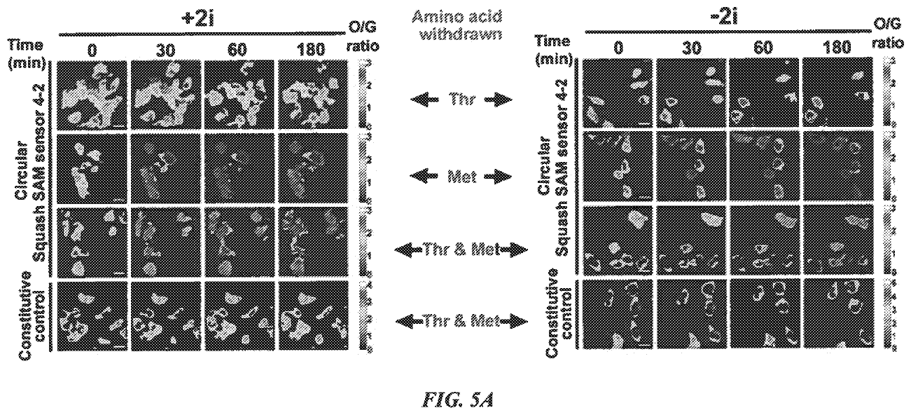

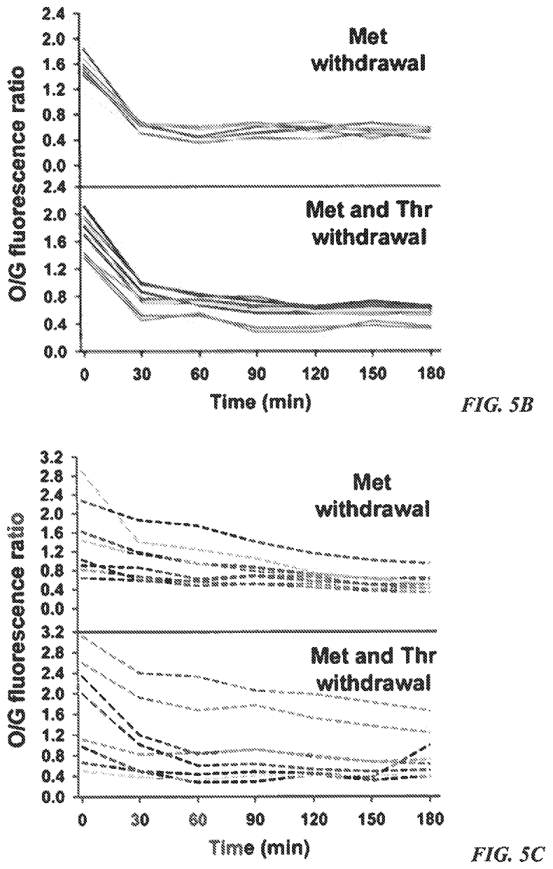

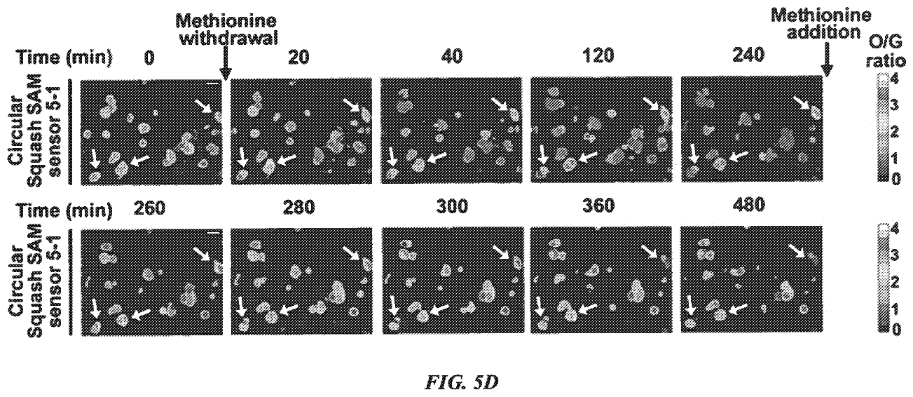

For SAM measurements in mES cells, Squash-SAM sensor 4-2 was used since it produced more fluorescence than the Squash-SAM sensor 5-1 (FIG. 14A). The intracellular SAM level in +2i media were homogeneous and varied between 40-70 μM (FIGS. 5A-5B; FIG. 13F). This is similar to HEK293T cells which showed relatively homogeneous SAM levels at 60-90 μM (FIG. 4F; FIG. 13F). However, mES cells cultured in −2i showed considerable cell-to-cell variability with SAM concentrations varying between 10-120 μM (FIG. 5A-5C; FIG. 13F).

Upon switching mES cells to threonine-depleted media, essentially no change in SAM levels in any mES cells examined over 3 hours using either culturing condition was observed (FIG. 5A). This suggests that threonine is not required to maintain SAM levels in mES cells.

Upon switching mES cells to methionine-free media, a drop in SAM at a rate that depended on the culturing conditions was observed (FIGS. 5A-5C). For +2i, the SAM levels dropped quickly within the first 30 minutes and then remained constant at a low level (FIGS. 5A-5B). The initial SAM concentration, as well as the rate of SAM depletion, was highly synchronous (FIGS. 5A-5B), suggesting that these cells were metabolically homogeneous.

In contrast, mES cells cultured in −2i showed considerable cell-to-cell metabolic heterogeneity upon methionine removal (FIGS. 5A-5C). mES cells that started with a low SAM concentration exhibited a minor drop in SAM levels. However, mES cells with higher SAM concentrations exhibited considerable heterogeneity in the rate of drop in SAM levels, although all cells showed a decline in SAM levels (FIG. 5C). These experiments indicate that mES cells cultured in −2i are metabolically distinct and that methionine derived from the media is the major source of SAM in mES cells under both culture conditions. Notably, in either +2i or −2i media, SAM levels were markedly depleted upon addition of cycloleucine (FIG. 14B), supporting the idea that MAT2A is the major enzyme responsible for SAM biosynthesis in mES cells.

Although threonine depletion has minimal effects on SAM levels, threonine depletion may cause cells to switch to exogenous methionine to generate SAM. To test this, mES cells were switched to media lacking both methionine and threonine. Here, a drop in SAM levels at a rate similar to when methionine alone was depleted was observed (FIGS. 5A-5C). When individual cells were quantitatively examined, the overall trend was essentially identical in cells cultured in methionine-free media or both methionine- and threonine-free media, regardless of the culturing conditions (FIGS. 5B-5C). These results suggest that threonine does not substantially contribute to overall intracellular SAM levels in the conditions used here.

Notably, the effect of amino acid removal on SAM levels was not due to cytotoxicity, since SAM levels were restored by reintroducing the amino acids (FIGS. 14C-14D).

Whether SAM levels show heterogeneity in HCT116 colon cancer cells was also examined. Although both the 4-2 and 5-1 ratiometric SAM sensor produced readily detectable signals (FIG. 5D; FIG. 15A), 5-1 was used since it exhibits a higher dynamic range. HCT-116 cells exhibited considerable cell-to-cell heterogeneity, with intracellular SAM levels ranging from 40-180 μM (FIG. 5D; FIG. 13F). To understand the basis for these differences, the SAM consumption rate by endogenous methyltransferases was determined by depleting methionine and monitoring the rate of SAM loss. Using this approach, it was found that HCT-116 cells exhibit three patterns of SAM consumption rates, which were related to the initial SAM concentration (FIGS. 5D-5E). For type I cells (initial SAM concentration 90-130 μM), SAM levels gradually dropped and substantial SAM levels were retained after 4 hours (FIG. 5E). By contrast, type II cells (60-90 μM) showed rapid SAM consumption with 90% depleted within 40 minutes. Type III cells (40-60 μM), exhibited a more gradual drop in SAM levels over 4 hours (FIG. 5E). These data suggest that individual HCT-116 cells exhibit distinct metabolic states.

Notably, addition of cycloleucine caused a slightly faster drop in SAM levels compared to methionine and threonine depletion in these cells (FIGS. 10C-10E). The fact that methionine/threonine depletion was not as effective as cycloleucine at reducing SAM levels (FIGS. 15B-15E) suggests that these cells may engage in threonine- and methionine-independent pathways for SAM biosynthesis (Su et al., “Metabolic Control of Methylation and Acetylation,” Curr. Opin. Chem. Biol. 30:52-60 (2016), which is hereby incorporated by reference in its entirety), albeit at a relatively low efficiency.

### Discussion of Examples 1-7

Here a generalizable strategy for ratiometric imaging of metabolite levels using an RNA-based sensor is described. The ratiometric sensor is an RNA comprising Broccoli, which provides constitutive green fluorescence to normalize for sensor expression, and a SAM-regulated Squash aptamer, which produces orange fluorescence in proportion to SAM levels in cells. Broccoli and Squash bind different fluorophores, with minimal interference between their fluorescent emissions (Li et al., “Fluorophore-Promoted RNA Folding and Photostability Enables Imaging of Single Broccoli-Tagged mRNAs in Live Mammalian Cells,” Angew. Chemie Int. Ed. 59:4511-4518 (2019), which is hereby incorporated by reference in its entirety). The sensor is expressed as a circular RNA, enabling expression levels that generate sufficient fluorescence signals for quantitative metabolite detection in diverse mammalian cells.

To create the ratiometric sensor, Squash, a fluorogenic aptamer with high folding, and a corresponding increase in cellular fluorescence, was generated. RNA folding limits the fluorescence of fluorogenic aptamers in mammalian cells (Li et al., “Fluorophore-Promoted RNA Folding and Photostability Enables Imaging of Single Broccoli-Tagged mRNAs in Live Mammalian Cells,” Angew. Chemie Int. Ed. 59:4511-4518 (2019); Strack et al., “A Superfolding Spinach2 Reveals the Dynamic Nature of Trinucleotide Repeat-Containing RNA,” Nat. Methods 10(12):1219-24 (2013); and Filonov et al., “In-Gel Imaging of RNA Processing Using Broccoli Reveals Optimal Aptamer Expression Strategies,” Chem. Biol. 22: 649-660 (2015), which are hereby incorporated by reference in their entirety). These aptamers likely fold poorly since they were created using fully randomized libraries that are selected only for their ability to bind to a fluorophore, rather than their ability to fold in the cytosol. These selected aptamers contrast with riboswitch aptamers which evolved to fold so efficiently that they can function as they are being transcribed (Frieda and Block, “Direct Observation of Cotranscriptional Folding in an Adenine Riboswitch,” Science 338:397-401 (2012) and Lemay et al., “Comparative Study between Transcriptionally- and Translationally-Acting Adenine Riboswitches Reveals Key Differences in Riboswitch Regulatory Mechanisms,” PLoS Genet. 7:e1001278 (2011), which are hereby incorporated by reference in their entirety. Therefore, the naturally occurring add A-riboswitch aptamer library was evolved into a fluorophore-binding fluorogenic aptamer. To do this, an RNA library comprising roughly 10¹⁵ library members in which randomization occurred exclusively in the ligand-binding pocket was used. The library members retained key structural features of the parental add A-riboswitch aptamer, including its kissing loop interaction and helical domains.

Squash was generated using a new approach for generating a randomized DNA library. Rather than simply randomizing the sequence of the nucleotides that comprise the ligand-binding pocket, the size was also randomized, thus allowing SELEX to sample larger and smaller ligand-binding pockets. Library members contain ligand-binding pockets that can theoretically range from 0 to 44 nucleotides in length, distributed across three junctional strands.

To cause random increases in the size of the ligand-binding pocket, synthetic steps were added using phosphoramidites at low concentration, resulting in stochastic “sprouts” at defined positions in the DNA library. Random shortenings, termed “clips,” were obtained by reducing the coupling time when using the standard phosphoramidite mixture. Using this approach, the ligand-binding pocket of Squash was expanded by 4 nucleotides relative to the parental aptamer. The sprouts/clips approach can be used to evolve any aptamer allowing it to expand or contract to accommodate ligands of different sizes.

Squash appears to have maintained the high folding efficiency of its parental aptamer based on its high folding in vitro and high fluorescence in cells. Riboswitch-derived aptamers have been used previously as templates for SELEX libraries based on the idea that their ligand-binding pockets may be readily evolved to bind different ligands (Porter et al., “Recurrent RNA Motifs as Scaffolds for Genetically Encodable Small-Molecule Biosensors,” Nat. Chem. Biol. 13:295-301 (2017), which is hereby incorporated by reference in its entirety). Here it was shown that this approach also results in an efficiently folded fluorogenic aptamer, thus overcoming a major shortcoming of previous fluorogenic aptamers.

The Squash-SAM sensors show faster fluorescence induction upon addition of SAM. These faster kinetics may reflect Squash's origin from a riboswitch, which normally undergoes adenine-dependent conformational changes (Lemay et al., “Comparative Study between Transcriptionally- and Translationally-Acting Adenine Riboswitches Reveals Key Differences in Riboswitch Regulatory Mechanisms,” PLoS Genet. 7:e1001278 (2011), which is hereby incorporated by reference in its entirety). Other natural aptamers may be suitable for developing fluorogenic aptamers, especially if their folding does not vary at different physiologic concentrations of magnesium. This helps to ensure that changes in the fluorescence signals reflect metabolite concentrations rather than changes in intracellular magnesium levels.

Ratiometric sensing has been previously demonstrated in E. coli using Broccoli and a dinitroaniline-binding aptamer, DNB (Wu et al., “Genetically Encoded Ratiometric RNA-Based Sensors for Quantitative Imaging of Small Molecules in Living Cells,” Angew. Chemie Int. Ed 58:18271-18275 (2019), which is hereby incorporated by reference in its entirety). However, this aptamer and its fluorophore, sulforhodamine B conjugated to dinitroaniline has only been used in bacterial cells (Wu et al., “Genetically Encoded Ratiometric RNA-Based Sensors for Quantitative Imaging of Small Molecules in Living Cells,” Angew. Chemie Int. Ed 58:18271-18275 (2019) and Sunbul and Jaschke, “SRB-2: A Promiscuous Rainbow Aptamer for Live-Cell RNA Imaging,” Nucleic Acids Res. 46(18):e110 (2018), which are hereby incorporated by reference in their entirety) possibly due to cellular background fluorescence. Thus, the approach described here provides a general strategy for ratiometric metabolite imaging in mammalian cells.

Intracellular SAM concentrations can vary in different disease contexts (Hao et al., “Immunoassay of S-adenosylmethionine and S-adenosylhomocysteine: The Methylation Index as a Biomarker for Disease and Health Status,” BMC Res. Notes 9:1-16 (2016), which is hereby incorporated by reference in its entirety). The Squash-SAM sensor could be adjusted to detect SAM at different concentrations by tuning the K_{d} of the sensor for SAM. This can be achieved either by changing the transducer sequence joining Squash and the SAM aptamer or by using a different SAM aptamer with suitable K_{d}.

Using the Squash-SAM sensor, it was found that cells can exist in distinct metabolic states with respect to SAM metabolism. This effect is dependent on culturing conditions and cell type. These findings underscore the importance of interrogating metabolism at a single-cell level and illustrate the power of RNA-based ratiometric sensors to identify previously unanticipated heterogeneity in cellular metabolic networks.

### Materials and Methods for Examples 8-10

### Reagents and Equipment

DFHBI-1T, DFHO, and BI fluorophores were synthesized as described previously (Paige et al., “RNA Mimics of Green Fluorescent Protein,” Science. 333:642-646 (2011); Song et al., “Imaging RNA Polymerase III Transcription using a Photostable RNA-Fluorophore Complex,” Nat. Chem. Biol. 13:1187-1194 (2017); and Li et al., “Fluorophore-Promoted RNA Folding and Photostability Enables Imaging of Single Broccoli-Tagged mRNAs in Live Mammalian Cells,” Angew. Chem. Weinheim Bergstr. Ger. 132:4541-4548 (2020), which are hereby incorporated by reference in their entirety). Absorbance spectra were measured using a Thermo Scientific NanoDrop 2000 spectrophotometer with cuvette capability. Fluorescence measurements were obtained using a FluoroMax-4 spectrofluorometer (Horiba Scientific) or a SpectraMax iD3 Multi-Mode Microplate Readers (Molecular Devices). Bacterial colony fluorescence on agar plates was measured using a ChemiDoc MP imager (Bio-Rad). FACS experiments were performed using FACSAria II instrument (BD Biosciences). Fluorescence images of cultured cells were taken using an Eclipse TE2000-E microscope (Nikon).

### Cloning

Polymerase chain reactions (PCR) were performed using Phusion® High-Fidelity DNA Polymerase (NEB M0530). Single stranded synthetic DNA oligonucleotides used in PCR were purchased from Integrated DNA Technologies. After PCR, 1% TAE agarose gels were used for separating PCR products with the correct size. The PCR products band with the correct size were excised and purified using the Qiaquick Gel Extraction kit (Qiagen 28704). Following PCR and gel purification, the purified PCR products were subjected to restriction digest with the appropriate restriction endonucleases purchased from New England Biolabs, following the manufacturer's recommended protocol. Quick Ligation™ Kit (NEB M2200L) was used for DNA ligation reactions. After DNA ligation, the ligated DNA plasmids were transformed and propagated using chemically competent E. coli (Agilent 200314). To extract DNA plasmids from E. coli, QIAprep Spin Plasmid Miniprep Kit (Qiagen 27106) was used according to the manufacturer's recommended protocol. The sequence of the extracted DNA plasmids was verified by DNA sequencing service from GENEWIZ.

### Preparation of DFAME-Affinity Matrix

Amine-functionalized DFAME was first dissolved in DMSO at a concentration of 40 mM and then diluted into 100 mM HEPES buffer pH 7.5 with a final concentration of 5% DMSO and 2 mM amine-functionalized DFAME. This fluorophore solution was then added to NHS-activated Sepharose (GE Life Sciences), which had been preequilibrated with two volumes of ice-cold buffer. The resin was then incubated with amine-functionalized DFAME solution overnight at 4° C. in the dark. The resin was washed with reaction buffer and incubated with 100 mM Tris pH 8.0 for 2 hours at 25° C. to block with any remaining NHS-activated sites. After thorough washing, the resin was stored in 1:1 ethanol:100 mM sodium acetate pH 5.4 at 4° C. The efficiency of sepharose coupling was monitored by measuring the absorbance at 500 nm of free DFAME in the flow-through. Using this approach, it is estimated that the resin contains approximately 5 μmol of fluorophore/ml.

### SELEX Procedure

The random library used for selecting Beetroot was generated before and previously used to isolate Spinach, Broccoli, and Corn (Paige et al., “RNA Mimics of Green Fluorescent Protein,” Science. 333:642-646 (2011); Song et al., “Imaging RNA Polymerase III Transcription using a Photostable RNA-Fluorophore Complex,” Nat. Chem. Biol. 13:1187-1194 (2017); and Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014), which are hereby incorporated by reference in their entirety). In brief, this library contains two 26-base random stretches separated by a 12-base fixed sequence and flanked from 5′ and 3′ ends with constant regions used for PCR amplification and in vitro transcription. The doped library used for directed evolution SELEX was contained a 64-base variable region flanked from 5′ and 3′ ends with constant regions used for PCR amplification and in vitro transcription. Doped libraries were described in detail previously (Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014), which is hereby incorporated by reference in its entirety). In brief, these libraries were created so that each encoded aptamer resembles the parent aptamer, except that there are on average seven mutations per sequence. To obtain this library, every position is chemically synthesized with a phosphoramidite nucleosides mixture that contains primarily the nucleotide that is found at that position in the parent aptamer, but also contains each of the other nucleotides at a lower concentration. Double-stranded DNA (dsDNA) encoding doped libraries were designed with 14% mutagenesis efficiency and were ordered from the Protein and Nucleic Acid Facility, Stanford University Medical Center.

1×10¹⁴ different sequences of dsDNA were transcribed in a 500 μl T7 RNA polymerase transcription reaction using the AmpliScribe T7-Flash Transcription Kit (Epicentre Biotechnologies). After treatment with DNase (Epicentre Biotechnologies) for 16 hours, RNAs were purified using RNeasy Mini Kit (Qiagen) following the manufacturer's recommendations. The doped library SELEX was conducted essentially as described previously (Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014), which is hereby incorporated by reference in its entirety). Briefly, during the first step RNA species capable of binding to the DFAME-sepharose matrix were removed by incubation with “mock” resin for 1 hour at room temperature. The resulting RNA solution was then incubated with DFAME-coupled matrix for 30 minutes at 37° C. RNA bound to DFAME resin was then washed 6 times with 0.5 ml of selection buffer at 37° C. Finally, specifically bound RNA was eluted with free DFAME 37° C.

The eluted RNAs were then ethanol precipitated, reverse transcribed, PCR amplified and in vitro transcribed to yield the pool for the next SELEX round. The presence of fluorescent RNA species in each pool was assessed by mixing 20 μM RNA and 10 μM DFAME and measuring fluorescence emission of this solution on a fluorometer in comparison with the fluorophore alone. At this point, RNAs were cloned into bacterial expression plasmids for FACS-based screening.

### Bacterial Library Generation and FACS Sorting

RNA libraries, expressed from the pBAD E plasmid (Filonov et al., “Broccoli: Rapid Selection of an RNA Mimic of Green Fluorescent Protein by Fluorescence-Based Selection and Directed Evolution,” J. Am. Chem. Soc. 136:16299-16308 (2014), which is hereby incorporated by reference in its entirety), were analyzed in LMG194 E. coli (ATCC). LMG194 cells then were grown in LB media overnight in presence of 0.002% arabinose and then collected for sorting. The diversity of the resulting bacterial library was assessed to contain ˜3.6×10⁷ individual members. Cells were preincubated with 20 μM DFAME and then sorted on a FACSAria II instrument (BD Biosciences). The sample compartment of the sorter was maintained at 37° C. to facilitate sorting of cells expressing the most thermostable aptamers. To isolate red fluorescent events, cells were excited with the 561-nm laser and their emission was collected using a 585±21 emission filter. Typically, the top 1,000 brightest cells were sorted into 1 ml SOC media and cultured at 37° C. for 1 hour. The cells were then plated on LB agar supplemented with carbenicillin, 0.002% arabinose and 10 μM DFAME.

The next day, the colonies on the LB-agar plate were imaged on a ChemiDoc MP imager (Bio-Rad). Yellow fluorescence was collected in a channel with 530±15 nm excitation and 607±25 nm emission. The Cy5 channel (630±15 nm excitation and 697±22.5 nm emission) was used to collect autofluorescence signal from bacterial colonies, which allows normalization for colony size. Images were processed and normalized in ImageJ software (NIH) to identify colonies expressing the brightest aptamers.

The top-performing RNA sequences were then subjected to further truncation and in vitro fluorescence characterization to identify the shortest sequence retaining the DFAME fluorescence activation capacity.

### In Vitro Characterization of RNA Aptamer-Fluorophore Complexes

dsDNA encoding the top-performing RNA sequences from the brightest bacterial colonies was PCR amplified from the purified plasmids. PCR products were subjected to gel electrophoresis in 2% TBE gel, and the PCR product band corresponding to the correct size was excised from gel and further purified with PCR purification columns (Qiagen) and in vitro transcribed using an AmpliScribe T7-Flash Transcription Kit (Epicenter). The resulting RNA was then purified using Zymo RNA concentrator columns and quantified by absorbance using a Thermo Scientific NanoDrop 2000 spectrophotometer. For absorption, excitation and emission spectra measurements, “excess RNA” conditions and limiting amount of fluorophore was used to ensure that no free fluorophore contributes to the absorbance or fluorescence signal. The RNA concentration used for fluorescence and absorbance measurements was 20 μM and 50 μM, respectively, while the dye concentration was 2 μM and 5 μM, respectively. All in vitro RNA properties were measured in 40 mM HEPES pH 7.4, 100 mM KCl, 5 mM MgCl₂ buffer, unless specified.

### Quantum Yield and Extinction Coefficient Measurements

Extinction coefficient was calculated based on Beer's law. Briefly, absorbance spectrum of 5 μM DFAME, Beetroot-DFAME, or Corn-DFAME was measured using a Thermo Scientific NanoDrop 2000 spectrophotometer with cuvette capability. Extinction coefficient was calculated by dividing peak absorbance value by the concentration of fluorophore alone, or RNA-fluorophore complex.

Quantum yield was determined by comparing the integral of the emission spectra for DFAME or RNA-DFAME complex with the corresponding integral obtained from Rhodamine B solution. These integral values were plotted against the absorbance values corresponding to the excitation wavelength. The slopes from the above plots were calculated for determining quantum yield. Quantum yield was calculated by multiplying the quantum yield of Rhodamine B by the slope of DFAME or RNA-DFAME complex, then divided by the slope of Rhodamine B.

### Non-Denaturing Gel Electrophoresis

Non-denaturing polyacrylamide gels were prepared by casting 10×10 cm 10% polyacrylamide ((29:1 acrylamide:bisacrylamide; Sigma) with a non-denaturing buffer condition (40 mM HEPES, pH 7.5, 100 mM KCl, 5 mM MgCl₂). Non-denaturing gel electrophoresis was performed at 90 V for 75 minutes at 4° C. For staining, gels were incubated in the same non-denaturing buffer with 10 μM DFHO or DFAME for 20 minutes in the dark. Gels were then imaged using a Biorad ChemiDoc system. For SYBR Gold staining, gels were incubated in the same non-denaturing buffer with 1×SYBR Gold for 15 minutes in the dark. The resulting SYBR Gold-stained gels were imaged using the same Biorad ChemiDoc system.

### Construction of DNA Plasmids Used for Imaging RNA Assembly in Mammalian Cells

A pAV-U6+27-Tornado vector backbone (Wu et al., “Live Imaging of mRNA using RNA-Stabilized Fluorogenic Proteins,” Nat. Methods. 16:862-865 (2019), which is hereby incorporated by reference in its entirety) was used for expressing 1× Corn, 3× Corn, 5× Corn, 5× Corn-MS2, 5× Beetroot-boxB. To construct these plasmids, EcoRI and NheI were inserted into the pAV-U6+27-Tornado-F30-Pepper(TAR Variant-2) (Addgene plasmid #129405), flanking F30-Pepper. The resulting plasmid was digested by the EcoRI and NheI restriction enzymes to get the pAV-U6+27-Tornado vector backbone. Double-stranded DNA insert of 1× Corn, 3× Corn, 5× Corn, 5× Corn-MS2, and 5× Beetroot-boxB were first digested by the same restriction enzymes, then ligated into the pAV-U6+27-Tornado vector backbone, respectively. DNA insert of 5× Corn-MS2 and 5× Beetroot-boxB were synthesized and purchased from GenScript.

To construct expression vectors for MCP-mCherry and GFP-N-peptide, miniCMV-(mNeonGreen)4-tDeg (Addgene plasmid #129402) was digested by the HindIII and XbaI restriction enzymes to get the pcDNA3.1V vector backbone with a miniCMV promoter. Genes encoding mCherry-stdMCP and GFP-N-peptide were first digested by the same restriction enzymes, then ligated to the pcDNA3.1⁺ vector backbone with a miniCMV promoter, respectively. The gene encoding stdMCP was synthesized and purchased from Integrated DNA Technologies according to sequence from pUbC-nls-ha-stdMCP-stdGFP (Addgene plasmid #98916).

### Fluorescence Imaging of RNA Assembly in Mammalian Cells

HEK293T cells (ATCC) were cultured in Dulbeco's modified Eagle's medium (Thermo Fisher Scientific, 11995-065) supplemented with 10% fetal bovine serum (Corning 35-010-CV), 100 U ml⁻¹ of penicillin and 100 g ml⁻¹ of streptomycin (Thermo Fisher Scientific, 15140122) under 37° C. with 5% CO₂.

For to live-cell fluorescence imagining experiments, HEK293T cells were seeded into 35-mm imaging dishes precoated with poly-D-lysine (Mattek Corporation, P35GC-1.5-14C) and mouse laminin I (Cultrex, 3401-010-02) with a cell density of 4.5×10⁵ cells per dish. On the next day, HEK293T cells were transfected with DNA plasmids as indicated in figures with fluorescence imaging experiments using FuGENE HD. Transfections were performed according FuGENE HD manufacturer's instructions. HEK293T cells were then cultured for two days before imaging.

Prior to imaging experiments, HEK293T cells were incubated with 1 μl of Hoechst 33342 (Thermo Fisher Scientific, H3570) per 2 ml of imaging media (phenol red-free Dulbeco's modified Eagle's medium (Thermo Fisher Scientific 31053-028) supplemented with 10% fetal bovine serum (Corning 35-010-CV), 100 U ml-1 of penicillin and 100 g ml⁻¹ of streptomycin, 1× GlutaMAX (Thermo Fisher Scientific, 35050-061) and 1 mM sodium pyruvate (Thermo Fisher Scientific, 11360-070)). For imaging experiments of RNA-fluorophore complex, HEK293T cells were incubated with imaging media supplemented with a final concentration of 10 μM fluorophore (DFHO or DFAME) and 5 mM MgCl₂ for one hour prior to imaging.

For all imaging experiments, an epifluorescence inverted microscope (Nikon Eclipse TE2000-E) was used equipped with a CoolSnap HQ2 CCD camera and a 130-W Nikon mercury lamp. The microscope and camera were controlled using the NIS-Elements Advanced Research software (Nikon). Cells were imaged with a 60× oil objective with a numerical aperture of 1.4 (Nikon) at 37° C. A FITC filter cube (with excitation filter 470±20 nm, dichroic mirror 495 nm (long pass) and emission filter 525±25 nm) was used for imaging GFP-N-peptide. A YFP filter cube (with excitation filter 500±12 nm, dichroic mirror 520 nm (long pass) and emission filter 542±13.5 nm) was used for imaging Corn-DFHO complex. A tetramethylrhodamine filter cube (with excitation filter 560±20 nm, dichroic mirror 585 nm (long pass) and emission filter 630±37.5 nm) was used for imaging mCherry-MCP and Beetroot-DFAME complex.

### Synthesis of Methyl (Z)-4-(3,5-difluoro-4-hydroxybenzylidene)-1-methyl-5-oxo-4,5-dihydro-1H-imidazole-2-carboxylate (DFAME)

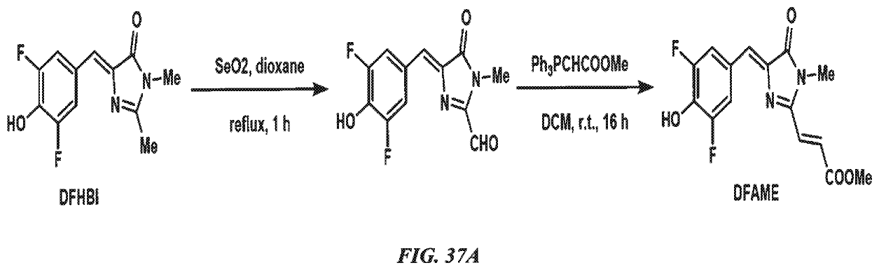

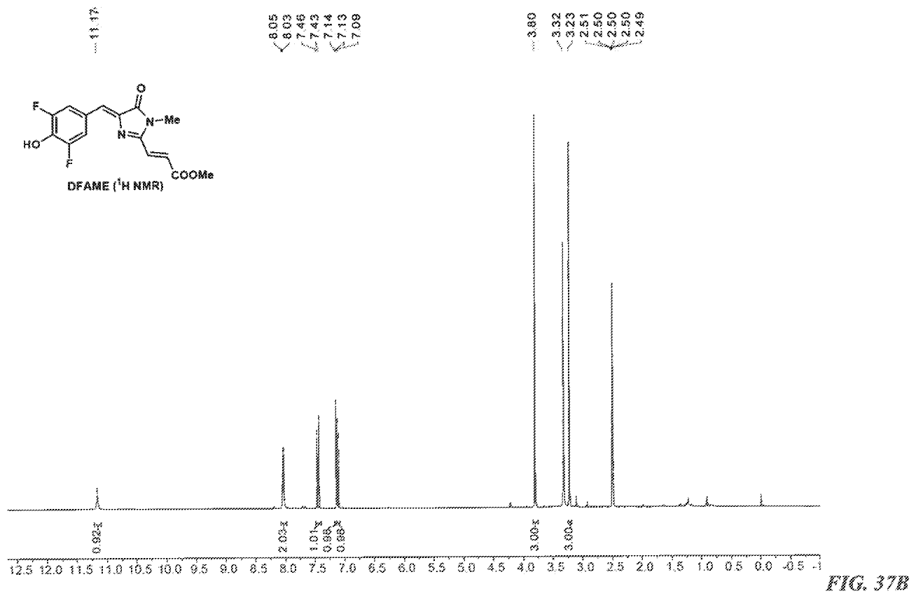

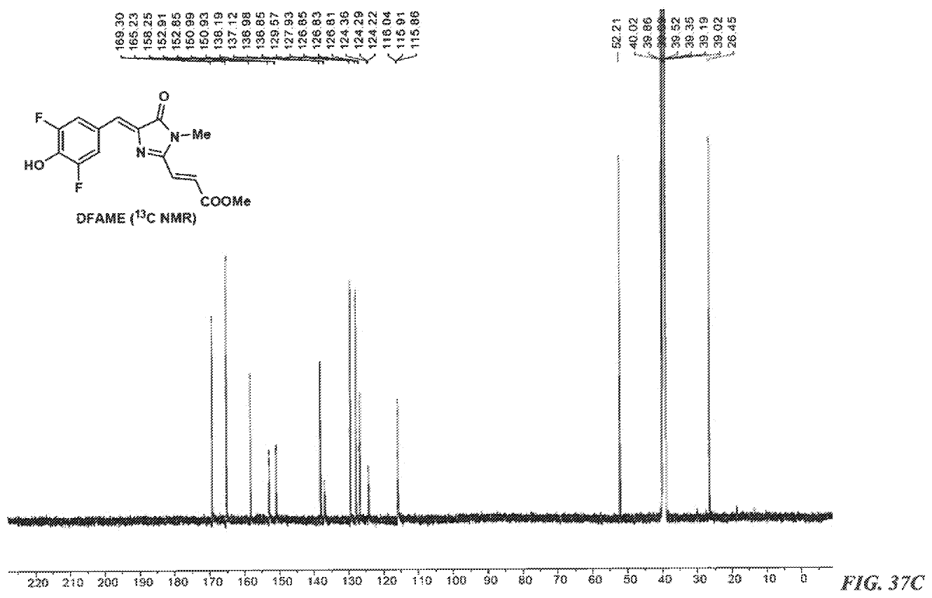

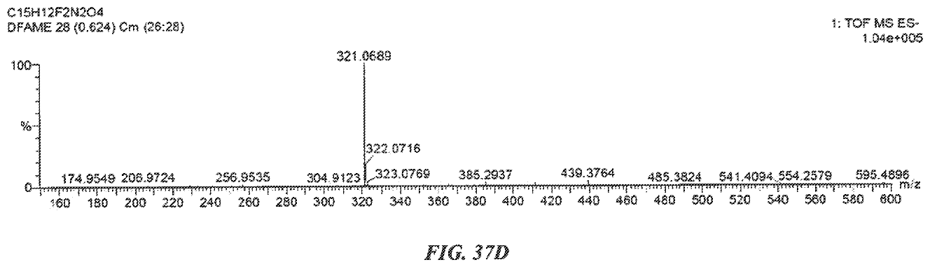

All chemicals and reagents were purchased from commercial sources without further purification, all organic solvents were used with ACS grade. DFHBI (50.4 mg, 0.2 mmol, 1.0 equiv.), and selenium dioxide (22.2 mg, 0.2 mmol, 1.0 equiv.), and anhydrous dioxane (1 mL) were stirred at reflux for 1 hour, then the solution was filtered by vacuum while hot (FIG. 37A). Next, the volatiles were removed in vacuo, the crude product was dissolved in dichloromethane (FIG. 37A). Methyl (triphenylphosphoranylidene)acetate (56.8 mg, 0.17 mmol, 0.9 equiv.) was added at 0° C., after stirring for 16 hours at room temperature, the volatiles were removed in vacuo and the residue was purified by column chromatography (silica gel, 1:1 petroleum ether:ethyl acetate) to afford DFAME as a red solid (25.0 mg, 39%) (FIG. 37A). ¹H NMR (500 MHz, DMSO) δ 11.17 (s, 1H), 8.04 (d, 2H), 7.45 (d, J=15.7 Hz, 1H), 7.14 (s, 1H), 7.11 (d, J=15.7 Hz, 1H), 3.80 (s, 3H), 3.23 (s, 3H) (FIG. 37B). ¹³C NMR (126 MHz, DMSO) δ 169.30, 165.23, 158.25, 151.95 (d, J=241.9 Hz), 151.89 (d, J=241.9 Hz), 138.19, 136.98 (t, J=16.4 Hz), 129.57, 127.93, 126.83 (t, J=3.2 Hz), 124.29 (t, J=8.8 Hz), 116.04 (d, J=6.2 Hz), 115.89 (d, J=6.2 Hz), 52.21, 26.45 (FIG. 37C). ESI-HR calcd for C₁₅H₁₁F₂N₂O₄⁻ ([M−H]⁻) 321.0692, found 321.0689 (FIG. 37D).

### INTRODUCTION

The ability to induce DNA self-assembly in vitro has prompted an interest in genetically encoding self-assembling nucleic acids in mammalian cells (Bujold et al., “DNA Nanostructures at the Interface with Biology,” Chem. 4:495-521 (2018), which is hereby incorporated by reference in its entirety). Genetically encodable self-assembling nucleic acids could provide a novel approach to organize biomolecules in cells and to endow cells with new functions, such as creating molecular proximity between enzymes and substrates. Formation of in vitro DNA self-assembly relies on single-stranded DNA oligonucleotides that undergo basepairing reactions within an oligonucleotide or between oligonucleotides. These programmable interactions have enabled the self-assembly of DNA into diverse and potentially useful structures (Castro et al., “A Primer to Scaffolded DNA Origami,” Nat. Methods 8:221-229 (2011), which is hereby incorporated by reference in its entirety).

However, these in vitro DNA-based self-assembly approaches cannot be genetically encoded to occur in a cell. DNA self-assembly requires single-stranded DNA, but mammalian cells contain primarily double-stranded DNA. Single-stranded DNA cannot be readily generated in mammalian cells. As a result, the key principles behind DNA-based macromolecular self-assembly cannot be applied to mammalian cells, and new approaches would be needed to genetically encode self-assembling nucleic acids.

Conceivably, the principles behind DNA self-assembly could be used to self-assemble structures from RNA, which can be genetically encoded and is synthesized in cells as a single stranded transcript. However, self-assembly currently relies on forming helical duplexes. Double-stranded RNA is particularly problematic in mammalian cells for two reasons. First, long double-stranded RNA is degraded by endogenous nucleases such as DICER (Macrae et al., “Structural Basis for Double-Stranded RNA Processing by Dicer.” Science 311:195-198 (2006), which is hereby incorporated by reference in its entirety), which would therefore prevent the stability of structures comprising long double-stranded RNA regions. Second, long double-stranded RNA induces an innate immune response by binding to proteins such as protein kinase (Yoneyama et al., “The RNA Helicase RIG-I Has an Essential Function in Double-Stranded RNA-Induced Innate Antiviral Responses,” Nat. Immunol. 5:730-737 (2004), which is hereby incorporated by reference in its entirety). Therefore, genetically encoded intracellular RNA self-assembly would require new strategies that do not rely on forming long double-stranded RNA.

Recently, Corn, an unusual RNA aptamer that homodimerizes to form a fluorogenic aptamer complex was described (Song et al., “Imaging RNA Polymerase III Transcription Using a Photostable RNA-Fluorophore Complex,” Nat. Chem. Biol. 13:1187-1194 (2017) and Warner et al., “Homodimer Interface without Base Pairs in an RNA Mimic of Red Fluorescent Protein,” Nat. Chem. Biol. 13:1195-1201 (2017), which are hereby incorporated by reference in their entirety). Importantly, each Corn monomer dimerizes without forming base pairs or helices with the other Corn monomer. Corn is technically a “pseudodimer” since each monomer within the dimer folds into a slightly different structure, rather than both dimers forming the same structure Warner et al., “Homodimer Interface without Base Pairs in an RNA Mimic of Red Fluorescent Protein,” Nat. Chem. Biol. 13:1195-1201 (2017), which is hereby incorporated by reference in its entirety). The Corn dimer is stable in nondenaturing gels and can be distinguished from the monomer since the dimer can bind and activate the fluorescence of 3,5-difluoro-4-hydroxybenzylidene imidazolinone-2-oxime (DFHO), a fluorogenic dye (Song et al., “Imaging RNA Polymerase III Transcription Using a Photostable RNA-Fluorophore Complex,” Nat. Chem. Biol. 13:1187-1194 (2017) and Warner et al., “Homodimer Interface without Base Pairs in an RNA Mimic of Red Fluorescent Protein,” Nat. Chem. Biol. 13:1195-1201 (2017), which are hereby incorporated by reference in their entirety). Since Corn can dimerize in a manner that does not involve forming double stranded RNA, Corn could potentially be used to guide self-assembly of multi-RNA complexes.

The examples of the present disclosure investigate the idea that intracellular macromolecular assemblies can be genetically encoded using RNA. A new approach for encoding a simple macromolecular assembly is investigated using the dimerization properties of both Corn and Beetroot, a fluorogenic RNA aptamer described herein. Beetroot exhibits partial sequence similarity to Corn, but Corn and Beetroot form homodimers that are completely orthogonal, which allowed us to genetically encode distinct Corn-based and Beetroot-based assemblies in the same cell. Whether these RNA assemblies could be functionalized with intracellular proteins was also investigated. The examples of the present disclosure demonstrate that genetically encoded RNA assemblies recruit specific proteins, thereby creating specific ribonucleoprotein assemblies. Overall, these results demonstrate that Corn and Beetroot can be used as orthogonal genetically encoded building blocks to guide the formation of RNA and RNA-protein macromolecular assemblies in cells.

### Example 7—Identification of Beetroot, a Red-Fluorescent Fluorogenic Aptamer

This project was initially begun this project with the goal of creating a fluorogenic aptamer that induces the fluorescence of 3,5-difluoro-4-hydroxybenzylidene imidazolinone-2-acrylate methyl (DFAME), a fluorophore that resembles fluorophores in some red-fluorescent proteins (FIG. 26A). DFAME was designed to contain a more extended π-electron conjugation system compared to DFHO, since extension of the π-electron conjugation system typically red shifts fluorescence emissions and excitations in fluorescent dyes (Ando et al., “Structural Characteristics and Optical Properties of a Series of Solvatochromic Fluorescent Dyes Displaying Long-Wavelength Emission,” Dyes Pigm. 83:198-206 (2009), which is hereby incorporated by reference in its entirety). The hydroxamic acid in DFHO was replaced with methyl acrylate to create DFAME (FIG. 26A).

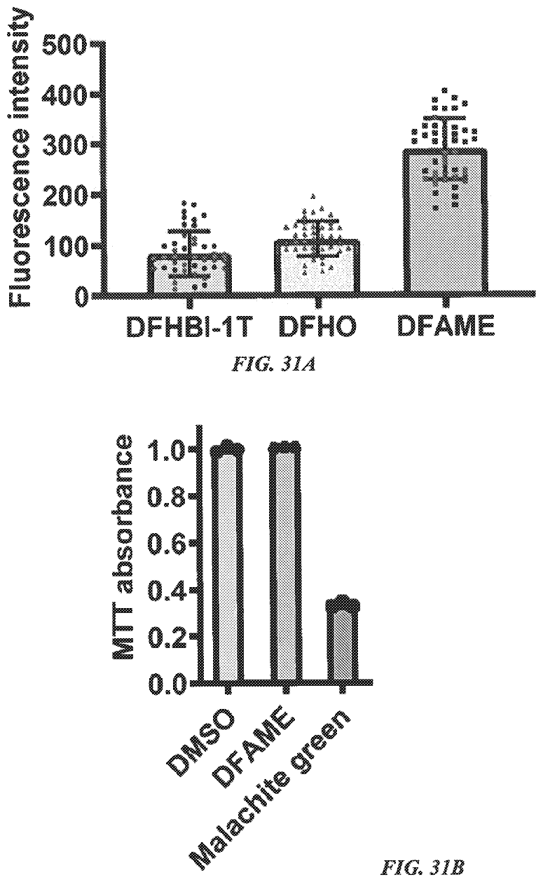

A fluorogenic dye needs to have minimal or no fluorescence in vitro and after addition to cells. In this way, fluorescence would only be attributed to the aptamer-fluorophore complex. Consistent with this, it was found that DFAME (10 μM) showed very low red fluorescence in solution (Table 7). Furthermore, incubation of 10 μM DFAME to cultured HEK293T cells resulted in measurable but low levels of red fluorescence (FIG. 31A). Although the background fluorescence in cells induced by DFAME was higher than DFHO or DFHBI-1T (FIG. 31A), the fluorophore for Spinach and Broccoli,8,9 it was reasoned that DFAME could still be used if enough fluorogenic aptamer was used in cells. DFAME also exhibited low cytotoxicity in HEK293T cells despite continuous irradiation in an epifluorescence microscope (excitation using a filter cube with a 565±20 nm filter) for 10 minutes (FIG. 31B), a treatment that is associated with toxicity when using other dyes such as malachite green.5,8 Together, these results suggest that DFAME could be used as a fluorogenic dye in cells once a suitable aptamer is identified.

TABLE 7
Photophysical and Biochemical Properties of DFAME,
DFHO, RNA-DFAME, and RNA-DFHO Complexes

| | Excitation | Emission | Extinction | Fluorescence | | |
|---|---|---|---|---|---|---|
| | maximum | maximum | coefficient | quantum | | K_{d} |
| | (nm) | (nm) | (M⁻¹cm⁻¹)¹ | yield | Brightness² | (nM)^{c} |
| DFAME alone | 508 | 641 | 32,500 | 0.0029 | 1.3 | |
| Beetroot-DFAME | 514 | 619 | 22,500 | 0.17 | 53 | 460 |
| Corn-DFAME | 531 | 621 | 27,500 | 0.020 | 7.6 | 3600 |
| DFHO alone^{d} | 473 | 561 | 19,800 | 0.0006 | 0.16 | |
| Beetroot-DFHO | 473 | 551 | 50,500 | 0.017 | 8.4 | 2300 |
| Corn-DFHO^{d} | 505 | 545 | 29,000 | 0.25 | 100 | 70 |

^{a}Extinction coefficients for DFAME alone and DFHO alone were measured at 514 and 505 nm in pH 7.4 buffer, respectively. RNA-DFHO complex extinction coefficients were all measured at a maximum excitation wavelength in the pH 7.4 buffer.
^{b}Brightness (extinction coefficient × quantum yield) is reported relatively to Corn-DFHO.
^{c}Measured by fluorescence.
^{d}Data from Song et al., “Imaging RNA Polymerase III Transcription Using a Photostable RNA-Fluorophore Complex,” Nat. Chem. Biol. 13: 1187-1194 (2017), which is hereby incorporated by reference in its entirety.

Next, aptamers that bind DFAME were generated using SELEX (systematic evolution of ligands by exponential enrichment) (Ellington and Szostak, “In Vitro Selection of RNA Molecules That Bind Specific Ligands,” Nature 346:818-822 (1990) and Tuerk and Gold, “Systematic Evolution of Ligands by Exponential Enrichment: RNA Ligands to Bacteriophage T4 DNA Polymerase,” Science 249:505-510 (1990), which are hereby incorporated by reference in their entirety). A DNA library containing ˜10¹⁴ random library members was utilized. DFAME was conjugated to agarose beads using an aminohexyl linker. After 10 rounds of SELEX, an RNA aptamer (designated 10-1) that binds DFAME and activates its fluorescence by threefold was identified.

Next, directed evolution was used to improve 10-1 (Table 8). For directed evolution experiments, a randomized DNA library based on 10-1 was used. The libraries were created using a doping strategy such that each nucleotide has a fixed probability of being converted into one of the other three nucleotides. The mutation frequency is predicted to result in a library containing all possible combinations of mutations that differ from the parent aptamer by 1, 2, 3, 4, 5, 6, 7, or 8 mutations (Filonov et al., “Rapid Selection of an RNA Mimic of Green Fluorescent Protein by Fluorescence-Based Selection and Directed Evolution,” J. Am. Chem. Soc. 136:16299-16308 (2014), which is hereby incorporated by reference in its entirety). Four rounds of SELEX were performed using this library to enrich binding to DFAME-agarose using a previously described directed evolution method (Song et al., “Imaging RNA Polymerase III Transcription Using a Photostable RNA-Fluorophore Complex,” Nat. Chem. Biol. 13:1187-1194 (2017), which is hereby incorporated by reference in its entirety). After cloning of library members into a bacterial expression plasmid, followed by expression in Escherichia coli and FACS (fluorescence-activated cell sorting), the brightest cells were collected to isolate aptamers with increased fluorescence. The top-performing aptamer (designated 10-1-4) exhibited approximately 40-fold fluorescence enhancement of DFAME in vitro (Table 7).

TABLE 8
RNA Sequences Used in Study

| | | SEQ |
|---|---|---|
| | | ID |
| Name | Sequence | NO: |
| 10-1 | GCUUCCGUGCGACAUCAUGCGCGCGACUGCUUCGGCAG | 95 |
| | AGGUGGGUGGUGUGGAGGAGUAUCUG | |
| Beetroot | GUUAGGCAGAGGUGGGUGGUGUGGAGGAGUAUCUGUC | 96 |
| Beetroot (107- | GGCCCGGAUAGCUCAGUCGGUAGAGCAGCGGCCGGUUA | 97 |
| nt, with tRNA | GGCAGAGGUGGGUGGUGUGGAGGAGUAUCUGUCCGGC | |
| scaffold) | CGCGGGUCCAGGGUUCAAGUCCCUGUUCGGGC | |
| Beetroot | GGCCCGGAUAGCUCAGUCGGUAGAGCAGCGGCCGGUUA | 98 |
| monomeric | GGCAGAACUACGUGGUGUACAACAGUAUCUGUCCGGCC | |
| mutant (107-nt, | GCGGGUCCAGGGUUCAAGUCCCUGUUCGGGC | |
| with tRNA | | |
| scaffold) | | |
| 1X Corn | AACCAUGCCGACUGAUGGCAGUUGCCAUGUGUAUGUG | 99 |
| (circular, with | GGAGGCCCGGAUAGCUCAGUCGGUAGAGCAGCGGCCGC | |
| F30 and tRNA | GCGAUUCGAGGAAGGAGGUCUGAGGAGGUCACUGAAU | |
| scaffold) | CGCGCGGCCGCGGGUCCAGGGUUCAAGUCCCUGUUCGG | |
| | GCCUCCCACAUACUCUGAUGAUCCAAAACAUGAGGAUC | |
| | ACCCAUGUCGAGGAUCAUUCAUGGCAACUGCCAUCAGU | |
| | CGGCGUGGACUGUAG | |
| 3X Corn | AACCAUGCCGACUGAUGGCAGUACCGUAGGCGAAUUCU | 100 |
| (circular, | UGCCAUGUGUAUGUGGGAGGCCCGGAUAGCUCAGUCG | |
| with F30 | GUAGAGCAGCGGCCGCGCGAUUCGAGGAAGGAGGUCU | |
| and tRNA | GAGGAGGUCACUGAAUCGCGCGGCCGCGGGUCCAGGGU | |
| scaffold) | UCAAGUCCCUGUUCGGGCCUCCCACAUACUCUGAUGAU | |
| | CCAAAACUGCCCGAGGAUCAUUCAUGGCAAACUCGACG | |
| | UUCGUCAGACUCCUCGCGAUGUCUAUGCGGGAGGCCCG | |
| | GAUAGCUCAGUCGGUAGAGCAGCGGCCGCGCGAUUCGA | |
| | GGAAGGAGGUCUGAGGAGGUCACUGAAUCGCGCGGCC | |
| | GCGGGUCCAGGGUUCAAGUCCCUGUUCGGGCCUCCCGC | |
| | AUAGUCUGAUCAUCCAAAACUGCCCGAGGGGAUGAUU | |
| | CAUCGCGAAUCGCGACGAUCGUCACUACCGGCGUCUGA | |
| | CGAACGUCGAACUCGCCGUGUGUGUGUAGGAGGCCCGG | |
| | AUAGCUCAGUCGGUAGAGCAGCGGCCGCGCGAUUCGAG | |
| | GAAGGAGGUCUGAGGAGGUCACUGAAUCGCGCGGCCG | |
| | CGGGUCCAGGGUUCAAGUCCCUGUUCGGGCCUCCUACA | |
| | CACUCUGACGAUCCAAAACUGCCCGAGGGGAUCGUUCA | |
| | CGGCGAACUCACGUCGGUGGAGCUAGCGCCUACGGUAC | |
| | UGCCAUCAGUCGGCGUGGACUGUAG | |
| 5X Corn | AACCAUGCCGACUGAUGGCAGUACCGUAGGCGAAUUCU | 101 |
| (circular, with | UGCCAUGUGUAUGUGGGAGGCCCGGAUAGCUCAGUCG | |
| F30 and tRNA | GUAGAGCAGCGGCCGCGCGAUUCGAGGAAGGAGGUCU | |
| scaffold, same | GAGGAGGUCACUGAAUCGCGCGGCCGCGGGUCCAGGGU | |
| as 5X Corn- | UCAAGUCCCUGUUCGGGCCUCCCACAUACUCUGAUGAU | |
| MS2) | CCAAAACUGCCCGAGGAUCAUUCAUGGCAAACUCGACG | |
| | UUCGUCAGACUCCUCGCGAUGUCUAUGCGGGAGGCCCG | |
| | GAUAGCUCAGUCGGUAGAGCAGCGGCCGCGCGAUUCGA | |
| | GGAAGGAGGUCUGAGGAGGUCACUGAAUCGCGCGGCC | |
| | GCGGGUCCAGGGUUCAAGUCCCUGUUCGGGCCUCCCGC | |
| | AUAGUCUGAUCAUCCAAAACUGCCCGAGGGGAUGAUU | |
| | CAUCGCGAAUCGCGACGAUCGUCACUACCGGUUCACUU | |
| | UGUAGACCAGGCUGGUGUACAACUUGCGACUUUGCCA | |
| | UGUGUAUGUGGGAGGCCCGGAUAGCUCAGUCGGUAGA | |
| | GCAGCGGCCGCGCGAUUCGAGGAAGGAGGUCUGAGGA | |
| | GGUCACUGAAUCGCGCGGCCGCGGGUCCAGGGUUCAAG | |
| | UCCCUGUUCGGGCCUCCCACAUACUCUGAUGAUCCACG | |
| | CGUAACAUGAGGAUCACCCAUGUCGAGGUACCAUCAUU | |
| | CAUGGCAAAGUCGCAAGUAUCGGUCAACCUAGUGGGCC | |
| | CAGUGACGAUCGUCGCCCAUCGCCUUGUGUCUGUUGGA | |
| | GGCCCGGAUAGCUCAGUCGGUAGAGCAGCGGCCGCGCG | |
| | AUUCGAGGAAGGAGGUCUGAGGAGGUCACUGAAUCGC | |
| | GCGGCCGCGGGUCCAGGGUUCAAGUCCCUGUUCGGGCC | |
| | UCCAACAGACUCUGACGAUCCAAAACUGCCCGAGGGGA | |
| | UCGUUCAAGGCGAGAGCUCGUCUGACGAACGUCGAACU | |
| | CGCCGUGUGUGUGUAGGAGGCCCGGAUAGCUCAGUCG | |
| | GUAGAGCAGCGGCCGCGCGAUUCGAGGAAGGAGGUCU | |
| | GAGGAGGUCACUGAAUCGCGCGGCCGCGGGUCCAGGGU | |
| | UCAAGUCCCUGUUCGGGCCUCCUACACACUCUGACGAU | |
| | CCAAAACUGCCCGAGGGGAUCGUUCACGGCGAACUCAC | |
| | GUCGGUGGAGCUAGCGCCUACGGUACUGCCAUCAGUCG | |
| | GCGUGGACUGUAG | |
| 5X Beetroot- | AACCAUGCCGACUGAUGGCAGUACCGUAGGCGAAUUCU | 102 |
| boxB (circular, | UGCCAUGUGUAUGUGGGAGGCCCGGAUAGCUCAGUCG | |
| with F30 and | GUAGAGCAGCGGCCGGUUAGGCAGAGGUGGGUGGUGU | |
| tRNA scaffold) | GGAGGAGUAUCUGUCCGGCCGCGGGUCCAGGGUUCAA | |
| | GUCCCUGUUCGGGCCUCCCACAUACUCUGAUGAUCCAA | |
| | AACUGCCCGAGGAUCAUUCAUGGCAAACUCGACGUUCG | |
| | UCAGACUCCUCGCGAUGUCUAUGCGGGAGGCCCGGAUA | |
| | GCUCAGUCGGUAGAGCAGCGGCCGGUUAGGCAGAGGU | |
| | GGGUGGUGUGGAGGAGUAUCUGUCCGGCCGCGGGUCC | |
| | AGGGUUCAAGUCCCUGUUCGGGCCUCCCGCAUAGUCUG | |
| | AUCAUCCAAAACUGCCCGAGGGGAUGAUUCAUCGCGAA | |
| | UCGCGACGAUCGUCACUACCGGUGACUCGCUAUCGGUU | |
| | AUGGCAUGUACAACUUGCGACUUUGCCAUGUGUAUGU | |
| | GGGAGGCCCGGAUAGCUCAGUCGGUAGAGCAGCGGCCG | |
| | GUUAGGCAGAGGUGGGUGGUGUGGAGGAGUAUCUGUC | |
| | CGGCCGCGGGUCCAGGGUUCAAGUCCCUGUUCGGGCCU | |
| | CCCACAUACUCUGAUGAUCCACGCGUACAUUGGCCCUG | |
| | AAAAAGGGCAAGCGAGGUACCAUCAUUCAUGGCAAAG | |
| | UCGCAAGUAUCGGUCAACCUAGUGGGCCCAGUGACGAU | |
| | CGUCGCCCAUCGCCUUGUGUCUGUUGGAGGCCCGGAUA | |
| | GCUCAGUCGGUAGAGCAGCGGCCGGUUAGGCAGAGGU | |
| | GGGUGGUGUGGAGGAGUAUCUGUCCGGCCGCGGGUCC | |
| | AGGGUUCAAGUCCCUGUUCGGGCCUCCAACAGACUCUG | |
| | ACGAUCCAAAACUGCCCGAGGGGAUCGUUCAAGGCGAG | |
| | AGCUCGUCUGACGAACGUCGAACUCGCCGUGUGUGUGU | |
| | AGGAGGCCCGGAUAGCUCAGUCGGUAGAGCAGCGGCCG | |
| | GUUAGGCAGAGGUGGGUGGUGUGGAGGAGUAUCUGUC | |
| | CGGCCGCGGGUCCAGGGUUCAAGUCCCUGUUCGGGCCU | |
| | CCUACACACUCUGACGAUCCAAAACUGCCCGAGGGGAU | |
| | CGUUCACGGCGAACUCACGUCGGUGGAGCUAGCGCCUA | |
| | CGGUACUGCCAUCAGUCGGCGUGGACUGUAG | |

Next, the spectral properties of 10-1-4 bound to DFAME were measured. 10-1-4-DFAME, prepared by mixing 10-1-4 (20 μM) and DFAME (2 μM), has fluorescence excitation and emission peaks at 514 and 619 nm, respectively (FIG. 26B, Table 7). The extinction coefficient (22,500 M⁻¹ cm⁻¹) and quantum yield (0.17) of 10-1-4-DFAME are similar to those of Corn-DFHO (Table 7). Because of its red fluorescence, this RNA aptamer was named Beetroot (Table 8), in keeping with previous nomenclature systems.

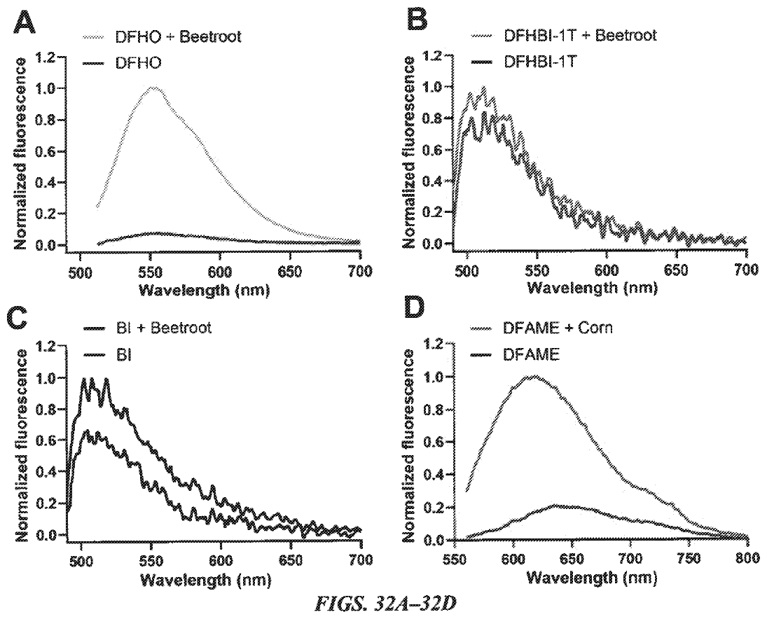

Since the goal was to use Beetroot simultaneously with Corn or other fluorogenic aptamers in the same cells, whether Beetroot interacts with other fluorogenic dyes was next investigated. It was found that Beetroot weakly binds and activates the fluorescence of DFHO (FIG. 32A and Table 7) but not DFHBI-1T or BI (FIGS. 32B-32C). It was also found that Corn weakly binds and activates the red fluorescence of DFAME (FIG. 32D and Table 7). Together, these results suggest that both Corn and Beetroot can bind and activate the fluorescence of DFHO and DFAME; however, Beetroot would only slightly be affected by DFHO and Corn would only slightly be affected by DFAME.

### Example 8—Beetroot is a Dimer In Vitro

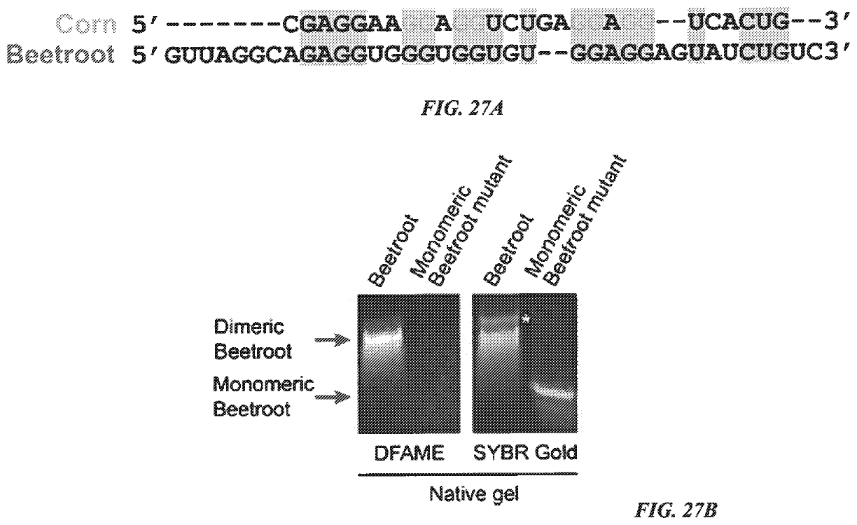

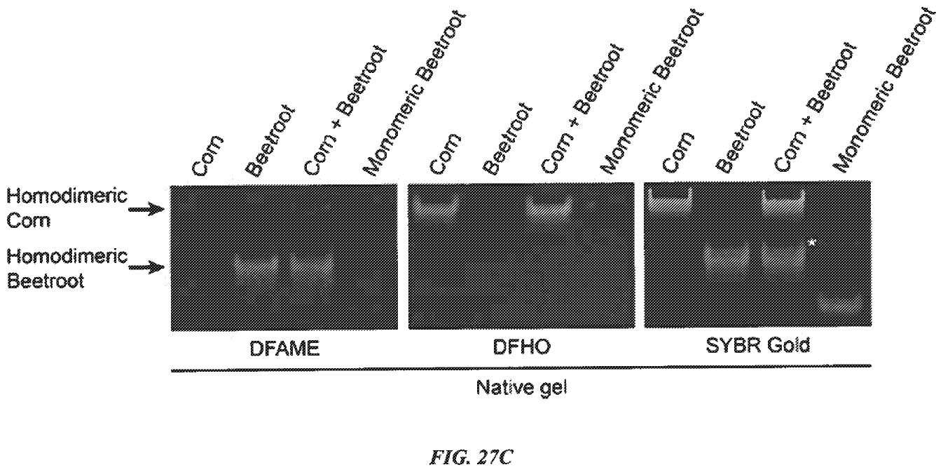

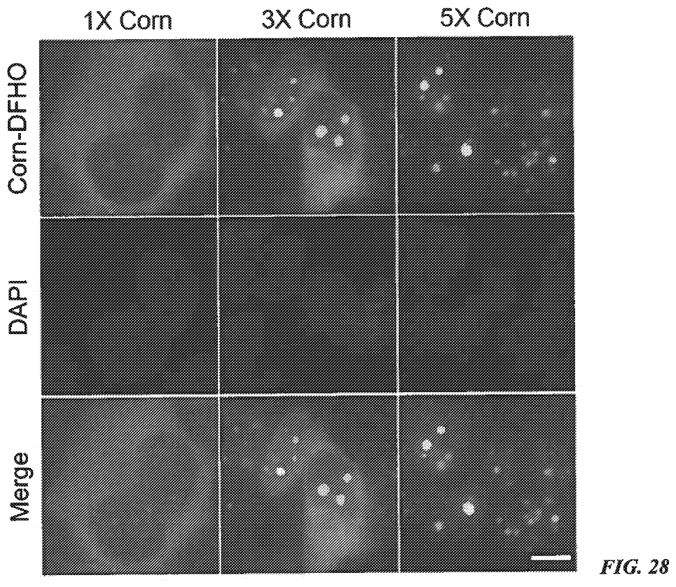

Alignment of Beetroot with Corn shows high similarity except for extra 7-nt and 2-nt extensions on the 5′ and 3′ ends of Beetroot, respectively (FIG. 27A). Since Corn is a dimeric RNA, whether Beetroot is also a dimer was investigated. In the case of dimeric Corn, the G-quadruplex of each Corn monomer interacts to form a dimeric interface (Warner et al., “Homodimer Interface without Base Pairs in an RNA Mimic of Red Fluorescent Protein,” Nat. Chem. Biol. 13:1195-1201 (2017), which is hereby incorporated by reference in its entirety). Disruption of the G-quadruplex of Corn abolishes Corn dimerization (Warner et al., “Homodimer Interface without Base Pairs in an RNA Mimic of Red Fluorescent Protein,” Nat. Chem. Biol. 13:1195-1201 (2017), which is hereby incorporated by reference in its entirety). It was reasoned that Beetroot might also contain a G-quadruplex since it shares each of the G residues that form the G-quadruplex in Corn (FIG. 27A). The presence of the G-quadruplex and the overall sequence similarity suggest that Beetroot may homodimerize.

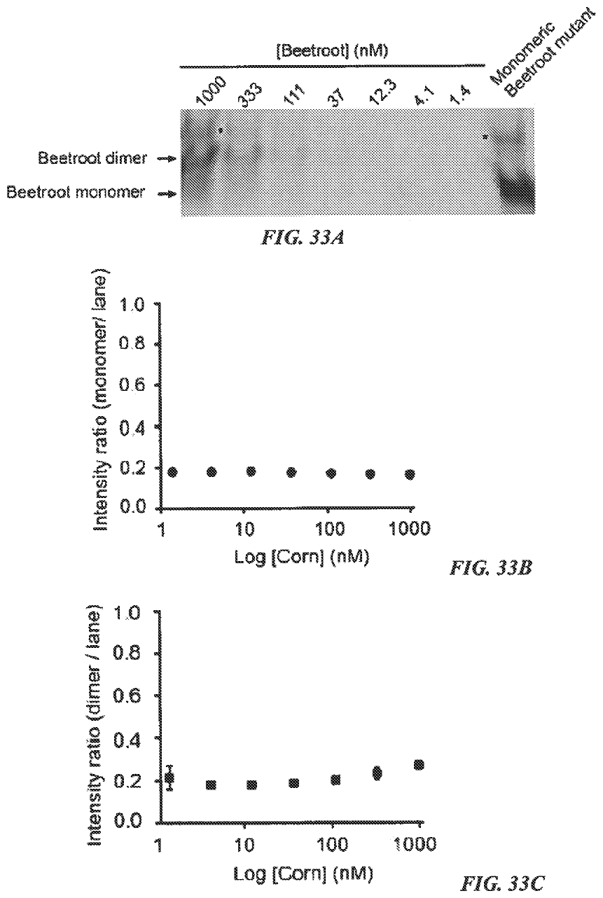

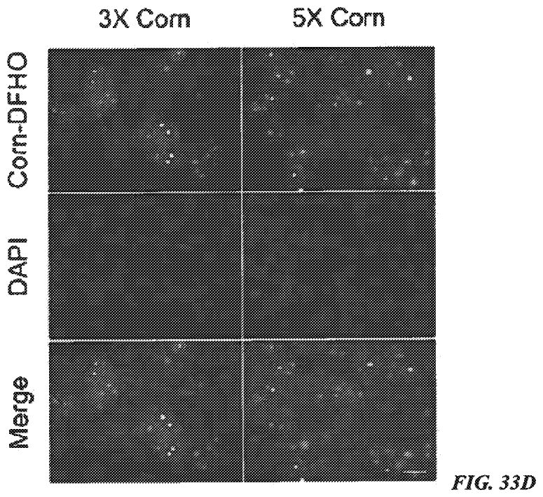

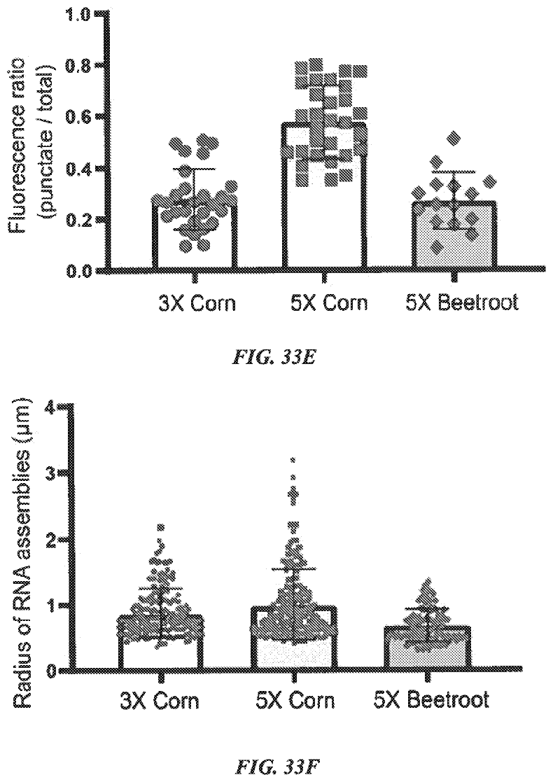

To determine if Beetroot is a dimer, Beetroot was resolved using nondenaturing gel electrophoresis. As a control, a mutant Beetroot with mutations at its putative G-quadruplex was used (FIG. 27B and Table 8). SYBR-Gold staining demonstrated that the G-quadruplex mutant Beetroot exhibits a clearly increased mobility in the gel (FIG. 27B), suggestive of a smaller size than the parental Beetroot aptamer. In-gel staining with DFAME confirmed that only the putative dimeric Beetroot band binds DFAME, while the putative monomer does not bind or activate the fluorescence of DFAME (FIG. 27B). Notably, Beetroot migrates as a dimer without the addition of DFAME, indicating that DFAME is not needed for dimer formation. Instead, Beetroot dimerization is needed for DFAME binding. This behavior is reminiscent of Corn, which also dimerizes independently of DFHO (Warner et al., “Homodimer Interface without Base Pairs in an RNA Mimic of Red Fluorescent Protein,” Nat. Chem. Biol. 13:1195-1201 (2017), which is hereby incorporated by reference in its entirety). Furthermore, similar to Corn (Warner et al., “Homodimer Interface without Base Pairs in an RNA Mimic of Red Fluorescent Protein,” Nat. Chem. Biol. 13:1195-1201 (2017), which is hereby incorporated by reference in its entirety), Beetroot has a homodimerization dissociation constant (K_{d}) that is likely less than 1 nM (FIGS. 33A-33C). Together, these results suggest that Beetroot, like Corn, forms a dimer.

Since Beetroot is a dimer and also shows sequence similarity to Corn, whether Beetroot and Corn can interact with each other to form mixed dimers or if they only form homodimers was next investigated. To test this, an equimolar mixture of Beetroot and Corn dimers was prepared. These aptamers were heatdenatured and then cooled to allow the monomers to fold in the presence of each other. In this way, each monomer has the ability to dimerize with either Corn or Beetroot. Next, nondenaturing electrophoretic analysis was performed to measure dimer formation. If Beetroot and Corn are orthogonal dimers, two distinct molecular species would be expected, i.e., dimeric Beetroot and dimeric Corn. On the other hand, if Beetroot-Corn heterodimers are formed, a third molecular species with a molecular weight in between the dimeric Beetroot and dimeric Corn would be expected. Notably, in this experiment, a 139-nt-long F30 (Filonov et al., “In-Gel Imaging of RNA Processing Using Broccoli Reveals Optimal Aptamer Expression Strategies,” Chem. Biol. 22:649-660 (2015), which is hereby incorporated by reference in its entirety) aptamer-folding scaffold was added to Corn so that it would have a clearly different length than Beetroot (107 and 250 nt for Beetroot and Corn, respectively) (Table 8). In this way, each possible dimeric form can be distinguished by its migration on a gel. Nondenaturing gel electrophoresis data showed Corn and Beetroot homodimers, but no mixed Corn-Beetroot heterodimers (FIG. 27C). In-gel staining with DFAME and DFHO also confirmed the identity of the putative dimeric species (FIG. 27C). Overall, these data suggest that Beetroot and Corn are orthogonal dimers in vitro.

### Example 9—Genetic Encoding of Macromolecular RNA Assemblies in Cells Using Corn

Construction of genetically encodable molecular assemblies in the cell could provide an approach to organize biomolecules in cells. Most molecular assemblies rely on the programmability of DNA base pairing to encode specific interactions of one DNA strand with at least one and often many more single-stranded DNAs (Rothemund, P. W. K., “Folding DNA to Create Nanoscale Shapes and Patterns,” Nature 440:297-302 (2006), which is hereby incorporated by reference in its entirety). It was next asked whether Corn and Beetroot could mimic the basic process of RNA self-assembly in cells.

To test this, whether RNA containing multiple Corns can form macromolecular assemblies in the cell was investigated. Circular RNAs containing 1, 3, or 5 copies of Corn were expressed in HEK293T cells (Table 8). These RNAs were expressed using the Tornado expression system (Litke and Jaffrey, “Highly Efficient Expression of Circular RNA Aptamers in Cells Using Autocatalytic Transcripts,” Nat. Biotechnol. 37:667-675 (2019), which is hereby incorporated by reference in its entirety), which causes the RNA to be expressed as a circular RNA. The Tornado expression system can promote RNA folding and increase RNA expression levels in cells due to their resistance to exonuclease-mediated degradation (Litke and Jaffrey, “Highly Efficient Expression of Circular RNA Aptamers in Cells Using Autocatalytic Transcripts,” Nat. Biotechnol. 37:667-675 (2019), which is hereby incorporated by reference in its entirety). When the Corn-containing circular RNA was expressed in HEK293T cells, diffuse yellow fluorescence as well as large puncta were observed (FIG. 28 and FIG. 33D). As the number of Corn aptamers increases in each circular RNA, an increase of yellow fluorescence was observed in these Corn-based RNA assemblies compared to the diffuse yellow fluorescence in the cytosol (FIG. 33E). Notably, when circular RNA containing five Corn monomers (5× Corn) was expressed, most yellow fluorescence was concentrated in the Corn-based RNA assemblies with minimal diffused yellow fluorescence in the cytosol compared to 1× Corn and 3× Corn (FIG. 28). Additionally, an increase of size of the 5× Corn assemblies compared to 3× Corn assemblies was observed (FIG. 33F). This suggests that the tendency to form Corn-based assemblies is proportional to the Corn valency in each RNA. Furthermore, the Corn-based assemblies mainly localize in the nucleus, likely reflecting their rapid formation upon transcription (FIG. 33D). Together, these results suggest that expressing multivalent RNA by concatenating multiple Corn aptamers can lead to self-assembly of RNAs to form macromolecular assemblies in the cell.

### Example 10—Self-Assembly of RNA-Protein Assemblies in Cells

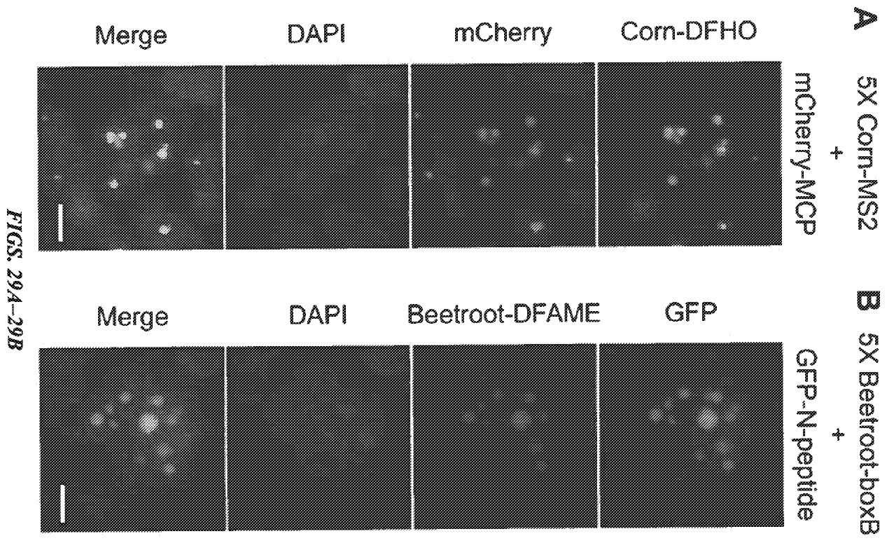

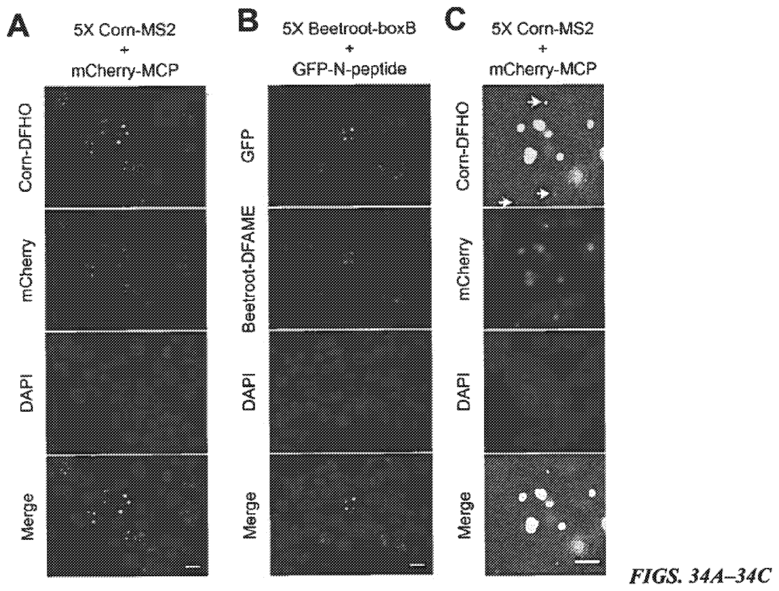

An important function of an RNA-based assembly would be to recruit and concentrate specific proteins, thus potentially creating assemblies with unique functions. It was herefore asked whether these Corn assemblies could be used to create platforms for protein assemblies. To test this, a 5× Corn circular RNA, which contained an MS2 hairpin (5× Corn-MS2), was expressed. The MS2 hairpin binds to the MS2 coat protein (MCP) from bacteriophage MS2 (Fouts et al., “Functional Recognition of Fragmented Operator Sites by R17/MS2 Coat Protein, a Translational Repressor,” Nucleic Acids Res. 25:4464-4473 (1997) and Valegård et al., “The Three-Dimensional Structures of Two Complexes between Recombinant MS2 Capsids and RNA Operator Fragments Reveal Sequence-Specific Protein-RNA Interactions,” J. Mol. Biol. 270:724-738 (1997), which are hereby incorporated by reference in their entirety). Therefore, 5× Corn-MS2 was coexpressed with MCP fused to mCherry (MCP-mCherry). Colocalization of the red fluorescence from MCPmCherry to the yellow fluorescence from 5×Corn-MS2 visualized by DFHO incubation with the cells was observed (FIG. 29 and FIGS. 34A-34C). Based on these results, RNA assemblies can be used to recruit proteins in the cell.

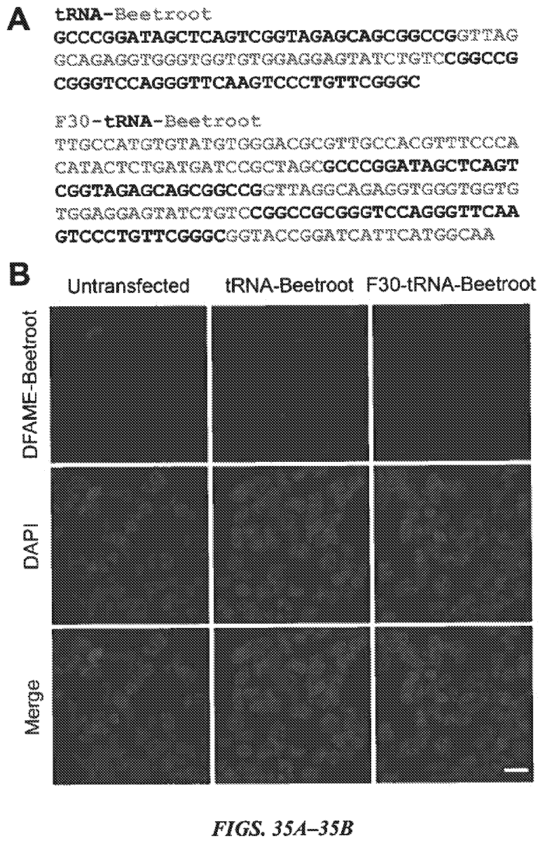

It was next asked whether RNA containing multiple Beetroot aptamers can also form macromolecular assemblies in the cells. To test this, a DNA plasmid encoding a circular RNA comprising 5× Beetroot fused to boxB was constructed (Austin et al., “Designed Arginine-Rich RNA-Binding Peptides with Picomolar Affinity,” J. Am. Chem. Soc. 124:10966-10967 (2002), which is hereby incorporated by reference in its entirety), a bacteriophage RNA hairpin that binds to a short peptide named N-peptide (5× Beetroot-boxB) (Table 8). Green-fluorescent protein fused to N-peptide (GFP-N-peptide) was coexpressed to bind to Beetroot-based assemblies. Green-fluorescent RNA assemblies were observed in the cells. After incubation with DFAME, red fluorescence from DFAME-Beetroot was also observed, which colocalized with the green fluorescence from GFP (FIG. 29B and FIGS. 34A-34C). Notably, a 1× Beetroot circular RNA is not fluorescent when Beetroot was incorporated into either the tRNA (Ponchon and Dardel, “Recombinant RNA Technology: The TRNA Scaffold,” Nat. Methods 4:571-576 (2007), which is hereby incorporated by reference in its entirety) or tRNA within an F30 (Filonov et al., “In-Gel Imaging of RNA Processing Using Broccoli Reveals Optimal Aptamer Expression Strategies,” Chem. Biol. 22:649-660 (2015), which is hereby incorporated by reference in its entirety) aptamer-folding scaffold (FIGS. 35A-35B). The ability to detect the 5× Beetroot circular RNA likely reflects the concentrated red fluorescence from DFAME-Beetroot in the RNA assemblies. Together, these results suggest that, similar to Corn, concatenating multiple Beetroots can form macromolecular assembly in the cell, and that Beetroot can be used to encode RNA-protein assemblies.

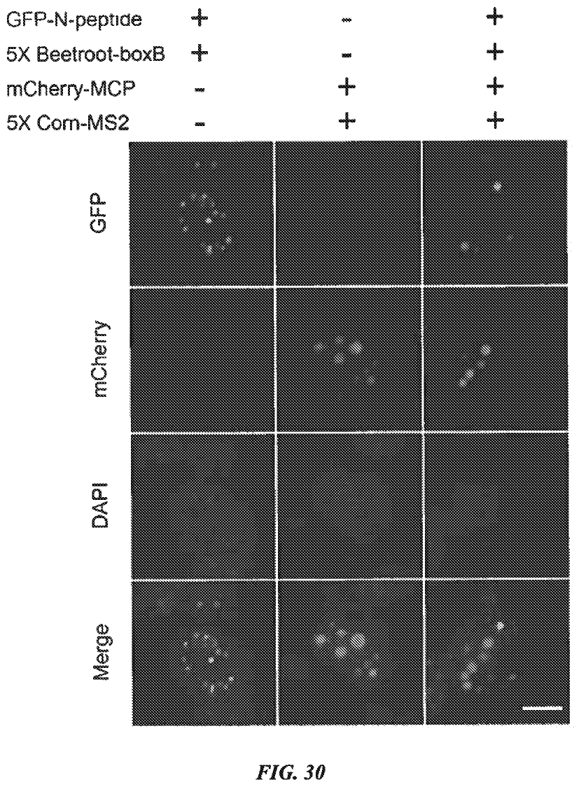

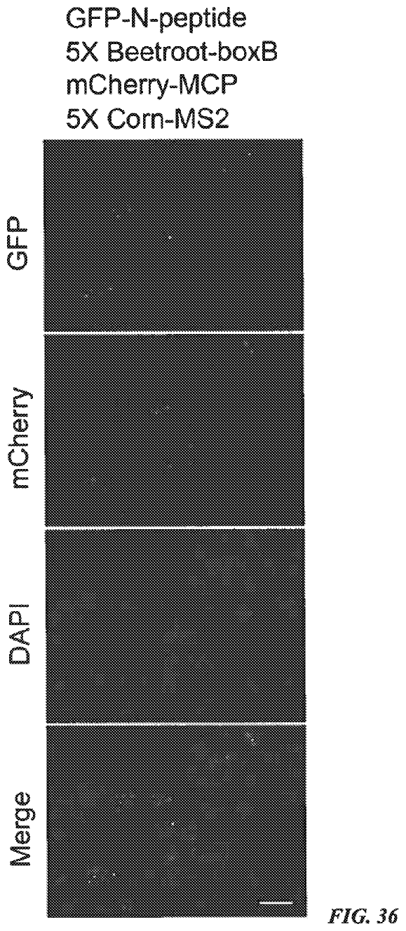

Since Corn and Beetroot are orthogonal dimers in vitro, it was next asked whether these RNA aptamers can form orthogonal assemblies in the same cell. To test this, circular 5× Corn-MS2 and 5× Beetroot-boxB were expressed in the same cells, and MCP-mCherry and GFP-N-peptide were coexpressed to visualize these RNA assemblies. It was reasoned that if Corn and Beetroot form orthogonal dimers in the cell, then green and red fluorescence overlap from MCP-mCherry and GFP-N-peptide would not be observed. Indeed, distinct green- and redfluorescent assemblies were observed (FIG. 30 and FIG. 36), likely corresponding to the Beetroot- and Corn-based assemblies. Notably, overlap between these fluorescent assembly species was not observed. Overall, these data suggest that Beetroot and Corn form orthogonal dimers, and they can be used for making genetically encodable molecular assemblies in the cell.

### Discussion of Examples 8-10

Creating RNA assemblies by dimerizing RNA is an emerging area in synthetic biology (Delebecque et al., “Organization of Intracellular Reactions with Rationally Designed RNA Assemblies,” Science 333:470-474 (2011) and Geary et al., “RNA Origami Design Tools Enable Cotranscriptional Folding of Kilobase-Sized Nanoscaffolds,” Nat. Chem. 13:549-558 (2021), which are hereby incorporated by reference in their entirety). These approaches primarily rely on RNA duplex formation. The examples of the present disclosure demonstrate that dimeric RNA aptamers can be used to form RNA assemblies in living cells. Corn was the first dimeric fluorogenic aptamer. Here, Beetroot, a new dimeric fluorogenic aptamer selected to bind and activate the red fluorescence of DFAME, a conditionally fluorescent dye, is described. It was found that Beetroot has sequence similarity to Corn, including conserved guanosine residues that form the G-quadruplex interface of the Corn dimer. It was found that Beetroot is also a dimer and notably does not heterodimerize with Corn. It was then shown that the dimerization properties of Corn and Beetroot can be used to induce genetically encoded RNA self-assembly in mammalian cells. It was shown that multivalent Corn and multivalent Beetroot circular RNAs can each assemble into larger assemblies, and these assemblies can recruit specific proteins, creating ribonucleoprotein assemblies in mammalian cells. Overall, these studies reveal Beetroot, a fluorogenic aptamer, and its use to genetically encode macromolecular RNA assemblies in cells.

The approach for inducing RNA assemblies described herein is to allow RNAs with multiple Corn or Beetroot aptamers to self-assemble. In this approach, an individual RNA can interact with one or more other RNAs. Each of these RNAs, in turn, can interact with more RNAs, which results in large puncta in HEK293T cells. Importantly, these assemblies are not highly organized like DNA origami. However, conceivable highly organized self-assembly could be encoded by inserting Corn or Beetroot into highly folded three-way junction RNAs such as F30 (Filonov et al., “In-Gel Imaging of RNA Processing Using Broccoli Reveals Optimal Aptamer Expression Strategies,” Chem. Biol. 22:649-660 (2015), which is hereby incorporated by reference in its entirety). By forcing Corn and Beetroot into specific angles defined by the three-way junction, larger assembly with predictable geometries may be possible. The RNA assemblies described here can be used to recruit and concentrate specific proteins. This could potentially be used to study proteins whose functions are regulated by local concentration, such as channels, synapses, or RNA-protein granules such as stress granules (You et al., “PhaSepDB: A Database of Liquid-Liquid Phase Separation Related Proteins,” Nucleic Acids Res. 48:D354-D359 (2020), which is hereby incorporated by reference in its entirety). Additionally, these clusters enable in-cell visualization of RNA-protein interactions by observing the colocalization of the RNA signal with either DFHO or DFAME and the protein, based on its fluorescence protein tag. Thus, the approach described here could be used to image the kinetics and regulation of RNA-protein interactions.

Beetroot constitutes the second dimeric fluorogenic aptamer. Although structural information is not yet available, the sequence similarity to Corn suggests that Beetroot uses a G-quadruplex to form the dimerization interface in Beetroot, as was seen previously in the crystal structure of Corn (Warner et al., “Homodimer Interface without Base Pairs in an RNA Mimic of Red Fluorescent Protein,” Nat. Chem. Biol. 13:1195-1201 (2017), which is hereby incorporated by reference in its entirety). However, the sequence and structure of Beetroot are sufficiently different from Corn to enable Beetroot and Corn to be fully orthogonal with no evidence of mixed Corn-Beetroot heterodimers. By taking advantage of this feature, distinct RNA assemblies were multiplexed in the same cells using Corn and Beetroot.

Although preferred embodiments have been depicted and described in detail herein, it will be apparent to those skilled in the relevant art that various modification, additions, substitutions, and the like can be made without departing from the spirit of the invention and theses are therefore considered to be within the scope of the invention as defined in the claims which follow.

1. A nucleic acid aptamer molecule comprising:
(1) the Squash nucleotide sequence of:
(i) GGC UAC AAG GUG AGC CCA AUA AUA CGG UUU GGG UUA GGA UAG GAA GUA GAG CCG UAA ACU CUC UAA GCG GUA GUC (SEQ ID NO: 1);
(ii) GGC UAC AAG GUG AGC CCA AUA AUA GGG UUU GGG UUA GGA UAG GAA GUA GAG CCC UAA ACU CUC UAA GCG GUA GUC (SEQ ID NO: 2);
(iii) GGC UAC AAG GUG AGC CCA AUA AUA NNN UUU GGG UUA GGA UAG GAA GUA GAG NNN UAA ACU CUC UAA GCG GUA GUC (SEQ ID NO: 3), wherein N at positions 25 and 54 are complementary to each other, N at positions 26 and 53 are complementary to each other, and N at positions 27 and 52 are complementary to each other; or
(iv) GGC UAC AAG GUG NNN NNN NUA AUA NNN UNN NNN NNA GGA UAG GAN NNN NNN NNN UAA ANN NNN NAA GCG GUA GUC (SEQ ID NO: 4),
wherein N at positions 25 and 54 are complementary to each other,
N at positions 26 and 53 are complementary to each other,
N at positions 27 and 52 are complementary to each other,
N at positions 13 and 35 are complementary to each other,
N at positions 14 and 34 are not complementary to each other,
N at positions 15 and 33 are complementary to each other,
N at positions 16 and 32 are complementary to each other,
N at positions 17 and 31 are complementary to each other,
N at positions 18 and 30 are complementary to each other,
N at positions 19 and 29 are complementary to each other
N at positions 45 and 64 are complementary to each other,
N at positions 46 and 63 are complementary to each other,
N at positions 48 and 62 are complementary to each other,
N at positions 49 and 61 are complementary to each other,
N at positions 50 and 60 are complementary to each other, and/or
N at positions 51 and 59 are complementary to each other,
(2) the core Squash nucleotide sequence of:
(i) AAG GUG AGC CCA AUA AUA CGG UUU GGG UUA GGA UAG GAA GUA GAG CCG UAA ACU CUC UAA GCG (SEQ ID NO: 5);
(ii) AAG GUG AGC CCA AUA AUA GGG UUU GGG UUA GGA UAG GAA GUA GAG CCC UAA ACU CUC UAA GCG (SEQ ID NO: 6);
(iii) AAG GUG AGC CCA AUA AUA NNN UUU GGG UUA GGA UAG GAA GUA GAG NNN UAA ACU CUC UAA GCG (SEQ ID NO: 7), wherein N at positions 19 and 48 are complementary to each other, N at positions 20 and 47 are complementary to each other, and N at positions 21 and 46 are complementary to each other: or
(iv) AAG GUG NNN NNN NUA AUA NNN UNN NNN NNA GGA UAG GAN NNN NNN NNN UAA ANN NNN NAA GCG (SEQ ID NO: 8),
wherein N at positions 19 and 48 are complementary to each other,
N at positions 20 and 47 are complementary to each other,
N at positions 21 and 46 are complementary to each other,
N at positions 7 and 29 are complementary to each other,
N at positions 8 and 28 are not complementary to each other,
N at positions 9 and 27 are complementary to each other,
N at positions 10 and 26 are complementary to each other,
N at positions 11 and 25 are complementary to each other,
N at positions 12 and 24 are complementary to each other,
N at positions 13 and 23 are complementary to each other
N at positions 39 and 58 are complementary to each other,
N at positions 40 and 57 are complementary to each other,
N at positions 41 and 56 are complementary to each other,
N at positions 43 and 55 are complementary to each other,
N at positions 44 and 54 are complementary to each other, and/or
N at positions 45 and 53 are complementary to each other;
(3) the extended Squash nucleotide sequence of:
(i)
(SEQ ID NO: 9)
GCC UAG GCU UCA AGG UGG CCC AAU GAU AUG GUU UGGG
UUA GGA UAG GAA UAA GAG CCU UAA ACU CUU CAA AGC
GGA AGU CUA GGC;
(ii)
(SEQ ID NO: 10)
GCC UAG GCU UCA AGG UGA GCC CAA UAA UAU GGU UUG
GGU UAG GAU AGG AAG AAG AGC CUU AAA CUC UCU AAG
CGG AAG UCU AGG C;
(iii)
(SEQ ID NO: 11)
GCC UAG GCU ACA AGG UGA GCC CAA UAA UAU GGU UUG
GGU UAG GAU AGG AAG UAG AGC CUU AAA CUC UCU AAG
CGG UAG UCU AGG C;
or
(iv)
(SEQ ID NO: 12)
GGU AGG CUA CAA GGU GAG CCC AAU AAU ACG GUU UGG
GUU AGG AUA GGA AGU AGA GCC GUA AAC UCU CUA AGC
GGU AGU CUA CC;
(4) the nucleotide sequence of:
(i) GGU AGG CUA CNN NNN NAG CCC AAU AAU ACG GUU UGG GUU NNN NNN NNN AGU AGA GCC GUA AAC UCU CUN NNN NGU AGU CUA CC (SEQ ID NO: 13), where N at each of positions 11-16, 40-48, and 69-73 can be any single nucleotide insertion of any length;
(ii) nNA GNU GnA GGA UAG GAn NAG CGn (SEQ ID NO: 14), wherein n at positions 1, 8, 18, and 24 can be a nucleotide insertion of 1-200 nucleotide bases and N at positions 2, 5, and 19 is any nucleotide base:
(iii) nNA GNU GnA GGA UAG GAn NAG CGn (SEQ ID NO: 14), wherein n at positions 1, 8, 18, and 24 can be a nucleotide insertion of 1-200 nucleotide bases and N at positions 2, 5, and 19 is any nucleotide base;
(iv) CUA CAA GGU GAG CCC AAU AAU ACG GUU UGG GUU AGG AUA GGA AGU AGA GCC GUA AAC UCU CUA AGC GGU AG (SEQ ID NO: 19);
(v) nNA GNU GnA GGA UAG GAn NAG CGn (SEQ ID NO: 14), wherein n at positions 1, 8, 18, and 24 can be a nucleotide insertion of 1-200 nucleotide bases and N at positions 2, 5, and 19 is any nucleotide base;
(vi) GGU CCA GAU GCC UUG UAA CCG AAA GGG ACA CAA GGU GAG CCC AAU AAU ACG GUU UGG GUU AGG AUA GGA AGU AGA GCC GUA AAC UCU CUA AGC GGU GUC GAA AGG AUG GAC C (SEQ ID NO: 26);
(vii) nNA GNU GAN NNN NNN NNN NNN NNN NNN NNU AGG AUA GGA ANN NNN NNN NNN NNN NNN NUN AGC Gn (SEQ ID NO: 27), wherein n at positions 1 and 65 can be a nucleotide insertion of 1-200 nucleotide bases: N at positions 2, 5, 9-29, 41-58, and 60 is any nucleotide base: N at positions 9-29 forms a step loop; and N at positions 41-58 forms a stem loop comprising a bulge in the stem;
(viii) nNA GNU GAN UAG GAU AGG AAN UNA GCG n (SEQ ID NO: 28), wherein n at positions 1 and 28 can be a nucleotide insertion of 1-200 nucleotide bases: N at positions 2, 5, and 23 are any nucleotide base: N at position 9 is a nucleotide insertion of 1-500 nucleotide bases where the insertion forms a stem loop; and N at position 21 is a nucleotide insertion of 1-500 nucleotide bases where the insertion forms a stem loop comprising a bulge in the stem;
(ix) nNA GNU GAN NNN NNN NNN NNN NNN NNN NNU AGG AUA GGA ANN NNN NNN NNN NNN NNN UNA GCG n (SEQ ID NO: 29), wherein n at positions 1 and 64 can be a nucleotide insertion of 1-200 nucleotide bases: N at positions 2, 5, 9-29, 41-57, and 59 is any nucleotide base: N at positions 9-29 forms a step loop; and N at positions 41-57 forms a stem loop;
(x) nNA GNU GAN UAG GAU AGG AAN UNA GCG n (SEQ ID NO: 30), wherein n at positions 1 and 28 can be a nucleotide insertion of 1-200 nucleotide bases: N at positions 2, 5, and 23 are any nucleotide base: N at position 9 is a nucleotide insertion of 1-500 nucleotide bases where the insertion forms a stem loop; and N at position 21 is a nucleotide insertion of 1-500 nucleotide bases where the insertion forms a stem loop: or
(xi) nAA GGU GAG CCC AAU AAU ACG GUU UGG GUU AGG AUA GGA AGU AGA GCC GUA AAC UCU CUA AGC Gn (SEQ ID NO: 31), wherein n at positions 1 and 65 can be a nucleotide insertion of 1-200 nucleotide bases: or
(5) the Beetroot nucleotide sequence of:
(i) GGU GGG UGG UGU GGA GGA GUA (SEQ ID NO: 32);
(ii) nGG UGG GUG GUG UGG AGG AGU An (SEQ ID NO: 33), wherein n at positions 1 and 23 is a nucleotide insertion of 1-200 nucleotide bases;
(iii) NNN NNN GGU GGG UGG UGU GGA GGA GUA NNN NNN (SEQ ID NO: 34), wherein N at positions 1 and 33 are complementary to each other and form a base pair,
N at positions 2 and 32 are complementary to each other and form a base pair,
N at positions 3 and 31 are complementary to each other and form a base pair,
N at positions 4 and 30 are complementary to each other and form a base pair,
N at positions 5 and 29 are complementary to each other and form a base pair, and/or
N at positions 6 and 28 are complementary to each other and form a base pair: or
(iv) nnn nNN NNN NGG UGG GUG GUG UGG AGG AGU ANN NNN N (SEQ ID NO: 35), wherein n at positions 1-4 is any nucleotide base,
N at positions 5 and 37 are complementary to each other and form a base pair,
N at positions 6 and 36 are complementary to each other and form a base pair,
N at positions 7 and 35 are complementary to each other and form a base pair,
N at positions 8 and 34 are complementary to each other and form a base pair,
N at positions 9 and 33 are complementary to each other and form a base pair, and/or
N at positions 10 and 32 are complementary to each other and form a base pair.

2.-4. (canceled)

5. A molecular complex comprising:
a fluorophore molecule comprising a methyne bridge between a substituted aromatic ring system and a substituted imidazol(thi)one, oxazol(thi)one, pyrrolin(thi)one, or furan(thi)one; and
the nucleic acid aptamer molecule according to claim 1 bound specifically to the fluorophore molecule;
wherein the fluorophore molecule has substantially enhanced fluorescence, in comparison to the fluorophore molecule prior to specific binding, upon exposure to radiation of suitable wavelength.

6. The molecular complex according to claim 5, wherein the fluorophore molecule is selected from the group consisting of 4-(3,5-difluoro-4-hydroxybenzylidene)-1-methyl-5-oxo-4,5-dihydro-1H-imidazole-2-carbaldehyde oxime (“DFHO”), (Z)-5-(3,5-difluoro-4-hydroxybenzylidene)-3-methyl-2-(trifluoromethyl)-3,5-dihydro-4H-imidazol-4-one (“DFHBI-2T”), (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”), (Z)-3-amino-5-(3,5-difluoro-4-hydroxybenzylidene)-2-thioxothiazolidin-4-one (“NRD5”), or methyl (E)-3-(4-((Z)-3,5-difluoro-4-hydroxybenzylidene)-1-methyl-5-oxo-4,5-dihydro-1H-imidazol-2-yl)acrylate (“DFAME”).

7. An isolated host cell comprising the molecular complex according to claim 5.

8. A kit comprising:
a fluorophore comprising a methyne bridge between a substituted aromatic ring system and a substituted imidazol(thi)one, oxazol(thi)one, pyrrolin(thi)one, or furan(thi)one ring; and
the nucleic acid aptamer molecule according to claim 1.

9. The kit according to claim 8, wherein the fluorophore molecule is 4-(3,5-difluoro-4-hydroxybenzylidene)-1-methyl-5-oxo-4,5-dihydro-1H-imidazole-2-carbaldehyde oxime (“DFHO”), (Z)-5-(3,5-difluoro-4-hydroxybenzylidene)-3-methyl-2-(trifluoromethyl)-3,5-dihydro-4H-imidazol-4-one (“DFHBI-2T”), (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”), (Z)-3-amino-5-(3,5-difluoro-4-hydroxybenzylidene)-2-thioxothiazolidin-4-one (“NRD5”), or methyl (E)-3-(4-((Z)-3,5-difluoro-4-hydroxybenzylidene)-1-methyl-5-oxo-4,5-dihydro-1H-imidazol-2-yl)acrylate (“DFAME”).

10. A constructed DNA molecule encoding the nucleic acid aptamer molecule according to claim 1.

11. An expression system comprising an expression vector into which is inserted a DNA molecule according to claim 10.

12. A transgenic host cell comprising the expression system of claim 11.

13. The transgenic host cell according to claim 12, wherein the transgenic host cell is either isolated, non-human, or both isolated and non-human.

14. A method of detecting a target molecule comprising:
forming a molecular complex according to claim 5;
exciting the fluorophore molecule with radiation of appropriate wavelength; and
detecting fluorescence by the fluorophore molecule, whereby fluorescence by the fluorophore identifies presence of the target molecule.

15. The method according to claim 14, wherein the fluorophore molecule is 4-(3,5-difluoro-4-hydroxybenzylidene)-1-methyl-5-oxo-4,5-dihydro-1H-imidazole-2-carbaldehyde oxime (“DFHO”), (Z)-5-(3,5-difluoro-4-hydroxybenzylidene)-3-methyl-2-(trifluoromethyl)-3,5-dihydro-4H-imidazol-4-one (“DFHBI-2T”), (E)-4-((Z)-3,5-difluoro-4-hydroxybenzylidene)-5-oxo-1-(2,2,2-trifluoroethyl)-4,5-dihydro-1H-imidazole-2-carbaldehyde oxime (“DFHO-1T”), (Z)-3-amino-5-(3,5-difluoro-4-hydroxybenzylidene)-2-thioxothiazolidin-4-one (“NRD5”), or methyl (E)-3-(4-((Z)-3,5-difluoro-4-hydroxybenzylidene)-1-methyl-5-oxo-4,5-dihydro-1H-imidazol-2-yl)acrylate (“DFAME”).

16. The method according to claim 14, wherein said forming is carried out in a cell.

17. An RNA-based metabolite sensor comprising:
(i) a metabolite-binding aptamer portion and
(ii) a regulated aptamer portion comprising the nucleic acid aptamer molecule according to claim 1 and a transducer domain, wherein the regulated aptamer portion is linked to the metabolite-binding aptamer portion by the transducer domain.

18. The RNA-based ratiometric sensor according to claim 17, wherein the transducer domain is a thermodynamically unstable helix.

19. The RNA-based ratiometric sensor according to claim 17, wherein the transducer domain is stabilized upon specific binding of the metabolite.

20. (canceled)

21. An RNA-based ratiometric metabolite sensor comprising:
(i) a regulated fluorescence activating aptamer comprising the RNA-based metabolite sensor of claim 17 and
(ii) a constitutive fluorescence activating aptamer.

22.-26. (canceled)

27. A system comprising:
the RNA-based ratiometric metabolite sensor according to claim 21;
a first fluorophore molecule; and
a second fluorophore molecule.

28.-29. (canceled)

30. A method of generating a randomized aptamer library:
providing a DNA sequence encoding a riboswitch aptamer and
modifying the DNA sequence encoding the riboswitch aptamer by introducing deletions, point mutations, and/or insertions or random nucleotides to generate a library comprising a plurality of modified sequences.

31.-56. (canceled)

57. A compound having a structure methyl (Z)-4-(3,5-difluoro-4-hydroxybenzylidene)-1-methyl-5-oxo-4,5-dihydro-1H-imidazole-2-carboxylate (DFAME).


SEQUENCE LISTING (105 sequences)

SEQ ID NO: 1 - RNA, 75 nt, synthetic construct
  source 1..75: mol_type = other RNA
ggctacaaggtgagcccaataatacggtttgggttaggataggaagtagagccgtaaactctctaagcggtagtc

SEQ ID NO: 2 - RNA, 75 nt, synthetic construct
  source 1..75: mol_type = other RNA
ggctacaaggtgagcccaataatagggtttgggttaggataggaagtagagccctaaactctctaagcggtagtc

SEQ ID NO: 3 - RNA, 75 nt, synthetic construct
  source 1..75: mol_type = other RNA
ggctacaaggtgagcccaataatannntttgggttaggataggaagtagagnnntaaactctctaagcggtagtc

SEQ ID NO: 4 - RNA, 75 nt, synthetic construct
  source 1..75: mol_type = other RNA
ggctacaaggtgnnnnnnntaatannntnnnnnnnaggataggannnnnnnnnntaaannnnnnaagcggtagtc

SEQ ID NO: 5 - RNA, 63 nt, synthetic construct
  source 1..63: mol_type = other RNA
aaggtgagcccaataatacggtttgggttaggataggaagtagagccgtaaactctctaagcg

SEQ ID NO: 6 - RNA, 63 nt, synthetic construct
  source 1..63: mol_type = other RNA
aaggtgagcccaataatagggtttgggttaggataggaagtagagccctaaactctctaagcg

SEQ ID NO: 7 - RNA, 63 nt, synthetic construct
  source 1..63: mol_type = other RNA
aaggtgagcccaataatannntttgggttaggataggaagtagagnnntaaactctctaagcg

SEQ ID NO: 8 - RNA, 63 nt, synthetic construct
  source 1..63: mol_type = other RNA
aaggtgnnnnnnntaatannntnnnnnnnaggataggannnnnnnnnntaaannnnnnaagcg

SEQ ID NO: 9 - RNA, 85 nt, synthetic construct
  source 1..85: mol_type = other RNA
gcctaggcttcaaggtggcccaatgatatggtttgggttaggataggaataagagccttaaactcttcaaagcggaagtctaggc

SEQ ID NO: 10 - RNA, 85 nt, synthetic construct
  source 1..85: mol_type = other RNA
gcctaggcttcaaggtgagcccaataatatggtttgggttaggataggaagaagagccttaaactctctaagcggaagtctaggc

SEQ ID NO: 11 - RNA, 85 nt, synthetic construct
  source 1..85: mol_type = other RNA
gcctaggctacaaggtgagcccaataatatggtttgggttaggataggaagtagagccttaaactctctaagcggtagtctaggc

SEQ ID NO: 12 - RNA, 83 nt, synthetic construct
  source 1..83: mol_type = other RNA
ggtaggctacaaggtgagcccaataatacggtttgggttaggataggaagtagagccgtaaactctctaagcggtagtctacc

SEQ ID NO: 13 - RNA, 83 nt, synthetic construct
  source 1..83: mol_type = other RNA
ggtaggctacnnnnnnagcccaataatacggtttgggttnnnnnnnnnagtagagccgtaaactctctnnnnngtagtctacc

SEQ ID NO: 14 - RNA, 24 nt, synthetic construct
  source 1..24: mol_type = other RNA
nnagntgnaggataggannagcgn

SEQ ID NO: 15 - RNA, 11 nt, synthetic construct
  source 1..11: mol_type = other RNA
gacgatcgaag

SEQ ID NO: 16 - RNA, 23 nt, synthetic construct
  source 1..23: mol_type = other RNA
agcccaataatacggtttgggtt

SEQ ID NO: 17 - RNA, 20 nt, synthetic construct
  source 1..20: mol_type = other RNA
agtagagccgtaaactctct

SEQ ID NO: 18 - RNA, 10 nt, synthetic construct
  source 1..10: mol_type = other RNA
cgaggttatt

SEQ ID NO: 19 - RNA, 71 nt, synthetic construct
  source 1..71: mol_type = other RNA
ctacaaggtgagcccaataatacggtttgggttaggataggaagtagagccgtaaactctctaagcggtag

SEQ ID NO: 20 - RNA, 31 nt, synthetic construct
  source 1..31: mol_type = other RNA
ggtccagatgccttgtaaccgaaagggacac

SEQ ID NO: 21 - RNA, 22 nt, synthetic construct
  source 1..22: mol_type = other RNA
agcccaataatacggtttgggt

SEQ ID NO: 22 - RNA, 11 nt, synthetic construct
  source 1..11: mol_type = other RNA
ggttctcaatt

SEQ ID NO: 23 - RNA, 11 nt, synthetic construct
  source 1..11: mol_type = other RNA
ttaaaacgctg

SEQ ID NO: 24 - RNA, 12 nt, synthetic construct
  source 1..12: mol_type = other RNA
tattttggggat

SEQ ID NO: 25 - RNA, 18 nt, synthetic construct
  source 1..18: mol_type = other RNA
gtgtcgaaaggatggacc

SEQ ID NO: 26 - RNA, 112 nt, synthetic construct
  source 1..112: mol_type = other RNA
ggtccagatgccttgtaaccgaaagggacacaaggtgagcccaataatacggtttgggttaggataggaagtagagccgtaaactctctaagcggtgtcgaaaggatggacc

SEQ ID NO: 27 - RNA, 65 nt, synthetic construct
  source 1..65: mol_type = other RNA
nnagntgannnnnnnnnnnnnnnnnnnnntaggataggaannnnnnnnnnnnnnnnnntnagcgn

SEQ ID NO: 28 - RNA, 28 nt, synthetic construct
  source 1..28: mol_type = other RNA
nnagntgantaggataggaantnagcgn

SEQ ID NO: 29 - RNA, 64 nt, synthetic construct
  source 1..64: mol_type = other RNA
nnagntgannnnnnnnnnnnnnnnnnnnntaggataggaannnnnnnnnnnnnnnnntnagcgn

SEQ ID NO: 30 - RNA, 28 nt, synthetic construct
  source 1..28: mol_type = other RNA
nnagntgantaggataggaantnagcgn

SEQ ID NO: 31 - RNA, 65 nt, synthetic construct
  source 1..65: mol_type = other RNA
naaggtgagcccaataatacggtttgggttaggataggaagtagagccgtaaactctctaagcgn

SEQ ID NO: 32 - RNA, 21 nt, synthetic construct
  source 1..21: mol_type = other RNA
ggtgggtggtgtggaggagta

SEQ ID NO: 33 - RNA, 23 nt, synthetic construct
  source 1..23: mol_type = other RNA
nggtgggtggtgtggaggagtan

SEQ ID NO: 34 - RNA, 33 nt, synthetic construct
  source 1..33: mol_type = other RNA
nnnnnnggtgggtggtgtggaggagtannnnnn

SEQ ID NO: 35 - RNA, 37 nt, synthetic construct
  source 1..37: mol_type = other RNA
nnnnnnnnnnggtgggtggtgtggaggagtannnnnn

SEQ ID NO: 36 - RNA, 67 nt, synthetic construct
  source 1..67: mol_type = other RNA
cttcatataatcctaatgatatggtttgggagtttctaccaagagccttaaactcttgattatgaag

SEQ ID NO: 37 - RNA, 11 nt, synthetic construct
  source 1..11: mol_type = other RNA
cgggacctcat

SEQ ID NO: 38 - RNA, 10 nt, synthetic construct
  source 1..10: mol_type = other RNA
agtttctacc

SEQ ID NO: 39 - RNA, 10 nt, synthetic construct
  source 1..10: mol_type = other RNA
actactcgcc

SEQ ID NO: 40 - RNA, 58 nt, Vibrio vulnificus
  source 1..58: mol_type = other RNA
tataatcctaatgatatggtttgggagtttctaccaagagccttaaactcttgattat

SEQ ID NO: 41 - RNA, 62 nt, synthetic construct
  source 1..62: mol_type = other RNA
aggtggcccaatgatatggtttgggttaggataggaataagagccttaaactcttcaaagcg

SEQ ID NO: 42 - RNA, 62 nt, synthetic construct
  source 1..62: mol_type = other RNA
aggtgagcccaataatatggtttgggttaggataggaagaagagccttaaactctctaagcg

SEQ ID NO: 43 - RNA, 62 nt, synthetic construct
  source 1..62: mol_type = other RNA
aggtgagcccaataatatggtttgggttaggataggaagtagagccttaaactctctaagcg

SEQ ID NO: 44 - RNA, 62 nt, synthetic construct
  source 1..62: mol_type = other RNA
aggtgagcccaataatacggtttgggttaggataggaagtagagccgtaaactctctaagcg

SEQ ID NO: 45 - RNA, 11 nt, synthetic construct
  source 1..11: mol_type = other RNA
tgtggtctatt

SEQ ID NO: 46 - RNA, 71 nt, Vibrio vulnificus
  source 1..71: mol_type = other RNA
ggcttcatataatcctaatgatatggtttgggagtttctaccaagagccttaaactcttgattatgaagtc

SEQ ID NO: 47 - RNA, 75 nt, synthetic construct
  source 1..75: mol_type = other RNA
ggcttcaaggtggcccaatgatatggtttgggttaggataggaataagagccttaaactcttcaaagcggaagtc

SEQ ID NO: 48 - RNA, 75 nt, synthetic construct
  source 1..75: mol_type = other RNA
ggcttcaaggtgagcccaataatatggtttgggttaggataggaagaagagccttaaactctctaagcggaagtc

SEQ ID NO: 49 - RNA, 75 nt, synthetic construct
  source 1..75: mol_type = other RNA
ggctacaaggtgagcccaataatatggtttgggttaggataggaagtagagccttaaactctctaagcggtagtc

SEQ ID NO: 50 - RNA, 11 nt, synthetic construct
  source 1..11: mol_type = other RNA
cgacggagtgg

SEQ ID NO: 51 - RNA, 11 nt, synthetic construct
  source 1..11: mol_type = other RNA
tggaagctttc

SEQ ID NO: 52 - RNA, 12 nt, synthetic construct
  source 1..12: mol_type = other RNA
acaggtgtcgtt

SEQ ID NO: 53 - RNA, 13 nt, synthetic construct
  source 1..13: mol_type = other RNA
aatgttgggcgtt

SEQ ID NO: 54 - RNA, 11 nt, synthetic construct
  source 1..11: mol_type = other RNA
taggggacttt

SEQ ID NO: 55 - RNA, 11 nt, synthetic construct
  source 1..11: mol_type = other RNA
acaacgatgct

SEQ ID NO: 56 - RNA, 11 nt, synthetic construct
  source 1..11: mol_type = other RNA
gcttcattctc

SEQ ID NO: 57 - RNA, 10 nt, synthetic construct
  source 1..10: mol_type = other RNA
gcgtctacaa

SEQ ID NO: 58 - RNA, 12 nt, synthetic construct
  source 1..12: mol_type = other RNA
cacattaggccc

SEQ ID NO: 59 - RNA, 12 nt, synthetic construct
  source 1..12: mol_type = other RNA
cgggatttggct

SEQ ID NO: 60 - RNA, 99 nt, synthetic construct
  source 1..99: mol_type = other RNA
gggcagcctaggcttcatataatcctaatgatatggtttgggagtttctaccaagagccttaaactcttgattatgaagtctaggctgccacagtctac

SEQ ID NO: 61 - RNA, 121 nt, synthetic construct
  source 1..121: mol_type = other RNA
gggcagcctaggcttcannnnnnnnnnnncctaatgatatggtttgggnnnnnnnnnnnnnnnnnnnnaagagccttaaactcttnnnnnnnnnnnngaagtctaggctgccacagtctac

SEQ ID NO: 62 - RNA, 42 nt, synthetic construct
  source 1..42: mol_type = other RNA
atcggtcgataatacgactcactatagggcagcctaggcttc

SEQ ID NO: 63 - RNA, 21 nt, synthetic construct
  source 1..21: mol_type = other RNA
gtagactgtggcagcctagac

SEQ ID NO: 64 - RNA, 85 nt, synthetic construct
  source 1..85: mol_type = other RNA
gcctaggcttcatggtgtgcctaatgatatggtttgggtctagagacgggaagagccttaaactcttcgatttggaagtctaggc

SEQ ID NO: 65 - RNA, 86 nt, synthetic construct
  source 1..86: mol_type = other RNA
gcctaggcttcacaggtggcccaatgatatggtttgggttaggataggaataagagccttaaacttttcaaagcggaagtctaggc

SEQ ID NO: 66 - RNA, 121 nt, synthetic construct
  source 1..121: mol_type = other RNA
ggcatcggcagcctaggcttcannnnnncccaatgatatggtttgggnnnnnnnnnnnngaagagccttaaactcttcnnnnnngaagtctaggctgccacagtctacgacctcgtaggct

SEQ ID NO: 67 - RNA, 43 nt, synthetic construct
  source 1..43: mol_type = other RNA
atcggtcgataatacgactcactataggcatcggcagcctagg

SEQ ID NO: 68 - RNA, 24 nt, synthetic construct
  source 1..24: mol_type = other RNA
agcctacgaggtcgtagactgtgg

SEQ ID NO: 69 - RNA, 85 nt, synthetic construct
  source 1..85: mol_type = other RNA
gcctaggcttcgaggtggcccaatgatatggtttgggttaggataggaagaagagccgtaaactcttcaaagcggaagtctaggc

SEQ ID NO: 70 - RNA, 84 nt, synthetic construct
  source 1..84: mol_type = other RNA
gcctaggcttcaaggtggcccaatgatatggtttgggttaggataggaagaagagccgtaaactctcaaagcggaagtctaggc

SEQ ID NO: 71 - RNA, 121 nt, synthetic construct
  source 1..121: mol_type = other RNA
ggcatcggcagcctaggctnnnnnnnnnnccnnnnnnnnnnnnnnnggnnnnnnnnnnnnnaannnnnnnnnnnnntnnnnnnnnnagtctaggctgccacagtctacgacctcgtaggct

SEQ ID NO: 72 - RNA, 43 nt, synthetic construct; identical to SEQ ID NO: 67
  source 1..43: mol_type = other RNA

SEQ ID NO: 73 - RNA, 24 nt, synthetic construct
  source 1..24: mol_type = other RNA
agcctacgaggtcgtagactgtgg

SEQ ID NO: 74 - RNA, 85 nt, synthetic construct
  source 1..85: mol_type = other RNA
gcctaggcttcaaggtgagccgaataatatggtttcggttaggataggaagaagagccttaaactctctaagcggaagtctaggc

SEQ ID NO: 75 - RNA, 85 nt, synthetic construct; identical to SEQ ID NO: 74
  source 1..85: mol_type = other RNA

SEQ ID NO: 76 - RNA, 85 nt, synthetic construct
  source 1..85: mol_type = other RNA
gcctaggcttcgaggtgagcccaataatatggtttgggttaggataggaagaagagccttaaactctctaagcggaagtctaggc

SEQ ID NO: 77 - RNA, 85 nt, synthetic construct
  source 1..85: mol_type = other RNA
gcctaggcttcaaggtgagcccaataatatggtttgggttaggataggaagaagagccgtaaactctctaagcggaagtctaggc

SEQ ID NO: 78 - RNA, 85 nt, synthetic construct; identical to SEQ ID NO: 77
  source 1..85: mol_type = other RNA

SEQ ID NO: 79 - RNA, 136 nt, synthetic construct
  source 1..136: mol_type = other RNA
gggttgccatgtgtatgtggctacaaggtgagcccaataatacggtttgggttaggataggaagtagagccgtaaactctctaagcggtagccacatactctgatgatccttcgggatcattcatggcaagctagc

SEQ ID NO: 80 - RNA, 63 nt, synthetic construct
  source 1..63: mol_type = other RNA
ggtaggcgagacggtcgggtccagatattcgtatctgtcgagtagagtgtgggctcgtctacc

SEQ ID NO: 81 - RNA, 114 nt, synthetic construct
  source 1..114: mol_type = other RNA
gggttgccatgtgtatgtgggagacggtcgggtccagatattcgtatctgtcgagtagagtgtgggctcccacatactctgatgatccttcgggatcattcatggcaagctagc

SEQ ID NO: 82 - RNA, 46 nt, synthetic construct
  source 1..46: mol_type = other RNA
ggtaggcgacgaggaaggaggtctgaggaggtcactgtcgtctacc

SEQ ID NO: 83 - RNA, 142 nt, synthetic construct
  source 1..142: mol_type = other RNA
ggtccagatgccttgtaaccgaaagggnnnnnnnnaaggtgagcccaataatacggtttgggttaggataggaagtagagccgtaaactctctaagcgnnnnnnnncgaaaggatggaccacagtctacgacctcgtaggct

SEQ ID NO: 84 - RNA, 49 nt, synthetic construct
  source 1..49: mol_type = other RNA
atcttcgtaatacgactcactataggtccagatgccttgtaaccgaaag

SEQ ID NO: 85 - RNA, 24 nt, synthetic construct
  source 1..24: mol_type = other RNA
agcctacgaggtcgtagactgtgg

SEQ ID NO: 86 - RNA, 112 nt, synthetic construct
  source 1..112: mol_type = other RNA
ggtccagatgccttgtaaccgaaagggaatcaaggtgagcccaataatacggtttgggttaggataggaagtagagccgtaaactctctaagcggattcgaaaggatggacc

SEQ ID NO: 87 - RNA, 76 nt, synthetic construct
  source 1..76: mol_type = other RNA
gggcgccagatgccttgtaaccgaaagggggaagaggaaggaggtctgaggaggtcactttcccgaaaggatggcg

SEQ ID NO: 88 - RNA, 252 nt, synthetic construct
  source 1..252: mol_type = other RNA
aaccatgccgactgatggcagttgccatgtgtatgtggccagatgccttgtaaccgaaagggacacaaggtgagcccaataatacggtttgggttaggataggaagtagagccgtaaactctctaagcggtgtcgaaaggatggccacatactctgatgatcgagacggtcgggtccagatattcgtatctgtcgagtagagtgtgggctcgatcattcatggcaactgccatcagtcggcgtggactgtag

SEQ ID NO: 89 - RNA, 252 nt, synthetic construct
  source 1..252: mol_type = other RNA
aaccatgccgactgatggcagttgccatgtgtatgtggccagatgccttgtaaccgaaagggaatcaaggtgagcccaataatacggtttgggttaggataggaagtagagccgtaaactctctaagcggattcgaaaggatggccacatactctgatgatcgagacggtcgggtccagatattcgtatctgtcgagtagagtgtgggctcgatcattcatggcaactgccatcagtcggcgtggactgtag

SEQ ID NO: 90 - RNA, 71 nt, synthetic construct; identical to SEQ ID NO: 19
  source 1..71: mol_type = other RNA

SEQ ID NO: 91 - RNA, 63 nt, synthetic construct
  source 1..63: mol_type = other RNA
nagntgannnnnnnnnnnnnnnnnnnnntaggataggaannnnnnnnnnnnnnnnnntnagcg

SEQ ID NO: 92 - RNA, 62 nt, synthetic construct
  source 1..62: mol_type = other RNA
nagntgannnnnnnnnnnnnnnnnnnnntaggataggaannnnnnnnnnnnnnnnntnagcg

SEQ ID NO: 93 - RNA, 63 nt, synthetic construct
  source 1..63: mol_type = other RNA
nagntgannnnnntnatanggtnnnnnntaggataggaannnnnnccnnnaannnnntnagcg

SEQ ID NO: 94 - RNA, 63 nt, synthetic construct; identical to SEQ ID NO: 5
  source 1..63: mol_type = other RNA

SEQ ID NO: 95 - RNA, 64 nt, synthetic construct
  source 1..64: mol_type = other RNA
gcttccgtgcgacatcatgcgcgcgactgcttcggcagaggtgggtggtgtggaggagtatctg

SEQ ID NO: 96 - RNA, 37 nt, synthetic construct
  source 1..37: mol_type = other RNA
gttaggcagaggtgggtggtgtggaggagtatctgtc

SEQ ID NO: 97 - RNA, 107 nt, synthetic construct
  source 1..107: mol_type = other RNA
ggcccggatagctcagtcggtagagcagcggccggttaggcagaggtgggtggtgtggaggagtatctgtccggccgcgggtccagggttcaagtccctgttcgggc

SEQ ID NO: 98 - RNA, 107 nt, synthetic construct
  source 1..107: mol_type = other RNA
ggcccggatagctcagtcggtagagcagcggccggttaggcagaactacgtggtgtacaacagtatctgtccggccgcgggtccagggttcaagtccctgttcgggc

SEQ ID NO: 99 - RNA, 241 nt, synthetic construct
  source 1..241: mol_type = other RNA
aaccatgccgactgatggcagttgccatgtgtatgtgggaggcccggatagctcagtcggtagagcagcggccgcgcgattcgaggaaggaggtctgaggaggtcactgaatcgcgcggccgcgggtccagggttcaagtccctgttcgggcctcccacatactctgatgatccaaaacatgaggatcacccatgtcgaggatcattcatggcaactgccatcagtcggcgtggactgtag

SEQ ID NO: 100 - RNA, 704 nt, synthetic construct
  source 1..704: mol_type = other RNA
aaccatgccgactgatggcagtaccgtaggcgaattcttgccatgtgtatgtgggaggcccggatagctcagtcggtagagcagcggccgcgcgattcgaggaaggaggtctgaggaggtcactgaatcgcgcggccgcgggtccagggttcaagtccctgttcgggcctcccacatactctgatgatccaaaactgcccgaggatcattcatggcaaactcgacgttcgtcagactcctcgcgatgtctatgcgggaggcccggatagctcagtcggtagagcagcggccgcgcgattcgaggaaggaggtctgaggaggtcactgaatcgcgcggccgcgggtccagggttcaagtccctgttcgggcctcccgcatagtctgatcatccaaaactgcccgaggggatgattcatcgcgaatcgcgacgatcgtcactaccggcgtctgacgaacgtcgaactcgccgtgtgtgtgtaggaggcccggatagctcagtcggtagagcagcggccgcgcgattcgaggaaggaggtctgaggaggtcactgaatcgcgcggccgcgggtccagggttcaagtccctgttcgggcctcctacacactctgacgatccaaaactgcccgaggggatcgttcacggcgaactcacgtcggtggagctagcgcctacggtactgccatcagtcggcgtggactgtag

SEQ ID NO: 101 - RNA, 1181 nt, synthetic construct
  source 1..1181: mol_type = other RNA
aaccatgccgactgatggcagtaccgtaggcgaattcttgccatgtgtatgtgggaggcccggatagctcagtcggtagagcagcggccgcgcgattcgaggaaggaggtctgaggaggtcactgaatcgcgcggccgcgggtccagggttcaagtccctgttcgggcctcccacatactctgatgatccaaaactgcccgaggatcattcatggcaaactcgacgttcgtcagactcctcgcgatgtctatgcgggaggcccggatagctcagtcggtagagcagcggccgcgcgattcgaggaaggaggtctgaggaggtcactgaatcgcgcggccgcgggtccagggttcaagtccctgttcgggcctcccgcatagtctgatcatccaaaactgcccgaggggatgattcatcgcgaatcgcgacgatcgtcactaccggttcactttgtagaccaggctggtgtacaacttgcgactttgccatgtgtatgtgggaggcccggatagctcagtcggtagagcagcggccgcgcgattcgaggaaggaggtctgaggaggtcactgaatcgcgcggccgcgggtccagggttcaagtccctgttcgggcctcccacatactctgatgatccacgcgtaacatgaggatcacccatgtcgaggtaccatcattcatggcaaagtcgcaagtatcggtcaacctagtgggcccagtgacgatcgtcgcccatcgccttgtgtctgttggaggcccggatagctcagtcggtagagcagcggccgcgcgattcgaggaaggaggtctgaggaggtcactgaatcgcgcggccgcgggtccagggttcaagtccctgttcgggcctccaacagactctgacgatccaaaactgcccgaggggatcgttcaaggcgagagctcgtctgacgaacgtcgaactcgccgtgtgtgtgtaggaggcccggatagctcagtcggtagagcagcggccgcgcgattcgaggaaggaggtctgaggaggtcactgaatcgcgcggccgcgggtccagggttcaagtccctgttcgggcctcctacacactctgacgatccaaaactgcccgaggggatcgttcacggcgaactcacgtcggtggagctagcgcctacggtactgccatcagtcggcgtggactgtag

SEQ ID NO: 102 - RNA, 1160 nt, synthetic construct
  source 1..1160: mol_type = other RNA
aaccatgccgactgatggcagtaccgtaggcgaattcttgccatgtgtatgtgggaggcccggatagctcagtcggtagagcagcggccggttaggcagaggtgggtggtgtggaggagtatctgtccggccgcgggtccagggttcaagtccctgttcgggcctcccacatactctgatgatccaaaactgcccgaggatcattcatggcaaactcgacgttcgtcagactcctcgcgatgtctatgcgggaggcccggatagctcagtcggtagagcagcggccggttaggcagaggtgggtggtgtggaggagtatctgtccggccgcgggtccagggttcaagtccctgttcgggcctcccgcatagtctgatcatccaaaactgcccgaggggatgattcatcgcgaatcgcgacgatcgtcactaccggtgactcgctatcggttatggcatgtacaacttgcgactttgccatgtgtatgtgggaggcccggatagctcagtcggtagagcagcggccggttaggcagaggtgggtggtgtggaggagtatctgtccggccgcgggtccagggttcaagtccctgttcgggcctcccacatactctgatgatccacgcgtacattggccctgaaaaagggcaagcgaggtaccatcattcatggcaaagtcgcaagtatcggtcaacctagtgggcccagtgacgatcgtcgcccatcgccttgtgtctgttggaggcccggatagctcagtcggtagagcagcggccggttaggcagaggtgggtggtgtggaggagtatctgtccggccgcgggtccagggttcaagtccctgttcgggcctccaacagactctgacgatccaaaactgcccgaggggatcgttcaaggcgagagctcgtctgacgaacgtcgaactcgccgtgtgtgtgtaggaggcccggatagctcagtcggtagagcagcggccggttaggcagaggtgggtggtgtggaggagtatctgtccggccgcgggtccagggttcaagtccctgttcgggcctcctacacactctgacgatccaaaactgcccgaggggatcgttcacggcgaactcacgtcggtggagctagcgcctacggtactgccatcagtcggcgtggactgtag

SEQ ID NO: 103 - RNA, 28 nt, synthetic construct
  source 1..28: mol_type = other RNA
cgaggaaggaggtctgaggaggtcactg

SEQ ID NO: 104 - RNA, 106 nt, synthetic construct
  source 1..106: mol_type = other RNA
gcccggatagctcagtcggtagagcagcggccggttaggcagaggtgggtggtgtggaggagtatctgtccggccgcgggtccagggttcaagtccctgttcgggc

SEQ ID NO: 105 - RNA, 188 nt, synthetic construct
  source 1..188: mol_type = other RNA
ttgccatgtgtatgtgggacgcgttgccacgtttcccacatactctgatgatccgctagcgcccggatagctcagtcggtagagcagcggccggttaggcagaggtgggtggtgtggaggagtatctgtccggccgcgggtccagggttcaagtccctgttcgggcggtaccggatcattcatggcaa